(19) United States
(12) Patent Application Publication
(10) Pub. No.: US 2014/0099354 A1
(43) Pub. Date: Apr. 10, 2014

(54) BIOPASSIVATING MEMBRANE STABILIZATION BY MEANS OF NITROCARBOXYLIC ACID-CONTAINING PHOSPHOLIPIDS IN PREPARATIONS AND COATINGS

(71) Applicant: Ulrich Dietz, Wiesbaden (DE)

(72) Inventors: Ulrich Dietz, Wiesbaden (DE); Udo Nubbemeyer, Mainz (DE)

(73) Assignee: Ulrich Dietz, Wiesbaden (DE)

(21) Appl. No.: 14/123,816

(22) PCT Filed: Jun. 6, 2012
(86) PCT No.: PCT/EP2012/060773

§ 371 Date: Dec. 4, 2013

(30) Foreign Application Priority Data
Jun. 6, 2011 (DE) 102011103948.5

(51) Int. Cl. A61L 31/08; A61K 31/685; A61L 27/28; A61L 15/20; A61L 27/54; A61L 31/16; A61L 17/14

(52) U.S. Cl. CPC A61L 31/08; A61L 31/16; A61K 31/685; A61L 17/145; A61L 15/20; A61L 27/54; A61L 27/28
USPC 424/423; 554/80; 514/77

(57) ABSTRACT

The present invention relates to nitro-carboxylic acid (s)-containing phospholipids, to be used for coating of medical devices such as stents, catheter balloons, wound pads or surgical suture material and for bio-passivating compositions, such as rinses, waterproofing solutions, coating solutions, cryoprotection solutions, cold preservation media, lyoprotection solutions, contrast media solutions, preservation and reperfusion solutions containing these compounds as well as preparing solutions thereof and coating medical devices as well as their uses.

### BACKGROUND OF THE INVENTION

The present invention is related to nitro-carboxylic acid (s)-containing phospholipids, medical devices such as for example stents, catheter balloons, wound insert or surgical suture material coated with said compounds and bio-passivating compositions, such as rinses, impregnation solutions, coating solutions, cryoprotection solutions, cryopreservation media, lyoprotection solutions, contrast agent solutions, preservation solutions and perfusion solutions containing these compounds as well as the production of these solutions and of the coated medical devices as well as their uses.

Any physical, chemical, or hypoxic cell alteration can lead to reactions of the affected cells that can induce migration, proliferation, matrix and cytokine production, apoptosis or necrosis. The extent of cell reaction depends essentially on the severity of the alteration, and whether one or more alterations of different kinds occur simultaneously which can lead to an exponential exaggeration of those cell reactions. The type of cell alteration is of minor importance as the cellular response patterns are basically the same.

The cellular response pattern to an alteration may be different under different clinical settings. Thus, the threshold for mast cell degranulation is reduced in the presence of adrenergic stimulation or mechanical fragility of erythrocytes is exaggerated while being suspended in a hypoosmolar medium, or in the presence of cellular toxins. On the other hand, cellular responses to hypoxia of cells are reduced while they are exposed to hypothermia. Mechanical alterations are transmitted by the cytoskeleton to the cell nucleus, which can trigger one of the above cell responses. Those mechanical alterations can be established in particular by force on adhesion molecules of the cell membrane. Another mechanism how cell responses are initiated is a change of the permeability of ion channels, e.g., calcium ion channels. Physical and chemical alterations are able to alter the integrity of the cell membrane, so that it comes to such ion currents, which cause cell activation. Another mechanism by which cells respond to changed environmental conditions is an activation of cell membrane proteins by mechanisms that are still not completely known. An aspect of the later condition is an increase of dimerisation of membrane proteins that consist of two or more subunits. Many membrane proteins only gain their functionally active form by dimerization. This can be achieved by a translocation of the protein subunits, e.g., by the cytoskeleton. The membrane fluidity has a significant impact on the translocalization of membrane proteins. Furthermore, the physical properties of cell membranes determine the conformation of membrane proteins and therefore their functionality. Physiological constitutes, e.g., cholesterol, as well as hydrophobic or amphiphilic molecules integrated into a cell membrane do impact the physical properties of cell membranes by changing hydrophobic adherence forces of the membrane phospholipid alkyl chains. These physical interactions lead to a change in the lateral membrane pressure which also affects the above mentioned interactions between the membrane phospholipids and the membrane proteins. In this respect, another well-known interaction mechanism between the physical properties of the membrane and the functionality of membrane proteins, are alkylated membrane protein subunits that are deposited in the phospholipid layer; the conformation of those subunit is influenced by physicochemical parameters of the phospholipid layer which determines the activity of the protein. Therefore the physical properties of the cell membrane contribute considerably to the type and readiness of cellular reactions to the above cell alterations.

Disintegration of tissue that is caused by trauma, chemicals/toxins or surgically or interventionally due to an alteration of cells, typically leads to overlapping of the above-mentioned damage mechanisms, e.g., mechanical trauma followed by a decreased blood supply (hypoxia) and the thereby resulting chemical alteration (acidosis). The extent of potential effects leading to an exaggerated cell respond to a trauma can not be predicted but is important for the immediate onset of the repair and healing process. Thus, repair mechanisms can be amplified leading to a production of cells or matrix proteins in an unphysiological extent which are more than needed to stabilize a defect. This results in connective tissue proliferation (e.g., fibrosis, capsule formation, celoid formation) which leads to a functional impairment of tissues/organs/body parts or to cosmetic/aesthetic issues. Cell alterations, which are caused by contact with noncellular foreign materials, are of utmost importance. In principle the same damage mechanisms as mentioned above also take place, whereas only a few cell layers that are in contact with the foreign materials are traumatized. However, the response of those cells is usually exaggerated compared to a sole trauma of comparable severity.

There is scientific evidence that local tissue alterations are essentially responsible for the observed non-physiological repair processes after contact with foreign materials. Thus, a central role for the clinical course is due to the foreign material surface that comes into close contact with alterated or damaged tissues. For external surfaces, which contribute to a physiological healing process, the term bio-compatibility was coined.

In the following, known pathophysiological processes, caused by a cell/tissue alteration in therapeutic procedures, shall be presented as an example on the basis of known processes that occur at or after vascular interventions. Percutaneous transluminal balloon angioplasty (PTCA) of an artery causes a baro-trauma of the cells of the affected blood vessel wall segment and a rupture of vessel wall structures. An extended wound surface is usually created, which is in direct contact with blood. A rapid deposition of plasma proteins and platelets thereafter is the consequence. The extent of this aggregate formation determines the amount of cytokines excreted, which encourage proliferation of vascular cells and further exaggerates thrombus formation. The latter condition can cause an occlusive thrombus formation with life-threatening consequences. In principle similar pathophysiological reactions occur after implantation of a vascular stent. Neither antithrombotic nor anti-inflammatory substances, which have been applied along with the stent, have shown that they reduce stent-induced cell proliferation or thrombus formation in clinically relevant dimensions. Thus, stents and catheter balloons were coated with antiproliferative agents, such as paclitaxel and rapamycin, to prevent stent-associated cell proliferation.

These active substances are released over a longer period from the coating of stent struts. Through dense contact of the device surface with the vessel wall, these substances are taken up by vascular smooth muscle cells and effectively inhibit their proliferation. However, this leads to an abnormal healing process that can result in a reduced stability of the vessel wall (aneurysm formation), or inhibit formation of a lining with intimal cells which are essential for a proper vessel surface functionality (inhibition of thromocyte aggregation/NO production and release). The same is true for the pathophysiological reactions that occur after ionization of the vessel wall. Therefore, undifferentiated inhibition of the repair processes by cytotoxic drugs or ionization, often results in an inadequate and unphysiological healing of damaged tissues. In addition, complete converge of the anti-proliferative drug releasing stent struts by a so-called neointima often takes many years, so that there is a risk of acute thrombosis and thus the development of organ infarction until then. In order to establish continuous and reproducible drug release from stent struts, polymer drug release systems have been developed. However, those polymers have limited biocompatibility and can by itself exaggerate proliferative response according to the above described reactions and therefore counteract the effect of anti-proliferative therapy.

Another aspect of the cellular “response to injury” of traumatized cells is the release of micro-particles, which comprise of phospholipids and proteins (Chironi et al., Cell Tissue Res 2009, 335, 143-151). Within the framework of an alteration of endothelial cells and platelets, they are able to shed phospholipid vesicles into the blood or surrounding fluids that contain molecules which can have signalling effects to other tissues. The ability of microparticle formation has also been described for other cell types. Microparticles can have local or systemic effects that can lead to an amplification or reduction of tissue responses (Mahendar et al., Pharmacol Rep 2008, 60, 75-84). Local up-take of those particles by directly surrounding cells can cause exaggeration of a proliferative response or mediate an inflammatory response. Microparticles play a major roll in pathophysiological mediation of immunological response in sepsis. The conditions under which these microparticles cause a physiological or pathological tissue reaction are still largely unknown.

The scientific literature documents that the biocompatibility of an artificial surface, which is in direct contact with cells is a crucial determinant of subsequent cellular reactions. While less biocompatible surfaces can induce cell dedifferentiation, migration, proliferation, or apoptosis, the ideal biocompatible surface would not induce such a cell response and rather maintain the physiological status and metabolism of adherent cells. Furthermore, biocompatibility of a surface is inversely proportional to the readiness to adsorb and the quantity of adsorbed organic molecules, such as albumin, fibronectin or complement factors, which are able to attach themselves to various kinds of artificial surfaces. This also applies to the amount of extra-cellular matrix proteins, produced by cells adhering on an artificial surface. In addition, the quantity and the quality of serum proteins that deposit onto such a surface determine which cells attach themselves as well as such a composition influences consecutive reactions of the adhering cells, e.g., migration and proliferation.

Biocompatibility of foreign materials can be different for the various mammalian cell types. Cells react to incompatibilities in their chemical environment (e.g. pH), the surface geometry (e.g. roughness of the surface), the fluidity of the interface (determined by the water content), and quality and quantity of cell contacts between the cell and the artificial surface.

In the sum of current knowledge, improvements of the biocompatibility of foreign surfaces can be accomplished by a reduction of friction energy of adhering cells, e.g. through a high content of water molecules at the interface, and absence of molecules that can lead to an immunological reaction due to interaction with cell membrane binding sites, as well as due to a chemical environment that represents physiological conditions.

Phospholipids meet these requirements for biocompatibility to a large extent, provided they have similar physicochemical properties at the interface to the adhering cell as the cell membrane itself. Phospholipids have the property to form membranous structures spontaneously, thus forming a homogeneous surface that has a high density of bound water molecules with the highest content when the head group bears a choline residue. Another advantage of phospholipids is their ability to create membranes spontaneously which enables to obtain coatings from those membranous structures (vesicles) that in addition close up to a homogeneous layer spontaneously while beeing entrenched with the support material and still allowing a free lateral movement of adhering phospholipids. Furthermore is it possible that phospholipid molecules dissolve into the surrounding medium and are taken up by adhering cells. For this reason, it is necessary to use phospholipids, which do not lead to undesirable effects when taken-up from the adhering cell. The melting point of natural phospholipids that form membranes in human cells is low, so that a mechanical or thermodynamically-driven separation of phospholipids out of a membrane-layer is facilitated by their high degree of mobility at body temperature conditions.

In order to generate phospholipid coatings that are stable in the air and resistant towards mechanical shear forces, phosphorylcholines were polymerized with a co-polymer (such as laurylmethacrylate). These phospholipid compounds do not occur in nature and have fundamentally different physicochemical properties as natural phospholipids; therefore they have to be named as synthetic phospholipids. Covalent anchorage of those coatings to the substrate was not intended; however, resistance to shear forces was achieved through a polymerization process, which resulted in a multilayer coating with a thickness of up to 50 μm. Polymerized phosphorylcholine coatings were more thrombus resistant than other hydrophilic polymers (van der Giessen, et al., Marked inflammatory sequelae to implantation of biodegradable polymers in porcine medical arteries, Circulation 1996; 94:1690-7). As polymerization increases the mechanical stability of such a coating, the lateral movement of the individual phospholipids was diminished, which resulted in a lower biocompatibility as compared to a surface with a fluid phospholipid coating and with the consequence of an increased cellular response of adhering cells. Coronary stents with a polymerized phosphorylcholine coating were investigated in animal studies. Both thrombus formation and tissue reaction did not differ in short- and long-term tissue studies as compared to uncoated stents.

This was also found in human clinical trials, demonstrating feasibility to use polymerized phosphotidylcholine coatings in a clinical setting; however, an improved biocompatibility was not reported so far compared to an already optimized metal substrate. Furthermore it was shown that coatings from polymerized phospholipids can tear and be delaminated during the expansion of the stent struts.

Vascular grafts are another medical challenge. So far, no long-term stable surface coating is known, which would protect an artificial surface from the adherence of serum proteins, an activation of the immune system or thrombus formation. Therefore, anticoagulation is required after implanting a synthetic vascular graft. Use of surface coatings that allow adherence of epithelial cells or bone marrow-derived pluripotent cells resulted in endothelialization; however, due to uncontrolled growth of endothelial cells (intima hyperplasia) coated synthetic grafts with diameters of <5 mm were very often stenosed or occluded. On the other hand, the only possible mode to prevent a clotting within such a graft is a closed endothelial lining. Conventionally used synthetic materials are not endothelazied at all. An antithrombotic coating that allows adhesion of endothelial cells at the same time is therefore desirable.

Thus there is still a great need to modify the cellular interaction with a foreign interface through appropriate measures, so that the contact does not cause cell activation. For this material property, we coined the term bio-passivation.

Bio-passivating coatings are advantageous not only for the use in cardiovascular implants, but also desirable for wound materials, wound inserts, surgical suture material or other implants such as facial and breast implants, as anti-fibrotic properties can also be expected. Surprisingly, it was found that nitro-fatty acid containing phospholipids have such bio-passivating properties.

### DESCRIPTION

The objective of the present invention is therefore to provide compounds preferably bio-passivating compounds which are suitable for the coating of medical devices, especially for the bio-passivating coating of medical devices as well as for the production of bio-passivating compositions, rinsing solutions, impregnation solutions, coating solutions, cryoprotection solutions, cryopreservation media, lyoprotection solutions, contrast agent solutions, preservation solutions and perfusion solutions, and the provision of such solutions und medical devices coated in such a way. Preferably, the medical devices are such that come into direct contact with cells/tissues and (can) lead to a cell/tissue alteration, which, without a surface coating, lead to an increased production of matrix proteins/fibrosis and/or cell proliferation/cell migration and/or apoptosis/necrosis. A suitable type of application represents also solutions or bio-passivating compositions in the form of rinsing solutions, impregnation solutions, coating solutions, cryoprotection solutions, cryopreservation media, lyoprotection solutions, contrast agent solutions, preservation solutions and perfusion solutions, if trauma/intoxication as well as medical/cosmetic interventions, which are accompanied by similar cell/tissue alterations and cell/tissue reactions, are difficult to reach, so the bio-passivation can be ensured by a direct coating of cells/tissues or can be performed by an immediate coating of medical devices which come into contact with tissues.

This problem is solved by the technical teaching of the independent claims. More advantageous embodiments of the invention result from the dependent claims, the description, the figures, as well as the examples.

Surprisingly, it was found that nitro-fatty acid containing phospholipids of the general structure of (I)

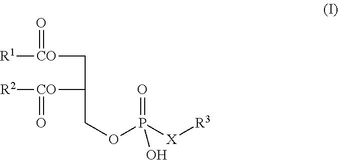

wherein

### X is O or S;

R¹ and R² independently of each other are selected from the group comprising or consisting of:
linear nitroalkyl residues with 5-30 carbon atoms, branched nitroalkyl residues with 5-30 carbon atoms, linear nitroalkenyl residues with 5-30 carbon atoms, branched nitroalkenyl residues with 5-30 carbon atoms, linear nitroalkynyl residues with 5-30 carbon atoms, branched nitroalkynyl residues with 5-30 carbon atoms, nitroalkyl residues with 5-30 carbon atoms, wherein the nitroalkyl residue contains a cycloalkyl residue or a heterocycloalkyl residue or a carbonyl group,
linear alkyl residues with 5-30 carbon atoms, branched alkyl residues with 5-30 carbon atoms, linear alkenyl residues with 5-30 carbon atoms, branched alkenyl residues with 5-30 carbon atoms, linear alkynyl residues with 5-30 carbon atoms, branched alkynyl residues with 5-30 carbon atom, alkyl residues with 5-30 carbon atoms, wherein the alkyl residue contains a cycloalkyl residue or a heterocycloalkyl residue or a carbonyl group,
wherein the alkyl residue, alkenyl residue and alkynyl residue can be substituted with two or three hydroxyl groups, thiol groups, halogen residues, carboxylate groups, C₁-C₅ alkoxycarbonyl groups, C₁-C₅ alkylcarbonyloxy groups, C₁-C₅ alkoxy groups, C₁-C₅ alkyl amine groups, C₁-C₅ dialkylamino groups and/or amine group can be substituted, and wherein the nitroalkyl residue, nitroalkenyl residue and nitroalkynyl residue can be substituted with one, two or three hydroxyl groups, thiol groups, halogen residues, carboxylate groups, C₁-C₅ alkoxycarbonyl groups, C₁-C₅-alkylcarbonyloxy groups, C₁-C₅ alkoxy groups, C₁-C₅ alkyl amine groups, C₁-C₅ dialkylamino groups and/or amino groups,
where at least one of the residues of R¹ and R² must contain at least a nitro group R³ stands for one of the following residues: —H, —CH₂—CH(COO⁻)—NH₃⁺, —CH₂—CH₂—NH₃⁺, —CH₂—CH₂—N(CH₃)₃⁺,

### —CH₂—CH₂—NH₃⁺, —CH₂—CH₂—N(CH₃)₃⁺,

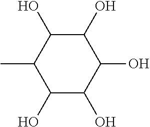

—CR⁴R⁵R⁶, —CR⁴R⁵—CR⁶R⁷R⁸, —CR⁴R⁵—CR⁶R⁷—CR⁸R⁹R¹⁰, —CR⁴R⁵—CR⁶R⁷—CR⁸R⁹—CR¹⁰R¹¹R¹², —CR⁴R⁵—CR⁶R⁷—CR⁸R⁹—CR¹⁰R¹¹—CR¹²R¹³R¹⁴;
R⁴-R¹⁴ represent independently of each other
—OH, —OP(O)(OH)₂, —P(O)(OH)₂, —P(O)(OCH₃)₂, —P(O)(OC₂H₅)₂, —OCH₃, —OC₂H₅, —OC₃H₇, —O-cyclo-C₃H₅, —OCH(CH₃)₂, —OC(CH₃)₃, —OC₄H₉, —OC₄H₉, —OC₅H₁₁, —OCH₂CH(CH₃)₂, —OCH(CH₃)C₂H₅, —OC₆H₁₃, —O-cyclo-C₄H₇, —O-cyclo-C₅H₉, —OPh, —OCH₂-Ph, —OCPh₃, —SH, —SCH₃, —SC₂H₅, —F, —Cl, —Br, —I, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CON(CH₃)₂, —CON(C₂H₅)₂, —CON(C₃H₇)₂, —NH₂, —NO₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(CH₃)₃⁺, —N(C₂H₅)₃⁺, —N(C₃H₇)₃⁺, —N(cyclo-C₃H₅)₃⁺, —N[CH(CH₃)₂]₃⁺, —N(cyclo-C₃H₅)₂, —N[CH(CH₃)₂]₂, —N[C(CH₃)₃]₂, —NH-cyclo-C₄H₇, —NH-cyclo-C₅H₁₁, —NH-cyclo-C₆H₁₃, —N(cyclo-C₄H₇)₂, —N(cyclo-C₅H₁₁)₂, —N(cyclo-C₆H₁₃)₂, —NH(Ph), —NPh₂, —SOCH₃, —SOC₂H₅, —SOC₃H₇, —SO₂CH₃, —SO₂C₂H₅, —SO₂C₃H₇, —SO₃H, —SO₃CH₃, —SO₃C₂H₅, —SO₃C₃H₇, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—N(CH₃)₂, —NH—CO—N(C₂H₅)₂, —O—CO—NH₂, —O—CO—NHCH₃, —O—CO—NHC₂H₅, —O—CO—NHC₃H₇, —O—CO—N(CH₃)₂, —O—CO—N(C₂H₅)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₃, —C₂H₅, —C₃H₇, -cyclo-C₃H₅, —CH(CH₃)₂, —C(CH₃)₃, —C₄H₉, -cyclo-C₄H₇, -cyclo-C₅H₉, -cyclo-C₆H₁₁, —CH₂—CH(CH₃)₂, —CH(CH₃)—C₂H₅, —C₅H₁₁, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH;

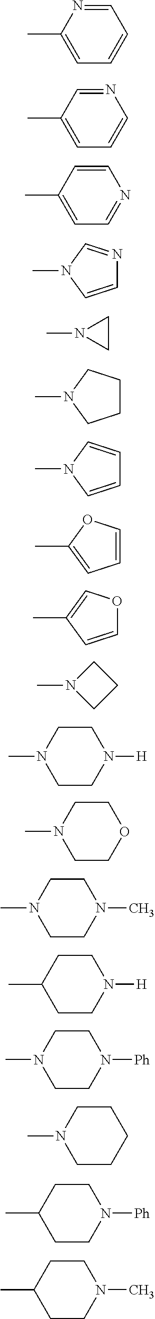

as well as salts, solvates, hydrates, enantiomers, diastereomers, racemates, enantiomeric mixtures, diastereomeric mixtures of the above compounds are also falling under the scope of the present invention.

Thus, another aspect of the present invention concerns the use of the invention nitro fatty acid containing phospholipids for the production of medical compositions and for the coating of medical devices.

The medical compositions are preferably bio-passivating compositions, e.g., rinsing solutions for medical devices, rinsing solutions for wounds, impregnation solutions for dressing wound and suture materials, coating solutions for medical apparatuses, cryoprotection solutions, cryopreservation media, lyoprotection solutions, contrast agent solutions, preservation solutions and perfusion solutions for cells, tissues and organs. These solutions and media, which are all preferably bio-passivation solutions and media, are described further below.

Herein, the terms “medical device” or “medical devices” are used as generic terms which include any implants, natural and artificial grafts, suture and bandage materials as well as parts of medical apparatuses such as catheters. The medical products which include cosmetic or partly cosmetic and medical implants, which are introduced into the body temporarily or permanently, preferably as medical apparatuses, further preferred are medical items that come into contact with cells/tissues, such as wound materials, suture materials, wound and body compartment closure systems, biological grafts, artificial grafts, biological implants, artificial implants, natural or artificial blood vessels, blood conduits, blood pumps, dialysers, dialysis machines, vascular prostheses, vascular supports, heart valves, artificial hearts, vascular clamps, autologous implants, bone implants, intraocular lenses, shunts, dental implants, infusion tubings, medical cuffs, ligatures, medical clamps, pumps, pacemakers including pacemaker probes, laboratory gloves, medical scissors, medical utensils, needles, cannulas, endoprostheses, exoprosthetheses, scalpels, lancets, soft tissue implants, breast implants, facial implants, catheters, guidewires, ports, stents, catheter balloons and catheter balloons with a crimped stent. The inventive surface coatings are suitable for all medical devices or medical apparatuses, which temporarily or permanently can come into contact with cells/tissues/organs and cause an irritation of cells/tissues/organs through this contact or coming into contact with said structures leading to an adverse reaction (as described in the following or above from page 7). This includes medical or cosmetic procedures, which have similar properties.

Vital cells can react to external stimuli by changing their metabolism and/or phenotype and/or genotype. The reaction behavior depends on the type and the intensity of an irritation, as well as the affected cell species and pre-conditioning factors such as the integrity of a cell network or the presence of mediators. The cellular response is dependent on the aforementioned determinates and can manifest itself in the production of mediators or extracellular matrix, cell migration or cell proliferation and also in necrosis or apoptosis. Therefore, the reaction to an irritation of a cell is not exactly predictable. Quantification of a change of the reaction to a cell/tissue irritation must be made by comparison of the response behavior at comparable clinical conditions.

The terms “bio-passivation” or “bio-passivating” which comprise the inventive effects of the nitro fatty acid containing phospholipids are described in more detail in the following. An inventive bio-passivation is present when the cell/tissue response to a physical, chemical, or hypoxic cell or tissue alteration will be limited to a level, as this would be expected/found under the same conditions but without an additional cell/tissue alteration. The cell/tissue alteration can be caused by insertion of foreign material, inflicting baro- or thermo-trauma, hypoxia, toxins or by radiation.

More specifically, an inventive bio-passivation of cells/tissues is present, if the cell/tissue responses to physical, chemical or hypoxic cell or tissue alterations, consisting of a production of mediators and/or matrix proteins, and/or cell migration/proliferation and/or necrosis/apoptosis, is reduced, preferably by at least 10%, preferably by at least 20%, further preferred by at least 30%, preferable by at least 40%, to at least 50%, preferable by at least 60% and most preferred by at least by 70% compared to the cell/tissue response to a similar alteration of cells/tissues, which did not come into contact with the inventive compounds.

In other words, bio-passivating effects are present, when cell or tissue response, which can be the production of mediators and/or matrix proteins, cell migration and/or proliferation, necrosis or apoptosis due to physical, chemical or hypoxic cell or tissue alterations, is reduced in their scale, preferably by at least 10%, preferably by at least 20%, further preferred by at least 30%, preferable by at least 40%, still preferred by at least 50%, more preferable by at least 60% and most favored by at least 70 percent.

Thus, the invention present invention relates to bio-passivating compounds with the general formula (I) bio-passivating compositions containing at least one bio-passivating compound of general formula (I), as well as bio-passivating coatings consisting of or containing the bio-passivating compounds of the general formula (I). These bio-passivating compounds, compositions and coatings are especially useful for direct or indirect contact with living cells, tissues and organs. Thus the present invention relates to bio-passivating compounds, bio-passivating compositions and bio-passivating coatings, where bio-passivation means that the cells or tissues which have come in contact with or have been treated with the bio-passivating compounds, coatings or compositions exhibit at least 10%, preferred at least 20%, further preferred at least 30%, further preferred at least 40%, further preferred at least 50%, further preferred at least 60% and the most preferred at least 70% less cell responses and/or tissue reactions (e.g., production of mediators and/or matrix proteins, cell migration or cell proliferation and/or necrosis or apoptosis) in response to physical, chemical, or hypoxic cell or tissue alterations as compared to cells and/or tissues that were not brought into contact with the bio-passivating compounds, coatings or composites as compared to cell responses and/or tissue reactions of those affected cells/tissues that have not been in contact with the inventive bio-passivating compounds, compositions or coatings.

Therefore bio-passivation does not mean preferably at least 10% lower production of mediators or the at least 10% lower production of matrix proteins, or that at least 10% lower cell migration, or the at least 10% reduced cell proliferation or at least 10% lower necrosis or at least 10% less apoptosis, but bio-passivation means preferably the at least 10% lower production of mediators and/or matrix proteins and that at least 10% lower cell migration and/or cell proliferation and at least 10% lower necrosis and/or apoptosis.

The aforementioned effects (production of mediators/matrix proteins, cell migration/proliferation and necrosis/apoptosis) can be reduced by at least 10% or more only if one of these effects actually occurs. Not all aforementioned effects occur in all biological processes at the same time, so that the aforementioned reduction of effects by at least 10% or more, relates to effects that actually occur during the examined biological processes. In addition the expression “at least 50% lower” (or at least 10%/20%/at least 30%/at least 40%/at least 60%/at least 70%) does not mean that all of the aforementioned effects must be reduced to this percentage. It is sufficient if one of the actually occurring effects is reduced by the same percentage, while the other effects may be reduced to the same, a higher or lower percentage, but preferably by a measurable reduction.

Science can prove this reduction by at least 10%, preferably at least 20%, further preferred at least 30%, further preferred at least 40%, further preferred at least 50%, favored further at least 60% and the most preferred by at least 70% by subsequent methods.

Cytokines and matrix proteins on surfaces can be detected by immunohistochemical detection procedures for in situ cell tissue preparations or in cultural/tissue liquids; assays utilizing densitometry, and elastography of in vitro and in vivo specimens, migration and proliferation assays for in vitro/ex vivo histochemical cell tissue analysis, determination of proliferative cell activity, histopathological determination of cell morphology/number, volumetric determination of tissue formation by means of ultrasonography/resonance imaging/radiology, as well as histochemical assays for a necrotic or apoptotic cell destruction as the live/dead staining, the MTT test, determination of the caspase activity and substances released by cell lyses such as LDH and creatine kinase, as well as by cell structure fragments such as vesicles/DNA, histopathological staining assays such as H&E, Nissel or Fuchsin dyes as well as in vivo diagnostics such as PET/CT/MRI.

The term “bio-compatibility” also encompasses that a surface coating with the bio-passivating compounds and compositions is for the most part chemically and biologically neutral. Such chemical and biological neutrality is present, if the cell/tissue reactions upon contact with an inventive bio-passivating surface with or without concurrent physical, chemical or hypoxic cell or tissue alterations show cell and/or tissue reactions that are not more than 30%, are preferably not more pronounced than 10%, and more preferred than less as 10% as compared to cell and/or tissue reactions of cells that do not come into contact with an inventive bio-passivating surface, whereas the cell and/or tissue reactions consist of a production of mediators and/or matrix proteins, cell migration and/or cell proliferation, and necrosis or apoptosis.

It is possible to prove this scientifically using the methods described above. Here, for example, an inventive biocompatibility of a coating or medical preparation is present when using the same in vitro/ex vivo/in vivo conditions, the cell and/or tissue responses expressed by production of mediators and/or matrix proteins, cell migration and/or cell proliferation, necrosis or apoptosis are not more pronounced than 30%, more preferred are not more pronounced than 10% and are not more reduced than 10% as compared to a similar cell/tissue alteration without a contact of those cells/tissues with the coated material or medical preparation under otherwise same conditions.

The term “proliferation reducing” is to be understood as an inhibitory effect on the migration, proliferation and the formation of extracellular matrix of/by cells/tissues coming in contact with the inventive compounds or composites. This occurs when the cell/tissue response to a physically, chemically, or hypoxia-related cell or tissue alteration, consisting of a production of matrix proteins, cell migration or cell proliferation are reduced between 55% and 100%, favored by 60-65% and more preferred by 75-85% as compared to the cell or tissue reactions induced by a similar alteration of similar cells and tissues, which come into contact with similar surfaces or medical preparations, which have not been coated with the inventive compounds or compositions or where the inventive compounds or compositions were not included in medical preparations.

Scientific proof for this can be achieved in comparable manner as described before. Thus, the inventive proliferation reduction of a coating or medical preparation is present when at the same in vitro/ex vivo/in vivo conditions the cell and/or tissue responses expressed by production of matrix proteins, migration of cells or cell proliferation are reduced between 55% and 100%, preferred by 60-65% and more favored by 75-85% as compared to a similar cell/tissue reaction to a contact with uncoated material or a medical preparation that does not contain inventive compounds or compositions.

As set out in the description and the examples, the bio-compatible and bio-passivating and proliferation-reducing effects can by tested by the use of appropriate investigations and reliably detected and quantified.

The term “nitro-carboxylic acid containing phospholipid” or “nitro-carboxylic acids containing phospholipid” or “nitro carbon acid(s)-containing phospholipid”, which are used interchangeably, is understood that at least one of the two lipid residues of R¹ or R² contain a nitro group (—NO₂). thus the acid residue R¹COO— or the residue of the acid R²COO— has at least a nitro group or both carboxylic acid residues R¹COO— and R²COO— have at least a nitro group.

R¹ corresponds to the carbon residue or the carbon chain of the carboxylic acid residue R¹COO—. The corresponding carboxylic acid is R¹COOH.

R² corresponds to the carbon residue or the carbon chain of the carboxylic acid residue R²COO—. The corresponding carboxylic acid is R²COOH.

Thus, when carboxylic acids or nitro-carboxylic acids, which are contained in the phospholipid, are spoken of, this concerns the residues of R¹COO and R²COO, which are derived from the corresponding carboxylic acids R¹COOH and R²COOH that are bound via an ester bond to the glycerine residue.

Phosphoglycerides are called also glycerophospholipid or phosphatides when a glycerol as a “skeleton” is present.

If in the present application of phospholipids (PL) are spoken of, phospholipids and preferably glycerophospholipids, with lipid residue(s) that do not contain a nitro group, are meant.

On the other hand, the inventive compounds are called nitro carboxylic acid(s)-containing phospholipids, expressing that at least one of the two carbon chains R¹ or R² carries at least a nitro group. Phospholipids containing only one unsaturated nitro-carboxylic acid residue are preferred.

The nitro group in R¹ and/or R² has no specific position. It can be located at each of the carbon atoms (α to ω), i.e., at any point of the carbon chain. If there are several nitro groups on R¹ and/or R², these can be located at arbitrary positions in the carbon chain of R¹ and R². Preferably at least one nitro group is located at a vinyl group of an unsaturated carbon chain. Accordingly, at least a nitro group is preferably located at a double bond of an unsaturated carbon chain. It is possible that the carbon chain contains more than one nitro group. In addition, an allylic position of the nitro group or the nitro groups to the double bond is preferred. Furthermore, preferred is a vicinal position of one or more nitro group(s) to hydroxyl groups in particular at saturated carbon atoms.

The carbon chain can also contain double or triple bonds, it can contain a carbocycle or a heterocycle or an aromatic ring or a heteroaromatic ring and a carbonyl group, and it can be linear or branched and may carry additional substituents. Thus, the term “carbon chain” refers not only to linear and saturated alkyl groups, but also to mono-unsaturated, multiple unsaturated, a cycle containing, branched, and higher substituted alkyl, alkenyl- or alkynyl groups. The single, double or multiple unsaturated carbon chains of unsaturated carboxylic acids are preferred. Double bonds in the carbon chain of carboxylic acids are the most preferred, while triple bonds and saturated carbon chains are less preferred.

Thus, the term “nitrated carbon chain” includes carbon chains consisting of 5-30 carbon atoms carrying at least one nitro group, wherein this carbon chain can contain one or more double bonds and/or one or more triple bonds and can be cyclic, a carbocycle, heterocyclic or aromatic ring and heteroaromatic ring, can be substituted by one or more nitro groups and one or more hydroxyl groups, thiol- or halogen residues, carboxylate groups, C₁-C₅-alkoxycarbonyl groups, C₁-C₅-alkylcarbonyloxy groups, C₁-C₅-alkoxy groups, C₁-C₅-alkyl amino groups, C₁-C₅-dialkylamino groups or an amine group may be substituted.

The term “branched” means that the carbon chain of the remainder of the carboxylic acid has at least one branch, i.e., it is not a linear carbon chain.

The term “nitroalkyl residue” or “nitrated alkyl residue” refers to a linear or branched and saturated carbon chain with 5-30 carbon atoms and at least a nitro group. Nitroalkyl residues can carry a maximum of 10 nitro groups. Preferably a nitroalkyl residue carries 1, 2 or 3 nitro groups if there are 5-10 carbon atoms, and preferably 1, 2, 3, 4 or 5 nitro groups if there are 11-20 carbon atoms, and preferably 1, 2, 3, 4, 5, 6 or 7 nitro groups if there are 21-30 carbon atoms, or furthermore, the nitroalkyl residue also preferably has between 8 and 28 carbon atoms, further preferably between 10 and 26 carbon atoms, yet further preferably between 12 and 24 carbon atoms, and most preferably between 14 and 22 carbon atoms.

The term “nitroalkenyl residue” or “nitrated alkenyl residue” refers to a linear or branched and with double bonds unsaturated carbon chain with 5-30 carbon atoms and at least a nitro group. Nitroalkenyl residues can carry a maximum of 10 nitro groups. Preferably the nitroalkenyl residue contains 1, 2 or 3 nitro groups if there are 5-10 carbon atoms, and preferably 1, 2, 3, 4 or 5 nitro groups if there are 11-20 carbon atoms, and preferably 1, 2, 3, 4, 5, 6 or 7 nitro groups if there are 21-30 carbon atoms. The most preferred nitroalkenyl residue contains one, two or three nitro groups. The nitroalkenyl residue contains at least one and a maximum of 15 double bonds. One, two or three double bonds are preferred, one or two double bonds are more preferred and one double bond is especially favored. The double bonds can each be E (“entgegen”; also known as “trans”) or Z (“zusammen”; known as “cis”), independently. Preferred are double bonds with a Z orientation. The nitroalkenyl residue contains also preferably between 8 and 28 carbon atoms, further preferably between 10 and 26 carbon atoms, yet further preferably between 12 and 24 carbons, and most preferably between 14 and 22 carbon atoms.

The term “nitroalkynyl residue” or “nitrated alkynyl residue” refers to a linear or branched carbon chain with 5-30 carbon atoms unsaturated with triple bonds and at least one nitro group. Nitroalkynyl residues can carry a maximum of 10 nitro groups. Preferably the nitroalkynyl residue consists of 1, 2 or 3 nitro groups, if it has 5-10 carbon atoms, and preferably 1, 2, 3, 4 or 5 nitro groups, if it has 11-20 carbon atoms, and preferably 1, 2, 3, 4, 5, 6 or 7 nitro groups, if it has 21-30 carbon atoms. Nitroalkynyl residue containing one, two or three nitro groups is most preferred. The nitroalkynyl residue contains at least one and no more than 10 triple bonds. One, two, or three triple bonds are preferred, one or two triple bonds are more preferred and in particular preferred is one triple bond. The nitroalkynyl residue also contains preferably between 8 and 28 carbon atoms, further preferably between 10 and 26 carbon atoms, yet further preferably between 12 and 24 carbon atoms, and at most preferably between 14 and 22 carbon atoms.

The term “nitro alkyl residue with 5-30 carbon atoms containing a cycloalkyl residue or a heterocycloalkyl residue or a carbonyl group” refers to a linear or branched carbon chain with 5-30 carbon atoms, with a cycloalkyl residue or a heterocycloalkyl residue or a carbonyl in the carbon chain. The carbon atoms of the cycloalkyl residue or the heterocycloalkyl residues or the carbonyl group are included in the total number of carbon atoms, thus are included in the 5-30 carbon atoms. The nitroalkyl residue with 5-30 carbon atoms containing a cycloalkyl residue or a heterocycloalkyl residue or a carbonyl group containing up to 10 nitro groups and preferably contains 1, 2 or 3 nitro groups if it has 5-10 carbon atoms, and preferably 1, 2, 3, 4 or 5 nitro groups if it has 11-20 carbon atoms, and or preferably 1, 2, 3, 4, 5, 6 or 7 nitro groups, if it has 21-30 carbon atoms. The most preferred is a nitroalkyl residue that contains one, two or three nitro groups. The nitroalkyl residue containing a cycloalkyl residue or a heterocycloalkyl residue or a carbonyl group is preferably between 8 and 28 carbon atoms, further preferably between 10 and 26 carbon atoms, yet further preferably between 12 and 24 carbons, and most preferably between 14 and 22 carbon atoms.

The term “alkyl residue” refers to a linear or branched and saturated carbon chain with 5-30 carbon atoms and without a nitro group. The alkyl residue also contains preferably between 8 and 28 carbon atoms, further preferably between 10 and 26 carbon atoms, yet further preferably between 12 and 24 carbons, and most preferably between 14 and 22 carbon atoms.

The term “alkenyl residue” refers to a linear or branched and with 20 double bonds unsaturated carbon chain with 5-30 carbon atoms and without nitro group. The alkenyl residue contains at least one and no more than 15 double bonds. One, two or three double bonds are preferred, one or two double bonds are more preferred and a single double bond is especially favored. The double bonds can be each independently of each other E (“entgegen”; known as “trans”) or Z (“zusammen”; known as “cis”). Z double bonds are preferred. The alkenyl residue contains also preferably between 8 and 28 carbon atoms, further preferably between 10 and 26 carbon atoms, yet further preferably between 12 and 24 carbons, and most preferably between 14 and 22 carbon atoms.

The term “alkynyl residue” refers to a linear or branched and unsaturated with triple bonds carbon chain with 5-30 carbon atoms and at least a nitro group. The alkynyl residue contains at least one and no more than 10 triple bonds. One, two, or three triple bonds are preferred, one or two triple bonds are more preferred and in particular preferred is a single triple bonds. The alkynyl residue contains also preferably between 8 and 28 carbon atoms further preferably between 10 and 26 carbon atoms, further preferably 12 and 24 carbons, and most preferably 14 and 22 carbon atoms.

The nitroalkyl residue, nitroalkenyl residue, nitroalkynyl residue, nitroalkyl residue with 5-30 carbon atoms containing a cycloalkyl residue or a heterocycloalkyl residue or a carbonyl group, alkyl residue, alkenyl residue and alkynyl residue can also be substituted with one, two or three hydroxyl groups, thiol groups, halogen residues (—F, —I, —Cl, —Br), carboxylate groups, C₁-C₅-alkoxycarbonyl groups, C₁-C₅-alkylcarbonyloxy groups, C₁-C₅-alkoxy groups, C₁-C₅-alkyl amino groups, C₁-C₅-dialkylamino groups and/or amino groups. Further preferred are hydroxyl- and C₁-C₅-alkoxy groups, whereas hydroxyl groups are particularly preferred.

Preferred are the inventive phospholipids and their uses, where R¹ is a nitroalkyl residue and R² a nitroalkyl residue or where R¹ is a nitroalkyl residue and R² nitroalkenyl residual or where R¹ is a nitroalkyl residue and R² an alkyl residue or where R¹ is a nitroalkyl residue and R² an alkenyl residue or where R¹ is a nitroalkenyl residue and R² a nitroalkyl residue or where R¹ is a nitroalkenyl residue and R² a nitroalkenyl residue or where R¹ is a nitroalkenyl residue and R² an alkyl residue or where R¹ is a nitroalkenyl residue and R² an alkenyl residue or where R¹ is an alkyl residue and R² a nitroalkyl residue or where R¹ is an alkyl residue and R² a nitroalkenyl residue or wherein R¹ is an alkenyl residue and R² is a nitroalkenyl residue, or where R¹ is an alkenyl residue and R² is a nitroalkyl residue.

Following carboxylic acids represented as a free acid R¹COOH and R²COOH are used preferable as residues R¹COO— and R²COO— in the nitro-carboxylic acid containing phospholipids according to formula (I). The following carboxylic acids are used preferably in the form of nitrated, i.e. with at least a nitro group and optionally another substituent listed above for the esterification of glycerol residue in the inventive phospholipids:

Hexanoic acid (Capronic acid), Octanoic acid (Caprylic acid), decanoic acid (Caprinic acid), dodecanoic acid (Lauric acid), tetradecanoic acid (Myristic acid), hexadecanoic acid (Palmitic acid), heptadecanoic acid (Margaric acid), Octadecanoic acid (Stearic acid), Eicosanoic acid (Arachidic acid), docosanoic acid (Behenic acid), tetracosanoic acid (Lignoceric acid), cis-9-tetradecenoic acid (Myristoleic acid), cis-9-hexadecenoic acid (Palmitoleic acid), cis-6-Octadecenoic acid (Petroselinic acid), cis-9-Octadecenoic acid (oleic acid), cis-11-Octadecenoic acid (Vaccenic acid), cis-9-Eicosenoic acid (Gadoleic acid), cis-11-Eicosenoic acid (Gondoic acid), cis-13-docosenoic acid (Erucic acid), cis-15-tetracosenoic acid (Nervonic acid), t9-Octadecenoic acid, t11-Octadecenoic acid, t3-hexadecenoic acid, 9,12-Octadecadienoic acid (Linoleic acid), 6,9,12-Octadecatrienoic acid (γ-Linolenic acid), 8,11,14-Eicosatrienoic acid (Dihomo-γ-linolenic acid), 5,8,11,14-Eicosatetraenoic acid (Arachidonic acid), 7,10,13,16-Docosatetraenoic acid, 4,7,10,13,16-Docosapentaenoic acid, 9,12,15-Octadecatrienoic acid (α-Linolenic acid), 6,9,12,15-Octadecatetraenoic acid (Stearidonic acid), 8,11,14,17-Eicosatetraenoic acid, 5,8,11,14,17-Eicosapentaenoic acid (EPA), 7,10,13,16,19-Docosapentaenoic acid (DPA), 4,7,10,13,16,19-Docosahexaenoic acid (DHA), 5,8,11-Eicosatrienoic acid (Mead acid), 9c11t13t-Octadecatrienoic acid, 8t10t12c-Octadecatrienoic acid, 9c11t13c-Catalpinic acid, 4,7,9,11,13,16,19-Docosaheptaenoic acid, Taxoleic acid, Pinolenic acid, Sciadonic acid, 6-Octadecynoic acid (Tariric acid), t11-Octadecen-9-ynoic acid (Santalbic acid as well as Ximeninic acid), 9-Octadecynoic acid (Stearolic acid), 6-Octadecen-9-ynoic acid, t10-Heptadecen-8-ynoic acid (Pyrulic acid), 9-Octadecen-12-ynoic acid (Crepenyic acid), t7,t11-Octadecadien-9-ynoic acid (Heisteric acid), t8,t10-Octadecadien-12-ynoic acid, 5,8,11,14-Eicosatetraynoic acid (ETYA), Retinoic acid, Isopalmitic acid, Pristanic acid, 3,7,11,15-Tetramethylhexadecanoic acid (Phytanic acid), 11,12-Methyleneoctadecanoic acid, 9,10-Methylene-hexadecanoic acid, Coronaric acid, (R,S)-Liponic acid, (S)-Liponic acid, (R)-Liponic acid, 6,8-(methylsulfanyl)-octanoic acid, 4,6-Bis(methylsulfanyl)-hexanoic acid, 2,4-bis(methylsulfanyl)-butanoic acid, 1,2-Dithiolan-carboxylic acid, (R,S)-6,8-dithiane octanoic acid, (S)-6,8-dithiane octanoic acid, 6,9-Octadecenynoic acid, t8,t10-Octadecadien-12-ynoic acid, Hydroxytetracosanoic acid (Cerebronic acid), 2-Hydroxy-15-tetracosenoic acid (Hydroxynervonic acid), 12-Hydroxy-9-octadecenoic acid (Ricinoleic acid), 14-Hydroxy-11-eicosenoic acid (Lesquerolic acid), Pimelic acid, Suberic acid, Azelaic acid, Sebacic acid, Brassylic acid and Thapsic acid. If just one carbon acid residue R¹COO— or R²COO— is nitrated, the other not nitrated carbon acid residue will be preferably chosen from the list above.

The use of the nitrated forms of the forementioned acids is also preferred. The residues of R¹COO— and R²COO— in the nitro-carboxylic acid containing phospholipids in accordance with the present invention, represented as a free acid R¹COOH and R²COOH, can represent a nitrated carboxylic acid here, selecting the appropriate carbon acid (s) from the above group.

This means that the above specifically named carboxylic acids are used preferably as residues R¹COO— or as a residue R²COO— in the inventive phospholipids in accordance with the general formula (I) and the nitrated form of the above specified carboxylic acids is preferably used as a second lipid residue R²COO— or R¹COO— or the nitrated form of this above specifically described carboxylic acid is preferably used for both lipid residues R¹COO— and R²COO—.

Especially preferred are lipid residues of the inventive phospholipids of the following nitrated carboxylic acid R¹COO— and R²COO—:

nitrohexadecanoyl, dinitrohexadecanoyl, trinitrohexadecanoyl, nitroheptadecanoyl, dinitroheptadecanoyl, trinitroheptadecanoyl, nitrooctadecanoyl, dinitrooctadecanoyl, trinitrooctadecanoyl, nitroeicosanoyl, dinitroeicosanoyl, trinitroeicosanoyl, 5 nitrodocosanoyl, dinitrodocosanoyl, trinitrodocosanoyl, nitrotetracosanoyl, dinitrotetracosanoyl, trinitrotetracosanoyl, nitro-cis-9-tetradecenoyl, dinitro-cis-9-tetradecenoyl, trinitro-cis-9-tetradecenoyl, nitro-cis-9-hexadecenoyl, dinitro-cis-9-hexadecenoyl, trinitro-cis-9-hexadecenoyl, nitro-cis-6-octadecenoyl, dinitro-cis-6-octadecenoyl, trinitro-cis-6-octadecenoyl, nitro-cis-9-octadecenoyl, dinitro-cis-9-10 octadecenoyl, trinitro-cis-9-octadecenoyl, nitro-cis-11-octadecenoyl, dinitro-cis-11-octadecenoyl, trinitro-cis-11-octadecenoyl, nitro-cis-9-eicosenoyl, dinitro-cis-9-eicosenoyl, trinitro-cis-9-eicosenoyl, nitro-cis-11-eicosenoyl, dinitro-cis-11-eicosenoyl, trinitro-cis-11-eicosenoyl, nitro-cis-13-docosenoyl, dinitro-cis-13-docosenoyl, trinitro-, cis-13-docosenoyl, nitro-cis-15-tetracosenoyl, dinitro-cis-15-tetracosenoyl, trinitro-cis-15-tetracosenoyl, nitro-t9-octadecenoyl, dinitro-t9-octadecenoyl, trinitro-t9-octadecenoyl, nitro-t11-octadecenoyl, dinitro-t11-octadecenoyl, trinitro-t11-octadecenoyl, nitro-t3-hexadecenoyl, dinitro-t3-hexadecenoyl, trinitro-t3-hexadecenoyl, nitro-9,12-octadecadienoyl, dinitro-9,12-octadecadienoyl, trinitro-9,12-octadecadienoyl, nitro-6,9,12-octadecatrienoyl, dinitro-6,9,12-octadecatrienoyl, trinitro-6,9,12-octadecatrienoyl, nitro-8,11,14-eicosatrienoyl, dinitro-8,11,14-eicosatrienoyl, trinitro-8,11,14-eicosatrienoyl, nitro-5,8,11,14-eicosatetraenoyl, dinitro-5,8,11,14-eicosatetraenoyl, trinitro-5,8,11,14-eicosatetraenoyl, nitro-7,10,13,16-docosatetraenoyl, dinitro-7,10,13,16-docosatetraenoyl, trinitro-7,10,13,16-docosatetraenoyl, nitro-4,7,10,13,16-docosapentaenoyl, dinitro-4,7,10,13,16-docosapentaenoyl, trinitro-4,7,10,13,16-docosapentaenoyl, nitro-9,12,15-octadecatrienoyl, dinitro-9,12,15-octadecatrienoyl, trinitro-9,12,15-octadecatrienoyl, nitro-6,9,12,15-octadecatetraenoyl, dinitro-6,9,12,15-octadecatetraenoyl, trinitro-6,9,12,15-octadecatetraenoyl, nitro-8,11,14,17-eicosatetraenoyl, dinitro-8,11,14,17-eicosatetraenoyl, trinitro-8,11,14,17-eicosatetraenoyl, nitro-5,8,11,14,17-eicosapentaenoyl, dinitro-5,8,11,14,17-eicosapentaenoyl, trinitro-5,8,11,14,17-eicosapentaenoyl, nitro-7,10,13,16,19-docosapentaenoyl, dinitro-7,10,13,16,19-docosapentaenoyl, trinitro-7,10,13,16,19-docosapentaenoyl, nitro-4,7,10,13,16,19-docosahexaenoyl, dinitro-4,7,10,13,16,19-docosahexaenoyl, trinitro-4,7,10,13,16,19-docosahexaenoyl, nitro-5,8,11-eicosatrienoyl, dinitro-5,8,11-eicosatrienoyl, trinitro-5,8,11-eicosatrienoyl, nitro-9c11t13t-eleostearinoyl, dinitro-9c11t13t-eleostearinoyl, trinitro-9c11t13t-eleostearinoyl, nitro-8t10t12c-calendulaoyl, dinitro-8t10t12c-calendulaoyl, trinitro-8t10t12c-calendulaoyl, nitro-9c11t13c-catalpinoyl, dinitro-9c11t13c-catalpinoyl, trinitro-9c11t13c-catalpinoyl, nitro-4,7,9,11,13,16,19-docosaheptaenoyl, dinitro-4,7,9,11,13,16,19-docosaheptaenoyl, trinitro-4,7,9,11,13,16,19-docosaheptaenoyl, nitrotaxoleinoyl, dinitrotaxoleinoyl, trinitrotaxoleinoyl, nitropinolenoyl, dinitropinolenoyl, trinitropinolenoyl, nitrosciadonoyl, dinitrosciadonoyl, trinitrosciadonoyl, nitro-6-octadecynoyl, dinitro-6-octadecynoyl, trinitro-6-octadecynoyl, nitro-t11-octadecen-9-ynoyl, dinitro-t11-octadecen-9-ynoyl, trinitro-t11-octadecen-9-ynoyl, nitro-9-octadecynoyl, dinitro-9-octadecynoyl, trinitro-9-octadecynoyl, nitro-6-octadecen-9-ynoyl, dinitro-6-octadecen-9-ynoyl, trinitro-6-octadecen-9-ynoyl, nitro-t10-heptadecen-8-ynoyl, dinitro-t10-heptadecen-8-ynoyl, trinitro-t10-heptadecen-8-ynoyl, nitro-9-octadecen-12-ynoyl, dinitro-9-octadecen-12-ynoyl, trinitro-9-octadecen-12-ynoyl, nitro-t7,t11-octadecadien-9-ynoyl, dinitro-t7,t11-octadecadien-9-ynoyl, trinitro-t7,t11-octadecadien-9-ynoyl, nitro-t8,t10-octadecadien-12-ynoyl, dinitro-t8,t10-octadecadien-12-ynoyl, trinitro-t8,t10-octadecadien-12-ynoyl, nitro-5,8,11,14-eicosatetraynoyl, dinitro-5,8,11,14-eicosatetraynoyl, trinitro-5,8,11,14-eicosatetraynoyl, nitroretinoyl, dinitroretinoyl, trinitroretinoyl, nitroisopalmitinoyl, dinitroisopalmitinoyl, trinitroisopalmitinoyl, nitropristanoyl, dinitropristanoyl, trinitropristanoyl, nitrophytanoyl, dinitrophytanoyl, trinitrophytanoyl, nitro-11,12-methylen-octadecanoyl, dinitro-11,12-methylen-octadecanoyl, trinitro-11,12-methylen-octadecanoyl, nitro-9,10-methylen-hexadecanoyl, dinitro-9,10-methylen-hexadecanoyl, trinitro-9,10-methylen-hexadecanoyl, nitrocoronarinoyl, dinitrocoronarinoyl, trinitrocoronarinoyl, nitro-6,9-octadecenynoyl, dinitro-6,9-octadecenynoyl, trinitro-6,9-octadecenynoyl, nitro-t8,t10-octadecadien-12-ynoyl, dinitro-t8,t10-octadecadien-12-ynoyl, trinitro-t8,t10-octadecadien-12-ynoyl, nitrohydroxytetracosanoyl, dinitrohydroxytetracosanoyl, trinitrohydroxytetracosanoyl, nitro-2-hydroxy-15-tetracosenoyl, dinitro-2-hydroxy-15-tetracosenoyl, trinitro-2-hydroxy-15-tetracosenoyl, nitrobrassylinoyl, dinitrobrassylinoyl, trinitrobrassylinoyl, nitrothapsinoyl, dinitrothapsinoyl and trinitrothapsinoyl.

Further preferred are nitrated carboxylic acid residues R¹COO and R²COO— as well as mixtures of carboxylic acids residues mentioned in the following but are not restricted to:
trans-9-nitro-9-octadecenoyl, trans-10-nitro-9-octadecenoyl.

Mixtures of trans-9-nitro-9-octadecenoyl and trans-10-nitro-9-octadecenoyl, trans-9-nitro-10-octadecenoyl, trans-10-nitro-8-octadecenoyl.

Mixtures of trans-9-nitro-10-octadecenoyl and trans-10-nitro-8-octadecenoyl, 10-hydroxy-9-nitrooctadecanoyl, 9-hydroxy-10-nitrooctadecanoyl.

Mixtures of 10-hydroxy-9-nitrooctadecanoyl and 9-hydroxy-10-nitrooctadecanoyl. trans-9-nitro-9-tetradecenoyl, trans-10-nitro-9-tetradecenoyl.

Mixtures of trans-9-nitro-9-tetradecenoyl and trans-10-nitro-9-tetradecenoyl, trans-9-nitro-10-tetradecenoyl, trans-10-nitro-8-tetradecenoyl.

Mixtures of trans-9-nitro-10-tetradecenoyl and trans-10-nitro-8-tetradecenoyl, 10-hydroxy-9-nitrotetradecanoyl, 9-hydroxy-10-nitrotetradecanoyl.

Mixtures of 10-hydroxy-9-nitrotetradecanoyl and 9-hydroxy-10-nitrotetradecanoyl. trans-9-nitro-9-hexadecenoyl, trans-10-nitro-9-hexadecenoyl.

Mixtures of trans-9-nitro-9-hexadecenoyl and trans-10-nitro-9-hexadecenoyl, trans-9-nitro-10-hexadecenoyl, trans-10-nitro-8-hexadecenoyl.

Mixtures of trans-9-nitro-10-hexadecenoyl and trans-10-nitro-8-hexadecenoyl, 10-hydroxy-9-nitrohexadecanoyl, 9-hydroxy-10-nitrohexadecanoyl.

Mixtures of 10-hydroxy-9-nitrohexadecanoyl and 9-hydroxy-10-nitrohexadecanoyl. trans-6-nitro-6-octadecenoyl, trans-7-nitro-6-octadecenoyl.

Mixtures of trans-6-nitro-6-octadecenoyl and trans-7-nitro-6-octadecenoyl, trans-6-nitro-7-octadecenoyl, trans-7-nitro-5-octadecenoyl.

Mixtures of trans-6-nitro-7-octadecenoyl and trans-7-nitro-5-octadecenoyl, 7-hydroxy-6-nitrooctadecanoyl, 6-hydroxy-7-nitrooctadecanoyl.

Mixtures of 7-hydroxy-6-nitrooctadecanoyl and 6-hydroxy-7-nitrooctadecanoyl. trans-11-nitro-11-octadecenoyl, trans-12-nitro-11-octadecenoyl.

Mixtures of trans-11-nitro-11-octadecenoyl and trans-12-nitro-11-octadecenoyl, trans-11-nitro-12-octadecenoyl, trans-12-nitro-10-octadecenoyl.

Mixtures of trans-11-nitro-12-octadecenoyl and trans-12-nitro-10-octadecenoyl, 12-hydroxy-11-nitrooctadecanoyl, 11-hydroxy-12-nitrooctadecanoyl.

Mixtures of 12-hydroxy-11-nitrooctadecanoyl and 11-hydroxy-12-nitrooctadecanoyl. trans-9-nitro-9-eicosenoyl, trans-10-nitro-9-eicosenoyl.

Mixtures of trans-9-nitro-9-eicosenoyl and trans-10-nitro-9-eicosenoyl, trans-9-nitro-10-eicosenoyl, trans-10-nitro-8-eicosenoyl.

Mixtures of trans-9-nitro-10-eicosenoyl and trans-10-nitro-8-eicosenoyl, 10-hydroxy-9-nitroeicosanoyl, 9-hydroxy-10-nitroeicosanoyl.

Mixtures of 10-hydroxy-9-nitroeicosanoyl and 9-hydroxy-10-nitroeicosanoyl. trans-11-nitro-11-eicosenoyl, trans-12-nitro-11-eicosenoyl.

Mixtures of trans-11-nitro-11-eicosenoyl and trans-12-nitro-11-eicosenoyl, trans-11-nitro-12-eicosenoyl, trans-12-nitro-10-eicosenoyl.

Mixtures of trans-11-nitro-12-eicosenoyl and trans-12-nitro-10-eicosenoyl, 12-hydroxy-11-nitroeicosanoyl, 11-hydroxy-12-nitroeicosanoyl.

Mixtures of 12-hydroxy-11-nitroeicosanoyl and 11-hydroxy-12-nitroeicosanoyl. trans-13-nitro-13-docosenoyl, trans-14-nitro-13-docosenoyl.

Mixtures of trans-13-nitro-13-docosenoyl and trans-14-nitro-13-docosenoyl, trans-13-nitro-14-docosenoyl, trans-14-nitro-12-docosenoyl.

Mixtures of trans-13-nitro-14-docosenoyl and trans-14-nitro-12-docosenoyl, 14-hydroxy-13-nitrodocosanoyl, 13-hydroxy-14-nitrodocosanoyl.

Mixtures of 14-hydroxy-13-nitrodocosenoyl and 13-hydroxy-14-nitrodocosenoyl. trans-15-nitro-15-docosenoyl, trans-16-nitro-15-docosenoyl.

Mixtures of trans-15-nitro-15-docosenoyl and trans-16-nitro-15-docosenoyl, trans-15-nitro-16-docosenoyl, trans-16-nitro-14-docosenoyl.

Mixtures of trans-15-nitro-16-docosenoyl and trans-16-nitro-14-docosenoyl, 16-hydroxy-15-nitrodocosanoyl, 15-hydroxy-16-nitrodocosanoyl.

Mixtures of 16-hydroxy-15-nitrodocosenoyl and 15-hydroxy-16-nitrodocosenoyl. (E,Z)-9-nitro-9,12-octadecadienoyl, (E,Z)-10-nitro-9,12-octadecadienoyl, (Z,E)-12-nitro-9,12-octadecadienoyl, (Z,E)-13-nitro-9,12-octadecadienoyl.

Mixtures of (E,E)-9-nitro-9,12-octadecadienoyl, (E,E)-10-nitro-9,12-octadecadienoyl, (E,E)-12-nitro-9,12-octadecadienoyl and (E,E)-13-nitro-9,12-octadecadienoyl. (E,E)-9,12-dinitro-9,12-octadecadienoyl, (E,E)-9,13-dinitro-9,12-octadecadienoyl, (E,E)-10,12-dinitro-9,12-octadecadienoyl, (E,E)-10,13-dinitro-9,12-octadecadienoyl.

Mixtures of (E,E)-9,12-dinitro-9,12-octadecadienoyl, (E,E)-9,13-dinitro-9,12-octadecadienoyl, (E,E)-10,12-dinitro-9,12-octadecadienoyl, (E,E)-10,13-dinitro-9,12-octadecadienoyl.

Mixtures of (E,E)-9-nitro-9,12-octadecadienoyl, (E,E)-10-nitro-9,12-octadecadienoyl, (E,E)-12-nitro-9,12-octadecadienoyl, (E,E)-13-nitro-9,12-octadecadienoyl, (E,E)-9,12-dinitro-9,12-octadecadienoyl, (E,E)-9,13-dinitro-9,12-octadecadienoyl, (E,E)-10,12-dinitro-9,12-octadecadienoyl, (E,E)-10,13-dinitro-9,12-octadecadienoyl.

(E,Z)-9-nitro-10,12-octadecadienoyl, (E,Z)-10-nitro-8,12-octadecadienoyl, (Z,E)-12-nitro-9,13-octadecadienoyl, (Z,E)-13-nitro-9,11-octadecadienoyl.

Mixtures of (E,Z)-9-nitro-10,12-octadecadienoyl, (E,Z)-10-nitro-8,12-octadecadienoyl, (Z,E)-12-nitro-9,13-octadecadienoyl, (Z,E)-13-nitro-9,11-octadecadienoyl.

(Z)-10-hydroxy-9-nitro-12-octadecenoyl, (Z)-9-hydroxy-10-nitro-12-octadecenoyl, (Z)-13-hydroxy-12-nitro-9-octadecenoyl, (Z)-12-hydroxy-13-nitro-9-octadecenoyl, 10,13-dihydroxy-9,12-dinitrooctadecanoyl, 9,13-dihydroxy-10,12-dinitrooctadecanoyl, 10,12-dihydroxy-9,13-dinitrooctadecanoyl, 9,12-dihydroxy-10,13-dinitrooctadecanoyl. Mixtures of (Z)-10-hydroxy-9-nitro-12-octadecenoyl, (Z)-9-hydroxy-10-nitro-12-octadecenoyl, (Z)-13-hydroxy-12-nitro-9-octadecenoyl, (Z)-12-hydroxy-13-nitro-9-octadecenoyl, 10,13-dihydroxy-9,12-dinitrooctadecanoyl, 9,13-dihydroxy-10,12-dinitrooctadecanoyl, 10,12-dihydroxy-9,13-dinitrooctadecanoyl, 9,12-di hydroxy-10,13-dinitrooctadecanoyl,

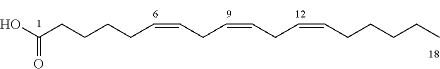 [SMILES: CCCCC/C=C\C/C=C\C/C=C\CCCCC(=O)O]

### (Z,Z,Z)-6,9,12-octadecatrienoic acid (γ-Linolenic acid)

(E,Z,Z)-6-nitro-6,9,12-octadecatrienoyl, (E,Z,Z)-7-nitro-6,9,12-octadecatrienoyl, (Z,E,Z)-9-nitro-6,9,12-octadecatrienoyl, (Z,E,Z)-10-nitro-6,9,12-octadecatrienoyl, (Z,Z,E)-12-nitro-6,9,12-octadecatrienoyl, (Z,Z,E)-13-nitro-6,9,12-octadecatrienoyl.

Mixtures of (E,Z,Z)-6-nitro-6,9,12-octadecatrienoyl, (E,Z,Z)-7-nitro-6,9,12-octadecatrienoyl, (Z,E,Z)-9-nitro-6,9,12-octadecatrienoyl, (Z,E,Z)-10-nitro-6,9,12-octadecatrienoyl, (Z,Z,E)-12-nitro-6,9,12-octadecatrienoyl, (Z,Z,E)-13-nitro-6,9,12-octadecatrienoyl.

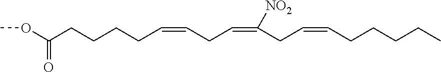

### (Z,E,Z)-10-nitro-6,9,12-octadecatrienoyl

(E,E,Z)-6,9-dinitro-6,9,12-octadecatrienoyl, (E,E,Z)-6,10-dinitro-6,9,12-octadecatrienoyl, (E,E,Z)-7,9-dinitro-6,9,12-octadecatrienoyl, (E,E,Z)-7,10-dinitro-6,9,12-octadecatrienoyl, (E,Z,E)-6,12-dinitro-6,9,12-octadecatrienoyl, (E,Z,E)-6,13-dinitro-6,9,12-octadecatrienoyl, (E,Z,E)-7,12-dinitro-6,9,12-octadecatrienoyl, (E,Z,E)-7,13-dinitro-6,9,12-octadecatrienoyl, (Z,E,E)-9,12-dinitro-6,9,12-octadecatrienoyl, (Z,E,E)-9,13-dinitro-6,9,12-octadecatrienoyl, (Z,E,E)-10,12-dinitro-6,9,12-octadecatrienoyl, (Z,E,E)-10,13-dinitro-6,9,12-octadecatrienoyl.

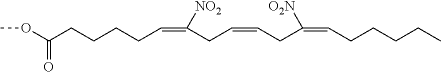

### (E,Z,E)-7,12-dinitro-6,9,12-octadecatrienoyl

Mixtures of (E,E,Z)-6,9-dinitro-6,9,12-octadecatrienoyl, (E,E,Z)-6,10-dinitro-6,9,12-octadecatrienoyl, (E,E,Z)-7,9-dinitro-6,9,12-octadecatrienoyl, (E,E,Z)-7,10-dinitro-6,9,12-octadecatrienoyl, (E,Z,E)-6,12-d i nitro-6,9,12-octadecatrienoyl, (E,Z,E)-6,13-dinitro-6,9,12-octadecatrienoyl, (E,Z,E)-7,12-dinitro-6,9,12-octadecatrienoyl, (E,Z,E)-7,13-dinitro-6,9,12-octadecatrienoyl, (Z,E,E)-9,12-dinitro-6,9,12-octadecatrienoyl, (Z,E,E)-9,13-dinitro-6,9,12-octadecatrienoyl, (Z,E,E)-10,12-dinitro-6,9,12-octadecatrienoyl and (Z,E,E)-10,13-dinitro-6,9,12-octadecatrienoyl.

(E,E,E)-6,9,12-trinitro-6,9,12-octadecatrienoyl, (E,E,E)-6,9,13-trinitro-6,9,12-octadecatrienoyl, (E,E,E)-6,10,12-trinitro-6,9,12-octadecatrienoyl, (E,E,E)-6,10,13-trinitro-6,9,12-octadecatrienoyl, (E,E,E)-7,9,12-trinitro-6,9,12-octadecatrienoyl, (E,E,E)-7,9,13-trinitro-6,9,12-octadecatrienoyl, (E,E,E)-7,10,12-trinitro-6,9,12-octadecatrienoyl, (E,E,E)-7,10,13-trinitro-6,9,12-octadecatrienoyl.

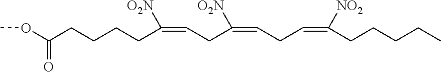

### (E,E,E)-6,9,13-trinitro-6,9,12-octadecatrienoyl,

Mixtures of (E,E,E)-6,9,12-trinitro-6,9,12-octadecatrienoyl, (E,E,E)-6,9,13-trinitro-6,9,12-octadecatrienoyl, (E,E,E)-6,10,12-trinitro-6,9,12-octadecatrienoyl, (E,E,E)-6,10,13-trinitro-6,9,12-octadecatrienoyl, (E,E,E)-7,9,12-trinitro-6,9,12-octadecatrienoyl, (E,E,E)-7,9,13-trinitro-6,9,12-octadecatrienoyl, (E,E,E)-7,10,12-trinitro-6,9,12-octadecatrienoyl and (E,E,E)-7,10,13-trinitro-6,9,12-octadecatrienoyl.

Mixtures of (E,Z,Z)-6-nitro-6,9,12-octadecatrienoyl, (E,Z,Z)-7-nitro-6,9,12-octadecatrienoyl, (Z,E,Z)-9-nitro-6,9,12-octadecatrienoyl, (Z,E,Z)-10-nitro-6,9,12-octadecatrienoyl, (Z,Z,E)-12-nitro-6,9,12-octadecatrienoyl, (Z,Z,E)-13-nitro-6,9,12-octadecatrienoyl, (E,E,Z)-6,9-dinitro-6,9,12-octadecatrienoyl, (E,E,Z)-6,10-dinitro-6,9,12-octadecatrienoyl, (E,E,Z)-7,9-dinitro-6,9,12-octadecatrienoyl, (E,E,Z)-7,10-dinitro-6,9,12-octadecatrienoyl, (E,Z,E)-6,12-dinitro-6,9,12-octadecatrienoyl, (E,Z,E)-6,13-dinitro-6,9,12-octadecatrienoyl, (E,Z,E)-7,12-dinitro-6,9,12-octadecatrienoyl, (E,Z,E)-7,13-dinitro-6,9,12-octadecatrienoyl, (Z,E,E)-9,12-dinitro-6,9,12-octadecatrienoyl, (Z,E,E)-9,13-dinitro-6,9,12-octadecatrienoyl, (Z,E,E)-10,12-dinitro-6,9,12-octadecatrienoyl, (Z,E,E)-10,13-dinitro-6,9,12-octadecatrienoyl, (E,E,E)-6,9,12-trinitro-6,9,12-octadecatrienoyl, (E,E,E)-6,9,13-trinitro-6,9,12-octadecatrienoyl, (E,E,E)-6,10,12-trinitro-6,9,12-octadecatrienoyl, (E,E,E)-6,10,13-trinitro-6,9,12-octadecatrienoyl, (E,E,E)-7,9,12-trinitro-6,9,12-octadecatrienoyl, (E,E,E)-7,9,13-trinitro-6,9,12-octadecatrienoyl, (E,E,E)-7,10,12-trinitro-6,9,12-octadecatrienoyl and (E,E,E)-7,10,13-trinitro-6,9,12-octadecatrienoyl.

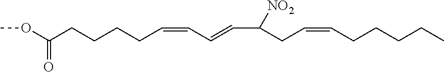

### (Z,E,Z)-10-nitro-6,8,12-octadecatrienoyl

(E,Z,Z)-6-nitro-7,9,12-octadecatrienoyl, (E,Z,Z)-7-nitro-5,9,12-octadecatrienoyl, (Z,E,Z)-9-nitro-6,10,12-octadecatrienoyl, (Z,E,Z)-10-nitro-6,8,12-octadecatrienoyl, (Z,Z,E)-12-nitro-6,9,13-octadecatrienoyl, (Z,Z,E)-13-nitro-6,9,11-octadecatrienoyl.

Mixtures of (E,Z,Z)-6-nitro-7,9,12-octadecatrienoyl, (E,Z,Z)-7-nitro-5,9,12-octadecatrienoyl, (Z,E,Z)-9-nitro-6,10,12-octadecatrienoyl, (Z,E,Z)-10-nitro-6,8,12-octadecatrienoyl, (Z,Z,E)-12-nitro-6,9,13-octadecatrienoyl, (Z,Z,E)-13-nitro-6,9,11-octadecatrienoyl,

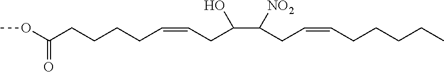

### (Z,Z)-9-hydroxy-10-nitro-6,12-octadecadienoyl

(Z,Z)-6-hydroxy-7-nitro-9,12-octadecadienoyl, (Z,Z)-7-hydroxy-6-nitro-9,12-octadecadienoyl, (Z,Z)-9-hydroxy-10-nitro-6,12-octadecadienoyl, (Z,Z)-10-hydroxy-9-nitro-6,12-octadecadienoyl, (Z,Z)-12-hydroxy-13-nitro-6,9-octadecadienoyl, (Z,Z)-13-hydroxy-12-nitro-6,9-octadecadienoyl.

Mixtures of (Z,Z)-6-hydroxy-7-nitro-9,12-octadecadienoyl, (Z,Z)-7-hydroxy-6-nitro-9,12-octadecadienoyl, (Z,Z)-9-hydroxy-10-nitro-6,12-octadecadienoyl, (Z,Z)-10-hydroxy-9-nitro-6,12-octadecadienoyl, (Z,Z)-12-hydroxy-13-nitro-6,9-octadecadienoyl, (Z,Z)-13-hydroxy-12-nitro-6,9-octadecadienoyl,

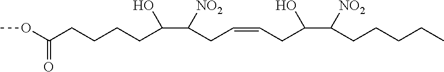

### (Z)-6,12-di hydroxy-7,13-dinitro-9-octadecenoyl

(Z)-6,9-dihydroxy-7,10-dinitro-12-octadecenoyl, (Z)-6,10-dihydroxy-7,9-dinitro-12-octadecenoyl, (Z)-7,9-dihydroxy-6,10-dinitro-12-octadecenoyl, (Z)-7,10-di hydroxy-6,9-dinitro-12-octadecenoyl, (Z)-6,12-dihydroxy-7,13-dinitro-9-octadecenoyl, (Z)-6,13-dihydroxy-7,12-dinitro-9-octadecenoyl, (Z)-7,12-dihydroxy-6,13-dinitro-9-octadecenoyl, (Z)-7,13-di hydroxy-6,12-dinitro-9-octadecenoyl, (Z)-9,12-dihydroxy-10,13-dinitro-6-octadecenoyl, (Z)-9,13-di hydroxy-10,12-dinitro-6-octadecenoyl, (Z)-10,12-dihydroxy-9,13-dinitro-6-octadecenoyl, (Z)-10,13-dihydroxy-9,12-dinitro-6-octadecenoyl.

Mixtures of (Z)-6,9-dihydroxy-7,10-dinitro-12-octadecenoyl, (Z)-6,10-di hydroxy-7,9-dinitro-12-octadecenoyl, (Z)-7,9-dihydroxy-6,10-dinitro-12-octadecenoyl, (Z)-7,10-dihydroxy-6,9-dinitro-12-octadecenoyl, (Z)-6,12-di hydroxy-7,13-dinitro-9-octadecenoyl, (Z)-6,13-di hydroxy-7,12-dinitro-9-octadecenoyl, (Z)-7,12-di hydroxy-6,13-dinitro-9-octadecenoyl, (Z)-7,13-dihydroxy-6,12-dinitro-9-octadecenoyl, (Z)-9,12-dihydroxy-10,13-dinitro-6-octadecenoyl, (Z)-9,13-dihydroxy-10,12-dinitro-6-octadecenoyl, (Z)-10,12-dihydroxy-9,13-dinitro-6-octadecenoyl, (Z)-10,13-dihydroxy-9,12-dinitro-6-octadecenoyl, 6,9,12-trihydroxy-7,10,13-trinitrooctadecanoyl, 6,9,13-trihydroxy-7,10,12-trinitrooctadecanoyl, 6,10,12-trihydroxy-7,9,13-trinitrooctadecanoyl, 6,10,13-trihydroxy-7,9,12-trinitrooctadecanoyl, 7,9,12-trihydroxy-6,10,13-trinitrooctadecanoyl, 7,9,13-trihydroxy-6,10,12-trinitrooctadecanoyl, 7,10,12-trihydroxy-6,9,13-trinitrooctadecanoyl, 7,10,13-trihydroxy-6,9,12-trinitrooctadecanoyl.

Mixtures of 6,9,12-trihydroxy-7,10,13-trinitrooctadecanoyl, 6,9,13-trihydroxy-7,10,12-trinitrooctadecanoyl, 6,10,12-trihydroxy-7,9,13-trinitrooctadecanoyl, 6,10,13-trihydroxy-7,9,12-trinitrooctadecanoyl, 7,9,12-trihydroxy-6,10,13-trinitrooctadecanoyl, 7,9,13-trihydroxy-6,10,12-trinitrooctadecanoyl, 7,10,12-trihydroxy-6,9,13-trinitrooctadecanoyl, 7,10,13-trihydroxy-6,9,12-trinitrooctadecanoyl,

Mixtures of (Z,Z)-6-hydroxy-7-nitro-9,12-octadecadienoyl, (Z,Z)-7-hydroxy-6-nitro-9,12-octadecadienoyl, (Z,Z)-9-hydroxy-10-nitro-6,12-octadecadienoyl, (Z,Z)-10-hydroxy-9-nitro-6,12-octadecadienoyl, (Z,Z)-12-hydroxy-13-nitro-6,9-octadecadienoyl, (Z,Z)-13-hydroxy-12-nitro-6,9-octadecadienoyl, (Z)-6,9-dihydroxy-7,10-dinitro-12-octadecenoyl, (Z)-6,10-dihydroxy-7,9-dinitro-12-octadecenoyl, (Z)-7,9-dihydroxy-6,10-dinitro-12-octadecenoyl, (Z)-7,10-dihydroxy-6,9-dinitro-12-octadecenoyl, (Z)-6,12-dihydroxy-7,13-dinitro-9-octadecenoyl, (Z)-6,13-dihydroxy-7,12-dinitro-9-octadecenoyl, (Z)-7,12-dihydroxy-6,13-dinitro-9-octadecenoyl, (Z)-7,13-dihydroxy-6,12-dinitro-9-octadecenoyl, (Z)-9,12-dihydroxy-10,13-dinitro-6-octadecenoyl, (Z)-9,13-dihydroxy-10,12-dinitro-6-octadecenoyl, (Z)-10,12-dihydroxy-9,13-dinitro-6-octadecenoyl, (Z)-10,13-dihydroxy-9,12-dinitro-6-octadecenoyl, 6,9,12-trihydroxy-7,10,13-trinitrooctadecanoyl, 6,9,13-trihydroxy-7,10,12-trinitrooctadecanoyl, 6,10,12-trihydroxy-7,9,13-trinitrooctadecanoyl, 6,10,13-trihydroxy-7,9,12-trinitrooctadecanoyl, 7,9,12-trihydroxy-6,10,13-trinitrooctadecanoyl, 7,9,13-trihydroxy-6,10,12-trinitrooctadecanoyl, 7,10,12-trihydroxy-6,9,13-trinitrooctadecanoyl, 7,10,13-trihydroxy-6,9,12-trinitrooctadecanoyl, 6-nitro-6-octadecen-9-inoyl, 7-nitro-6-octadecen-9-inoyl.

Mixtures of 6-nitro-6-octadecen-9-ynoyl and 7-nitro-6-octadecen-9-ynoyl, 6-nitro-7-octadecen-9-ynoyl, 7-nitro-5-octadecen-9-ynoyl.

Mixtures of 6-nitro-7-octadecen-9-ynoyl and 7-nitro-5-octadecen-9-ynoyl, 6-hydroxy-7-nitro-9-octadecynoyl, 7-hydroxy-6-nitro-9-octadecynoyl.

Mixtures of 6-hydroxy-7-nitro-9-octadecynoyl and 7-hydroxy-6-nitro-9-octadecynoyl. 11-nitro-11-octadecen-9-ynoyl, 12-nitro-11-octadecen-9-ynoyl.

Mixtures of 11-nitro-11-octadecen-9-ynoyl and 12-nitro-11-octadecen-9-ynoyl, 11-nitro-12-octadecen-9-ynoyl, 12-nitro-10-octadecen-9-ynoyl,

Mixtures of 11-nitro-12-octadecen-9-ynoyl and 12-nitro-10-octadecen-9-ynoyl, 11-hydroxy-12-nitro-9-octadecynoyl, 12-hydroxy-11-nitro-9-octadecynoyl.

Mixtures of 11-hydroxy-12-nitro-9-octadecynoyl and 12-hydroxy-11-nitro-9-octadecynoyl.

10-nitro-10-heptadecen-8-ynoyl, 11-nitro-10-heptadecen-8-ynoyl.

Mixtures of 10-nitro-10-heptadecen-8-ynoyl and 11-nitro-10-heptadecen-8-ynoyl, 10-nitro-11-heptadecen-8-ynoyl, 11-nitro-9-heptadecen-8-ynoyl.

Mixtures of 10-nitro-11-heptadecen-8-ynoyl and 11-nitro-9-heptadecen-8-ynoyl, 10-hydroxy-11-nitro-8-heptadecynoyl, 11-hydroxy-10-nitro-8-heptadecynoyl.

Mixtures of 10-hydroxy-11-nitro-8-heptadecynoyl and 11-hydroxy-10-nitro-8-heptadecynoyl.

9-nitro-9-octadecen-12-ynoyl, 10-nitro-9-octadecen-12-ynoyl.

Mixtures of 9-nitro-9-octadecen-12-ynoyl and 10-nitro-9-octadecen-12-ynoyl, 9-nitro-10-octadecen-12-ynoyl, 10-nitro-8-octadecen-12-ynoyl.

Mixtures of 9-nitro-10-octadecen-12-ynoyl and 10-nitro-8-octadecen-12-ynoyl, 9-hydroxy-10-nitro-12-octadecynoyl, 10-hydroxy-9-nitro-12-octadecynoyl.

Mixtures of 9-hydroxy-10-nitro-12-octadecynoyl and 10-hydroxy-9-nitro-12-octadecynoyl.

(E,Z,Z)-8-nitro-8,11,14-eicosatrienoyl, (E,Z,Z)-9-nitro-8,11,14-eicosatrienoyl, (Z,E,Z)-11-nitro-8,11,14-eicosatrienoyl, (Z,E,Z)-12-nitro-8,11,14-eicosatrienoyl, (Z,Z,E)-14-nitro-8,11,14-eicosatrienoyl, (Z,Z,E)-15-nitro-8,11,14-eicosatrienoyl.

Mixtures of (E,Z,Z)-8-nitro-8,11,14-eicosatrienoyl, (E,Z,Z)-9-nitro-8,11,14-eicosatrienoyl, (Z,E,Z)-11-nitro-8,11,14-eicosatrienoyl, (Z,E,Z)-12-nitro-8,11,14-eicosatrienoyl, (Z,Z,E)-14-nitro-8,11,14-eicosatrienoyl and (Z,Z,E)-15-nitro-8,11,14-eicosatrienoyl.

(E,E,Z)-8,11-dinitro-8,11,14-eicosatrienoyl, (E,E,Z)-8,12-dinitro-8,11,14-eicosatrienoyl, (E,E,Z)-9,11-dinitro-8,11,14-eicosatrienoyl, (E,E,Z)-9,12-dinitro-8,11,14-eicosatrienoyl, (E,Z,E)-8,14-dinitro-8,11,14-eicosatrienoyl, (E,Z,E)-8,15-dinitro-8,11,14-eicosatrienoyl, (E,Z,E)-9,14-dinitro-8,11,14-eicosatrienoyl, (E,Z,E)-9,15-dinitro-8,11,14-eicosatrienoyl, (Z,E,E)-11,14-dinitro-8,11,14-eicosatrienoyl, (Z,E,E)-11,15-dinitro-8,11,14-eicosatrienoyl, (Z,E,E)-12,14-dinitro-8,11,14-eicosatrienoyl, (Z,E,E)-13,15-dinitro-8,11,14-eicosatrienoyl.

Mixtures of (E,E,Z)-8,11-dinitro-8,11,14-eicosatrienoyl, (E,E,Z)-8,12-dinitro-8,11,14-eicosatrienoyl, (E,E,Z)-9,11-dinitro-8,11,14-eicosatrienoyl, (E,E,Z)-9,12-dinitro-8,11,14-eicosatrienoyl, (E,Z,E)-8,14-dinitro-8,11,14-eicosatrienoyl, (E,Z,E)-8,15-dinitro-8,11,14-eicosatrienoyl, (E,Z,E)-9,14-dinitro-8,11,14-eicosatrienoyl, (E,Z,E)-9,15-dinitro-8,11,14-eicosatrienoyl, (Z,E,E)-11,14-dinitro-8,11,14-eicosatrienoyl, (Z,E,E)-11,15-dinitro-8,11,14-eicosatrienoyl, (Z,E,E)-12,14-dinitro-8,11,14-eicosatrienoyl and (Z,E,E)-13,15-dinitro-8,11,14-eicosatrienoyl.

(E,E,E)-8,11,14-trinitro-8,11,14-eicosatrienoyl, (E,E,E)-8,11,15-trinitro-8,11,14-eicosatrienoyl, (E,E,E)-8,12,14-trinitro-8,11,14-eicosatrienoyl, (E,E,E)-8,12,15-trinitro-8,11,14-eicosatrienoyl, (E,E,E)-9,11,14-trinitro-8,11,14-eicosatrienoyl, (E,E,E)-9,11,15-trinitro-8,11,14-eicosatrienoyl, (E,E,E)-9,12,14-trinitro-8,11,14-eicosatrienoyl, (E,E,E)-9,12,15-trinitro-8,11,14-eicosatrienoyl.

Mixtures of (E,E,E)-8,11,14-trinitro-8,11,14-eicosatrienoyl, (E,E,E)-8,11,15-trinitro-8,11,14-eicosatrienoyl, (E,E,E)-8,12,14-trinitro-8,11,14-eicosatrienoyl, (E,E,E)-8,12,15-trinitro-8,11,14-eicosatrienoyl, (E,E,E)-9,11,14-trinitro-8,11,14-eicosatrienoyl, (E,E,E)-9,11,15-trinitro-8,11,14-eicosatrienoyl, (E,E,E)-9,12,14-trinitro-8,11,14-eicosatrienoyl and (E,E,E)-9,12,15-trinitro-8,11,14-eicosatrienoyl.

Mixtures of (E,Z,Z)-8-nitro-8,11,14-eicosatrienoyl, (E,Z,Z)-9-nitro-8,11,14-eicosatrienoyl, (Z,E,Z)-11-nitro-8,11,14-eicosatrienoyl, (Z,E,Z)-12-nitro-8,11,14-eicosatrienoyl, (Z,Z,E)-14-nitro-8,11,14-eicosatrienoyl, (Z,Z,E)-15-nitro-8,11,14-eicosatrienoyl, (E,E,Z)-8,11-dinitro-8,11,14-eicosatrienoyl, (E,E,Z)-8,12-dinitro-8,11,14-eicosatrienoyl, (E,E,Z)-9,11-dinitro-8,11,14-eicosatrienoyl, (E,E,Z)-9,12-dinitro-8,11,14-eicosatrienoyl, (E,Z,E)-8,14-dinitro-8,11,14-eicosatrienoyl, (E,Z,E)-8,15-dinitro-8,11,14-eicosatrienoyl, (E,Z,E)-9,14-dinitro-8,11,14-eicosatrienoyl, (E,Z,E)-9,15-dinitro-8,11,14-eicosatrienoyl, (Z,E,E)-11,14-dinitro-8,11,14-eicosatrienoyl, (Z,E,E)-11,15-dinitro-8,11,14-eicosatrienoyl, (Z,E,E)-12,14-dinitro-8,11,14-eicosatrienoyl, (Z,E,E)-13,15-dinitro-8,11,14-eicosatrienoyl, (E,E,E)-8,11,14-trinitro-8,11,14-eicosatrienoyl, (E,E,E)-8,11,15-trinitro-8,11,14-eicosatrienoyl, (E,E,E)-8,12,14-trinitro-8,11,14-eicosatrienoyl, (E,E,E)-8,12,15-trinitro-8,11,14-eicosatrienoyl, (E,E,E)-9,11,14-trinitro-8,11,14-eicosatrienoyl, (E,E,E)-9,11,15-trinitro-8,11,14-eicosatrienoyl, (E,E,E)-9,12,14-trinitro-8,11,14-eicosatrienoyl and (E,E,E)-9,12,15-trinitro-8,11,14-eicosatrienoyl.

(Z,Z)-8-hydroxy-9-nitro-11,14-eicosadienoyl, (Z,Z)-9-hydroxy-8-nitro-11,14-eicosadienoyl, (Z,Z)-11-hydroxy-12-nitro-8,14-eicosadienoyl, (Z,Z)-12-hydroxy-11-nitro-8,14-eicosadienoyl, (Z,Z)-14-hydroxy-15-nitro-8,11-eicosadienoyl, (Z,Z)-15-hydroxy-14-nitro-8,11-eicosadienoyl.

Mixtures of (Z,Z)-8-hydroxy-9-nitro-11,14-eicosadienoyl, (Z,Z)-9-hydroxy-8-nitro-11,14-eicosadienoyl, (Z,Z)-11-hydroxy-12-nitro-8,14-eicosadienoyl, (Z,Z)-12-hydroxy-11-nitro-8,14-eicosadienoyl, (Z,Z)-14-hydroxy-15-nitro-8,11-eicosadienoyl and (Z,Z)-15-hydroxy-14-nitro-8,11-eicosadienoyl.

(Z)-8,11-dihydroxy-9,12-dinitro-14-eicosenoyl, (Z)-8,12-dihydroxy-9,11-dinitro-14-eicosenoyl, (Z)-9,11-dihydroxy-8,12-dinitro-14-eicosenoyl, (Z)-9,12-dihydroxy-8,11-dinitro-14-eicosenoyl, (Z)-8,14-dihydroxy-9,15-dinitro-11-eicosenoyl, (Z)-8,15-dihydroxy-9,14-dinitro-11-eicosenoyl, (Z)-9,14-dihydroxy-8,15-dinitro-11-eicosenoyl, (Z)-9,15-dihydroxy-8,14-dinitro-11-eicosenoyl, (Z)-11,14-dihydroxy-12,15-dinitro-8-eicosenoyl, (Z)-11,15-dihydroxy-12,14-dinitro-8-eicosenoyl, (Z)-12,14-dihydroxy-11,15-dinitro-8-eicosenoyl, (Z)-12,15-dihydroxy-11,14-dinitro-8-eicosenoyl.

Mixtures of (Z)-8,11-dihydroxy-9,12-dinitro-14-eicosenoyl, (Z)-8,12-dihydroxy-9,11-dinitro-14-eicosenoyl, (Z)-9,11-dihydroxy-8,12-dinitro-14-eicosenoyl, (Z)-9,12-dihydroxy-8,11-dinitro-14-eicosenoyl, (Z)-8,14-dihydroxy-9,15-dinitro-11-eicosenoyl, (Z)-8,15-dihydroxy-9,14-dinitro-11-eicosenoyl, (Z)-9,14-dihydroxy-8,15-dinitro-11-eicosenoyl, (Z)-9,15-dihydroxy-8,14-dinitro-11-eicosenoyl, (Z)-11,14-dihydroxy-12,15-dinitro-8-eicosenoyl, (Z)-11,15-dihydroxy-12,14-dinitro-8-eicosenoyl, (Z)-12,14-dihydroxy-11,15-dinitro-8-eicosenoyl and (Z)-12,15-dihydroxy-11,14-dinitro-8-eicosenoyl.

8,11,14-trihydroxy-9,12,15-trinitroeicosanoyl, 8,11,15-trihydroxy-9,12,14-trinitroeicosanoyl, 8,12,14-trihydroxy-9,11,15-trinitroeicosanoyl, 8,12,15-trihydroxy-9,11,14-trinitroeicosanoyl, 9,11,14-trihydroxy-8,12,15-trinitroeicosanoyl, 9,11,15-trihydroxy-8,12,14-trinitroeicosanoyl, 9,12,14-trihydroxy-8,11,15-trinitroeicosanoyl, 9,12,15-trihydroxy-8,11,14-trinitroeicosanoyl.

Mixtures of 8,11,14-trihydroxy-9,12,15-trinitroeicosanoyl, 8,11,15-trihydroxy-9,12,14-trinitroeicosanoyl, 8,12,14-trihydroxy-9,11,15-trinitroeicosanoyl, 8,12,15-trihydroxy-9,11,14-trinitroeicosanoyl, 9,11,14-trihydroxy-8,12,15-trinitroeicosanoyl, 9,11,15-trihydroxy-8,12,14-trinitroeicosanoyl, 9,12,14-trihydroxy-8,11,15-trinitroeicosanoyl and 9,12,15-trihydroxy-8,11,14-trinitroeicosanoyl.

Mixtures of (Z,Z)-8-hydroxy-9-nitro-11,14-eicosadienoyl, (Z,Z)-9-hydroxy-8-nitro-11,14-eicosadienoyl, (Z,Z)-11-hydroxy-12-nitro-8,14-eicosadienoyl, (Z,Z)-12-hydroxy-11-nitro-8,14-eicosadienoyl, (Z,Z)-14-hydroxy-15-nitro-8,11-eicosadienoyl, (Z,Z)-15-hydroxy-14-nitro-8,11-eicosadienoyl, (Z)-8,11-dihydroxy-9,12-dinitro-14-eicosenoyl, (Z)-8,12-dihydroxy-9,11-dinitro-14-eicosenoyl, (Z)-9,11-dihydroxy-8,12-dinitro-14-eicosenoyl, (Z)-9,12-dihydroxy-11,15-dinitro-14-eicosenoyl, (Z)-8,14-dihydroxy-9,15-dinitro-11-eicosenoyl, (Z)-8,15-dihydroxy-9,14-dinitro-11-eicosenoyl, (Z)-9,14-dihydroxy-8,15-dinitro-11-eicosenoyl, (Z)-9,15-dihydroxy-8,14-dinitro-11-eicosenoyl, (Z)-11,14-dihydroxy-12,15-dinitro-8-eicosenoyl, (Z)-11,15-dihydroxy-12,14-dinitro-8-eicosenoyl, (Z)-12,14-dihydroxy-11,15-dinitro-8-eicosenoyl, (Z)-12,15-dihydroxy-11,14-dinitro-8-eicosenoyl, 8,11,14-trihydroxy-9,12,15-trinitroeicosanoyl, 8,11,15-trihydroxy-9,12,14-trinitroeicosanoyl, 8,12,14-trihydroxy-9,11,15-trinitroeicosanoyl, 8,12,15-trihydroxy-9,11,14-trinitroeicosanoyl, 9,11,14-trihydroxy-8,12,15-trinitroeicosanoyl, 9,11,15-trihydroxy-8,12,14-trinitroeicosanoyl, 9,12,14-trihydroxy-8,11,15-trinitroeicosanoyl and 9,12,15-trihydroxy-8,11,14-trinitroeicosanoyl.

(E,Z,Z)-5-nitro-5,8,11-eicosatrienoyl, (E,Z,Z)-6-nitro-5,8,11-eicosatrienoyl, (Z,E,Z)-8-nitro-5,8,11-eicosatrienoyl, (Z,E,Z)-9-nitro-5,8,11-eicosatrienoyl, (Z,Z,E)-11-nitro-5,8,11-eicosatrienoyl, (Z,Z,E)-12-nitro-5,8,11-eicosatrienoyl.

Mixtures of (E,Z,Z)-5-nitro-5,8,11-eicosatrienoyl, (E,Z,Z)-6-nitro-5,8,11-eicosatrienoyl, (Z,E,Z)-8-nitro-5,8,11-eicosatrienoyl, (Z,E,Z)-9-nitro-5,8,11-eicosatrienoyl, (Z,Z,E)-11-nitro-5,8,11-eicosatrienoyl and (Z,Z,E)-12-nitro-5,8,11-eicosatrienoyl.

(E,E,Z)-5,8-dinitro-5,8,11-eicosatrienoyl, (E,E,Z)-5,9-dinitro-5,8,11-eicosatrienoyl, (E,E,Z)-6,8-dinitro-5,8,11-eicosatrienoyl, (E,E,Z)-6,9-dinitro-5,8,11-eicosatrienoyl, (E,Z,E)-5,11-dinitro-5,8,11-eicosatrienoyl, (E,Z,E)-5,12-dinitro-5,8,11-eicosatrienoyl, (E,Z,E)-6,11-dinitro-5,8,11-eicosatrienoyl, (E,Z,E)-6,12-dinitro-5,8,11-eicosatrienoyl, (Z,E,E)-8,11-dinitro-5,8,11-eicosatrienoyl, (Z,E,E)-8,12-dinitro-5,8,11-eicosatrienoyl, (Z,E,E)-9,11-dinitro-5,8,11-eicosatrienoyl, (Z,E,E)-9,12-dinitro-5,8,11-eicosatrienoyl.

Mixtures of (E,E,Z)-5,8-dinitro-5,8,11-eicosatrienoyl, (E,E,Z)-5,9-dinitro-5,8,11-eicosatrienoyl, (E,E,Z)-6,8-dinitro-5,8,11-eicosatrienoyl, (E,E,Z)-6,9-dinitro-5,8,11-eicosatrienoyl, (E,Z,E)-5,11-dinitro-5,8,11-eicosatrienoyl, (E,Z,E)-5,12-dinitro-5,8,11-eicosatrienoyl, (E,Z,E)-6,11-dinitro-5,8,11-eicosatrienoyl, (E,Z,E)-6,12-dinitro-5,8,11-eicosatrienoyl, (Z,E,E)-8,11-dinitro-5,8,11-eicosatrienoyl, (Z,E,E)-8,12-dinitro-5,8,11-eicosatrienoyl, (Z,E,E)-9,11-dinitro-5,8,11-eicosatrienoyl and (Z,E,E)-9,12-dinitro-5,8,11-eicosatrienoyl.

(E,E,E)-5,8,11-trinitro-5,8,11-eicosatrienoyl, (E,E,E)-5,8,12-trinitro-5,8,11-eicosatrienoyl, (E,E,E)-5,9,11-trinitro-5,8,11-eicosatrienoyl, (E,E,E)-5,9,12-trinitro-5,8,11-eicosatrienoyl, (E,E,E)-6,8,11-trinitro-5,8,11-eicosatrienoyl, (E,E,E)-6,8,12-trinitro-5,8,11-eicosatrienoyl, (E,E,E)-6,9,11-trinitro-5,8,11-eicosatrienoyl, (E,E,E)-6,9,12-trinitro-5,8,11-eicosatrienoyl.

Mixtures of (E,E,E)-5,8,11-trinitro-5,8,11-eicosatrienoyl, (E,E,E)-5,8,12-trinitro-5,8,11-eicosatrienoyl, (E,E,E)-5,9,11-trinitro-5,8,11-eicosatrienoyl, (E,E,E)-5,9,12-trinitro-5,8,11-eicosatrienoyl, (E,E,E)-6,8,11-trinitro-5,8,11-eicosatrienoyl, (E,E,E)-6,8,12-trinitro-5,8,11-eicosatrienoyl and (E,E,E)-6,9,11-trinitro-5,8,11-eicosatrienoyl, (E,E,E)-6,9,12-trinitro-5,8,11-eicosatrienoyl.

Mixtures of (E,Z,Z)-5-nitro-5,8,11-eicosatrienoyl, (E,Z,Z)-6-nitro-5,8,11-eicosatrienoyl, (Z,E,Z)-8-nitro-5,8,11-eicosatrienoyl, (Z,E,Z)-9-nitro-5,8,11-eicosatrienoyl, (Z,Z,E)-11-nitro-5,8,11-eicosatrienoyl, (Z,Z,E)-12-nitro-5,8,11-eicosatrienoyl, (E,E,Z)-5,8-dinitro-5,8,11-eicosatrienoyl, (E,E,Z)-5,9-dinitro-5,8,11-eicosatrienoyl, (E,E,Z)-6,8-dinitro-5,8,11-eicosatrienoyl, (E,E,Z)-6,9-dinitro-5,8,11-eicosatrienoyl, (E,Z,E)-5,11-dinitro-5,8,11-eicosatrienoyl, (E,Z,E)-5,12-dinitro-5,8,11-eicosatrienoyl, (E,Z,E)-6,11-dinitro-5,8,11-eicosatrienoyl, (E,Z,E)-6,12-dinitro-5,8,11-eicosatrienoyl, (Z,E,E)-8,11-dinitro-5,8,11-eicosatrienoyl, (Z,E,E)-8,12-dinitro-5,8,11-eicosatrienoyl, (Z,E,E)-9,11-dinitro-5,8,11-eicosatrienoyl, (Z,E,E)-13,12-dinitro-5,8,11-eicosatrienoyl, (E,E,E)-5,8,11-trinitro-5,8,11-eicosatrienoyl, (E,E,E)-5,8,12-trinitro-5,8,11-eicosatrienoyl, (E,E,E)-5,9,11-trinitro-5,8,11-eicosatrienoyl, (E,E,E)-5,9,12-trinitro-5,8,11-eicosatrienoyl, (E,E,E)-6,8,11-trinitro-5,8,11-eicosatrienoyl, (E,E,E)-6,8,12-trinitro-5,8,11-eicosatrienoyl and (E,E,E)-6,9,11-trinitro-5,8,11-eicosatrienoyl, (E,E,E)-6,9,12-trinitro-5,8,11-eicosatrienoyl.

(Z,Z)-5-hydroxy-6-nitro-8,11-eicosadienoyl, (Z,Z)-6-hydroxy-5-nitro-8,11-eicosadienoyl, (Z,Z)-8-hydroxy-9-nitro-5,11-eicosadienoyl, (Z,Z)-9-hydroxy-8-nitro-5,11-eicosadienoyl, (Z,Z)-11-hydroxy-12-nitro-5,8-eicosadienoyl, (Z,Z)-12-hydroxy-11-nitro-5,8-eicosadienoyl.

Mixtures of (Z,Z)-5-hydroxy-6-nitro-8,11-eicosadienoyl, (Z,Z)-6-hydroxy-5-nitro-8,11-eicosadienoyl, (Z,Z)-8-hydroxy-9-nitro-5,11-eicosadienoyl, (Z,Z)-9-hydroxy-8-nitro-5,11-eicosadienoyl and (Z,Z)-11-hydroxy-12-nitro-5,8-eicosadienoyl, (Z,Z)-12-hydroxy-11-nitro-5,8-eicosadienoyl.

(Z)-5,8-dihydroxy-6,9-dinitro-11-eicosenoyl, (Z)-5,9-dihydroxy-6,8-dinitro-11-eicosenoyl, (Z)-6,8-dihydroxy-5,9-dinitro-11-eicosenoyl, (Z)-6,9-dihydroxy-5,8-dinitro-11-eicosenoyl, (Z)-5,11-dihydroxy-6,12-dinitro-8-eicosenoyl, (Z)-5,12-dihydroxy-6,11-dinitro-8-eicosenoyl, (Z)-6,11-dihydroxy-5,12-dinitro-8-eicosenoyl, (Z)-6,12-dihydroxy-5,11-dinitro-8-eicosenoyl, (Z)-8,11-dihydroxy-9,12-dinitro-5-eicosenoyl, (Z)-8,12-dihydroxy-9,11-dinitro-5-eicosenoyl, (Z)-9,11-dihydroxy-8,12-dinitro-5-eicosenoyl, (Z)-9,12-dihydroxy-8,11-dinitro-5-eicosenoyl.

Mixtures of (Z)-5,8-dihydroxy-6,9-dinitro-11-eicosenoyl, (Z)-5,9-dihydroxy-6,8-dinitro-11-eicosenoyl, (Z)-6,8-dihydroxy-5,9-dinitro-11-eicosenoyl, (Z)-6,9-dihydroxy-5,8-dinitro-11-eicosenoyl, (Z)-5,11-dihydroxy-6,12-dinitro-8-eicosenoyl, (Z)-5,12-dihydroxy-6,11-dinitro-8-eicosenoyl, (Z)-6,11-dihydroxy-5,12-dinitro-8-eicosenoyl, (Z)-6,12-dihydroxy-5,11-dinitro-8-eicosenoyl, (Z)-8,11-dihydroxy-9,12-dinitro-5-eicosenoyl, (Z)-8,12-dihydroxy-9,11-dinitro-5-eicosenoyl, (Z)-9,11-dihydroxy-8,12-dinitro-5-eicosenoyl and (Z)-9,12-dihydroxy-8,11-dinitro-5-eicosenoyl.

5,8,11-trihydroxy-6,9,12-trinitroeicosanoyl, 5,8,12-trihydroxy-6,9,11-trinitroeicosanoyl, 5,9,11-trihydroxy-6,8,12-trinitroeicosanoyl, 5,9,12-trihydroxy-6,8,11-trinitroeicosanoyl, 6,8,11-trihydroxy-5,9,12-trinitroeicosanoyl, 6,8,12-trihydroxy-5,9,11-trinitroeicosanoyl, 6,9,11-trihydroxy-5,8,12-trinitroeicosanoyl, 6,9,12-trihydroxy-5,8,11-trinitroeicosanoyl.

Mixtures of 5,8,11-trihydroxy-6,9,12-trinitroeicosanoyl, 5,8,12-trihydroxy-6,9,11-trinitroeicosanoyl, 5,9,11-trihydroxy-6,8,12-trinitroeicosanoyl, 5,9,12-tri hydroxy-6,8,11-trinitroeicosanoyl, 6,8,11-trihydroxy-5,9,12-trinitroeicosanoyl, 6,8,12-tri hydroxy-5,9,11-trinitroeicosanoyl, 6,9,11-trihydroxy-5,8,12-trinitroeicosanoyl and 6,9,12-trihydroxy-5,8,11-trinitroeicosanoyl.

Mixtures of (Z,Z)-5-hydroxy-6-nitro-8,11-eicosadienoyl, (Z,Z)-6-hydroxy-5-nitro-8,11-eicosadienoyl, (Z,Z)-8-hydroxy-9-nitro-5,11-eicosadienoyl, (Z,Z)-9-hydroxy-8-nitro-5,11-eicosadienoyl, (Z,Z)-11-hydroxy-12-nitro-5,8-eicosadienoyl, (Z,Z)-12-hydroxy-11-nitro-5,8-eicosadienoyl, (Z)-5,8-dihydroxy-6,9-dinitro-11-eicosenoyl, (Z)-5,9-dihydroxy-6,8-dinitro-11-eicosenoyl, (Z)-6,8-dihydroxy-5,9-dinitro-11-eicosenoyl, (Z)-6,9-dihydroxy-5,8-dinitro-11-eicosenoyl, (Z)-5,11-dihydroxy-6,12-dinitro-8-eicosenoyl, (Z)-5,12-dihydroxy-6,11-dinitro-8-eicosenoyl, (Z)-6,11-dihydroxy-5,12-dinitro-8-eicosenoyl, (Z)-6,12-dihydroxy-5,11-dinitro-8-eicosenoyl, (Z)-8,11-dihydroxy-9,12-dinitro-5-eicosenoyl, (Z)-8,12-dihydroxy-9,11-dinitro-5-eicosenoyl, (Z)-9,11-dihydroxy-8,12-dinitro-5-eicosenoyl, (Z)-9,12-dihydroxy-8,11-dinitro-5-eicosenoyl, 5,8,11-trihydroxy-6,9,12-trinitroeicosanoyl, 5,8,12-trihydroxy-6,9,11-trinitroeicosanoyl, 5,9,11-trihydroxy-6,8,12-trinitroeicosanoyl, 5,9,12-trihydroxy-6,8,11-trinitroeicosanoyl, 6,8,11-trihydroxy-5,9,12-trinitroeicosanoyl, 6,8,12-trihydroxy-5,9,11-trinitroeicosanoyl, 6,9,11-trihydroxy-5,8,12-trinitroeicosanoyl and 6,9,12-trihydroxy-5,8,11-trinitroeicosa noyl.

(E,Z,Z)-9-nitro-9,12,15-octadecatrienoyl, (E,Z,Z)-10-nitro-9,12,15-octadecatrienoyl, (Z,E,Z)-12-nitro-9,12,15-octadecatrienoyl, (Z,E,Z)-13-nitro-9,12,15-octadecatrienoyl, (Z,Z,E)-15-nitro-9,12,15-octadecatrienoyl, (Z,Z,E)-16-nitro-9,12,15-octadecatrienoyl.

Mixtures of (E,Z,Z)-9-nitro-9,12,15-octadecatrienoyl, (E,Z,Z)-10-nitro-9,12,15-octadecatrienoyl, (Z,E,Z)-12-nitro-9,12,15-octadecatrienoyl, (Z,E,Z)-13-nitro-9,12,15-octadecatrienoyl, (Z,Z,E)-15-nitro-9,12,15-octadecatrienoyl and (Z,Z,E)-16-nitro-9,12,15-octadecatrienoyl.

(E,E,Z)-9,12-dinitro-9,12,15-octadecatrienoyl, (E,E,Z)-9,13-dinitro-9,12,15-octadecatrienoyl, (E,E,Z)-10,12-dinitro-9,12,15-octadecatrienoyl, (E,E,Z)-10,13-dinitro-9,12,15-octadecatrienoyl, (E,Z,E)-9,15-dinitro-9,12,15-octadecatrienoyl, (E,Z,E)-9,16-dinitro-9,12,15-octadecatrienoyl, (E,Z,E)-10,15-dinitro-9,12,15-octadecatrienoyl, (E,Z,E)-10,16-dinitro-9,12,15-octadecatrienoyl, (Z,E,E)-12,15-dinitro-9,12,15-octadecatrienoyl, (Z,E,E)-12,16-dinitro-9,12,15-octadecatrienoyl, (Z,E,E)-13,15-dinitro-9,12,15-octadecatrienoyl, (Z,E,E)-13,16-dinitro-9,12,15-octadecatrienoyl.

Mixtures of (E,E,Z)-9,12-dinitro-9,12,15-octadecatrienoyl, (E,E,Z)-9,13-dinitro-9,12,15-octadecatrienoyl, (E,E,Z)-10,12-dinitro-9,12,15-octadecatrienoyl, (E,E,Z)-10,13-dinitro-9,12,15-octadecatrienoyl, (E,Z,E)-9,15-dinitro-9,12,15-octadecatrienoyl, (E,Z,E)-9,19-dinitro-9,12,15-octadecatrienoyl, (E,Z,E)-10,15-dinitro-9,12,15-octadecatrienoyl, (E,Z,E)-10,16-dinitro-9,12,15-octadecatrienoyl, (Z,E,E)-12,15-dinitro-9,12,15-octadecatrienoyl, (Z,E,E)-12,16-dinitro-9,12,15-octadecatrienoyl, (Z,E,E)-13,15-dinitro-9,12,15-octadecatrienoyl and (Z,E,E)-13,16-dinitro-9,12,15-octadecatrienoyl.

(E,E,E)-9,12,15-trinitro-9,12,15-octadecatrienoyl, (E,E,E)-9,12,16-trinitro-9,12,15-octadecatrienoyl, (E,E,E)-9,13,15-trinitro-9,12,15-octadecatrienoyl, (E,E,E)-9,13,16-trinitro-9,12,15-octadecatrienoyl, (E,E,E)-10,12,15-trinitro-9,12,15-octadecatrienoyl, (E,E,E)-10,12,19-trinitro-9,12,15-octadecatrienoyl, (E,E,E)-10,13,15-trinitro-9,12,15-octadecatrienoyl, (E,E,E)-10,13,16-trinitro-9,12,15-octadecatrienoyl.

Mixtures of (E,E,E)-9,12,15-trinitro-9,12,15-octadecatrienoyl, (E,E,E)-9,12,16-trinitro-9,12,15-octadecatrienoyl, (E,E,E)-9,13,15-trinitro-9,12,15-octadecatrienoyl, (E,E,E)-9,13,16-trinitro-9,12,15-octadecatrienoyl, (E,E,E)-10,12,15-trinitro-9,12,15-octadecatrienoyl, (E,E,E)-10,12,16-trinitro-9,12,15-octadecatrienoyl, (E,E,E)-10,13,15-trinitro-9,12,15-octadecatrienoyl and (E,E,E)-10,13,16-trinitro-9,12,15-octadecatrienoyl.

Mixtures of (E,Z,Z)-9-nitro-9,12,15-octadecatrienoyl, (E,Z,Z)-10-nitro-9,12,15-octadecatrienoyl, (Z,E,Z)-12-nitro-9,12,15-octadecatrienoyl, (Z,E,Z)-13-nitro-9,12,15-octadecatrienoyl, (Z,Z,E)-15-nitro-9,12,15-octadecatrienoyl, (Z,Z,E)-19-nitro-9,12,15-octadecatrienoyl, (E,E,Z)-9,12-dinitro-9,12,15-octadecatrienoyl, (E,E,Z)-9,13-dinitro-9,12,15-octadecatrienoyl, (E,E,Z)-10,12-dinitro-9,12,15-octadecatrienoyl, (E,E,Z)-10,13-dinitro-9,12,15-octadecatrienoyl, (E,Z,E)-9,15-dinitro-9,12,15-octadecatrienoyl, (E,Z,E)-9,19-dinitro-9,12,15-octadecatrienoyl, (E,Z,E)-10,15-dinitro-9,12,15-octadecatrienoyl, (E,Z,E)-10,16-dinitro-9,12,15-octadecatrienoyl, (Z,E,E)-12,15-dinitro-9,12,15-octadecatrienoyl, (Z,E,E)-12,16-dinitro-9,12,15-octadecatrienoyl, (Z,E,E)-13,15-dinitro-9,12,15-octadecatrienoyl, (Z,E,E)-13,16-dinitro-9,12,15-octadecatrienoyl, (E,E,E)-9,12,15-trinitro-9,12,15-octadecatrienoyl, (E,E,E)-9,12,16-trinitro-9,12,15-octadecatrienoyl, (E,E,E)-9,13,15-trinitro-9,12,15-octadecatrienoyl, (E,E,E)-9,13,16-trinitro-9,12,15-octadecatrienoyl, (E,E,E)-10,12,15-trinitro-9,12,15-octadecatrienoyl, (E,E,E)-10,12,16-trinitro-9,12,15-octadecatrienoyl, (E,E,E)-10,13,15-trinitro-9,12,15-octadecatrienoyl and (E,E,E)-10,13,16-trinitro-9,12,15-octadecatrienoyl.

(Z,Z)-9-hydroxy-10-nitro-12,15-octadecadienoyl, (Z,Z)-10-hydroxy-9-nitro-12,15-octadecadienoyl, (Z,Z)-12-hydroxy-13-nitro-9,15-octadecadienoyl, (Z,Z)-13-hydroxy-12-nitro-9,15-octadecadienoyl, (Z,Z)-15-hydroxy-16-nitro-9,12-octadecadienoyl, (Z,Z)-19-hydroxy-15-nitro-9,12-octadecadienoyl.

Mixtures of (Z,Z)-9-hydroxy-10-nitro-12,15-octadecadienoyl, (Z,Z)-10-hydroxy-9-nitro-12,15-octadecadienoyl, (Z,Z)-12-hydroxy-13-nitro-9,15-octadecadienoyl, (Z,Z)-13-hydroxy-12-nitro-9,15-octadecadienoyl, (Z,Z)-15-hydroxy-16-nitro-9,12-octadecadienoyl and (Z,Z)-16-hydroxy-15-nitro-9,12-octadecadienoyl.

(Z)-9,12-dihydroxy-10,13-dinitro-15-octadecenoyl, (Z)-9,13-dihydroxy-10,12-dinitro-15-octadecenoyl, (Z)-10,12-dihydroxy-9,13-dinitro-15-octadecenoyl, (Z)-10,13-dihydroxy-9,12-dinitro-15-octadecenoyl, (Z)-9,15-dihydroxy-10,16-dinitro-12-octadecenoyl, (Z)-9,16-dihydroxy-10,15-dinitro-12-octadecenoyl, (Z)-10,15-dihydroxy-9,16-dinitro-12-octadecenoyl, (Z)-10,16-dihydroxy-9,15-dinitro-12-octadecenoyl, (Z)-12,15-dihydroxy-13,16-dinitro-9-octadecenoyl, (Z)-12,16-dihydroxy-13,15-dinitro-9-octadecenoyl, (Z)-13,15-dihydroxy-12,16-dinitro-9-octadecenoyl, (Z)-13,16-dihydroxy-12,15-dinitro-9-octadecenoyl.

Mixtures of (Z)-9,12-dihydroxy-10,13-dinitro-15-octadecenoyl, (Z)-9,13-dihydroxy-10,12-dinitro-15-octadecenoyl, (Z)-10,12-dihydroxy-9,13-dinitro-15-octadecenoyl, (Z)-10,13-dihydroxy-9,12-dinitro-15-octadecenoyl, (Z)-9,15-dihydroxy-10,16-dinitro-12-octadecenoyl, (Z)-9,16-dihydroxy-10,15-dinitro-12-octadecenoyl, (Z)-10,15-dihydroxy-9,16-dinitro-12-octadecenoyl, (Z)-10,16-dihydroxy-9,15-dinitro-12-octadecenoyl, (Z)-12,15-dihydroxy-13,16-dinitro-9-octadecenoyl, (Z)-12,16-dihydroxy-13,15-dinitro-9-octadecenoyl, (Z)-13,15-dihydroxy-12,16-dinitro-9-octadecenoyl and (Z)-13,16-dihydroxy-12,15-dinitro-9-octadecenoyl.

9,12,15-trihydroxy-10,13,16-trinitrooctadecanoyl, 9,12,16-trihydroxy-10,13,15-trinitrooctadecanoyl, 9,13,15-trihydroxy-10,12,16-trinitrooctadecanoyl, 8,13,16-trihydroxy-10,12,15-trinitrooctadecanoyl, 10,12,15-trihydroxy-9,13,16-trinitrooctadecanoyl, 10,12,16-trihydroxy-9,13,15-trinitrooctadecanoyl, 10,13,15-trihydroxy-9,12,16-trinitrooctadecanoyl, 10,13,16-trihydroxy-9,12,15-trinitrooctadecanoyl.

Mixtures of 9,12,15-trihydroxy-10,13,16-trinitrooctadecanoyl, 9,12,16-trihydroxy-10,13,15-trinitrooctadecanoyl, 9,13,15-trihydroxy-10,12,16-trinitrooctadecanoyl, 9,13,16-trihydroxy-10,12,15-trinitrooctadecanoyl, 10,12,15-trihydroxy-9,13,16-trinitrooctadecanoyl, 10,12,16-trihydroxy-9,13,15-trinitrooctadecanoyl, 10,13,15-trihydroxy-9,12,16-trinitrooctadecanoyl and 10,13,16-trihydroxy-9,12,15-trinitrooctadecanoyl.

Mixtures of (Z,Z)-9-hydroxy-10-nitro-12,15-octadecadienoyl, (Z,Z)-10-hydroxy-9-nitro-12,15-octadecadienoyl, (Z,Z)-12-hydroxy-13-nitro-9,15-octadecadienoyl, (Z,Z)-13-hydroxy-12-nitro-9,15-octadecadienoyl, (Z,Z)-15-hydroxy-16-nitro-9,12-octadecadienoyl, (Z,Z)-16-hydroxy-15-nitro-9,12-octadecadienoyl, (Z)-9,12-dihydroxy-10,13-dinitro-15-octadecenoyl, (Z)-9,13-dihydroxy-10,12-dinitro-15-octadecenoyl, (Z)-10,12-dihydroxy-9,13-dinitro-15-octadecenoyl, (Z)-10,13-dihydroxy-9,12-dinitro-15-octadecenoyl, (Z)-9,15-dihydroxy-10,16-dinitro-12-octadecenoyl, (Z)-9,16-dihydroxy-10,15-dinitro-12-octadecenoyl, (Z)-10,15-dihydroxy-9,16-dinitro-12-octadecenoyl, (Z)-10,16-dihydroxy-9,15-dinitro-12-octadecenoyl, (Z)-12,15-dihydroxy-13,16-dinitro-9-octadecenoyl, (Z)-12,16-dihydroxy-13,15-dinitro-9-octadecenoyl, (Z)-13,15-dihydroxy-12,16-dinitro-9-octadecenoyl, (Z)-13,16-dihydroxy-12,15-dinitro-9-octadecenoyl, 9,12,15-trihydroxy-10,13,16-trinitrooctadecanoyl, 9,12,16-trihydroxy-10,13,15-trinitrooctadecanoyl, 9,13,15-trihydroxy-10,12,16-trinitrooctadecanoyl, 9,13,16-trihydroxy-10,12,15-trinitrooctadecanoyl, 10,12,15-trihydroxy-9,13,16-trinitrooctadecanoyl, 10,12,16-trihydroxy-9,13,15-trinitrooctadecanoyl, 10,13,15-trihydroxy-9,12,16-trinitrooctadecanoyl and 10,13,16-trihydroxy-9,12,15-trinitrooctadecanoyl.

(E,Z,Z,Z)-5-nitro-5,8,11,14-eicosatetraenoyl, (E,Z,Z,Z)-6-nitro-5,8,11,14-eicosatetraenoyl, (Z,E,Z,Z)-8-nitro-5,8,11,14-eicosatetraenoyl, (Z,E,Z,Z)-9-nitro-5,8,11,14-eicosatetraenoyl, (Z,Z,E,Z)-11-nitro-5,8,11,14-eicosatetraenoyl, (Z,Z,E,Z)-12-nitro-5,8,11,14-eicosatetraenoyl. (Z,Z,Z,E)-14-nitro-5,8,11,14-eicosatetraenoyl, (Z,Z,Z,E)-15-nitro-5,8,11,14-eicosatetraenoyl.

Mixtures of (E,Z,Z,Z)-5-nitro-5,8,11,14-eicosatetraenoyl, (E,Z,Z,Z)-6-nitro-5,8,11,14-eicosatetraenoyl, (Z,E,Z,Z)-8-nitro-5,8,11,14-eicosatetraenoyl, (Z,E,Z,Z)-9-nitro-5,8,11,14-eicosatetraenoyl, (Z,Z,E,Z)-11-nitro-5,8,11,14-eicosatetraenoyl, (Z,Z,E,Z)-12-nitro-5,8,11,14-eicosatetraenoyl. (Z,Z,Z,E)-14-nitro-5,8,11,14-eicosatetraenoyl, (Z,Z,Z,E)-15-nitro-5,8,11,14-eicosatetraenoyl.

(E,E,Z,Z)-5,8-dinitro-5,8,11,14-eicosatetraenoyl, (E,E,Z,Z)-5,9-dinitro-5,8,11,14-eicosatetraenoyl, (E,E,Z,Z)-6,8-dinitro-5,8,11,14-eicosatetraenoyl, (E,E,Z,Z)-6,9-dinitro-5,8,11,14-eicosatetraenoyl, (E,Z,E,Z)-5,11-dinitro-5,8,11,14-eicosatetraenoyl, (E,Z,E,Z)-5,12-dinitro-5,8,11,14-eicosatetraenoyl, (E,Z,E,Z)-6,11-dinitro-5,8,11,14-eicosatetraenoyl, (E,Z,E,Z)-6,12-dinitro-5,8,11,14-eicosatetraenoyl, (E,Z,Z,E)-5,14-dinitro-5,8,11,14-eicosatetraenoyl, (E,Z,Z,E)-5,15-dinitro-5,8,11,14-eicosatetraenoyl, (E,Z,Z,E)-6,14-dinitro-5,8,11,14-eicosatetraenoyl, (E,Z,Z,E)-6,15-dinitro-5,8,11,14-eicosatetraenoyl, (Z,E,E,Z)-8,11-dinitro-5,8,11,14-eicosatetraenoyl, (Z,E,E,Z)-8,12-dinitro-5,8,11,14-eicosatetraenoyl, (Z,E,E,Z)-9,11-dinitro-5,8,11,14-eicosatetraenoyl, (Z,E,E,Z)-9,12-dinitro-5,8,11,14-eicosatetraenoyl. (Z,E,Z,E)-8,14-dinitro-5,8,11,14-eicosatetraenoyl, (Z,E,Z,E)-8,15-dinitro-5,8,11,14-eicosatetraenoyl, (Z,E,Z,E)-9,14-dinitro-5,8,11,14-eicosatetraenoyl, (Z,E,Z,E)-9,15-dinitro-5,8,11,14-eicosatetraenoyl. (Z,Z,E,E)-11,14-dinitro-5,8,11,14-eicosatetraenoyl, (Z,Z,E,E)-11,15-dinitro-5,8,11,14-eicosatetraenoyl, (Z,Z,E,E)-12,14-dinitro-5,8,11,14-eicosatetraenoyl, (Z,Z,E,E)-12,15-dinitro-5,8,11,14-eicosatetraenoyl.

Mixtures of (E,E,Z,Z)-5,8-dinitro-5,8,11,14-eicosatetraenoyl, (E,E,Z,Z)-5,9-dinitro-5,8,11,14-eicosatetraenoyl, (E,E,Z,Z)-6,8-dinitro-5,8,11,14-eicosatetraenoyl, (E,E,Z,Z)-6,9-dinitro-5,8,11,14-eicosatetraenoyl, (E,Z,E,Z)-5,11-dinitro-5,8,11,14-eicosatetraenoyl, (E,Z,E,Z)-5,12-dinitro-5,8,11,14-eicosatetraenoyl, (E,Z,E,Z)-6,11-dinitro-5,8,11,14-eicosatetraenoyl, (E,Z,E,Z)-6,12-dinitro-5,8,11,14-eicosatetraenoyl, (E,Z,Z,E)-5,14-dinitro-5,8,11,14-eicosatetraenoyl, (E,Z,Z,E)-5,15-dinitro-5,8,11,14-eicosatetraenoyl, (E,Z,Z,E)-6,14-dinitro-5,8,11,14-eicosatetraenoyl, (E,Z,Z,E)-6,15-dinitro-5,8,11,14-eicosatetraenoyl, (Z,E,E,Z)-8,11-dinitro-5,8,11,14-eicosatetraenoyl, (Z,E,E,Z)-8,12-dinitro-5,8,11,14-eicosatetraenoyl, (Z,E,E,Z)-9,11-dinitro-5,8,11,14-eicosatetraenoyl, (Z,E,E,Z)-9,12-dinitro-5,8,11,14-eicosatetraenoyl. (Z,E,Z,E)-8,14-dinitro-5,8,11,14-eicosatetraenoyl, (Z,E,Z,E)-8,15-dinitro-5,8,11,14-eicosatetraenoyl, (Z,E,Z,E)-9,14-dinitro-5,8,11,14-eicosatetraenoyl, (Z,E,Z,E)-9,15-dinitro-5,8,11,14-eicosatetraenoyl. (Z,Z,E,E)-11,14-dinitro-5,8,11,14-eicosatetraenoyl, (Z,Z,E,E)-11,15-dinitro-5,8,11,14-eicosatetraenoyl, (Z,Z,E,E)-12,14-dinitro-5,8,11,14-eicosatetraenoyl and (Z,Z,E,E)-12,15-dinitro-5,8,11,14-eicosatetraenoyl.

Mixtures of (E,Z,Z,Z)-5-nitro-5,8,11,14-eicosatetraenoyl, (E,Z,Z,Z)-6-nitro-5,8,11,14-eicosatetraenoyl, (Z,E,Z,Z)-8-nitro-5,8,11,14-eicosatetraenoyl, (Z,E,Z,Z)-9-nitro-5,8,11,14-eicosatetraenoyl, (Z,Z,E,Z)-11-nitro-5,8,11,14-eicosatetraenoyl, (Z,Z,E,Z)-12-nitro-5,8,11,14-eicosatetraenoyl. (Z,Z,Z,E)-14-nitro-5,8,11,14-eicosatetraenoyl, (Z,Z,Z,E)-15-nitro-5,8,11,14-eicosatetraenoyl, (E,E,Z,Z)-5,8-dinitro-5,8,11,14-eicosatetraenoyl, (E,E,Z,Z)-5,9-dinitro-5,8,11,14-eicosatetraenoyl, (E,E,Z,Z)-6,8-dinitro-5,8,11,14-eicosatetraenoyl, (E,E,Z,Z)-6,9-dinitro-5,8,11,14-eicosatetraenoyl, (E,Z,E,Z)-5,11-dinitro-5,8,11,14-eicosatetraenoyl, (E,Z,E,Z)-5,12-dinitro-5,8,11,14-eicosatetraenoyl, (E,Z,E,Z)-6,11-dinitro-5,8,11,14-eicosatetraenoyl, (E,Z,E,Z)-6,12-dinitro-5,8,11,14-eicosatetraenoyl, (E,Z,Z,E)-5,14-dinitro-5,8,11,14-eicosatetraenoyl, (E,Z,Z,E)-5,15-dinitro-5,8,11,14-eicosatetraenoyl, (E,Z,Z,E)-6,14-dinitro-5,8,11,14-eicosatetraenoyl, (E,Z,Z,E)-6,15-dinitro-5,8,11,14-eicosatetraenoyl, (Z,E,E,Z)-8,11-dinitro-5,8,11,14-eicosatetraenoyl, (Z,E,E,Z)-8,12-dinitro-5,8,11,14-eicosatetraenoyl, (Z,E,E,Z)-9,11-dinitro-5,8,11,14-eicosatetraenoyl, (Z,E,E,Z)-9,12-dinitro-5,8,11,14-eicosatetraenoyl. (Z,E,Z,E)-8,14-dinitro-5,8,11,14-eicosatetraenoyl, (Z,E,Z,E)-8,15-dinitro-5,8,11,14-eicosatetraenoyl, (Z,E,Z,E)-9,14-dinitro-5,8,11,14-eicosatetraenoyl, (Z,E,Z,E)-9,15-dinitro-5,8,11,14-eicosatetraenoyl. (Z,Z,E,E)-11,14-dinitro-5,8,11,14-eicosatetraenoyl, (Z,Z,E,E)-11,15-dinitro-5,8,11,14-eicosatetraenoyl, (Z,Z,E,E)-12,14-dinitro-5,8,11,14-eicosatetraenoyl and (Z,Z,E,E)-12,15-dinitro-5,8,11,14-eicosatetraenoyl.

(Z,Z,Z)-5-hydroxy-6-nitro-8,11,14-eicosatrienoyl, (Z,Z,Z)-6-hydroxy-5-nitro-8,11,14-eicosatrienoyl, (Z,Z,Z)-8-hydroxy-9-nitro-5,11,14-eicosatrienoyl, (Z,Z,Z)-9-hydroxy-8-nitro-5,11,14-eicosatrienoyl, (Z,Z,Z)-11-hydroxy-12-nitro-5,8,14-eicosatrienoyl, (Z,Z,Z)-12-hydroxy-11-nitro-5,8,14-eicosatrienoyl. (Z,Z,Z)-14-hydroxy-15-nitro-5,8,14-eicosatrienoyl, (Z,Z,Z)-15-hydroxy-14-nitro-5,8,14-eicosatrienoyl.

Mixtures of (Z,Z,Z)-5-hydroxy-6-nitro-8,11,14-eicosatrienoyl, (Z,Z,Z)-6-hydroxy-5-nitro-8,11,14-eicosatrienoyl, (Z,Z,Z)-8-hydroxy-9-nitro-5,11,14-eicosatrienoyl, (Z,Z,Z)-9-hydroxy-8-nitro-5,11,14-eicosatrienoyl, (Z,Z,Z)-11-hydroxy-12-nitro-5,8,14-eicosatrienoyl, (Z,Z,Z)-12-hydroxy-11-nitro-5,8,14-eicosatrienoyl. (Z,Z,Z)-14-hydroxy-15-nitro-5,8,14-eicosatrienoyl and (Z,Z,Z)-15-hydroxy-14-nitro-5,8,14-eicosatrienoyl.

(Z,Z)-5,8-dihydroxy-6,9-dinitro-11,14-eicosadienoyl, (Z,Z)-5,9-dihydroxy-6,8-dinitro-11,14-eicosadienoyl, (Z,Z)-6,8-dihydroxy-5,9-dinitro-11,14-eicosadienoyl, (Z,Z)-6,9-dihydroxy-5,8-dinitro-11,14-eicosadienoyl, (Z,Z)-5,11-dihydroxy-6,12-dinitro-8,14-eicosadienoyl, (Z,Z)-5,12-dihydroxy-6,11-dinitro-8,14-eicosadienoyl, (Z,Z)-6,11-dihydroxy-5,12-dinitro-8,14-eicosadienoyl, (Z,Z)-6,12-dihydroxy-5,11-dinitro-8,14-eicosadienoyl, (Z,Z)-8,11-dihydroxy-9,12-dinitro-5,14-eicosadienoyl, (Z,Z)-8,12-dihydroxy-9,11-dinitro-5,14-eicosadienoyl, (Z,Z)-9,11-dihydroxy-8,12-dinitro-5,14-eicosadienoyl, (Z,Z)-9,12-dihydroxy-8,11-dinitro-5,14-eicosadienoyl, (Z,Z)-5,14-dihydroxy-6,15-dinitro-8,11-eicosadienoyl, (Z,Z)-5,15-dihydroxy-6,14-dinitro-8,11-eicosadienoyl, (Z,Z)-6,14-dihydroxy-5,15-dinitro-8,11-eicosadienoyl, (Z,Z)-6,15-dihydroxy-5,14-dinitro-8,11-eicosadienoyl, (Z,Z)-8,14-dihydroxy-9,15-dinitro-5,11-eicosadienoyl, (Z,Z)-8,15-dihydroxy-9,14-dinitro-5,11-eicosadienoyl, (Z,Z)-9,14-dihydroxy-8,15-dinitro-5,11-eicosadienoyl, (Z,Z)-9,15-dihydroxy-8,14-dinitro-5,11-eicosadienoyl, (Z,Z)-11,14-dihydroxy-12,15-dinitro-5,8-eicosadienoyl, (Z,Z)-11,15-dihydroxy-12,14-dinitro-5,8-eicosadienoyl, (Z,Z)-12,14-dihydroxy-11,15-dinitro-5,8-eicosadienoyl, (Z,Z)-12,15-dihydroxy-11,14-dinitro-5,8-eicosadienoyl.

Mixtures of (Z,Z)-5,8-dihydroxy-6,9-dinitro-11,14-eicosadienoyl, (Z,Z)-5,9-dihydroxy-6,8-dinitro-11,14-eicosadienoyl, (Z,Z)-6,8-dihydroxy-5,9-dinitro-11,14-eicosadienoyl, (Z,Z)-6,9-dihydroxy-5,8-dinitro-11,14-eicosadienoyl, (Z,Z)-5,11-dihydroxy-6,12-dinitro-8,14-eicosadienoyl, (Z,Z)-5,12-dihydroxy-6,11-dinitro-8,14-eicosadienoyl, (Z,Z)-6,11-dihydroxy-5,12-dinitro-8,14-eicosadienoyl, (Z,Z)-6,12-dihydroxy-5,11-dinitro-8,14-eicosadienoyl, (Z,Z)-8,11-dihydroxy-9,12-dinitro-5,14-eicosadienoyl, (Z,Z)-8,12-dihydroxy-9,11-dinitro-5,14-eicosadienoyl, (Z,Z)-9,11-dihydroxy-8,12-dinitro-5,14-eicosadienoyl, (Z,Z)-9,12-dihydroxy-8,11-dinitro-5,14-eicosadienoyl, (Z,Z)-5,14-dihydroxy-6,15-dinitro-8,11-eicosadienoyl, (Z,Z)-5,15-dihydroxy-6,14-dinitro-8,11-eicosadienoyl, (Z,Z)-6,14-dihydroxy-5,15-dinitro-8,11-eicosadienoyl, (Z,Z)-6,15-dihydroxy-5,14-dinitro-8,11-eicosadienoyl, (Z,Z)-8,14-dihydroxy-9,15-dinitro-5,11-eicosadienoyl, (Z,Z)-8,15-dihydroxy-9,14-dinitro-5,11-eicosadienoyl, (Z,Z)-9,14-dihydroxy-8,15-dinitro-5,11-eicosadienoyl, (Z,Z)-9,15-dihydroxy-8,14-dinitro-5,11-eicosadienoyl, (Z,Z)-11,14-dihydroxy-12,15-dinitro-5,8-eicosadienoyl, (Z,Z)-11,15-dihydroxy-12,14-dinitro-5,8-eicosadienoyl, (Z,Z)-12,14-dihydroxy-11,15-dinitro-5,8-eicosadienoyl and (Z,Z)-12,15-dihydroxy-11,14-dinitro-5,8-eicosadienoyl.

Mixtures of (Z,Z,Z)-5-hydroxy-6-nitro-8,11,14-eicosatrienoyl, (Z,Z,Z)-6-hydroxy-5-nitro-8,11,14-eicosatrienoyl, (Z,Z,Z)-8-hydroxy-9-nitro-5,11,14-eicosatrienoyl, (Z,Z,Z)-9-hydroxy-8-nitro-5,11,14-eicosatrienoyl, (Z,Z,Z)-11-hydroxy-12-nitro-5,8,14-eicosatrienoyl, (Z,Z,Z)-12-hydroxy-11-nitro-5,8,14-eicosatrienoyl. (Z,Z,Z)-14-hydroxy-15-nitro-5,8,14-eicosatrienoyl, (Z,Z,Z)-15-hydroxy-14-nitro-5,8,14-eicosatrienoyl, (Z,Z)-5,8-dihydroxy-6,9-dinitro-11,14-eicosadienoyl, (Z,Z)-5,9-dihydroxy-6,8-dinitro-11,14-eicosadienoyl, (Z,Z)-6,8-dihydroxy-5,9-dinitro-11,14-eicosadienoyl, (Z,Z)-6,9-dihydroxy-5,8-dinitro-11,14-eicosadienoyl, (Z,Z)-5,11-dihydroxy-6,12-dinitro-8,14-eicosadienoyl, (Z,Z)-5,12-dihydroxy-6,11-dinitro-8,14-eicosadienoyl, (Z,Z)-6,11-dihydroxy-5,12-dinitro-8,14-eicosadienoyl, (Z,Z)-6,12-dihydroxy-5,11-dinitro-8,14-eicosadienoyl, (Z,Z)-8,11-dihydroxy-9,12-dinitro-5,14-eicosadienoyl, (Z,Z)-8,12-dihydroxy-9,11-dinitro-5,14-eicosadienoyl, (Z,Z)-9,11-dihydroxy-8,12-dinitro-5,14-eicosadienoyl, (Z,Z)-9,12-dihydroxy-8,11-dinitro-5,14-eicosadienoyl, (Z,Z)-5,14-dihydroxy-6,15-dinitro-8,11-eicosadienoyl, (Z,Z)-5,15-dihydroxy-6,14-dinitro-8,11-eicosadienoyl, (Z,Z)-6,14-dihydroxy-5,15-dinitro-8,11-eicosadienoyl, (Z,Z)-6,15-dihydroxy-5,14-dinitro-8,11-eicosadienoyl, (Z,Z)-8,14-dihydroxy-9,15-dinitro-5,11-eicosadienoyl, (Z,Z)-8,15-dihydroxy-9,14-dinitro-5,11-eicosadienoyl, (Z,Z)-9,14-dihydroxy-8,15-dinitro-5,11-eicosadienoyl, (Z,Z)-9,15-dihydroxy-8,14-dinitro-5,11-eicosadienoyl, (Z,Z)-11,14-dihydroxy-12,15-dinitro-5,8-eicosadienoyl, (Z,Z)-11,15-dihydroxy-12,14-dinitro-5,8-eicosadienoyl, (Z,Z)-12,14-dihydroxy-11,15-dinitro-5,8-eicosadienoyl and (Z,Z)-12,15-dihydroxy-11,14-dinitro-5,8-eicosadienoyl.

(E,Z,Z,Z)-8-nitro-8,11,14,17-eicosatetraenoyl, (E,Z,Z,Z)-9-nitro-8,11,14,17-eicosatetraenoyl, (Z,E,Z,Z)-11-nitro-8,11,14,17-eicosatetraenoyl, (Z,E,Z,Z)-12-nitro-8,11,14,17-eicosatetraenoyl, (Z,Z,E,Z)-14-nitro-8,11,14,17-eicosatetraenoyl, (Z,Z,E,Z)-15-nitro-8,11,14,17-eicosatetraenoyl. (Z,Z,Z,E)-17-nitro-8,11,14,17-eicosatetraenoyl, (Z,Z,Z,E)-18-nitro-8,11,14,17-eicosatetraenoyl.

Mixtures of (E,Z,Z,Z)-8-nitro-8,11,14,17-eicosatetraenoyl, (E,Z,Z,Z)-9-nitro-8,11,14,17-eicosatetraenoyl, (Z,E,Z,Z)-11-nitro-8,11,14,17-eicosatetraenoyl, (Z,E,Z,Z)-12-nitro-8,11,14,17-eicosatetraenoyl, (Z,Z,E,Z)-14-nitro-8,11,14,17-eicosatetraenoyl, (Z,Z,E,Z)-15-nitro-8,11,14,17-eicosatetraenoyl. (Z,Z,Z,E)-17-nitro-8,11,14,17-eicosatetraenoyl and (Z,Z,Z,E)-18-nitro-8,11,14,17-eicosatetraenoyl.

(E,E,Z,Z)-8,11-dinitro-8,11,14,17-eicosatetraenoyl, (E,E,Z,Z)-8,12-dinitro-8,11,14,17-eicosatetraenoyl, (E,E,Z,Z)-9,11-dinitro-8,11,14,17-eicosatetraenoyl, (E,E,Z,Z)-9,12-dinitro-8,11,14,17-eicosatetraenoyl, (E,Z,E,Z)-8,14-dinitro-8,11,14,17-eicosatetraenoyl, (E,Z,E,Z)-8,15-dinitro-8,11,14,17-eicosatetraenoyl, (E,Z,E,Z)-9,14-dinitro-8,11,14,17-eicosatetraenoyl, (E,Z,E,Z)-9,15-dinitro-8,11,14,17-eicosatetraenoyl, (E,Z,Z,E)-8,17-dinitro-8,11,14,17-eicosatetraenoyl, (E,Z,Z,E)-8,18-dinitro-8,11,14,17-eicosatetraenoyl, (E,Z,Z,E)-9,17-dinitro-8,11,14,17-eicosatetraenoyl, (E,Z,Z,E)-9,18-dinitro-8,11,14,17-eicosatetraenoyl, (Z,E, E,Z)-11,14-dinitro-8,11,14,17-eicosatetraenoyl, (Z,E,E,Z)-11,15-dinitro-8,11,14,17-eicosatetraenoyl, (Z,E,E,Z)-12,14-dinitro-8,11,14,17-eicosatetraenoyl, (Z,E, E,Z)-12,15-dinitro-8,11,14,17-eicosatetraenoyl. (Z,E,Z,E)-11,17-dinitro-8,11,14,17-eicosatetraenoyl, (Z,E,Z,E)-11,18-dinitro-8,11,14,17-eicosatetraenoyl, (Z,E,Z,E)-12,17-dinitro-8,11,14,17-eicosatetraenoyl, (Z,E,Z,E)-12,18-dinitro-8,11,14,17-eicosatetraenoyl. (Z,Z,E,E)-14,17-dinitro-8,11,14,17-eicosatetraenoyl, (Z,Z,E,E)-14,18-dinitro-8,11,14,17-eicosatetraenoyl, (Z,Z,E,E)-15,17-dinitro-8,11,14,17-eicosatetraenoyl, (Z,Z,E,E)-15,18-dinitro-8,11,14,17-eicosatetraenoyl.

Mixtures of (E,E,Z,Z)-8,11-dinitro-8,11,14,17-eicosatetraenoyl, (E,E,Z,Z)-8,12-dinitro-8,11,14,17-eicosatetraenoyl, (E,E,Z,Z)-9,11-dinitro-8,11,14,17-eicosatetraenoyl, (E,E,Z,Z)-9,12-dinitro-8,11,14,17-eicosatetraenoyl, (E,Z,E,Z)-8,14-dinitro-8,11,14,17-eicosatetraenoyl, (E,Z,E,Z)-8,15-dinitro-8,11,14,17-eicosatetraenoyl, (E,Z,E,Z)-9,14-dinitro-8,11,14,17-eicosatetraenoyl, (E,Z,E,Z)-9,15-dinitro-8,11,14,17-eicosatetraenoyl, (E,Z,Z,E)-8,17-dinitro-8,11,14,17-eicosatetraenoyl, (E,Z,Z,E)-8,18-dinitro-8,11,14,17-eicosatetraenoyl, (E,Z,Z,E)-9,17-dinitro-8,11,14,17-eicosatetraenoyl, (E,Z,Z,E)-9,18-diniio-8,11,14,17-eicosatetraenoyl, (Z,E,E,Z)-11,14-dinitro-8,11,14,17-eicosatetraenoyl, (Z,E, E,Z)-11,15-dinitro-8,11,14,17-eicosatetraenoyl, (Z,E,E,Z)-12,14-dinitro-8,11,14,17-eicosatetraenoyl, (Z,E, E,Z)-12,15-dinitro-8,11,14,17-eicosatetraenoyl. (Z,E,Z,E)-11,17-dinitro-8,11,14,17-eicosatetraenoyl, (Z,E,Z,E)-11,18-dinitro-8,11,14,17-eicosatetraenoyl, (Z,E,Z,E)-12,17-dinitro-8,11,14,17-eicosatetraenoyl, (Z,E,Z,E)-12,18-dinitro-8,11,14,17-eicosatetraenoyl. (Z,Z,E,E)-14,17-dinitro-8,11,14,17-eicosatetraenoyl, (Z,Z,E,E)-14,18-dinitro-8,11,14,17-eicosatetraenoyl, (Z,Z,E,E)-15,17-dinitro-8,11,14,17-eicosatetraenoyl and (Z,Z,E,E)-15,18-dinitro-8,11,14,17-eicosatetraenoyl.

Mixtures of (E,Z,Z,Z)-8-nitro-8,11,14,17-eicosatetraenoyl, (E,Z,Z,Z)-9-nitro-8,11,14,17-eicosatetraenoyl, (Z,E,Z,Z)-11-nitro-8,11,14,17-eicosatetraenoyl, (Z,E,Z,Z)-12-nitro-8,11,14,17-eicosatetraenoyl, (Z,Z,E,Z)-14-nitro-8,11,14,17-eicosatetraenoyl, (Z,Z,E,Z)-15-nitro-8,11,14,17-eicosatetraenoyl. (Z,Z,Z,E)-17-nitro-8,11,14,17-eicosatetraenoyl, (Z,Z,Z,E)-18-nitro-8,11,14,17-eicosatetraenoyl, (E,E,Z,Z)-8,11-dinitro-8,11,14,17-eicosatetraenoyl, (E,E,Z,Z)-8,12-dinitro-8,11,14,17-eicosatetraenoyl, (E,E,Z,Z)-9,11-dinitro-8,11,14,17-eicosatetraenoyl, (E,E,Z,Z)-9,12-dinitro-8,11,14,17-eicosatetraenoyl, (E,Z,E,Z)-8,14-dinitro-8,11,14,17-eicosatetraenoyl, (E,Z,E,Z)-8,15-dinitro-8,11,14,17-eicosatetraenoyl, (E,Z,E,Z)-9,14-dinitro-8,11,14,17-eicosatetraenoyl, (E,Z,E,Z)-9,15-dinitro-8,11,14,17-eicosatetraenoyl, (E,Z,Z,E)-8,17-dinitro-8,11,14,17-eicosatetraenoyl, (E,Z,Z,E)-8,18-dinitro-8,11,14,17-eicosatetraenoyl, (E,Z,Z,E)-9,17-dinitro-8,11,14,17-eicosatetraenoyl, (E,Z,Z,E)-9,18-dinitro-8,11,14,17-eicosatetraenoyl, (Z,E,E,Z)-11,14-dinitro-8,11,14,17-eicosatetraenoyl, (Z,E,E,Z)-11,15-dinitro-8,11,14,17-eicosatetraenoyl, (Z,E,E,Z)-12,14-dinitro-8,11,14,17-eicosatetraenoyl, (Z,E,E,Z)-12,15-dinitro-8,11,14,17-eicosatetraenoyl. (Z,E,Z,E)-11,17-dinitro-8,11,14,17-eicosatetraenoyl, (Z,E,Z,E)-11,18-dinitro-8,11,14,17-eicosatetraenoyl, (Z,E,Z,E)-12,17-dinitro-8,11,14,17-eicosatetraenoyl, (Z,E,Z,E)-12,18-dinitro-8,11,14,17-eicosatetraenoyl. (Z,Z,E,E)-14,17-dinitro-8,11,14,17-eicosatetraenoyl, (Z,Z,E,E)-14,18-dinitro-8,11,14,17-eicosatetraenoyl, (Z,Z,E,E)-15,17-dinitro-8,11,14,17-eicosatetraenoyl and (Z,Z,E,E)-15,18-dinitro-8,11,14,17-eicosatetraenoyl.

(Z,Z,Z)-8-hydroxy-9-nitro-11,14,17-eicosatrienoyl, (Z,Z,Z)-9-hydroxy-8-nitro-11,14,17-eicosatrienoyl, (Z,Z,Z)-11-hydroxy-12-nitro-8,14,17-eicosatrienoyl, (Z,Z,Z)-12-hydroxy-11-nitro-8,14,17-eicosatrienoyl, (Z,Z,Z)-14-hydroxy-15-nitro-8,11,17-eicosatrienoyl, (Z,Z,Z)-15-hydroxy-14-nitro-8,11,17-eicosatrienoyl. (Z,Z,Z)-17-hydroxy-18-nitro-8,11,17-eicosatrienoyl, (Z,Z,Z)-18-hydroxy-17-nitro-8,11,17-eicosatrienoyl.

Mixtures of (Z,Z,Z)-8-hydroxy-9-nitro-11,14,17-eicosatrienoyl, (Z,Z,Z)-9-hydroxy-8-nitro-11,14,17-eicosatrienoyl, (Z,Z,Z)-11-hydroxy-12-nitro-8,14,17-eicosatrienoyl, (Z,Z,Z)-12-hydroxy-11-nitro-8,14,17-eicosatrienoyl, (Z,Z,Z)-14-hydroxy-15-nitro-8,11,17-eicosatrienoyl, (Z,Z,Z)-15-hydroxy-14-nitro-8,11,17-eicosatrienoyl, (Z,Z,Z)-17-hydroxy-18-nitro-8,11,17-eicosatrienoyl and (Z,Z,Z)-18-hydroxy-17-nitro-8,11,17-eicosatrienoyl.

(Z,Z)-8,11-dihydroxy-9,12-dinitro-14,17-eicosadienoyl, (Z,Z)-8,12-dihydroxy-9,11-dinitro-14,17-eicosadienoyl, (Z,Z)-9,11-dihydroxy-8,12-dinitro-14,17-eicosadienoyl, (Z,Z)-9,12-dihydroxy-8,11-dinitro-14,17-eicosadienoyl, (Z,Z)-8,14-dihydroxy-9,15-dinitro-11,17-eicosadienoyl, (Z,Z)-8,15-dihydroxy-9,14-dinitro-11,17-eicosadienoyl, (Z,Z)-9,14-dihydroxy-8,15-dinitro-11,17-eicosadienoyl, (Z,Z)-9,15-dihydroxy-8,14-dinitro-11,17-eicosadienoyl, (Z,Z)-11,14-dihydroxy-12,15-dinitro-8,17-eicosadienoyl, (Z,Z)-11,15-dihydroxy-12,14-dinitro-8,17-eicosadienoyl, (Z,Z)-12,14-dihydroxy-11,15-dinitro-8,17-eicosadienoyl, (Z,Z)-12,15-dihydroxy-11,14-dinitro-8,17-eicosadienoyl, (Z,Z)-8,17-dihydroxy-9,18-dinitro-11,14-eicosadienoyl, (Z,Z)-8,18-dihydroxy-9,17-dinitro-11,14-eicosadienoyl, (Z,Z)-9,17-dihydroxy-8,18-dinitro-11,14-eicosadienoyl, (Z,Z)-9,18-dihydroxy-8,17-dinitro-11,14-eicosadienoyl, (Z,Z)-11,17-dihydroxy-12,18-dinitro-8,14-eicosadienoyl, (Z,Z)-11,18-dihydroxy-12,17-dinitro-8,14-eicosadienoyl, (Z,Z)-12,17-dihydroxy-11,18-dinitro-8,14-eicosadienoyl, (Z,Z)-12,18-dihydroxy-11,17-dinitro-8,14-eicosadienoyl, (Z,Z)-14,17-dihydroxy-15,18-dinitro-8,11-eicosadienoyl, (Z,Z)-14,18-dihydroxy-15,17-dinitro-8,11-eicosadienoyl, (Z,Z)-15,17-dihydroxy-14,18-dinitro-8,11-eicosadienoyl, (Z,Z)-15,18-dihydroxy-14,17-dinitro-8,11-eicosadienoyl.

Mixtures of (Z,Z)-8,11-dihydroxy-9,12-dinitro-14,17-eicosadienoyl, (Z,Z)-8,12-dihydroxy-9,11-dinitro-14,17-eicosadienoyl, (Z,Z)-9,11-dihydroxy-8,12-dinitro-14,17-eicosadienoyl, (Z,Z)-9,12-dihydroxy-8,11-dinitro-14,17-eicosadienoyl, (Z,Z)-8,14-dihydroxy-9,15-dinitro-11,17-eicosadienoyl, (Z,Z)-8,15-dihydroxy-9,14-dinitro-11,17-eicosadienoyl, (Z,Z)-9,14-dihydroxy-8,15-dinitro-11,17-eicosadienoyl, (Z,Z)-9,15-dihydroxy-8,14-dinitro-11,17-eicosadienoyl, (Z,Z)-11,14-dihydroxy-12,15-dinitro-8,17-eicosadienoyl, (Z,Z)-11,15-dihydroxy-12,14-dinitro-8,17-eicosadienoyl, (Z,Z)-12,14-dihydroxy-11,15-dinitro-8,17-eicosadienoyl, (Z,Z)-12,15-dihydroxy-11,14-dinitro-8,17-eicosadienoyl, (Z,Z)-8,17-dihydroxy-9,18-dinitro-11,14-eicosadienoyl, (Z,Z)-8,18-dihydroxy-9,17-dinitro-11,14-eicosadienoyl, (Z,Z)-9,17-dihydroxy-8,18-dinitro-11,14-eicosadienoyl, (Z,Z)-9,18-dihydroxy-8,17-dinitro-11,14-eicosadienoyl, (Z,Z)-11,17-dihydroxy-12,18-dinitro-8,14-eicosadienoyl, (Z,Z)-11,18-dihydroxy-12,17-dinitro-8,14-eicosadienoyl, (Z,Z)-12,17-dihydroxy-11,18-dinitro-8,14-eicosadienoyl, (Z,Z)-12,18-dihydroxy-11,17-dinitro-8,14-eicosadienoyl, (Z,Z)-14,17-dihydroxy-15,18-dinitro-8,11-eicosadienoyl, (Z,Z)-14,18-dihydroxy-15,17-dinitro-8,11-eicosadienoyl, (Z,Z)-15,17-dihydroxy-14,18-dinitro-8,11-eicosadienoyl and (Z,Z)-15,18-dihydroxy-14,17-dinitro-8,11-eicosadienoyl.

Mixtures of (Z,Z,Z)-8-hydroxy-9-nitro-11,14,17-eicosatrienoyl, (Z,Z,Z)-9-hydroxy-8-nitro-11,14,17-eicosatrienoyl, (Z,Z,Z)-11-hydroxy-12-nitro-8,14,17-eicosatrienoyl, (Z,Z,Z)-12-hydroxy-11-nitro-8,14,17-eicosatrienoyl, (Z,Z,Z)-14-hydroxy-15-nitro-8,11,17-eicosatrienoyl, (Z,Z,Z)-15-hydroxy-14-nitro-8,11,17-eicosatrienoyl, (Z,Z,Z)-17-hydroxy-18-nitro-8,11,17-eicosatrienoyl, (Z,Z,Z)-18-hydroxy-17-nitro-8,11,17-eicosatrienoyl, (Z,Z)-8,11-dihydroxy-9,12-dinitro-14,17-eicosadienoyl, (Z,Z)-8,12-dihydroxy-9,11-dinitro-14,17-eicosadienoyl, (Z,Z)-9,11-dihydroxy-8,12-dinitro-14,17-eicosadienoyl, (Z,Z)-9,12-dihydroxy-8,11-dinitro-14,17-eicosadienoyl, (Z,Z)-8,14-dihydroxy-9,15-dinitro-11,17-eicosadienoyl, (Z,Z)-8,15-dihydroxy-9,14-dinitro-11,17-eicosadienoyl, (Z,Z)-9,14-dihydroxy-8,15-dinitro-11,17-eicosadienoyl, (Z,Z)-9,15-dihydroxy-8,14-dinitro-11,17-eicosadienoyl, (Z,Z)-11,14-dihydroxy-12,15-dinitro-8,17-eicosadienoyl, (Z,Z)-11,15-dihydroxy-12,14-dinitro-8,17-eicosadienoyl, (Z,Z)-12,14-dihydroxy-11,15-dinitro-8,17-eicosadienoyl, (Z,Z)-12,15-dihydroxy-11,14-dinitro-8,17-eicosadienoyl, (Z,Z)-8,17-dihydroxy-9,18-dinitro-11,14-eicosadienoyl, (Z,Z)-8,18-dihydroxy-9,17-dinitro-11,14-eicosadienoyl, (Z,Z)-9,17-dihydroxy-8,18-dinitro-11,14-eicosadienoyl, (Z,Z)-9,18-dihydroxy-8,17-dinitro-11,14-eicosadienoyl, (Z,Z)-11,17-dihydroxy-12,18-dinitro-8,14-eicosadienoyl, (Z,Z)-11,18-dihydroxy-12,17-dinitro-8,14-eicosadienoyl, (Z,Z)-12,17-dihydroxy-11,18-dinitro-8,14-eicosadienoyl, (Z,Z)-12,18-dihydroxy-11,17-dinitro-8,14-eicosadienoyl, (Z,Z)-14,17-dihydroxy-15,18-dinitro-8,11-eicosadienoyl, (Z,Z)-14,18-dihydroxy-15,17-dinitro-8,11-eicosadienoyl, (Z,Z)-15,17-dihydroxy-14,18-dinitro-8,11-eicosadienoyl and (Z,Z)-15,18-dihydroxy-14,17-dinitro-8,11-eicosadienoyl.

(E,Z,Z,Z)-7-nitro-7,10,13,16-docosatetraenoyl, (E,Z,Z,Z)-8-nitro-7,10,13,16-docosatetraenoyl, (Z,E,Z,Z)-10-nitro-7,10,13,16-docosatetraenoyl, (Z,E,Z,Z)-11-nitro-7,10,13,16-docosatetraenoyl, (Z,Z,E,Z)-13-nitro-7,10,13,16-docosatetraenoyl, (Z,Z,E,Z)-14-nitro-7,10,13,16-docosatetraenoyl, (Z,Z,Z,E)-16-nitro-7,10,13,16-docosatetraenoyl, (Z,Z,Z,E)-17-nitro-7,10,13,16-docosatetraenoyl.

Mixtures of (E,Z,Z,Z)-7-nitro-7,10,13,16-docosatetraenoyl, (E,Z,Z,Z)-8-nitro-7,10,13,16-docosatetraenoyl, (Z,E,Z,Z)-10-nitro-7,10,13,16-docosatetraenoyl, (Z,E,Z,Z)-11-nitro-7,10,13,16-docosatetraenoyl, (Z,Z,E,Z)-13-nitro-7,10,13,16-docosatetraenoyl, (Z,Z,E,Z)-14-nitro-7,10,13,16-docosatetraenoyl, (Z,Z,Z,E)-16-nitro-7,10,13,16-docosatetraenoyl and (Z,Z,Z,E)-17-nitro-7,10,13,16-docosatetraenoyl.

(Z,Z,Z)-7-hydroxy-8-nitro-10,13,16-docosatrienoyl, (Z,Z,Z)-8-hydroxy-7-nitro-10,13,16-docosatrienoyl, (Z,Z,Z)-10-hydroxy-11-nitro-8,13,16-docosatrienoyl, (Z,Z,Z)-11-hydroxy-10-nitro-8,13,16-docosatrienoyl, (Z,Z,Z)-13-hydroxy-14-nitro-8,10,16-docosatrienoyl, (Z,Z,Z)-14-hydroxy-13-nitro-8,10,16-docosatrienoyl, (Z,Z,Z)-16-hydroxy-17-nitro-8,10,13-docosatrienoyl, (Z,Z,Z)-17-hydroxy-16-nitro-8,10,13-docosatrienoyl, as well as mixtures of the aforedescribed residues.

(E,Z,Z,Z)-6-nitro-6,9,12,15-octadecatetraenoyl, (E,Z,Z,Z)-7-nitro-7,10,13,16-docosatetraenoyl, (Z,E,Z,Z)-9-nitro-7,10,13,16-docosatetraenoyl, (Z,E,Z,Z)-10-nitro-7,10,13,16-docosatetraenoyl, (Z,Z,E,Z)-12-nitro-7,10,13,16-docosatetraenoyl, (Z,Z,E,Z)-13-nitro-7,10,13,16-docosatetraenoyl, (Z,Z,Z,E)-15-nitro-7,10,13,16-docosatetraenoyl, (Z,Z,Z,E)-16-nitro-7,10,13,16-docosatetraenoyl as well as mixtures of the aforedescribed residues.

(Z,Z,Z)-6-hydroxy-7-nitro-9,12,15-octadecatrienoyl, (Z,Z,Z)-7-hydroxy-6-nitro-9,12,15-octadecatrienoyl, (Z,Z,Z)-9-hydroxy-10-nitro-6,12,15-octadecatrienoyl, (Z,Z,Z)-10-hydroxy-9-nitro-6,12,15-octadecatrienoyl, (Z,Z,Z)-12-hydroxy-13-nitro-6,9,15-octadecatrienoyl, (Z,Z,Z)-13-hydroxy-12-nitro-6,9,15-octadecatrienoyl, (Z,Z,Z)-15-hydroxy-16-nitro-6,9,12-octadecatrienoyl, (Z,Z,Z)-16-hydroxy-15-nitro-6,9,12-octadecatrienoyl as well as mixtures of the aforedescribed residues.

(E,Z,Z,Z,Z)-4-nitro-4,7,10,13,16-docosapentaenoyl, (E,Z,Z,Z,Z)-5-nitro-4,7,10,13,16-docosapentaenoyl, (Z,E,Z,Z,Z)-7-nitro-4,7,10,13,16-docosapentaenoyl, (Z,E,Z,Z,Z)-8-nitro-4,7,10,13,16-docosapentaenoyl, (Z,Z,E,Z,Z)-10-nitro-4,7,10,13,16-docosapentaenoyl, (Z,Z,E,Z,Z)-11-nitro-4,7,10,13,16-docosapentaenoyl, (Z,Z,Z,E,Z)-13-nitro-4,7,10,13,16-docosapentaenoyl, (Z,Z,Z,E,Z)-14-nitro-4,7,10,13,16-docosapentaenoyl, (Z,Z,Z,Z,E)-16-nitro-4,7,10,13,16-docosapentaenoyl, (Z,Z,Z,Z,E)-17-nitro-4,7,10,13,16-docosapentaenoyl as well as mixtures of the aforedescribed residues.

(Z,Z,Z,Z)-4-hydroxy-5-nitro-7,10,13,16-docosatetraenoyl, (Z,Z,Z,Z)-5-hydroxy-4-nitro-7,10,13,16-docosatetraenoyl, (Z,Z,Z,Z)-7-hydroxy-8-nitro-4,10,13,16-docosatetraenoyl, (Z,Z,Z,Z)-8-hydroxy-7-nitro-4,10,13,16-docosatetraenoyl, (Z,Z,Z,Z)-10-hydroxy-11-nitro-4,7,13,16-docosatetraenoyl, (Z,Z,Z,Z)-11-hydroxy-10-nitro-4,7,13,16-docosatetraenoyl, (Z,Z,Z,Z)-13-hydroxy-14-nitro-4,7,10,16-docosatetraenoyl, (Z,Z,Z,Z)-14-hydroxy-13-nitro-4,7,10,16-docosatetraenoyl, (Z,Z,Z,Z)-16-hydroxy-17-nitro-4,7,10,13-docosatetraenoyl, (Z,Z,Z,Z)-17-hydroxy-16-nitro-4,7,10,13-docosatetraenoyl as well as mixtures of the avoredescribed residues.

(E,Z,Z,Z,Z)-5-nitro-5,8,11,14,17-eicosapentaenoyl, (E,Z,Z,Z,Z)-6-nitro-5,8,11,14,17-eicosapentaenoyl, (Z,E,Z,Z,Z)-8-nitro-5,8,11,14,17-eicosapentaenoyl, (Z,E,Z,Z,Z)-9-nitro-5,8,11,14,17-eicosapentaenoyl, (Z,Z,E,Z,Z)-11-nitro-5,8,11,14,17-eicosapentaenoyl, (Z,Z,E,Z,Z)-12-nitro-5,8,11,14,17-eicosapentaenoyl, (Z,Z,Z,E,Z)-14-nitro-5,8,11,14,17-eicosapentaenoyl, (Z,Z,Z,E,Z)-15-nitro-5,8,11,14,17-eicosapentaenoyl, (Z,Z,Z,Z,E)-17-nitro-5,8,11,14,17-eicosapentaenoyl, (Z,Z,Z,Z,E)-18-nitro-5,8,11,14,17-eicosapentaenoyl as well as mixtures of the aforemetioned residues.

(Z,Z,Z,Z)-5-hydroxy-6-nitro-8,11,14,17-eicosatetraenoyl, (Z,Z,Z,Z)-6-hydroxy-5-nitro-8,11,14,17-eicosatetraenoyl, (Z,Z,Z,Z)-8-hydroxy-9-nitro-5,11,14,17-eicosatetraenoyl, (Z,Z,Z,Z)-9-hydroxy-8-nitro-5,11,14,17-eicosatetraenoyl, (Z,Z,Z,Z)-11-hydroxy-12-nitro-5,8,14,17-eicosatetraenoyl, (Z,Z,Z,Z)-12-hydroxy-11-nitro-5,8,14,17-eicosatetraenoyl, (Z,Z,Z,Z)-14-hydroxy-15-nitro-5,8,11,17-eicosatetraenoyl, (Z,Z,Z,Z)-15-hydroxy-14-nitro-5,8,11,17-eicosatetraenoyl, (Z,Z,Z,Z)-17-hydroxy-18-nitro-5,8,11,14-eicosatetraenoyl, (Z,Z,Z,Z)-18-hydroxy-17-nitro-5,8,11,14-eicosatetraenoyl as well as mixtures of the avoredescribed residues.

(E,Z,Z,Z,Z)-7-nitro-7,10,13,16,19-docosapentaenoyl, (E,Z,Z,Z,Z)-8-nitro-7,10,13,16,19-docosapentaenoyl, (Z,E,Z,Z,Z)-10-nitro-7,10,13,16,19-docosapentaenoyl, (Z,E,Z,Z,Z)-11-nitro-7,10,13,16,19-docosapentaenoyl, (Z,Z,E,Z,Z)-13-nitro-7,10,13,16,19-docosapentaenoyl, (Z,Z,E,Z,Z)-14-nitro-7,10,13,16,19-docosapentaenoyl, (Z,Z,Z,E,Z)-16-nitro-7,10,13,16,19-docosapentaenoyl, (Z,Z,Z,E,Z)-17-nitro-7,10,13,16,19-docosapentaenoyl, (Z,Z,Z,Z,E)-19-nitro-7,10,13,16,19-docosapentaenoyl, (Z,Z,Z,Z,E)-20-nitro-7,10,13,16,19-docosapentaenoyl as well as mixtures of the aforedescribed residues.

(Z,Z,Z,Z)-7-hydroxy-8-nitro-10,13,16,19-docosatetraenoyl, (Z,Z,Z,Z)-8-hydroxy-7-nitro-10,13,16,19-docosatetraenoyl, (Z,Z,Z,Z)-10-hydroxy-11-nitro-7,13,16,19-docosatetraenoyl, (Z,Z,Z,Z)-11-hydroxy-10-nitro-7,13,16,19-docosatetraenoyl, (Z,Z,Z,Z)-13-hydroxy-14-nitro-7,11,16,19-docosatetraenoyl, (Z,Z,Z,Z)-14-hydroxy-13-nitro-7,11,16,19-docosatetraenoyl, (Z,Z,Z,Z)-16-hydroxy-17-nitro-7,11,13,19-docosatetraenoyl, (Z,Z,Z,Z)-17-hydroxy-16-nitro-7,11,13,19-docosatetraenoyl, (Z,Z,Z,Z)-19-hydroxy-20-nitro-7,11,13,16-docosatetraenoyl, (Z,Z,Z,Z)-20-hydroxy-19-nitro-7,11,13,16-docosatetraenoyl as well as mixtures of the aforedescribed residues. (E,Z,Z,Z,Z,Z)-4-nitro-4,7,10,13,16,19-docosahexaenoyl, (E,Z,Z,Z,Z,Z)-5-nitro-4,7,10,13,16,19-docosahexaenoyl, (Z,E,Z,Z,Z,Z)-7-nitro-4,7,10,13,16,19-docosahexaenoyl, (Z,E,Z,Z,Z,Z)-8-nitro-4,7,10,13,16,19-docosahexaenoyl, (Z,Z,E,Z,Z,Z)-10-nitro-4,7,10,13,16,19-docosahexaenoyl, (Z,Z,E,Z,Z,Z)-11-nitro-4,7,10,13,16,19-docosahexaenoyl, (Z,Z,Z,E,Z,Z)-13-nitro-4,7,10,13,16,19-docosahexaenoyl, (Z,Z,Z,E,Z,Z)-14-nitro-4,7,10,13,16,19-docosahexaenoyl, (Z,Z,Z,Z,E,Z)-16-nitro-4,7,10,13,16,19-docosahexaenoyl, (Z,Z,Z,Z,E,Z)-17-nitro-4,7,10,13,16,19-docosahexaenoyl, (Z,Z,Z,Z,Z,E)-19-nitro-4,7,10,13,16,19-docosahexaenoyl, (Z,Z,Z,Z,Z,E)-20-nitro-4,7,10,13,16,19-docosahexaenoyl as well as mixtures of the aforedescribed residues.

(Z,Z,Z,Z,Z)-4-hydroxy-5-nitro-7,10,13,16,19-docosapentaenoyl, (Z,Z,Z,Z,Z)-5-hydroxy-4-nitro-7,10,13,16,19-docosapentaenoyl, (Z,Z,Z,Z,Z)-7-hydroxy-8-nitro-4,10,13,16,19-docosapentaenoyl, (Z,Z,Z,Z,Z)-8-hydroxy-7-nitro-4,10,13,16,19-docosapentaenoyl, (Z,Z,Z,Z,Z)-10-hydroxy-11-nitro-4,7,13,16,19-docosapentaenoyl, (Z,Z,Z,Z,Z)-11-hydroxy-10-nitro-4,7,13,16,19-docosapentaenoyl, (Z,Z,Z,Z,Z)-13-hydroxy-14-nitro-4,7,10,16,19-docosapentaenoyl, (Z,Z,Z,Z,Z)-14-hydroxy-13-nitro-4,7,10,16,19-docosa pentaenoyl, (Z,Z,Z,Z,Z)-16-hydroxy-17-nitro-4,7,10,13,19-docosapentaenoyl, (Z,Z,Z,Z,Z)-17-hydroxy-16-nitro-4,7,10,13,19-docosapentaenoyl, (Z,Z,Z,Z,Z)-19-hydroxy-20-nitro-4,7,10,13,16-docosapentaenoyl, (Z,Z,Z,Z,Z)-20-hydroxy-19-nitro-4,7,10,13,16-docosapentaenoyl as well as mixtures of the aforedescribed residues.

(E,Z,Z,Z,Z,Z,Z)-4-nitro-4,7,9,11,13,16,19-docosaheptaenoyl, (E,Z,Z,Z,Z,Z,Z)-5-nitro-4,7,9,11,13,16,19-docosaheptaenoyl, (Z,E,Z,Z,Z,Z,Z)-7-nitro-4,7,9,11,13,16,19-docosaheptaenoyl, (Z,E,Z,Z,Z,Z,Z)-8-nitro-4,7,9,11,13,16,19-docosaheptaenoyl, (Z,Z,E,Z,Z,Z,Z)-9-nitro-4,7,9,11,13,16,19-docosaheptaenoyl, (Z,Z,E,Z,Z,Z,Z)-10-nitro-4,7,9,11,13,16,19-docosaheptaenoyl, (Z,Z,Z,E,Z,Z,Z)-11-nitro-4,7,9,11,13,16,19-docosaheptaenoyl, (Z,Z,Z,E,Z,Z,Z)-12-nitro-4,7,9,11,13,16,19-docosaheptaenoyl, (Z,Z,Z,Z,E,Z,Z)-13-nitro-4,7,9,11,13,16,19-docosaheptaenoyl, (Z,Z,Z,Z,E,Z,Z)-14-nitro-4,7,9,11,13,16,19-docosaheptaenoyl, (Z,Z,Z,Z,Z,E,Z)-16-nitro-4,7,9,11,13,16,19-docosaheptaenoyl, (Z,Z,Z,Z,Z,E,Z)-17-nitro-4,7,9,11,13,16,19-docosaheptaenoyl, (Z,Z,Z,Z,Z,Z,E)-19-nitro-4,7,9,11,13,16,19-docosaheptaenoyl, (Z,Z,Z,Z,Z,Z,E)-20-nitro-4,7,9,11,13,16,19-docosaheptaenoyl as well as mixtures of the aforedescribed residues.

(Z,Z,Z,Z,Z,Z)-4-hydroxy-5-nitro-7,9,11,13,16,19-docosahexaenoyl, (Z,Z,Z,Z,Z,Z)-5-hydroxy-4-nitro-7,9,11,13,16,19-docosahexaenoyl, (Z,Z,Z,Z,Z,Z)-7-hydroxy-8-nitro-4,9,11,13,16,19-docosahexaenoyl, (Z,Z,Z,Z,Z,Z)-8-hydroxy-7-nitro-4,9,11,13,16,19-docosahexaenoyl, (Z,Z,Z,Z,Z,Z)-9-hydroxy-10-nitro-4,7,11,13,16,19-docosahexaenoyl, (Z,Z,Z,Z,Z,Z)-10-hydroxy-9-nitro-4,7,11,13,16,19-docosahexaenoyl, (Z,Z,Z,Z,Z,Z)-11-hydroxy-12-nitro-4,7,9,13,16,19-docosahexaenoyl, (Z,Z,Z,Z,Z,Z)-12-hydroxy-11-nitro-4,7,9,13,16,19-docosahexaenoyl, (Z,Z,Z,Z,Z,Z)-13-hydroxy-14-nitro-4,7,9,11,16,19-docosahexaenoyl, (Z,Z,Z,Z,Z,Z)-14-hydroxy-13-nitro-4,7,9,11,16,19-docosahexaenoyl, (Z,Z,Z,Z,Z,Z)-16-hydroxy-17-nitro-4,7,9,11,13,19-docosahexaenoyl, (Z,Z,Z,Z,Z,Z)-17-hydroxy-16-nitro-4,7,9,11,13,19-docosahexaenoyl, (Z,Z,Z,Z,Z,Z)-19-hydroxy-20-nitro-4,7,9,11,13,16-docosahexaenoyl, (Z,Z,Z,Z,Z,Z)-20-hydroxy-19-nitro-4,7,9,11,13,16-docosahexaenoyl as well as mixtures of the aforedescribed residues.

Nitrohexanoly, dinitrohexanoly, trinitrohexanoly, nitrooctanoly, dinitrooctanoly, trinitrooctanoly, nitrodecanoyl, dinitrodecanoyl, trinitrodecanoyl, nitrododecanoyl, dinitrododecanoyl, trinitrododecanoyl, nitrotetradecanoyl, dinitrotetradecanoyl, trinitrotetradecanoyl, nitrohexadecanoyl, dinitrohexadecanoyl, trinitrohexadecanoyl, nitroheptadecanoyl, dinitroheptadecanoyl, trinitroheptadecanoyl, nitrooctadecanoyl, dinitrooctadecanoyl, trinitrooctadecanoyl, nitroeicosanoyl, dinitroeicosanoyl, trinitroeicosanoyl, nitrodocosanoyl, dinitrodocosanoyl, trinitrodocosanoyl, nitrotetracosanoyl, dinitrotetracosanoyl, trinitrotetracosanoyl, nitroisopalmitinoyl, dinitroisopalmitinoyl, trinitroisopalmitinoyl, nitro-11,12-methylen-octadecanoyl, dinitro-11,12-methylen-octadecanoyl, trinitro-11,12-methylen-octadecanoyl, nitro-9,10-methylen-hexadecanoyl, dinitro-9,10-methylen-hexadecanoyl, trinitro-9,10-methylen-hexadecanoyl, nitroretinoyl, dinitroretinoyl, trinitroretinoyl, nitrophytanoyl, dinitrophytanoyl as well as finalizing trinitrophytanoyl.

Since nitration reactions and particularly the acidic or radical nitration cannot be performed selectively and the nitrated carboxylic acids are sometimes difficult to separate, the use of mixtures of nitrated carboxylic acids is preferred. These mixtures comprise preferably regioisomers of carboxylic acids with several double bonds, as well as mixtures of singly, doubly, triply or multiply nitrated carboxylic acids. Nitrated carboxylic acids as a pure substance (i.e. no mixtures) can be prepared best by a substitution reaction, as shown in the following:

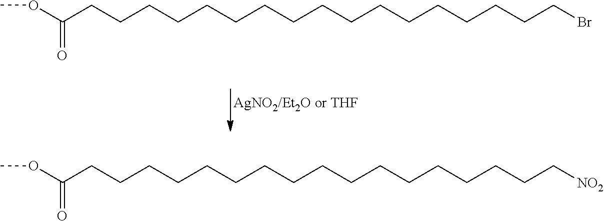

If free halogen carboxylic acids, i.e. not yet bound to a phospholipid are reacted with silver nitrate, it may be advantageous to protect the carboxylate group.

Non-selective nitration reactions are described in Gorczynski, Michael J., Huang, Jinming, King, S. Bruce; Organic Letters, 2006, 8, 11, 2305-2308 and are depicted in the following scheme:

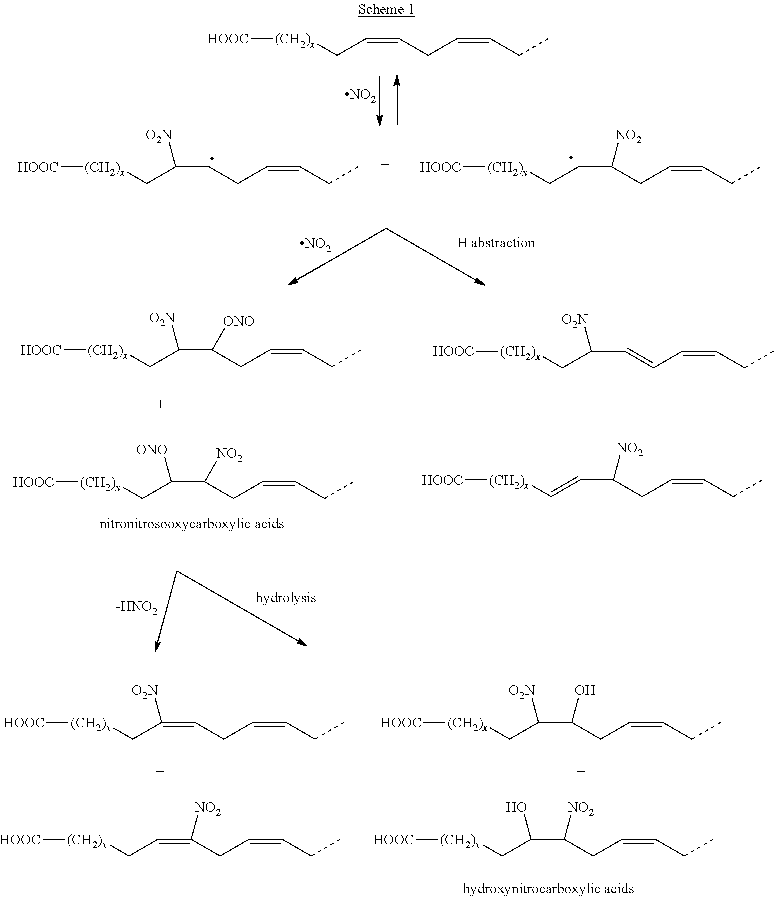

The reaction of unsaturated carboxylic acids with NO₂ radicals provides, via a radical nitro-carboxylic acid, an unsaturated nitro-carboxylic acid by H abstraction, where the nitro group is introduced at the allylic position of the double bond. The original double bond is migrating. However, a free radical addition of NO₂ results in the formation of nitro-nitrite-carboxylic acids, which can be transformed by hydrolysis into hydroxy nitro carboxylic acids, where in fact a hydroxyl group and a nitro group on a double bond have been formed or an unsaturated nitro-carboxylic acid with the introduced nitro group in vinylic position is formed by removal of HNO₂, i.e. on the double bond. The original double bond is present in its unchanged location. These reactions can be repeated several times and in the presence of several double bonds in theory until all double bounds have been reacted, so that dinitrocarboxylic acids, trinitrocarboxylic acids and polynitrocarboxylic acids are formed, which are almost always mixtures.

The unselective nitration of one double bond of the carboxylic acid can also be performed in an acidic environment, as shown in the following (scheme 2):

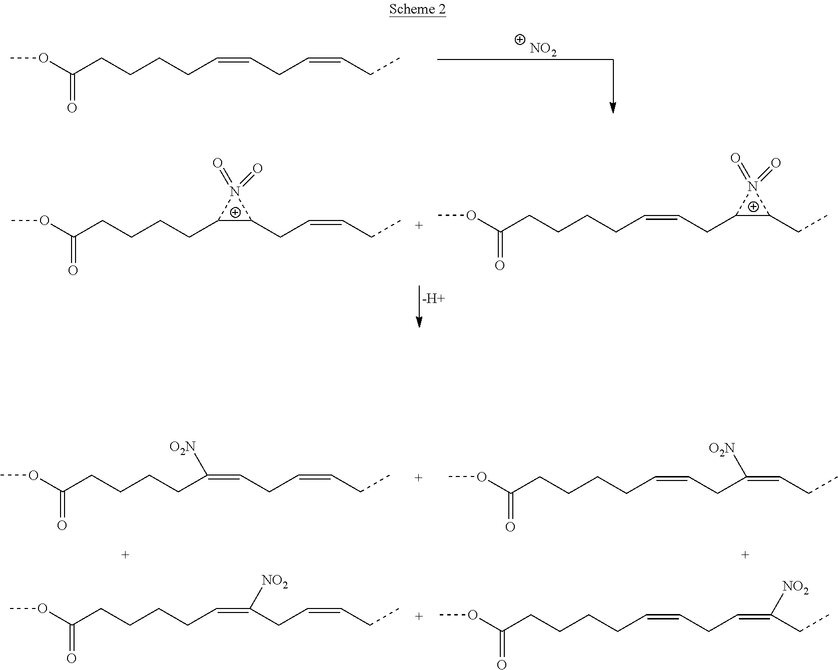

Another possibility of nitrating (scheme 3) an unsaturated carboxylic acid is shown in the following reaction diagram and using PhSeBr

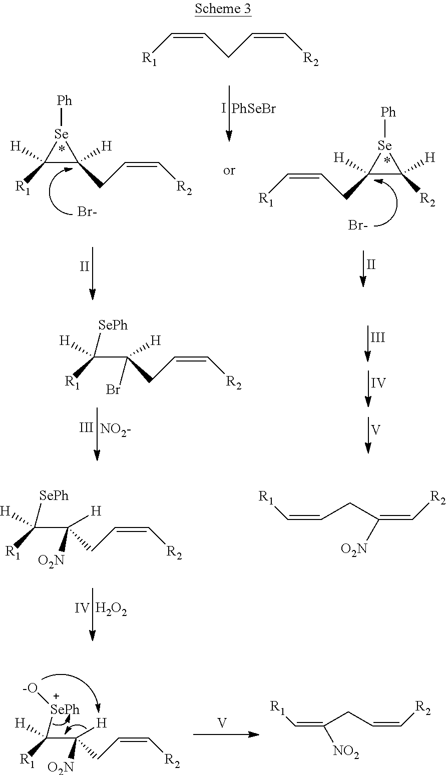

The inventive nitro carboxylic acid(s)-containing phospholipids can be obtained by esterification of the two OH groups of the glycerol unit with the same nitro-carboxylic acid. In this case, R¹COOH is identical to R²COOH and R¹ is identical to R² (see scheme 4).

If different nitro-carboxylic acids are used, the esterification reactions must be sequentially performed wherein preferably, first the primary OH group should be selectively esterified and then the secondary OH group should be esterified. Thus, the first esterification reaction can be performed with a nitro-carboxylic acid and the second with a non-nitrated carboxylic acid, or the first esterification reaction can be performed with a non-nitrated carboxylic acid and the second with a nitro-carboxylic acid or each of the two esterification reactions is performed with a different nitro-, carboxylic acid (see scheme 4). The esterification reactions are performed according to standard reaction known by a person skilled in the art.

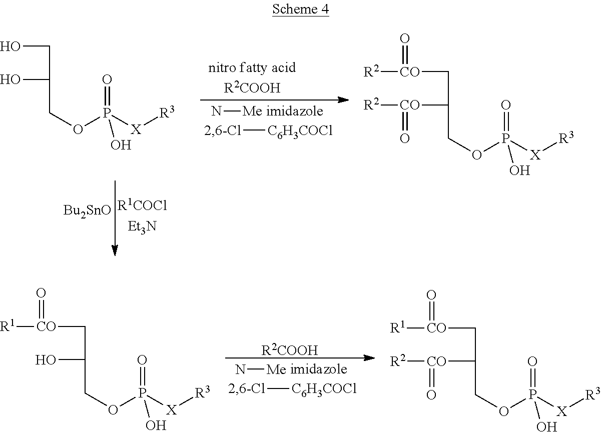

Another possibility to synthesize the inventive nitro carboxylic acid(s)-containing phospholipids is shown in scheme 5. When mixtures of nitro-carboxylic acids are used, PL with different residues at positions 1 and 2, i.e. R¹COO—≠R²COO— are mainly formed. Moreover, the reaction sequence shown in scheme 5 allows also the introduction of a non-nitrated carboxylic acid residue (R¹COO—) at position 1, and one nitrated carboxylic acid residue (R²COO—) at position 2. The reaction sequence shown in scheme 5 can also be applied when R¹COO— represents a nitrated saturated carboxylic acid residue. In this case, R²COO— can be any nitrated or non-nitrated, as well as saturated or unsaturated carboxylic acid residues. However, when R¹COO— is a nitrated unsaturated carboxylic acid residue and especially, an unsaturated carboxylic acid residue nitrated at the vinylic position (i.e. on the double bond) the reaction sequence shown in scheme 5 is not or only with difficulties possible. Subsequent esterification with R²COO— is carried out, only under drastic loss of yield. For such case, a new synthesis that is shown in scheme 6 was developed. Herein, first both positions 1 and 2 are esterified with non-nitrated carboxylic acid residues or nitrated saturated monocarboxylic acid residues and then position 1 is selectively cleaved enzymatically using phospholipase, so that a nitrated unsaturated carboxylic acid residue can then be introduced at the position 1. General as well as specific reaction procedures to perform the syntheses are given in the experimental section.

As stated above, the residues R¹COO— and R²COO— of nitrocarboxylic acid(s)-containing phospholipids can vary widely, where at least one of the residues of R¹COO— and R²COO— represents one of the aforementioned nitrocarboxylic acid residues.

The residue R³ can be, for example, hydrogen, serine, choline, a sugar such as inositol or colamin (ethanolamine). If a choline residue is esterified, lecithin (also named phosphatidylcholine) is created; by esterification with ethanolamine a Phosphatidylethanolamine is formed. Phosphatidylcholines are preferred.

Well-known examples, of phospholipids are:

### Phosphatidic Acid

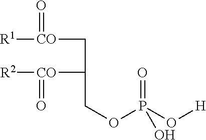

### Phosphatidylethanolamine (or Kephalin, Abr PE)

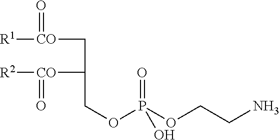

### Phosphatidylcholine (or Lecithin, Abr. PC)

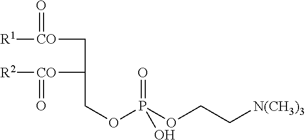

### Phosphatidylserine (Abr PS)

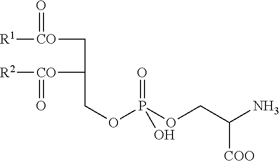

### Phosphatidylinositol

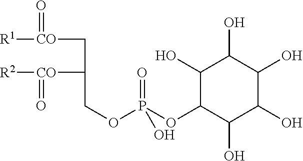

The existence of phospholipids which carry a nitro-carboxylic acid as a glycerine substituent have not been proven so far in cells/organisms and there is also no biosynthetic route known for this purpose.

Surprisingly it could be showed now that nitro-carboxylic acids-containing phospholipids do interact with other phospholipids, which resulted in an increase in the degree of order of phospholipid-layer-forming membrane-like structures. The same applies to the up-take of nitrated phospholipids into a phospholipid layer, which consists of physiologically occurring phospholipids. These effects have not been described in the literature nor were they predictable by a person skilled in the art.

The nitro carboxylic acid(s)-containing phospholipids can be used as pure substances, diastereomeric mixtures, regioisomeric mixtures, or mixtures of different nitro carboxylic acid(s)-containing phospholipids for the inventive bio-passivating compositions and the inventive bio-passivating coatings. Many nitration reactions result in mixtures of nitro carboxylic acids, which contain regioisomers and single or multiple nitrated carboxylic acids (as described here in detail). Such product mixtures of different nitrated carboxylic acids obtained from the nitration reaction can be used for esterification with the phospholipid residue, e.g. sn-glycero-3-phosphocholine, obtaining mixtures of different nitrated carboxylic acid(s)-containing phospholipids that can be used without requirement for separation of pure substances. Furthermore, the pure nitro carboxylic acid(s)-containing phospholipids as well as the mixtures of nitro carboxylic acid(s)-containing phospholipids can be used in combination with or as mixture with non-nitrated PL.

The inventive coatings can also consist of mixtures of nitrated phospholipids with phospholipids not containing a nitro group, which can be present in single or multiple layers. A high proportion of nitrated phospholipids in a PL layer is advantageous due to improved physico-chemical properties of phospholipid compositions on surfaces compared to layers of phospholipid compositions not containing nitrated phospholipids, e.g., by a high level of coverage, a low rate of multiple layer formation and a high strength to adhere to an artificial surface. Furthermore, the inventive phospholipid coating enables a spontaneous closure of remaining gaps of a phospholipid self-assembled monolayer (SAM) coating when phospholipid mixtures are used with a high content of nitrated phospholipids and moderate heat (up to 50° C.) and high humidity (example 1). It was shown also that SAM with a significant proportion of nitro-phospholipids exhibited a greater resistance against mechanical and chemical alterations than in SAM, which were made of comparable non-nitrated phospholipids. The lateral mobility of SAM with nitrated phospholipids is lower than of SAM from normitrated phospholipids, but the lateral mobility of pure nitrated phospholipids in monolayers is still measurable. Hydrophilic polymers such as PEG were physisosorbed in the presence of electrolytes such as calcium on the inventive phospholipid coatings. A top coating with hydrophilic polymers extended the shelflife of a closed formation of the inventive phospholipid coatings. The physisosorbed polymers could be quickly removed by rinsing. If such a combined coating was applied onto catheter balloons, no defect of the phospholipid monolayer was observed after balloon expansion.

Surprisingly it was shown that nitrated phospholipids exhibited a low dissociation rate from a mono-layer assembly compared to non-nitrated phospholipids. This effect can be explained by a denser packing of nitrated phospholipids and increased intermolecular hydrophobic forces (example 3). In this context, nothing is known about a relevant effect of nitric oxide. It was found that the number of nitro groups before and after the cell experiments were almost identical to the initial values. It is also unlikely that a pharmacologically relevant concentration of nitrated phospholipids diffuse into the cell membranes of the adhering cells, as was shown by experiments with radiolabelled nitrated phospholipids (example 4).

SAM of phospholipids with varying amounts of nitrated phospholipids showed a negligible amount of plasma protein adsorption, which was still the case when only nitro-carboxylic acids-containing phospholipids were used (example 5).

Cell adhesion experiments show significant differences from SAM without nitro-carboxylic acids-containing phospholipids on artificial surfaces as compared with SAM coatings containing nitro-carboxylic acids-containing phospholipids. While non-nitro-carboxylic acids-containing phospholipids with a choline head-group inhibited the adhesion of smooth vascular muscle cells and endothelial cells, SAM coatings of nitrated phospholipids with a cholin head group enabled cell adhesion which also allowed cell migration. This effect can amplified by the addition of hydrophobic molecules like cholesterol to SAM coatings of nitro carboxylic acid-containing phospholipids. In addition, endothelial cells could be immobilized, which grew together to form a closed layer of cells. In addition, those endothelial cells, which adhered to a SAM with nitrated phospholipids exhibited significantly reduced expression of cell adhesion molecules compared to cells that adhered onto a phospholipid-coated surfaces without nitrated carboxylic acids. Furthermore, it was shown that vascular smooth muscle cells (VSMC), which were allowed to grow on a synthetic surface with a phospholipid SAM and which have been stimulated during cell migration and proliferation by growth factors, exhibited a significantly lower proliferation rate and a lower production of extracellular matrix proteins, when the SAM coating contained nitro-carboxylic acids-containing phospholipids (example 6).

Bio-passivating properties of nitrated phospholipids cause an increased survival rate of cells (e.g., macrophages) by a reduced induction of apoptosis compared to a coating of non-nitrated phospholipids. The higher survival rate was paralleled by lower cytokine production of those cells; therefore it is likely that immune reactions at the site of the implant interface are low in cells adhering to the inventive nitro carboxylic acid-containing phospholipid coatings, thereby eliminating a proliferation stimulus of suchlike surfaces (example 7).

Thus, the results show surprisingly and unexpectedly that coatings of SAM with nitrated phospholipids improve cell homing of cells such as VSMC and endothelial cells, while cell responses to the artificial surface are reduced thereby providing a highly bio-compatible interface without pharmacological effects. Still surprisingly it was found that cells adhering to SAM-containing nitrated phospholipids were less responsive to cytokines and immunological stimuli than was the case in comparable SAM of phospholipids without nitrated carboxylic acid-containing phospholipids. Therefore, such a coating can be used for bio-passivation.

Surprisingly, it is possible due to the physico-chemical properties of phospholipids which have at least one nitrated carboxylic acid, not only to stabilize man-made phospholipid SAM-coatings but also phospholipid membranes of cells and thereby ensure bio-passivation, which on the one hand establishes a decreased cell/tissue reaction in response to contact with suchlike coated implant surfaces and on the other hand can also be used for protection and treatment of a cell/tissue damage. Synthetic and natural phospholipids containing no nitro groups on their free carbon chains, i.e., non-nitrated fatty acid residues, do not show such effects or comparable effects are present to a significantly lower extent. This aspect is elucidated further in the following.

### Differences in Physical and Biological Effects of Nitro-Carboxylic Acid(s)-Containing Phospholipids Compared to Reference Compositions.

Naturally occurring and synthetic phospholipids (PL) have been proposed for the coating of implant materials. In another disclosure, free nitrated fatty acids have been proposed to inhibit an aggressive healing pattern. For suchlike coatings of medical implant materials, improvement of biocompatibility compared with the use of uncoated materials was proposed. However, for coatings of phospholipids a clinically demonstrable improvement could not be demonstrated so far, as explained above. For phospholipids covalently-bound or immobilized by polymerization bio-passivating effects, as disclosed here have neither been documented nor would have been expected by a person skilled in the art. To obtain the disclosed inventive bio-passivating effects interaction between the inventive phospholipids and the cell/tissue structures it is essential, therefore these compounds are provided and used in an unbound form. There are some studies on the effects of non-nitrated phospholipids on model phospholipid membranes. Investigations for the inventive nitro carboxylic acid(s)-containing phospholipids indicated that the membrane melting point as well as the other physico-chemical parameters of model membranes made thereof, as well as on membranes of living cells behave completely different as compared to similar phospholipids without a nitrated of fatty acid residue. This applies in particular to the changes found for the membrane anisotropy, which behaves even in contrary to described changes that were found for the distribution of a NO— radical within a model membrane.

For the clarification of similarities, differences and peculiarities, the inventive nitro carboxylic acid(s)-containing phospholipids were set in direct comparison with comparable natural phospholipids, and fatty acids with or without nitration. Key findings are summarized in the following:

1. Free fatty acids (native or nitrated) are taken up by cells to a much greater extent as was recorded for phospholipids. The up-take of the free fatty acids is associated with a significantly lower limit of toxicity and a significant increase of the apoptosis rate. However, native phospholipids are also absorbed by cells and dissolute in the cell membrane, causing enlargement of the cells. This can promote the readiness for a cell division. Such a behavior was not seen after cell contact with the inventive nitro carboxylic acid(s)-containing phospholipids (examples 8 and 16).
2. The up-take of native free fatty acids and phospholipids into a cell is associated with an increase in cell proliferation. Only at toxic concentrations is the proliferation inhibited by the native fatty acids. The nitro carboxylic acid(s)-containing phospholipids show an inhibitory effect on cell proliferation being detectable already far below the toxicity threshold (examples 6, 8, 9). Compared to the reference substances cells incubated with nitro carboxylic acid(s)-containing phospholipids were significantly stronger adherent onto a surface coated thereof.
3. The physico-chemical properties of the nitro carboxylic acid (s)-containing phospholipids differ significantly from the corresponding non-nitrated phospholipids but also in respect to the free nitro fatty acid. In one aspect it was shown that SAM coatings with nitro carboxylic acid (s)-containing phospholipids have a significantly higher adherence onto the coated materials, thereby gaining a significantly stronger sliding ability that was also maintained longer than in the case with similar PL or fatty acids not containing a nitro group (examples 10, 12).
4. Above stated biological differences between nitro carboxylic acid (s)-containing phospholipids and PL not containing a nitro group can be explained in part by the difference found in adsorption of serum proteins (example 5). Those differences included the quality and quantity of serum proteins which had a significant effect on subsequent cell adhesion (examples 6, 9) as well as on consecutive immunologic reactions measurable by a reduced release of cytokines (example 7).
5. Different physico-chemical properties also receive or protect and stabilise those membranes when incubated with nitro carboxylic acid (s)-containing phospholipids as compared to non-nitrated PL and also when compared to incubation with free nitro fatty acids. Thus, it could be shown that cell membranes which had incorporated nitro carboxylic acid (s)-containing phospholipids are more resistance against osmotic pressure differences, membrane destructing toxins, as well as against hydrodynamic pressure differences and exhibit a higher thermal stability than those incubated with PL not containing a nitro-group (examples 10, 11, 13, 14, 15, 16, 18, 20, 21, 22).
6. Shedding of micro-particles requires evagination of the outer layer of the cell membrane and therefore is strongly dependent on the physico-chemical properties of the membrane. Shedding of micro-particles was significantly reduced when cells were exposed to nitro carboxylic acid (s)-containing phospholipids, compared to exposure of natural PLs or free nitro fatty acids; the finding can be explained with the aforementioned changes of membrane properties (examples 10, 14, 15).
7. Nociception and transmembraneous signal transduction are influenced by the physico-chemical properties of the cell membrane. Hindrance of the ability to open or close ion channels plays an important role in this regard. It could be shown that transmembraneous ion channel opening, which is observed in various conditions, e.g. in the context of hypoxia, a mechanical alteration of the cell, or due to receptor excitation, is reduced to a considerable extent after exposure of those membranes to nitro carboxylic acid (s)-containing phospholipids as compared to an exposure with PL without containing a nitro fatty acid (examples 11, 13, 16, 17, 19, 20).
8. Cell membrane permeability is dramatically increased after exposure to heat or freezing leading to cytolysis. This kind of injury is responsible for the damage of tissues/organs during storage in the cold. The additional use of natural phospholipids before cryo-conservation has no influence on this. For the inventive nitrated phospholipids a significant protective effect on cells that were exposed to freezing during cryopreservation was found, yielding a high viability of the treated cells after rewarming, whereby rewarmed cells treated with nitro carboxylic acid(s) regained normal functionality in a large part as compared to a pretreatment with PL not having nitrated fatty acids (examples 18, 21, 22).
9. Investigations concerning long-term stability of the natural and the nitro carboxylic acid (s)-containing phospholipids showed that the nitro carboxylic acid (s)-containing phospholipids had a significantly lower tendency to form trans-isomers as compared to the fatty acids in PL not containing a nitrated fatty acid (example 12). Because adverse biological effects are described for the cellular uptake of trans-fatty acids, avoiding the deployment of trans-fatty acids is beneficial.

The inventive nitro carboxylic acid (s)-containing phospholipids showed biocompatible, bio-passivating, but also proliferation reducing effects under various conditions. Therefore, it can be assumed that these effects also are transferable on other cell lines and other clinical conditions, where a bio-passivating effect is particularly desired.

One aspect of the effects of nitro carboxylic acid (s)-containing phospholipids is its bio-passivating effect on the reaction of the organism to trauma/Intoxication or a foreign surface. The bio-passivating effect conditions cells or tissues that come into contact with suchlike coated surfaces, in that way that typical pathological reactions preferably do not occur or are almost absent. This results, for example, in a healing pattern after contact with foreign materials that is characterized by low tissue proliferation. In one aspect, this conditions formation of an endothelial lining in vascular structures comprisied of a physiologically required number of cells and matrix proteins. Thus, an anti-restenotic effect is indirectly obtained, so that the bio-passivating properties of such coated implants also retain the ability to prevent symptoms of restenosis in vascular or luminal structures, so the overgrowth of an implanted stent and formation of scar tissue at the position that is stabilized by the stent is passively inhibited or stopped.

Surprisingly, it was found that phospholipids containing nitro-carboxylic acids, are suitable for coating of medical devices, due to their physico-chemical properties that generate favorable coating effects and because of resulting passive mechanisms in regard to prevention of restenosis or overgrowth of the medical device or on blood clotting and on cell damage after the implantation of the medical device.

Another aspect of the effects of phospholipids with nitro-carboxylic acids is their effect on the release and composition of micro-particles, which are shed from traumatised cells. Partition of the inventive compounds within cell membranes are possibly responsible for this finding, which results clinically in a local and systemic passivating effect on the responses of tissues to various kinds of alteration/traumatization. Furthermore, modification of proteins that are included in those micro-particles can also be assumed. This especially concerns glycoprotein tissue factors, by passively inhibiting their biological activity.

Another effect is the stabilization of the cell membrane (resistance against mechanical, chemical, osmotic or electrical irritation, as well as against mechanical, chemical, osmotic or electrical trauma) and maintenance of their functionality (i.e. membrane potential, regulation of ion channels, signal transduction).

Tissue hypoxia rapidly leads to changes within cell membranes. Consecutively phospholipases are activated, which cleave fatty acids out of membrane phospholipids. This conditions extent a later reperfusion injury which is related to the concentration of lysophospholipids. While exposure of natural phospholipids to cell membranes had no relevant influence on ischemia tolerance or the extent of reperfusion injury, surprisingly a significant reduction of hypoxia-induced cell damage was documented in cells that were exposed to the inventive nitro carboxylic acid (s)-containing phospholipids. The analysis conducted for the evaluation of re-perfusion injury is restricted on the basis of tests that were carried out under ex vivo conditions. However, it is documented in the literature that the extent of re-perfusion damage is correlated with the loss of NAD⁺ in the cytoplasm and intramitochondrial. Therefore, a reduction of hypoxia/reperfusion damage by rendering nitro carboxylic acid (s)-containing phospholipids into cell membranes can be assumed. It can also be assumed that the documented reduction in cell metabolism that occurs after exposure of cells to nitro carboxylic acid (s)-containing phospholipids contributes to the reduction of hypoxia-induced cell damage.

The inventive nitro carboxylic acid (s)-containing phospholipids have advantageous properties, which can be summarised as cell protective, bio-compatible and bio-passivating, where the common inventive concept can be summarized under the term bio-passivation.

Thus, the inventive nitro carboxylic acid (s)-containing phospholipids are suitable for bio-passivating or bio-compatible uses such as for instance for the production of medical compositions and for the coating of medical devices.

Due to their physical characteristics nitro carboxylic acid (s)-containing phospholipids are especially useful for bio-compatible mono-/bi- or multi-layer coatings of medical implants.

Accordingly the present invention also applies to medical devices, which are coated with at least a nitro carboxylic acid (s)-containing phospholipid. Such a coating is preferably a monolayer, bilayer or multiple layers and has bio-passivating features. On or within suchlike coatings active substances can still be included, for example, anti-restenotic substances, which are advantageous with catheter balloons and cardiovascular implants and is described further in the following.

Medical devices

### 1. Implant Coatings

Preferred implant materials are stents that are inserted into hollow organs to keep them open. Stents are conveniently produced in the form of tubes and classically are made of a lattice framework from struts. Stents are used for vessel stabilization, particularly to keep them open, which is of special importance in blood vessels, particularly for coronary arteries, or after dilatation of a vessel segment by balloon catheter to prevent renewed closure (restenosis). Stents can furthermore serve for the stabilization in the respiratory tract, esophagus, or the biliary tract. The coating of stents is a preferred use according to the invention.

Stents that are inserted into blood vessels are particularly preferred in accordance with the invention.

Balloon catheters are also preferred medical devices according to the invention.

Also preferred are instrument and implant materials that can cause tissue trauma while performing surgical, reconstructive or cosmetic procedures, which are related to cuts, tears, resections, connections and closures, graft- or prosthesis insertion. Implants are particularly but not exclusively soft tissue implants, especially breast implants, joint and cartilage implants, grafts of biological or artificial origin, intraocular lenses, surgical meshes and adhesion barriers, nerve regeneration conduits, shunts, vascular tubings of biological or artificial origin, catheters, probes, ports, drainages, stoma connections, endoluminal tubes, suture materials, ligatures, implantable apparatuses such as defibrillators, surgical instruments, such as hooks and forceps.

### 2. Wound Dressing Materials

A further field of application is the provision of nitro-carboxylic acid (s) containing phospholipids for tissue/organ systems for enhanced tolerance to metabolic, physical and chemical alterations by up-take of these phospholipids or by coating its surface with the inventive substances. By this means, passivation of these cells towards body's defence mechanisms (e.g., cytokines, and growth factors) or exogenous irritants (e.g. toxins) that could lead to a cell proliferation and/or apoptosis/necrosis is also accomplished. These documented effects are substantially different from the effects of naturally occurring phospholipids as well as of effects of nitrated fatty acids. The found effects for the inventive phospholipids comprise reduction of cell destructive effects and reduction of endogenous or exogenous effects that cause a non-physiological or hyper-regeneratory healing pattern. It is irrelevant, whether the tissue/organ trauma is/was caused by physical (e.g. mechanical, thermal), chemical or electrical mechanisms. As examples should be stated here: cut and crush wounds, burns, frostbite, alkali burns, ulcerations, radiation, allergen and toxin exposure.

This includes wound care preparations which are mounted on substrates or included in pharmaceutical preparations superficially applied to a tissue surface or introduced into the body.

In addition to the documented anti-adhesive effects of materials that are coated with nitro-carboxylic acid (s) containing phospholipids, lead to a better back off solubility of wound materials. In addition, these effects contribute to a reduction of adhesions/bondings (clamps).

The physico-chemical properties of the nitro-carboxylic acid (s) containing phospholipids can be used also, to cause a delayed resolution/release of compounds from a mixture, as well as to bring about their physical stabilization.

Especially preferred are applications that relate to surgical treatments, in which tissue is dissected or connected, especially if this dissection/connection is associated with trauma or another chemical or physical irritation of the tissue and includes in particular reconstructive and cosmetic surgery.

Preferred wound care materials are: wound dressings in the form of gels, tablets, colloids, adhesives, aglinates, foams, adsorbents, gauzes, cotton swabs and bandages.

### 3. Preparations for the Tissue/Organ Protection

Medical interventions often involve the need for a transient interruption of the blood supply of tissues or organs. Examples include the treatment of vessels or extraction/implantation of organ transplants. Pretreatment of tissues/organs with nitro-carboxylic acid (s) containing phospholipids causes an extension of ischemia tolerance so that pretreatment of such tissues is useful in those instances. This can be accomplished by applying a perfusion of the affected tissue/organ or by an installation or soaking of the tissue/organ with/in a solution with nitro-carboxylic acid (s)-containing phospholipids prior, during or after treatment of those tissues/organs. The inventive phospholipids are rapidly absorbed from the ambient medium or deposited on the cell or tissue surface.

The protection of vital tissues from cell destruction for a longer period of hypoxia is a special situation. To do this, the affected tissue/organs are cooled or frozen. At temperatures below 10° C. it comes to a demise of cells depending on the type of tissue and the preservation conditions. It has been shown that nitro-carboxylic acid (s)-containing phospholipids exhibit the ability to also preserve the integrity of the cell wall during the cooling and re-warming period significantly better than this is the case with natural phospholipids. Thus, nitro-carboxylic acid (s)-containing phospholipids are useful in preparations for cryopreservation of tissues/organs according to the previously reported effects on cell membranes and cell metabolism.

### 4. Preparations for the Stabilization of Cell Membrane Functions and Cell Integrity.

Cell membranes have a variety of functions and tasks. Many of these features are accomplished due to physical properties of cell membranes or changes thereof. This includes the mechanical resistance to physical and chemical alterations. But also interactions between the alkyl chains and membrane proteins have an influence on the functionality of ion channels and receptor proteins.

By up-take of nitro-carboxylic acid (s)-containing phospholipids and the resulting changes of membrane properties, it is possible to influence some of the membrane functions. It could be shown that alterations which lead to disruption of the membrane potential can be reduced by nitro-carboxylic acid (s)-containing phospholipids, thereby gaining anti-arhythmogenic properties. In addition, it could be demonstrated that the up-take of substances which are principally cell toxic and which are absorbed through various mechanisms through an intact cell membrane can be reduced substantially by prior incubation with nitro-carboxylic acid (s)-containing phospholipids, thereby reducing/eliminating up-take and/or effects of such toxins. In addition, up-take of nitro-carboxylic acid (s)-containing phospholipids in cell membranes exhibited an increased resistance toward an osmotic gradient of those cells. In addition, stabilization of the cell membranes has a significant effect on ion channels, especially on those that cause a change of in intracellular calcium concentrations, which can e.g. accomplish anti-arrhythmic effects and inhibited degranulation of eosinophlic cells.

In addition, it could be demonstrated that due to the changes of membrane properties induced by nitro-carboxylic acid (s)-containing phospholipids an increased resistance against an osmotic pressure gradient can be achieved. In addition the membrane stabilizing effects also influence sensitivity and function of membrane receptors and ion channels; therefore exogenous or endogenous stimuli have less or more effectiveness when nitro-carboxylic acid (s) containing-phospholipids were taken-up by a cell membrane. Thus, the reduced nociception of TNF alpha or TGF-β of fibroblasts is one example, thereby exhibiting an anti-fibrotic effect.

Pharmaceutical formulations that provide deploying of nitro-carboxylic acid (s)-containing phospholipids for tissues or organs in order to achieve stabilization of cell membranes that can be used for prophylactic and therapeutic tissue effects. These effects are not limited to the following indications:

Frost bite and burn injuries, acid or alkaline chemical burns, chronic overuse injuries such as tendonitis and fasziculitis, fibroting disorders such as the osteomyelofibrosis or interstitial pulmonary fibrosis, cardiac arrhythmias such as atrial flutter/fibrillation, ventricular premature beats, ventricular fibrillation, atrial ectopie, allergic reactions such as urticaria, allergic rhinitis/conjunctivitis, bronchial asthma, anaphylaxis; gastro-enteropathies such as tropical sprue or celiac disease, intoxication with animal or plant poisons, chemicals, as well as toxin-forming bacteria, or micro-organisms; chronic hyperreproductive diseases such as psoriasis, giant cell arteriitis, but also primary atrophic disorders such as the atrophic dermatitis and the Sudeck atrophy. Also included are pain syndromes such as neuropathies or meralgia paresthesia.

With the help of the impregnation solutions for dressings, wound and suture materials containing the inventive nitrated phospholipids, all nonrigid carriers which adapt to a given surface and largely cover this can be coated with the inventiive nitro-carboxylic acid (s)-containing phospholipids in addition to the previously mentioned devices. The inventive medical devices, which are preferably biodegradable are selectable from the group comprehending or consisting of: medical cellulose, dressing materials, wound inserts, surgical suture material, compresses, sponges, medical textiles, ointments, gels or film-forming sprays.

The medical cellulose and the medical textiles are preferably two-dimensional structures that are not very thick, which are impregnated with the nitro-carboxylic acid (s)-containing phospholipids. The nitro-carboxylic acid (s)-containing phospholipids accumulate on the fiber structures of these medical devices, which can be used in wet or dry form.

Sponges or generally biodegradable porous three-dimensional structures, which are a preferred form of the inventive application since nitro-carboxylic acid (s) containing phospholipids can be applied on both the inside and on the surface of porous structure of the cavities. These sponges can be used after an operation, e.g., to fill large wound cavities. From these spongy structures, the nitro-carboxylic acid (s)-containing phospholipids can be released, where the nitro-carboxylic acid (s)-containing phospholipids can also be present in a volatile or in the tightly-bound form. They can be released by both diffusion of loosely-bound nitro-carboxylic acid (s)-containing phospholipids out of the cavities of the porous structure as well as by biological degradation of the sponge structures.

A suitable medical device is also a carrier of the nitro-carboxylic acid (s)-containing phospholipids, “carriers” include the tissues that are described in detail herein, as cellulose, gels, film-forming compositions, etc., which can be biodegradable or bio-stable. The carrier may also consist of living matter or can contain radiopaque contrast agents. In addition, pharmacological agents can also be inserted in the medical device, which can be released by diffusion from or biodegradation of the carrier, as described below.

Any medically used textile or cellulose suitable to manufacture wound pads or dressings, bandages or other medical tissues or meshes is called “fabric” herein.

Polyhydroxybutyrate and cellulose derivatives, chitosan derivates as well as collagens, polyethylene glycol, polyethylene oxide, and polylactic acid are preferred materials for medical cellulose and textiles. If alginate is used as a wound dressing, calcium alginate with sodium carboxymethylcellulose products are preferentially used. SeaSorb® soft made by the company Coloplast is one example.

When nitro-carboxylic acid (s)-containing phospholipids are applied on wound dressings and/or or wound inserts, especially products of Tabotamp® and Spongostan® made by the company Johnson and Johnson shall be mentioned. These products are produced by controlled oxidation of regenerated cellulose.

Surgical suture material can be characterized with regard to its construction in monofilament and multifilament threads. Multifilament threads can show a so-called wick effect. This means that tissue fluid can migrate along the thread by capillary forces. This can enhance migration of bacteria and thereby spread an infection. It is therefore desirable to prepare surgical suture material to prevent bacterial propagation along the artificial surface effectively. Therefore, it is advantageous to coat or impregnate surgical suture materials in order to reduce bacterial colonization and migration. This can be achieved using solutions, e.g., a methanol solution where nitro-carboxylic acid (s)-containing phospholipids are homogeneously dissolved, that are used to wet the suture material, then the methanol is allowed to evaporate, thereby forming a homogeneous coating. Instead of methanol, other lower alcohols can also be used, such as ethanol, propanol, and isopropanol or their mixtures with methanol. It is further preferred to use nitro-carboxylic acids-containing phospholipids in disinfectant solutions, such as octenidindichloride solutions (sold under the name OcteniseptB®, made by the company SchOle & Mayr) or to dequaliniumchloride solutions. The weight ratio of octenidindichloride or dequaliniumchloride to nitro-carboxylic acid (s)-containing phospholipids is preferably 1:0.1 up to 1:5, whereby 1:1 is particularly preferred. If surgical suture materials are considered for a coating with the nitro-carboxylic acid (s)-containing phospholipids, suture materials should consist preferably of polyglycolic acid, polycaprolactone coglycolide, or poly-p-dioxanone. Examples here include products like Marlin®, PCL and Marisorb® made by the company Catgut GmbH should be named.

If compresses should be impregnated with the nitro-carboxylic acid (s)-containing phospholipids, sterile gauze from 100% cotton should be used particularly. Examples here are Stericomp® and Askina product lines.

If medical cellulose is used, then it is preferred that it have a proportion of more than 90% cellulose. If medical textiles are used, Trevira® products are preferred.

The medical textiles and cellulose are dipped in or sprayed with a solution of nitro-carboxylic acid (s)-containing phospholipids in an appropriate concentration in water, organic solvents such as ethanol or mixtures thereof, whereby the immersion or spraying can be repeated several times after drying of the medical device. Per cm² surface are of the medical device, 10 μg to 100 mg of nitro-carboxylic acid (s)-containing can be applied to the surface.

The medical sponges are bio-resorbable implants with sponge-like, porous structures. Preferred materials for medical sponges are collagens, oxidized cellulose, chitosan, thrombin, fibrin, chitin, alginates, hyaluronic acid, PLGA, PGA, PLA, polysaccharides and globin. If medical sponges are used, those which contain more than 90% collagen are preferred.

If the nitro-carboxylic acid (s)-containing phospholipids are used as an ingredient of ointments, an ointment base containing or consisting of purified water preferred in a quantity of 5-50 weight %, especially favored of 10-40 weight (wt) % and the most favored of 20-30 wt % can be used. It is also preferred when the ointment contains Vaseline in a quantity of 40-90 wt %, especially favored of 50-80 wt % and most preferred is 20-60 wt %. In addition, the ointment can also contain viscous paraffin in a quantity of 5-50 wt %, especially favoured of 10-40 wt %, and the most favoured of 20-30 wt %. Still preferred are geling agents and/or film formers in an amount of up to 30 wt %. In addition, polymers such as cellulose, chitosan, thrombin, fibrinogen, chitin, alginates, albumin, hyaluronic acid, hyaluronan, polysaccharides, globin, polylactide, glycoside, polylactide co-glycolid, polyhydroxybutyrate, cellulose derivatives, chitosan derivates, polyethylene glycol and polyethylene oxide in amounts up to 30 wt % can be added.

Phospholipids containing the inventive nitro carboxylic acids can be also implemented in paints or be part of film-forming sprays.To better stabilize the film-forming sprays, the nitro carboxylic acids-containing phospholipids described herein can be combined with gel or film formers. Film-forming sprays contain at least one or more film formers.

Appropriate film formers are preferably substances on basis of cellulose such as cellulose nitrate or ethyl cellulose or physiologically harmless polymers thereof, polyvinyl acetate, partially saponified polyvinyl acetate, mixed polymers of vinyl acetate and acrylic acid, cotronic acid or maleic acid mono alkyl esters, ternary mixed polymers of vinyl acetate and cotronic acid and vinyl decanoate, or cotronic acid and vinyl propionate, mixing polymers of methyl vinyl ether and maleic acid mono alkyl esters, in particular as maleic acid monoesters butyl, mixing polymers of fatty acid vinyl ester and acrylic acid or methacrylic acid, mixed polymers of poly-N vinylpyrrolidone, methacrylic acid and methacryl acic alkylester, mixied polymers of acrylic acid and methacrylic acid or acrylic acid alkyl ester or methacryl acid alkylester, in particular with a content of quaternary ammonium groups, or polymers, copolymers or blends containing ethyl acrylate, methyl methacrylate or trimethylammonioethylmethacrylat chloride, or polyvinylacetals and polyvinyl butyrals, alkyl-substituted poly-N-vinylpyrrolidone, alkyl ester of mixed polymers of olefins and maleic anhydride, reaction products of rosin with acrylic acid and benzoin resin, chitosan, Luvimer 100®, aluminium stearate, carbomers, Cocamide MEA, carboxymethyl dextran, carboxymethylhydroxypropyl guar or red algae carrageenans. In the aforementioned esters the alkyl residues are usually short chained and have usually no more than four C atoms.

Film formers also include water soluble polymers such as for example ionic polyamide, polyurethane and polyester as well as homo- and copolymers of ethylenic unsaturated monomers. Examples of such substances are available under the trade names, Acronal®, Acudyne®, Amerhold®, Amphome®, Eastman AQ®, Ladival®, Lovocryl®, Luviflex VBM®, Luvimer®, Luviset P. U. R.®, Luviskol®, Luviskol Plus®, Stepanhold®, Ultrahold®, Ultrahold Strong® or Versatyl®. Luvimer® it is a polyacrylate as hair styling polymer developed by the company BASF AG.

As a solvent, water, ethanol or water-ethanol mixtures are preferred. The production of the nitro-carboxylic acid (s)-containing phospholipids coated implants is accomplished by dipping or spraying techniques. The implant products dipped or sprayed with an appropriate solution in which the nitro-carboxylic acid (s)-containing phospholipids are suspended. The implants are then dried and sterilized. Gels, ointments, solutions and sprays can prepared by the appropriate pharmaceutical preparation made according to standard methods and preferably in a last step with the desired amount of nitro-carboxylic acids-containing phospholipids-. Also those available inventive medicine devices are part of the present invention.

The inventive rinsing solutions for medical apparatuses containing at least one inventive nitro-carboxylic acid (s)-containing phospholipids can be used for flushing and cleaning of instruments and accessories, to moisten wound tamponades, towels, bandages, filling of the respiratory humidification devices or for checking the permeability of catheters, nasal irrigation and of intra- and postoperative fluid during endoscopic procedures, flushing and cleaning of wound drainage catheters. The inventive rinses for wounds containing at least an inventive nitro-carboxylic acid (s)-containing phospholipids can be used for rinsing and cleaning, used in surgical procedures, flushing and cleaning in stoma care, flushing of wounds and burns, and the mechanical rinsing of the eye. Wound rinsing solutions generally serve for the removal of cell residues, necrosis, blood and pus but also of wound dressing's residues.

When using the rinses with inventive nitro-carboxylic acids-containing phospholipids in order to wash surfaces, a coating or a film can be formed and so a bio-passivation effect of suchlike treated surfaces can occur. Suitable basic solutions or formulations which are suitable for mixing with the inventive nitro-carboxylic acid (s)-containing phospholipids can be used, e.g. saline, Ringer or Ringer's lactate solution, solutions containing polyhexanid or polyethylene glycol, and commercially available wound rinsing solutions like Prontosan® or Lavanid®.

A further aspect of the invention relates to cryopreservation of biological samples. The expression “biological samples”, as used herein, includes cells, both eucaryotic and also prokaryotic, organs and tissues and biologically active molecules, such as macromolecules such as nucleic acids or proteins. The cryopreservation of human embryos and embryos of other mammals is particularly preferred. The storage of cells in the frozen state (cryo or cold storage) is a procedure which is usually used to achieve long-term maintenance of viable cell material and genetic stability. One embodiment for the use of nitro-carboxylic acid (s)-containing phospholipids comprises of provision of cryoprotection solution or a cryopreservation medium. The inventive nitro-carboxylic acid (s)-containing phospholipids have this positive effect on the survival rate of cells/micro-organisms.

Cryoprotection solutions or cryopreservation media generally obtain their ability to preserve viable cells from cold damage by amorphously solidifying disaccharides and polymers, such as glycerol, dimethyl sulfoxide (DMSO). Cryoprotection solutions or cryopreservation media, which should be suitable for the freezing of cells, organs and tissues have a culture medium as a basis. All popular media can be used as a culture medium to culture micro-organisms, cells, and tissues.

They may also include: buffer substances, indicators, dyes, inhibitors (for example, antibiotics) or growth auxiliary substances (hormones, vitamins and the like).

Cryoprotection solutions for the freezing of macromolecules contain no media as a basis but aqueous buffer solutions are preferred. Amorphous solidifying disaccharides and polymers are being use preferentially as cryo-protectants for macromolecules.

Lyophilization of pure protein solutions is occasionally accompanied by instability of the proteins, which can be prevented by the addition of lyoprotection solutions. The inventive lyoprotection solutions differ from the above describes cryoprotection solutions only by the stabilizing additives used. While the cryo-protectants ensure stability of cells during freezing, lyoprotectors are used during drying. Lyoprotectors form hydrogen bonds to functional polar groups of macromolecules, thereby forming a matrix which acts as water replacement. Therefore, molecules that have hydrophilic groups are favorable to fulfil this task. Disaccharides and mannitol are preferred here, to name a few.

Because lyophilization includes both freezing and drying steps, the present invention also includes solutions containing at least one inventive nitro-carboxylic acid (s)-containing phospholipids as well as a lyoprotector and a cryoprotectant.

Contrast agent solutions containing at least one of the inventive nitro-carboxylic acid (s)-containing phospholipids and a contrast agent or contrast agent analogue are of particular interest. Such contrast agents or contrast agent analogues mostly contain barium, iodine, manganese, iron, lanthanum, cerium, praseodymium, neodymium, samarium, europium, gadolinium, terbium, dysprosium, holmium, erbium, thulium, ytterbium and/or lutetium; preferred are ions in bound or complexed forms.

Preferred are X-ray contrast agents that are used for the diagnostic imaging of the joints (arthrography) or during CT (computed tomography). They can be used otherwise during X-ray diagnostics or interventions, computed tomography (CT), magnetic resonance imaging (MRI) or ultrasound, whereby magnetic resonance imaging (MRI) is preferred. Furthermore, iodine-containing contrast agent used in the vascular imaging (angiography and venography) and CT (computed tomography) are preferred. The following examples can be cited as iodine-containing contrast agents: Amidotrizoe acid, lotrolan, lopamidol, Iodoxamin acid, Jod-Lipiodol® and amidotrizoat. The paramagnetic contrast agent represent another class of preferred contrast agents, which are usually a lanthanide, e.g. gadolinium (Gd³⁺), europium (Eu²⁺, Eu³⁺), contain dysprosium (Dy³⁺) or holmium (Ho³⁺). Gadolinium diethylene triamine pentaacetic acid, gadopentet acid (GaDPTA), gadodiamide, meglumine gadoterat and gadoteridol are examples of gadolinium-containing contrast agents.

The term “medical composition” or “solution” as herein is understood as mixtures of at least one inventive nitro-carboxylic acid (s)-containing phospholipids and a solvent and/or excipient and/or carrier, so an actual solution, dispersion, suspension or emulsion of a nitro-carboxylic acid (s)-containing phospholipids or a mixture of various nitro-carboxylic acid-containing phospholipid and at least a further component selected from the solvents oils, fatty acids, fatty-acid esters, phospholipids, amino acids, vitamins, contrast agents, salts or membrane-forming substances. The term “solution” should also clarify that it is a liquid mixture, which however may also be gel-like, viscous or pasty (thick viscous or highly viscous).

According to another aspect of the invention a perfusion and preservation solution containing at least one inventive nitro-carboxylic acid (s)-containing phospholipids are provided for the preservation of cells in the absence of a blood supply in particular in the preservation of complex cell systems such as organs or living tissue.

Organ transplantation is now available for kidney, liver, heart, lung, pancreas, intestine, cornea and skin. During removal of the organ from the donor, the vascular system of an organ transplant is perfused with a preservation solution. This solution is designed, to facilitate reduction of the organ temperature, to prevent the swelling of cells, to eliminate oxygen free radicals, to control the pH, to reduce ischemic damage, to extend the safe time during which the organs are kept alive while they are kept outside the body, and to facilitate the recovery during reperfusion when implanted in the body. There are several commercially available preservative solutions e.g., Eurocollins-® solution, the University of Wisconsin Solution®, Celsior Solution® and the low-potassium-dextran solution (Perfadex® solution).

The inventive perfusion or conservation solutions are basically an aqueous pH buffered system, preferably from a sodium phosphate buffer and a potassium phosphate buffer with a pH in the range from 6.8 to 7.4 are selected, such as for example Krebs-Henseleit buffer (KHB).

In general terms, the inventive perfusion or preservation solution may contain further ingredients in addition to the inventive nitro-carboxylic acid (s)-containing phospholipids such as: water for injections, sucrose, at least one component with pH-buffering capacity, calcium ion channel blocker, calcium ions, coagulation inhibitor, such as acetylsalicylic acid, colloid-osmotic complexes such as polyethyenglycol (PEG) or chelators such as amino acids.

### Coating of Medical Devices

Medical devices are of particular interest for the use with the inventive nitro-carboxylic acid (s)-containing phospholipids with a preferred use for the coating of stents used in blood vessels, which are single- or double-layer coatings with nitro-carboxylic acid-containing phospholipids. This form of embodiment is particularly advantageous, since the bio-passivating coating can be easily manufactured and since the thickness of the stent struts only marginally increases due to this coating, and at the same time an anti-restenotic effect can be achieved. Preferred are phospholipid coatings providing a single layer (monolayer), where the height of layer is only that of a molecule as suggested by the term. A monolayer according to the intervention is preferably fully covering the medical device. It is however also possible that only parts of the medical device are covered with a monolayer.

Still preferred are stents coated by a double layer of nitro-carboxylic acid-containing phospholipids. Stent with a monolayer or double layer of nitro-carboxylic acid-containing phospholipids which have a layer of at least a bio-absorbable polymer is a more preferred embodiment.

Still preferred medical devices are also coated balloon catheters having a pure layer of nitro-carboxylic acid-containing phospholipids. Balloon catheters with a double layer of nitro-carboxylic acid-containing phospholipids are still preferred. Balloon catheters with a monolayer or double layer of nitro-carboxylic acid-containing phospholipids which have a layer of at least a bio-absorbable polymer is a more preferred embodiment. These double layer systems are preferred for balloon catheters.

Balloon catheters and stents, which have a pure coating with nitro-carboxylic acid-containing phospholipids and a surface layer of contrast agent are still preferred.

Also preferred are coatings of stents, balloon catheters, and other artificial implants using layers of nitro-carboxylic acid (s)-containing phospholipid layers and layers of contrast agents or similar substances. Coatings with 2-10 nitro-carboxylic acid-containing phospholipid layers are preferred, more preferred 2-6 layers and 2-4 layers the most preferred embodiments.

Basically all body implants are suitable for a coating with nitro-carboxylic acid (s)-containing phospholipids, because of the documented effects of an improved healing compared to native phospholipids or non-coated implant material. Included are implants for reconstructive and plastic surgery such as surgical meshes, implants for tissue replacement or reconstruction, implantable ports and indwelling catheters, and drains are especially suitable for an inventive coating.

The phospholipid layers are naturally very thin. The thickness of a single phospholipid layer can be specified with 2-4 nm. Accordingly, the thickness of phospholipid double layer is 4-8 nm and with more layers the respective values sum up.

Optionally, the stents can have a hemo-compartible layer of the bio-compatible substances mentioned below, which are preferably bound to the surface thereof.

In a preferred embodiment the coating consists of at least one layer of nitro-carboxylic acid (s)-containing phospholipids and a pharmacological active substance which has preferably an anti-proliferative or anti-restenotic effect, to be used as a pure active substance solely, or in combination with an excipient. Particularly mentioned here are: rapamycin, tacrolimus, bleomycin, mitomycin, methotrexate, fludarabine, fludarabine-5′-dihydrogenphosphate, cladribine, mercaptopurine, thioguanine, cytarabine, fluorouracil, capecitabine, docetaxel, carboplatin, cisplatin, oxaliplatin, irinotecan, topotecan, hydroxycarbamide, adriamycin, azithromycin, bromocriptine, SMC proliferation inhibitor-2w, mitoxanthrone, azathioprine, dacarbazine, fluorblastin, probucol, colchicine, tamoxifen, estradiol, tranilast, taxanes and derivatives, such as paclitaxel and carboplatin, taxotere, synthetically manufactured and extracted from native sources macrocyclic oligomers of coal-sub oxide (MCS) and its derivatives, heparin, hirudin, histamine antagonists, tocopherol, corticosteroids, non-steroidal substances (NSAIDS) and mixtures of diastereomeres, metabolites and mixtures of above-mentioned substances.

The active substances can be used individually or combined in the same or a different concentration. Active substances are particularly preferred which have in addition to their anti-restenotic effect more supportive properties, e.g., anti-proliferative, anti-migratory, anti-angiogenic, anti-inflammatory, cytostatic, cytotoxic or antithrombotic effects.

It is preferred that the active substance or the active substances is/are included in a pharmaceutically active concentration of 0.001-10 mg/cm² stent surface area.

In addition, solutions of excipients and the active substance altogether can be top-coated onto a layering of nitro-carboxylic acid (s)-containing phospholipids which can be useful for example as contrast agent providing visualization of the medical device or can act as so-called transport intermediaries and accelerate the up-take of the active substance into a cell. These include also vasodilators, including endogenous substances such as kinins, substances of plant origin as Gingko biloba, DMSO, xanthones, flavonoids, terpenoids, animal and vegetable dyes, contrast agents and contrast agent analogues, as well as cholesterols also included into the group of pharmaceutical additives but can also be used as an active component or have an synergistic effect.

Further substances that are a preferred embodiment are 2-pyrrolidone, tributyltin and triethyl citrate as well as acetylated derivatives, dibutylphtalate, and benzoic acid benzyl ester, diethanolamine, diethylphtalate, isopropylmyristate-palmitate, triacetin, etc.

In a more preferred embodiment, the nitro-carboxylic acid-containing phospholipid layer (s) can be onto or beneath a layer (s) of polymers and polysaccharides which can be as arranged in a sandwich fashion as well, with or without a layer of an active substance. The presence of a layer of active substance besides the phospholipid layer is a preferred embodiment. The polymer layer can consist of stable organic or bio-degradable polymers. The biodegradable polymer layer however is preferred. This preferred embodiment is advantageous, because during the decomposition of the polymer the active substance can be delivered slowly over an initial period to the surrounding tissues. Thereby, the bio-passivating stent would also have an anti-proliferative long-lasting effect. Furthermore, the polymer layer can be used to bind at least one active substance which is a preferred embodiment.

Typically bio-degradable or absorbable polymers can be used, for example: polyvalerolactone, poly-ε-decalactone, polylactic acid, polyglycolide, copolymers of polylactic acid and poly-ε-caprolacton, polyglycolide, polyhydroxybutyric acid, polyhydroxybutyrate, polyhydroxyvalerate, polyhydroxybutyrate-co-valerate, poly(1,4-dioxan-2,3-dione), poly(1,3-dioxan-2-one), poly-para-dioxanone, poly anhydrides such as poly maleic anhydride, polyhydroxymethacrylate, poly(lactic-co-glycolic)acid, fibrin, polycyanoacrylate, polycaprolactone dimethylacrylate, poly-b-maleic acid, poly caprolactone butyl-acrylate, oligocaprolactone diol, multi block polymers such as poly ether ester-multi block polymers such as PEG and poly(butylenterephtalate). and oligodioxanondiol, polypivotolactone, polyglycolic acid trimethyl carbonate polycaprolactone glycolide, poly(g-ethylglutamate), poly(dth-iminocarbonate), poly(DTE-co-DT-carbonate), poly(bisphenol α-iminocarbonate), polyorthoester, polyglycolic acid trimethyl carbonate, polytrimethylcarbonate, polyiminocarbonate, poly(N-vinyl)-pyrolidone, polyvinyl alcohol, polyester amide, glycolysed polyester, polyphosphoester, polyphosphazene, poly [(p-carboxyphenoxy) propane], polyhydroxypentanoic acid, polyethylene oxide propylenoxid, soft polyurethanes, polyurethanes with amino acid residues in the backbone, poly ether ester such as the polyethylene oxide, polyalkenoxalate, polyorthoester and their copolymers, carrageenans, fibrinogen, starch, collagen, protein-based polymers, poly-amino acids, synthetic poly-amino acids, zein, polyhydroxyalkanoates, pectin acid, actinic acid, fibrin, casein, carboxymethylsulfate, albumin, hyaluronic acid, heparan sulfate, heparin, chondroitin sulfate, dextran, β-cyclodextrins, copolymers with PEG and polypropylene glycol, rubber of Acacia gum, guar, gelatine, collagen, collagen N-hydroxysuccinimide, lipids and lipoids, polymerizable oils with low level of networking, modifications and copolymers or blends of the above substances.

### Uses

The inventive coatings for medical devices and compositions for medical or cosmetic procedures are particularly suitable to prevent, reduce or treat vascular stenoses, or restenosis, and to be used in vascular lesions, vascular interventions, bypass graftings, and treatment of coronary heart disease or peripheral artery disease, heart valve disease, varicose veins, vasculitis, lymphangitis, erysipelas, during extracorporeal circulation, to maintain patency of artificial or natural conduits or orifices (e.g. stomata), cuts and contusions, blunt ulcers, canker sores, necroses, dermatitis, urticaria, pruritus, burns, frost damage, radiation damage, abrasions and lacerations, crushing and laceration trauma, connective tissue diseases like dermatomyositis, Sudek syndrome and fibromyalgia, nerve irritations such as carpal tunnel syndrome and Meralgia paresthetica, bronchial chronic obstructive lung diseases including asthma, anaphylaxis, including toxic shock syndrome, allergies, including hay fever, intoxication, tissue connections by adaptation, suture, clamping, cauterization or tissue welding, tissue removal, organ transplantation, tissue thightening, tissue and skin reconstruction, scar revision, hernia repair, glaucoma drainage, polyps, alopecia, atrophy and barotrauma. Especially preferred are the medical devices according to the invention for the treatment and prevention of restenosis.

Using the inventive medical devices for the treatment in artificial blood conduits or pumps, these include but are not restricted to endo-prostheses from PTFE, PET, polyester etc. to be used instead of a natural blood vessel or pump system for intra- or extracorporeal circulation, as well as their connections, which can be made from polyurethane, PTFE, polyester, etc. This includes the application of patch materials which may consist for example of polyester or allogeneic or xenogenic tissue.

In a preferred embodiment, the medical devices are covered with a visco-elastic layer consisting of the inventive phospholipids. It turned out that suchlike coatings of medical devices improve their lubricity and traceability within the vascular system. This property reduces injuries of the endothelial layer during the approach of the medical device on the one hand and promotes the healing process on the other hand, while reducing unwanted reaction to the foreign body contact.

The nitro-carboxylic acid (s)-containing phospholipids disclosed herein can be used for coating of medical devices to provide in particular an improved sliding ability of medical devices. Preferably, the sliding ability is improved in medical devices that come in contact with tissues which are in particular catheters, dilatation catheters, catheter balloons, guide wires, guiding catheters, stents and other medical devices used in blood vessels. This effect is also provided for coatings of wound supplies such as suture material, needles, cerclages, wires and surgical meshes but also tissue replacement materials such as artificial tendons and bone replacement materials. Also, improvement of slippage can be beneficial during implantation of a device but also of soft tissue implants such as breast implants.

“Improvement of lubricity” as used herein refers to a sliding ability of coated medical device for insertion and propagation in preformed or artificially created body cavities, which is better than the sliding ability of the uncoated medical device during the insertion into or through those tissues or cavities.

### Coating Processes

The monolayers, double layer systems or multilayer systems on a stent, a balloon catheter or other implants are preferred produced by spraying, spin-coating method, dipping, pipettiing procedures, chemical vapor deposition (CVD) and the atom layer deposition (ALD), but especially preferred are dipping and vapor deposition. This is preferably performed in uncoated or coated surfaces covered with a biocompatible layer of a stent or balloon catheter using nitro-carboxylic acid (s)-containing phospholipids as a coating solution.

### Dip Coating

Phospholipids can form self assembling monolayers (SAM) by dip coating on appropriate surfaces. SAM spontaneously form on a surface while dipping into a solution or suspension of surface-active substances or organic substances. The layer can be supported by a prior coating of the surface with a covalently-bound alkyl layer, e.g. in the form of alkanthiols, preserving a high physical adherence of the physiosorbed carbon chains of the phospholipids. Hydrophobic molecules such as cholesterol, incorporated in suchlike phospholipid layers were considered suitable for the creation of focal adhesion points, which are necessary for cell adhesion and cell migration on a surface. Cholesterol and related substances can easily be integrated into an artificial phospholipid layer on the basis of known methods. Another method, which allows the development of focal adhesions of anchoring cells is the implementation of cell adhesion proteins such as such as RDG tripeptide. However, these must be attached covalently to the surface of the device to ensure their physical stability, while complex hydrophobic molecules, which are integrated into a phospholipid layer, can not easily be removed.

Various phospholipids with nitrated phospholipids can be used for the coating of metallic and polymeric surfaces via physisorption methods, as shown by the examples. Physisorption is the common form of adsorption where an absorbed molecule is bound onto a substrate by physical forces.

For the coating process, the implant is dipped into the solution-containing tank. The procedure is repeated until a complete and homogeneous distribution of the coating on the surface of the implant is achieved. For better distribution of the coating, the implant can optionally be dipped under constant movement of its position in the tank, e.g., by rotation.

Immersion is also a suitable procedure for polymers such as for phospholipids. After applying the polymer layer by dipping, the coated implant can be dried by rotary drying.

### Chemical Vapour Deposition Method

Vapor deposition is a more preferred coating method. This method is particularly useful for the production of very thin layers, where the layer thickness and uniformity of the coating can be well controlled, which is of need when creating monolayers of phospholipids or of an active substance.

### Pipetting Method—Capillary Method

This preferred method uses a fine nozzle or needle positioned next to the medical device to spray the coating solution onto the medical device. This procedure allows an exact and precise coating of the surface of the device, and is suitable for inventive nitro-carboxylic acid-containing phospholipids, active substances and polymers. The process of coating medical devices with active substance solutions and polymers is particularly preferred.

This process is possible with each coating solution, which is still so viscous that it is attached by adhesion forces or in addition taking advantage of gravity over the device surface within 5 minutes, preferably within 2 minutes, thereby completely covering the medical device.

### Langmuir-Blodgett Procedure

Here, the medical device is dipped in a liquid in a vertically orientation and pulled out slowly again. By this means the so-called Langmuir-Blodgett layer which is self arranging on the surface of the liquid containing amphiphilic or hydrophobic molecules adheres to the surface of the medical device due to adhesion forces, thereby creating a monolayer of the organic molecules on their surface. The inventive organic molecules form a film on the surface of the liquid repetively and spontaneously. Ideally, the number of monolayers and thus the thickness of the coating can be controlled by the number of immersion operations. If it is an aqueous solution, the hydrophobic end of the organic molecule aligns towards the hydrophobic surface of the medical device, while the hydrophilic end of the organic molecule is water-orientated. Repetition of dipping and pulling out will take up the organic molecules in an opposite orientation since the surface has a hydrophilic surface. In this instance, the molecules of the Langmuir-Blodgett layer are turned and adhere to the surface by hydrophilic adhesion forces. Therefore, organic molecules are preferred for this coating process, which have both a hydrophilic and hydrophobic residue. This technique is ideal for coating with phospholipids and long-chain fatty acids.

To achieve optimum results, the molecule layer on top of the aqueous solution is influenced by a so-called film balance. This keeps the so-called transfer pressure and thus the surface area concentration of the organic molecules constant.

### Procedure According to Langmuir-Schaefer

This method is a variant of the Langmuir-Blodgett procedure previously described. Using highly viscous films or in case of formation of aggregates or crystals, vertical immersion can be problematic. Better results in this case can be obtained by the Langmuir-Schaefer method, in which the medical device is horizontally dipped. The process steps that follow are equivalent to those used in the Langmuir-Blodgett technique.

### Solvent Dissolution Method

In this process, non-polar long-chain molecules, for example, the aforementioned phospholipids are forced to form micelles by using an appropriate detergent solution. Suitable detergents for this are, for example, cholate, desoxycholate, octyl glucoside, heptyl glucoside, and Triton X-100. This solution is then dialyzed to remove the detergent. The phospholipids can form in this way liposomes that bind to the surface of the medical device.

### Rotary Coating (Spin Coating)

Here, the medical device is attached to the bottom on a turntable using vacuum suction. The desired quantity of the solution is applied through a metering device positioned above the center of the medical device. An appropriate choice of acceleration, maximum speed and the rotation period achieves a uniform coating with a film. Excess coating solution is removed, however, by centrifugal forces.

### Painting

Furthermore various methods for painting and creating coatings on substrates which are known in the art can be used for the inventive coating of medical devices with the inventive phospholipids. The use of decane or hexane as a solvent is preferably suitable.

### DESCRIPTION OF THE FIGURES

FIG. 1 shows studies on effects of nitro carboxylic acid of containing phospholipids on cell physiology. The first line shows the results of the lipid staining, the second line of the results of the MTT assay, the third line the change of cell volume and the fourth line the rate of viable cells assessed by the live/dead assay. The substances tested are: PC: phosphatidylcholine, DOPC (1,2-dioleoyl-sn-glycero-3-PC), POPC (1-palmitoyl-2-oleoyl-sn-glycero-3-PC), SOPC (1-stearoyl-2-oleoyl-sn-glycero-3-PC), ONOPC (1-oleoyl-2-(E-9-nitrooleoyl)-sn-glycero-3-PC), PNLPC (1-palmitoyl-2-(E-9-nitrolinoleoyl-sn-glycero-3-PC), as well as the free fatty acids (oleic acid) OA, LA (linoleic acid), NOOA (E-9 nitro oleic) and NOLA (E-9 nitro linoleic).

FIG. 2 shows studies on effects of nitro carboxylic acid-containing phospholipids on adhesion, migration, and proliferation of cells. The substances tested are: SOPC, DOPC, POPC, ONOPC, PNLPC, as well as the free fatty acids of OA, LA, NOOA, and NOLA. The first two lines show the effect of the substances on proliferation of the cells tested after incubation with concentrations of 10 μmol and 100 μmol after 24 h, 48 h or 72 h. The relative numbers (%) of cells compared to the untreated control are shown. The following two lines show the cell detachment after 24 h and 72 h of pre-incubation with the substances at 10 and 100 μmol. In the last lines, the results of the cell migration assay are shown after each 24 h, 48 h or 72 h of incubation with the substances. Here too, the relative numbers of cells (%) as compared to the untreated controls are shown. The values refer to the results from incubation at concentrations of 10 or 100 μmol of the respective substances.

FIG. 2a When comparable results were obtained within the specified compound classes they were pooled. The calculated values were analyzed to revealed statistically significant differences between the natural PL POPC and DOPC compared to the results of the nitrated PL ONOPC and PNLPC. Mixtures of substances that have yield comparable results were pooled accordingly as specified. In the presence of statistical differences between results using natural PL or nitrated PL, it was determined whether the values found for nitrated PL were within the standard deviation of the values obtained (=o), were above (=+), significantly higher (=++) or were lower (=−). Values that were not statistically different from the values of the natural PL are specified n.s.

FIG. 3 shows the investigations on the stability of nitro carboxylic acid phospholipids and their effects in phospholipid mixtures. The natural phospholipids POPC (1-palmitoyl-2-oleoyl-sn-glycero-3-PC), SLPE (1-stearoyl-2-linoleoyl-sn-3-glycero-phosphatidylethanolamine) as well as their analogue phospholipids with a nitrated unsaturated fatty acids (1-palmitoyl-2-(E-9-nitrooleoyl)-sn-glycero-3-PC (example C) and 1-stearoyl-2-(E-9-nitrolinoleoyl)-sn-3-glycero-phosphatidylethanolamin (example R) were coated as mono substance as well as a combination of the native phospholipids and the corresponding nitro-carboxylic acid (s)-containing phospholipids in a mixing ratio of 1:1 on balloon catheters using the Langmuir-Schaefer procedure. The table shows the relative (%) changes of substance quantities after 24 hours, heat treatment and after performance of the sliding tests, mechanical examination as well as the relative change of the tractive work load compared to an uncoated balloon catheter.

FIG. 4a shows studies on the long-term stability of erythrocytes after pre-treatment with the natural phospholipids SOPC and PLPC, as well as their nitrated analogues SNOPC (1-stearoyl-2-(E-9-nitrooleoyl)-sn-glycero-3-PC and PNLPC (1-palmitoyl-2-(E-9-nitrolinoleoyl-sn-glycero-3-PC) that were subsequently stored for 2 days at 4° C. Then samples were rewarmed up to 30° C., one sample served as blank in each. The heated samples were agitated on a shaking plate with a low rotation rate at 30° C. for 24 to 48 hours. This was followed by the preparation of samples. The rate of haemolysis expressed in percentage is shown.

FIG. 4b shows results of investigations where mast cells were activated with Mastoparan. Cells were incubated with natural phospholipids SOPC and PLPC, as well as their analogue nitrated phospholipids SNOPC and PNLPC for one hour. Thereafter they were incubated with 5 or 25 μmol Mastoparan. The Ca²⁺ inward flux was determined, and normalized to the calcium influx of the respective base measurement and expressed as percentage of increase. Furthermore, the release of histamine from the C2 cells using a histamine-ELISA was determined and is expressed in ng/ml.

FIG. 4c shows investigations assessing the impact pre-treatment of erythrocytes with the natural phospholipids SOPC and PLPC, as well as the analogue nitrated phospholipids SNOPC and PNLPC on the mechanical stability of their cell membranes. Carefully prepared erythrocytes were suspended in physiological saline solution, and treated in an ultrasonic bath applying 10 Watts at a temperature of 30° and 50° C. for two to five minutes. Then, the samples were centrifuged and the supernatant analyzed. The percentages rates of haemolysis are shown.

FIG. 4d shows investigations assessing the impact of pre-treatment of erythrocytes with the natural phospholipids SOPC and PLPC, as well as with the analogue nitrated phospholipids SNOPC and PNLPC on their stability to osmotic gradients. Carefully prepared erythrocytes were suspended in distilled water and NaCl solutions with an increasing concentration thereof from 0.1 to 1.0 g/dl. The photometric determined haemoglobin concentration in a completely lysed sample was used for reference of the haemolysis in a given sample expressed as relative proportion. On the y-axis, the relative proportion of haemolysis is specified.

FIG. 5 shows investigations assessing the impact of pre-treatment of erythrocytes with the natural phospholipids SOPC and PLPC, as well as with the analogue nitrated phospholipids SNOPC and PNLPC at a concentration of 10 or 50 μmol/l or the pre-treatment with the nitro fatty acids nitro-oleate (NOA) and nitro-linolate (NLA) at a concentration of 10 or 30 μmol on cell viability. Incubated cells were exposed to cisplatin (25 and 50 μmol/l), cyclosporine (50 and 100 μmol/l) or lipopolysaccharide (LPS) which was added to the medium cultivated herein for 24 hours. The number of dead cells for PL pre-treatment at a concentration of 10/50 μmol, as well as for the fatty acids pre-treated with 10/30 μmol in relation to the total number of cell determined are shown in the table.

FIG. 6 shows results of investigations assessing the vitality of iliac artery specimens from pigs, after pre-incubation with natural phospholipids POPC and SLPC, as well as their nitrated analogue phospholipids (1-palmitoyl-2-(E-9-nitrooleoyl)-sn-glycero-3-PC and 1-stearoyl-2-(E-9-nitrolinoleoyl)-sn-3-glycero-phosphatidylcholine for one hour. The pre-incubated specimens were exposed to 15 bar air pressure in a hyperbaric chamber. Analyses were carried out using a TUNEL staining (TUNEL-positive cells in %) and by determination of the amount of microparticles in the cell culture supernatants (microparticles/μl).

FIG. 6a Values determined were analyzed for statistically significant difference between results obtained using natural PL POPC and SLPC, and the comparable nitrated PL PNOPC and SNLPC. Substance mixtures exhibiting a similar behavior have been combined as specified. For statistical differences between results of the natural PL and those of the nitrated PL, it was determined whether the values found for nitrated PL were within the standard deviation of the values obtained (=o), were above (=+), significantly higher (=++) or were lower (=−). Values that were not statistically different from the values of the natural PL are specified n.s.

FIG. 7 shows investigations of the membrane-stabilizing effects of nitro carboxylic acid-containing phospholipids in the cryopreservation of tissues. The vascular segments were placed in a bath of saline, a saline solution with the natural phospholipids SOPC and PLPC, as well as a solution containing analogue phospholipids with nitrated unsaturated fatty acids, SNOPC and PNLPC, at a concentration of 200 mmol/l for one hour before freezing the specimens. After rewarming, isometric force generation was measured in response to stimulation with noradrenaline (arteries) or histamine (veins) (tensile force in grams) as well as for the vascular relaxation under administration of acetylcholine. For the calculation of the relaxation capacity vessel segment that have been frozen to the vasodilatation measured in unfrozen and not incubated reference segment was measured and set into relation to the determined values.

FIG. 7a Values determined were analyzed for statistically significant difference between results obtained using natural PL SOPC and PLPC, and the comparable nitrated PL SNOPC and PNLPC. Substance mixtures exhibiting a similar behavior have been combined as specified. For statistical differences between results of the natural PL and those of the nitrated PL, it was determined whether the values found for nitrated PL were within the standard deviation of the values obtained (=o), were above (=+), significantly higher (=++) or were lower (=−). Values that were not statistically distinguishable from the values of the natural PL are specified n.s

FIG. 8 shows results of effects of nitro-carboxylic acid (s)-containing phospholipids on membrane receptors of the TRP membrane protein family. Transmembraneous inward current measurements were performed in oocytes, expressing TRPV 1, 2 or 4 or TRPA1 receptors, during stimulation with capsaicin (10 μmol), cannabiol (10 μmol), 4α-PDD (50 μmol) or cinnamaldehyde (50 μmol). The oocytes were previously incubated with the phospholipids SOPC and PLPC, as well as the analogue phospholipids with nitration of the unsaturated fatty acids SNOPC and PNLPC at a concentration of 50 mmol/l, as well as with the natural fatty acids oleic acid and linoleic acid, and the nitrate analogues nitro oleic acid and nitro linoleic acid at a concentration of 30 μmol for 10 and 60 minutes. Untreated oocytes were used as controls, the results of those measurements served as reference values. After pretreatment, increase or decrease of inducible ion current as compared to the reference measurements was determined, respectively.

FIG. 8a Values determined were analyzed to determine whether the results obtained using natural PL SOPC and PLPC, and the comparable nitrated PL SNOPC and PNLPC were statistically differenct. Substance mixtures exhibiting a similar behavior were combined as specified. For statistical differences between results of the natural PL and those of the nitrated PL, it was determined whether the values found for nitrated PL were within the standard deviation of the values obtained (=o), were above (=+), significantly higher (=++) or were lower (=−). Values that were not statistically distinguishable from the values of the natural PL are specified n.s

FIG. 9 shows studies on the effects of coating soft tissue implant material with nitro carboxylic acid-containing phospholipids concerning tissue response, in vivo. Sterile silicone cushions were used as implant materials which were coated by spray coating of two layers with the natural phospholipids SOPC and PLPC, as well as their nitrated analogue phospholipids SNOPC and PNLPC; uncoated samples served as controls. The silicone cushions were implanted in Wistar rats and the cellular response and the fibrous tissue formation were assessed after resection of the treated areas according to the following key.
A) cellular reaction: A1: none; A2: occasional monocytic cells or lymphocytes; A3: moderate numbers or groups of monocytic cells or lymphocytes; A4: dense infiltration of monocytes, eosinophils, or giant cells.
B) fibrous tissue formation: B1: none; B2: slight collagen-rich layer around the implant; B3: thick (>1 mm) and density of collagen-rich tissue formation around the implant.

FIG. 9a A=summary of all cellular reactions for each category and expression; B=summary of all fibrous tissue formations and comparison of each expression. Calculated values were statistically analyzed to identify differences in the results for the natural PL SOPC and PLPC as compared to those of the nitrated PL SNOPC (example P14) and PNLPC (example P7). Mixtures with similar behavior have been combined as specified. If there was a statistical difference to the natural PL, it was determined whether the value found was within the standard deviation of the values obtained for the nitrated PL (=o) or above (=+), significantly higher (=++) or lower (=−), or significantly less (=−−). Values that were not statistically distinguishable from the values of the natural PL were specified n.s.

FIG. 10a shows results of investigations concerning effects on dimerization on membrane proteins after incubation with nitro carboxylic acid-containing phospholipids. Incubation was performed with the native phospholipids SOPC and SLPC, as well as the nitrated analogue phospholipids SNOPC (1-stearoyl-2-(E-9-nitrooleoyl)-sn-glycero-3-PC) (example 14) and SNLPC (1-stearoyl-2-(9-nitrolinoleoyl)-sn-3-glycero-phosphocholin) (example 13), which were in a mixture with the phospholipid DSPC (di-stearoyl-PC). The values on the y-axis represent the normalized values of the FRET measurement of DSPC vesicles without adding other phospholipids. Values of the x-axis represent the relative proportion of added PL in percent.

FIG. 10b shows results of investigations concerning effects on the anisotropy depending on the temperature of model membranes from DSPC vesicles in a comparable set of experiments. The anisotropy is plotted on the y-axis and the temperature on the x-axis.

### LIST OF ABBREVIATIONS

DOPC 1,2-dioleoyl-sn-glycero-3-PC
DSPC 1,2-distearoyl-sn-glycero-3-PC
LA Linoleic acid
NOLA E-9-nitrolinoleic acid
NOOA E-9-nitrooleic acid
OA Oleic acid
ONOPC 1-Oleoyl-2-(E-9-nitrooleoyl)-sn-glycero-3-PC
PC Phosphatidylcholin,Glycerophosphatidylcholin, respectively
PE Phosphatidylethanolamine
PL Phospholipid(s)
PLPC 1-Palmitoyl-2-linoleoyl-sn-glycero-3-PC
PNLPC 1-Palmitoyl-2-(E-9-nitrolinoleoyl-sn-glycero-3-PC
PNOPC 1-Palmitoyl-2-(E-9-nitrooleoyl)-sn-glycero-3-PC
PNOPE 1-Palmitoyl-2-(E-9-nitrooleoyl)-sn-3-glycero-phosphatidylethanolamine
PNOPI 1-Palmitoyl-2-(E-9-nitrooleoyl)-sn-3-glycero-phosphatidylinositol
POPC 1-Palmitoyl-2-oleoyl-sn-glycero-3-PC
POPE 1-Palmitoyl-2-oleoyl-sn-glycero-3-phosphatidylethanolamine
POPI 1-Palmitoyl-2-oleoyl-sn-3-glycero-phosphatidylinositol
SLPC 1-Stearoyl-2-linoleoyl-sn-glycero-3-PC
SLPE 1-Stearoyl-2-linoleoyl-sn-3-glycero-phosphatidylethanolamine
SNLPC 1-Stearoyl-2-(9-nitrolinoleoyl)-sn-3-glycero-phosphocholine
SNOPC 1-Stearoyl-2-(E-9-nitrooleoyl)-sn-glycero-3-PC
SNLPE 1-Stearoyl-2-(E-9-nitrolinoleoyl)-sn-3-glycero-phosphatidylethanolamine
SOPC 1-Stearoyl-2-oleoyl-sn-glycero-3-PC

### SYNTHESES (EXAMPLES)

### Example A

### Standard Synthesis of NPL

Below, the standard synthesis of nitrocarboxylic acid containing phospholipids (alternatively termed as NPL and nitrophospholipids) is described. The NPL are generated by esterification of sn-glycero-3-phosphatides 1.

If both OH groups have to be acylated with one nitro fatty acid 2 (R²_{(NO)}CO₂H=nitro fatty acid), the starting material 1 is reacted with two equivalents of nitro fatty acid 2 using intermediately formed activated esters (such as acyl-2,6-dichlorobenzoate) The so called “symmetric” (i.e. R¹═R²) 1,2-di-(nitroacyl-sn-3-glycerophosphatides 3 are derived after purification with good results.

The glycerophosphatides substituted with different acyl groups within position 1 and 2 are generated via two consecutive esterifications. Initially, the sn-1 position is activated by means of a condensation with dibutyltin oxide. The so formed intermediate cyclic tin ester is treated with acid chloride 4 in presence of triethylamine to afford 1-acyl-2-lyso-sn-3-glycerophosphatide 5 (for a general procedure to synthesise such lyso phosphatides see D'Arrigo, Servi, Molecules 2010,15, 1354). The proceeding acylation of the 2^{nd} OH group involving a (nitro) fatty acid succeeded applying the activated ester method. Finally, the “unsymmetrically” substituted 1-“nitro”acyl-2-(“nitro”acyl)-sn-3-glycerophosphatide 6 is obtained after careful purification ready for use in coating processes. Within the present sequence, the acid chloride 4 represents a nitro acid chloride or a non-nitro acid chloride. The fatty acid 2 represents a non-nitro fatty acid or a nitro fatty acid, respectively. Prerequisite is, that at least one of acid chloride 4 and fatty acid 2, respectively, contains a nitro group. Overall, the products as mentioned below are formed: 1-nitroacyl-2-(acyl)-sn-3-glycero-phosphatide 6, 1-acyl-2-(nitroacyl)-sn-3-glycerophosphatide 6 and 1-nitroacyl-2-(nitroacyl)-sn-3-glycerophosphatide 6. This robust procedure is used to synthesise of almost stable nitro fatty acid containing phosphatides. Intending to introduce more sensitive nitrocarboxylic acids an appropriate attachment within the final (2^{nd}) acylation is required. Generally, esterification fails applying well known classical methods because the product α,β unsaturated nitro functions represent excellent acceptor groups for various nucleophiles. Therefore, work-up and purification using standard basic and aqueous basic procedures are not recommended. For transformations incorporating nitrocarboxylic acids the esterification procedure published by R. G. Salomon (Salomon, Biorg. & Med. Chem. 2011, 19, 580) for similar acids bearing vinyl ketone moieties can be adapted. It is crucial to avoid any intramolecular competing processes, e.g. transesterifications (for intramolecular ester group shifts within diols see Adlercreutz, Biocatal. Biotransfor. 2000, 18, 1 and Biotechnol. Bioeng. 2002, 78, 403).

The synthesis of nitro fatty acid containing phospholipids incorporating R¹COO— as a non-nitro carboxylic acid fragment and R²COO— as a nitrocarboxylic acid fragment requires two consecutive regioselective esterifications. Because of the base-sensitive nitrocarboxylic acid derivative in sn-2 position, the sn-1 position is acylated selectively within the first step introducing an appropriate fatty acid segment. Adapting a procedure published by Servi, sn-glycero-3-phosphatidylcholine 1a and dibutyl tin oxide are condensed for an in situ generation of a cyclic dibutyltin diester, which then as directly treated with the carboxylic acid chloride 4a and triethylamine affording monoester 5a (in analogy to Servi, Org. Biomol. Chem. 2006, 4, 2974 and Servi, Chem. Phys. Lipids 2007, 147, 113). Here, for work-up and purification standard procedures can be used. Then, the so formed 1-acyl-2-lyso-sn-3-glycero-phospholipides 5a are subjected to a second esterification introducing the nitro fatty acid moiety applying an optimised procedure developed from a method published by Salomon to give lipids 6a (Salomon, Biorg. & Med. Chem. 2011, 19, 580). The syntheses mentioned above are summarised in Scheme 5:

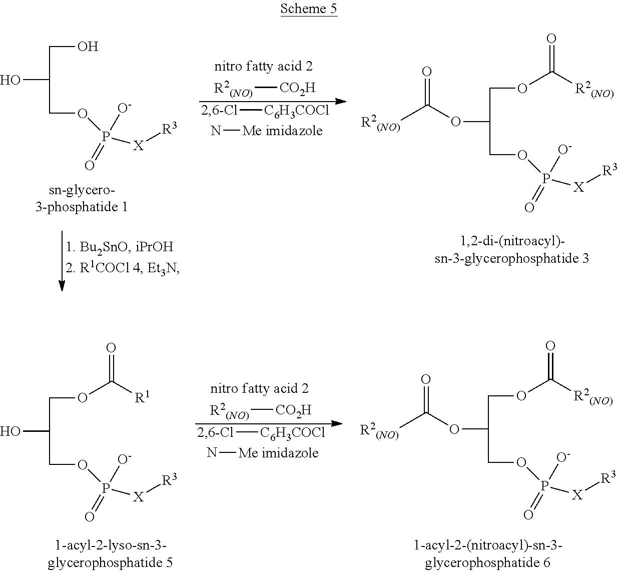

The synthesis of glycerophosphatides 10 bearing different acyl groups including a sensitive nitroacyl group in sn-1 position and a non-nitroacyl group in sn-2 position is started from the symmetric diester 7 (easily obtained from sn-glycero-3-phosphatide 1 via standard twofold esterification: in analogy to B. Smith, J. Org: Chem. 2008, 73, 6058). Upon treatment with phospholipase A (PLA₂) and related lipase, respectively, the sn-1 position of diester 7 is cleaved regioselectively to afford 1-lyso phosphatide 10′ (in analogy to J. Sakakibara, Tetrahedron Lett. 1993, 34, 2487). A final ester formation delivers the unsymmetrically substituted 1-nitroacyl-2-(acyl)-sn-3-glycero-phosphatide 10 ready for coatings after careful purification in analogy to the Salomon procedure (Biorg. & Med. Chem. 2011, 19, 580, Scheme 6)

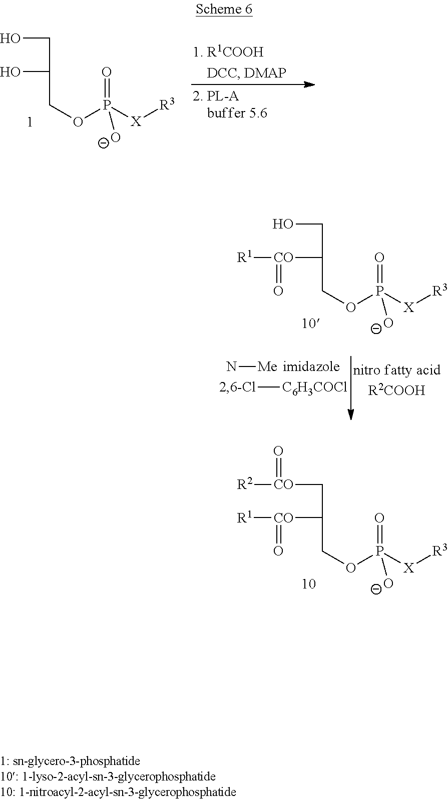

### Example B

### Synthesis of 1,2-di-(9-nitrooleoyl)-sn-3-glycerophosphocholine 3a

sn-Glycero-3-phosphatidylcholine 1a is commercially available or is obtained via known procedures form soya and egg yolk lecithin, respectively. 1a has been synthesized according the method developed by R. G. Salomon (Salomon, Biorg. & Med. Chem. 2011, 19, 580) using nitrooleic acid. The synthesis of 1,2-di-(9-nitrooleoyl)-sn-3-glycerophosphocholine (β,γ-di-(9-nitrooleoyl)-L-α-phosphatidylcholine) 3a was carried out successfully, the careful avoiding of any basic nucleophilic conditions was mandatory.

A suspension of 0.5 g (1.95 mmol, 1 eq.) sn-glycero-3-phosphocholine 1a in 100 mL dry dichloromethane was treated with 1.93 g (5.85 mmol, 3 eq.) 9-nitrooleic acid (commercially available or produced via known literature procedures), 0.48 g (5.85 mmol, 3 eq.) 1 methyl imidazole and 1.224 g (5.85 mmol, 3 eq.) 2,6-dichlorobenzoyl chloride. After three days of stirring at 23° C. the suspension dissolved slowly. The solvent was removed in vacuum and the residue was purified via preparative column chromatography and preparative HPLC (Phenomenex Gemini NX 5μ C18 110 Å, 95% MeOH/H₂O). Yield: 1.21 g (1.38 mmol, 71%) of 3a (purity control via HPLC, ¹H and ¹³C NMR spectroscopy).

### 1,2-Di-(9-nitrooleoyl)-sn-3-glycero-phosphatidylcholine 3a

173.5, 173.0 (C═O), 150.0, 149.5 (2×C—NO₂), 134.5, 133.5 (2×HC═), 71.5 (d), 67.0 (d), 64.5 (d), 63.0, 60 (d), 54.5 (NMe₃), 34.5-20.5 (28×CH₂), 14.0 (2×CH₃).

### Example C

### Synthesis of (E)-1-Palmitoyl-2-(9-nitrooleoyl)-sn-3-glycerophosphocholine 6a

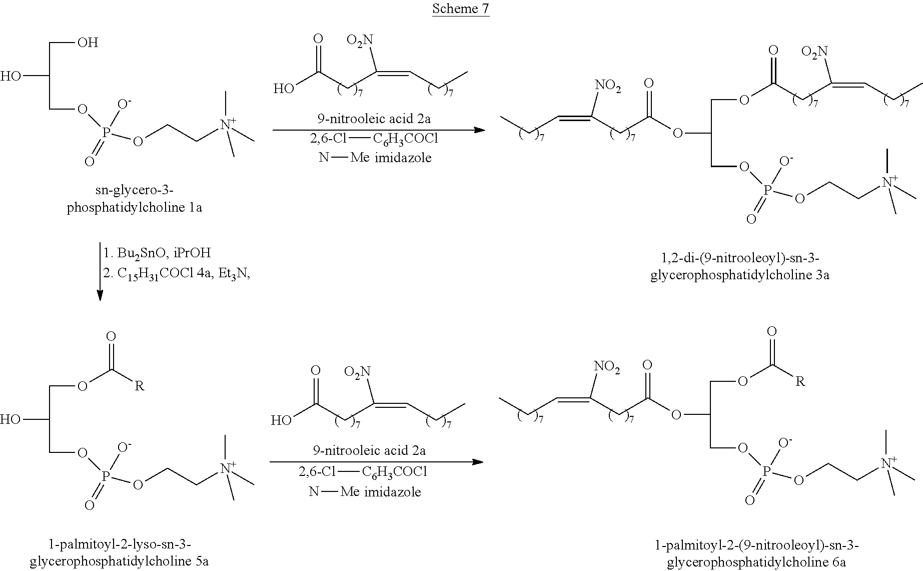

9-Nitrooleic acid has been synthesized using a literature sequence (Woodcock, Org. Lett. 2006, 8, 3931 and King, Org. Lett. 2006, 8, 2305)

### Synthesis of 1-palmitoyl-2-lyso-sn-3-glycerophosphocholine (β-lyso-γ-palmitoyl-L-α-phosphatidylcholine) 5a

1 g (3.9 mmol, 1 eq) sn-glycero-3-phosphocholine 1a and 1.1 g (4.3 mmol, 1.1 eq.) of dibutyltin oxide were suspended in 100 ml of isopropanol and heated to reflux for 1 h. The now formed solution is cooled to 25° C. and 0.25 mL (7.8 mmol, 2 eq.) triethylamine and 2.4 mL (7.8 mmol, 2 eq.) palmitoyl chloride are added. After 15 min. 100 mL of water were added and the reaction mixture was extracted with heptane (4×50 mL). The solvents were removed in vacuum and the crude oily residue was dissolved in ethanol. Lyso 5a is precipitated by addition of acetone (40 mL) at 10° C. Purification can be carried out using preparative HPLC (Phenomenex Gemini NX 5μ C18 110 Å, 95% MeOH/H₂O).

Yield: 0.9 g (1.8 mmol, 45%) 5a as a white solid (purity control via HPLC, ¹H and ¹³C NMR spectroscopy).

### Synthesis of (E)-1-palmitoyl-2-(9-nitrooleoyl)-sn-3-glycerophosphocholine (β-(9-nitrooleoyl)-γ-palmitoyl-L-α-phosphatidylcholine) 6a

A solution of 0.135 g (0.412 mmol, 2.04 eq.) (E)-9-nitrooleic acid 25a (obtained according Example L1) and 0.1 g (0.202 mmol) 1-palmitoyl-2-lyso-sn-3-glycerophosphocholine 5a in 10 mL of dry CH₂Cl₂ was treated with 0.5 g (0.05 mL, 0.6 mmol, 2.97 eq.) 1-methyl imidazole and 0.4 g (0.01 mL, 0.67 mmol 3.32 eq.) 2,6-dichlorobenzoyl chloride. After three days of stirring at 23° C. the solvent was removed in vacuum and the residue was purified via preparative column chromatography and preparative HPLC (Phenomenex Gemini NX 5μ C18 110 Å, 95% MeOH/H₂O). Yield: 127.4 mg (0.155 mmol, 77%) of 6a as a white solid (purity control via HPLC, ¹H and ¹³C NMR spectroscopy).

### 1-Palmitoyl-2-(9-nitrooleoyl)-3-glycerophosphatidylcholine 6a

174.0, 173.5 (C═O), 150.0 (C—NO₂), 134.0 (HC═), 71.0 (d), 66.0 (d), 63.5 (d), 63.0, 59.5 (d), 54.5 (NMe₃), 34.5-21.0 (28×CH₂), 14.5, 14.0 (2×CH₃).

### Synthesis of Phosphatide Acids, Phosphatide Esters, Phosphatidyl-ethanolamine and phosphatidyl serine

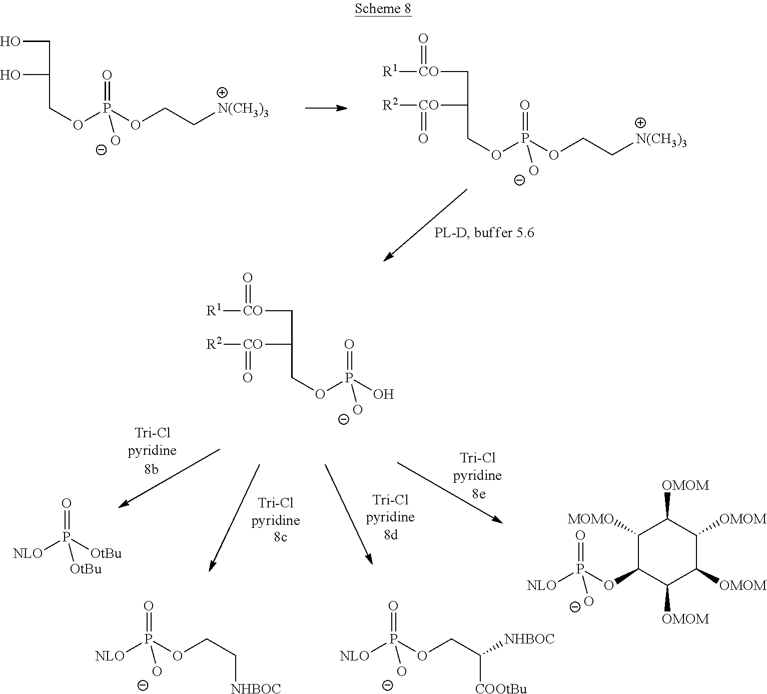

wherein 8b, 8c, 8d, 8e and NL are denoted as follows:

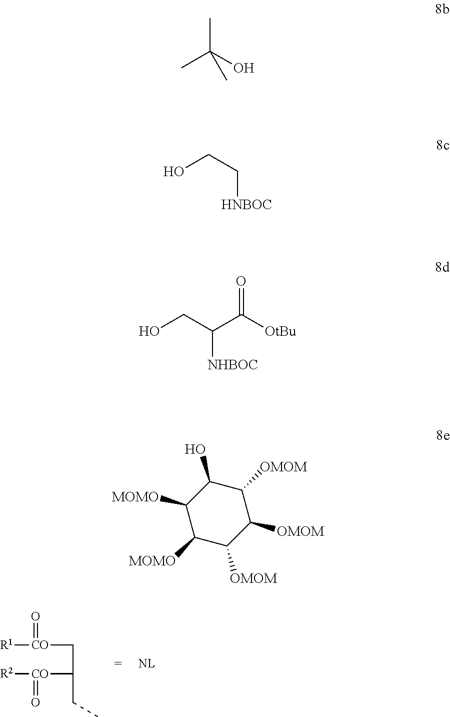

wherein R¹COO— and R²COO— represent a nitro and a non-nitro and, preferentially an non-nitrocarboxylic acid substituent. Because of the fact that nitrocarboxylates R¹COO— and/or R²COO— are sensitive in the presence of bases and might be destroyed upon reactions in pyridine, it is recommended to use non-nitro carboxylates as substituents in both, R¹COO— and R²COO—. The non-nitro carboxylates can be easily removed by means of a cleavage with sodium methoxide in methanol. Then, the nitrocarboxylates can be introduced as described before.

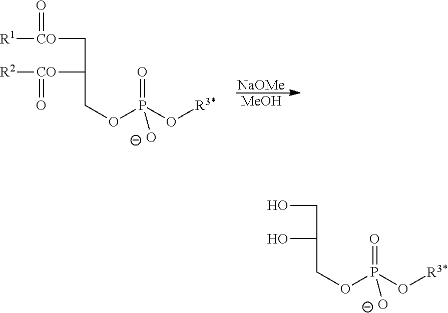

Here, R^{3*} refers to one of the protected head groups listed under 8b, 8c, 8d and 8.

Below, synthesis examples are described for R¹COO— and R²COO— are palmitoyl (H₃₁C₁₅CO₂—)

### Synthesis of 1,2-di-(palmitoyl-sn-3-glycerophosphate 7 (in analogy to B. Smith, J. Org: Chem. 2008, 73, 6058)

A solution of 1,2-di-(palmitoyl-sn-3-glycerophosphatidylcholine (1.023 g 1.52 mmol) in chloroform/acetate buffer (pH 5.6, 80 mM CaCl₂, 100/140 mL) was treated with phospholipase D (PL-D, 0.5 mg, Str.) and the mixture was heated to 40° C. with stirring overnight. Then, the layers were separated and the aqueous phase was extracted with chloroform/methanol (2:1). The organic layers were washed with water and dried (Na₂SO₄). After removal of the solvent the residue was purified by preparative column chromatography (silica gel, CHCl₃/MeOH/H₂O). Yield: 831.2 mg (1.41 mmol, 93%) of 7 (purity control via HPLC, ¹H and ¹³C NMR spectroscopy).

### Example D

### Synthesis of 1,2-di-(palmitoyl-sn-3-glycerophosphatidyl-(N-boc)-ethanolamine 9c (in analogy to R. Aneja, Tetrahedron Lett. 2000, 41, 847)

To a solution of triisopropylbenzolsulfonyl chloride (Tris-Cl, 257 mg, 0.85 mmol) and N-(boc)-ethanolamine (137 mg, 0.85 mmol) in dry pyridine was added 1,2-di-(palmitoyl-sn-3-glycerophosphate 7 (500 mg, 0.85 mmol) by means of a syringe pump over a period of 1 h. The mixture was stirred at 35° C. for 6 h. Then, the reaction was stopped by hydrolysis with water. The layers were separated and the aqueous phase was extracted with CH₂Cl₂. After drying (Na₂SO₄), the solvents were removed in vacuum (co-distillation with toluene to remove the pyridine) and the residue was purified by preparative column chromatography (silica gel, EtOAc/hexanes). Yield: 559.2 mg (0.765 mmol, 90%) 9c (purity control via HPLC, ¹H and ¹³C NMR spectroscopy).

### Synthesis of sn-3-glycerophosphatidyl-(N-boc)-ethanolamine 1c

Reaction and scale as described for the synthesis of 1b using 1,2-di-(palmitoyl-sn-3-glycerophosphatidyl-(N-boc)-ethanolamine 9c (500 mg, 0.684 mmol). Purification by crystallization or column chromatography (silica gel, CHCl₃/MeOH gradient) or preparative HPLC (Phenomenex Gemini NX 5μ C18 110 Å, gradient MeOH/H₂O). Yield: 187.4 mg (0.595 mmol, 87%) 1c (purity control via HPLC, ¹H and ¹³C NMR spectroscopy).

### Synthesis of 1,2-di-(9-nitrooleoyl)-sn-3-glycero-phosphatidyl-N-(boc)-ethanolamine (β,γ-di-(9-nitrooleoyl)-L-α-phosphatidyl-N-(boc)-ethanolamine) 3c′

Reaction and scale as described for the synthesis of 3a using sn-3-glycero-phosphatidyl-N-(boc)-ethanolamine 1c (150 mg, 0.476 mmol) and 9-nitrooleic acid (467 mg, 1.43 mmol). Purification by column chromatography (silica gel, CH₂Cl₂/MeOH gradient) or preparative HPLC (Phenomenex Gemini NX 5μ C18 110 Å, gradient MeOH/H₂O). Yield: 333.1 mg (0.357 mmol, 75%) 3c′ (purity control via HPLC, ¹H and ¹³C NMR spectroscopy).

### Synthesis of 1,2-di-(9-nitrooleoyl)-sn-3-glycero-phosphatidylethanolamine (β,γ-di-(9-nitrooleoyl)-L-α-phosphatidylethanolamine) 3c

Reaction and scale as described for the synthesis of 3b using 1,2-di-(9-nitrooleoyl)-sn-3-glycero-phosphatidyl-N-(boc)-ethanolamine 3c′ (300 mg, 0.322 mmol). Purification by column chromatography (silica gel, CH₂Cl₂/MeOH gradient) or preparative HPLC (Phenomenex Gemini NX 5μ C18 110 Å, gradient MeOH/H₂O). Yield: 260.2 mg (0.312 mmol, 97%) 3c (purity control via HPLC, ¹H and ¹³C NMR spectroscopy).

### 1,2-Di-(9-nitrooleoyl)-3-glycerophosphatidylethanolamine 3c

174.0, 173.0 (C═O), 150.0 (2×C—NO₂), 134.5 (2×HC═), 70.5 (d), 63.5 (d), 63.0, 62.0, 41.0 (d), 34.0-22.0 (28×CH₂), 14.0 (2×CH₃).

### Example E

### Synthesis of 1,2-di-(palmitoyl-sn-3-glycerophosphatidyl-N-(boc)-(S)-serin tert-butylester 9d Reaction and scale as described for the synthesis of 9c using di-(palmitoyl-sn-3-glycerophosphate 7 (500 mg, 0.85 mmol) and N-(boc)-(S)-serine tert-butylester (222 mg, 0.85 mmol). Purification by column chromatography (silica gel, EtOAc/hexanes). Yield: 652.1 mg (0.773 mmol, 91%) 9d (purity control via HPLC, ¹H and ¹³C NMR spectroscopy)

### Synthesis of sn-3-glycerophosphatidyl-N-(boc)-(S)-serine tert-butylester 1d

Reaction and scale as described for the synthesis of 1b using 1,2-di-(palmitoyl-sn-3-glycerophosphatidyl-N-(boc)-(S)-serine tert-butylester 9d (500 mg, 0.602 mmol). Purification by crystallization or column chromatography (silica gel, CHCl₃/MeOH gradient) or preparative HPLC (Phenomenex Gemini NX 5μ C18 110 Å, gradient MeOH/H₂O). Yield: 227.0 mg (0.547 mmol, 91%) 1d (purity control via HPLC, ¹H and ¹³C NMR spectroscopy).

### Synthesis of 1,2-di-(9-nitrooleoyl)-sn-3-glycero-phosphatidyl-N-(boc)-(S)-serine tert-butylester (β,γ-di-(9-nitrooleoyl)-L-α-phosphatidyl-N-(boc)-(S)-serine tert-butylester 3d′

Reaction and scale as described for the synthesis of 3a using sn-3-glycero-phosphatidyl-N-(boc)-(S)-serine tert-butylester 1d (150 mg, 0.361 mmol) and 9-nitrooleic acid (355 mg, 1.08 mmol). Purification by column chromatography (silica gel, CH₂Cl₂/MeOH) or preparative HPLC (Phenomenex Gemini NX 5μ C18 110 Å, gradient MeOH/H₂O). Yield: 287.2 mg (0.278 mmol, 77%) 3d′ (purity control via HPLC, ¹H and ¹³C NMR spectroscopy).

### Synthesis of 1,2-di-(9-nitrooleoyl)-sn-3-glycerophosphatidylserine nitrooleoyl)-L-α-phosphatidylserine) 3d

Reaction and scale as described for the synthesis of 3b using 1,2-di-(9-nitrooleoyl)-sn-3-glycerophosphatidyl-N-(boc)-(S)-serine tert-butylester 3d′ (250 mg, 0.242 mmol). Purification by column chromatography (silica gel, CH₂Cl₂/MeOH, gradient) or preparative HPLC (Phenomenex Gemini NX 5μ C18 110 Å, gradient MeOH/H₂O). Yield: 210.1 mg (0.240 mmol, 99%) 3d (purity control via HPLC, ¹H and ¹³C NMR spectroscopy).

### Example F

### Synthesis of 1,2-di-palmitoyl-sn-3-glycerophosphatidyl penta-(O-methoxymethyl) inositol 9e

Reaction and scale as described for the synthesis of 9c using di-(palmitoyl-sn-3-glycerophosphate 7 (500 mg, 0.85 mmol) and penta-(O-methoxymethyl) inositol (320 mg, 0.85 mmol). Purification by column chromatography (silica gel, EtOAc/hexanes). Yield: 692.6 mg (0.714 mmol, 84%) 9e (purity control via HPLC, ¹H and ¹³C NMR spectroscopy).

### Synthesis of sn-3-glycerophosphatidyl penta-(O-methoxymethyl) inositol 9e

Reaction and scale as described for the synthesis of 1b using 1,2-di-(palmitoyl-sn-3-glycerophosphatidyl penta-(O-methoxymethyl) inositol 9e (500 mg, 0.515 mmol). Purification by crystallization or column chromatography (silica gel, CHCl₃/MeOH gradient) or preparative HPLC (Phenomenex Gemini NX 5μ C18 110 Å, gradient MeOH/H₂O). Yield: 257.1 mg (0.464 mmol, 90%) le (purity control via HPLC, ¹H and ¹³C NMR spectroscopy).

### Synthesis of 1,2-di-(9-nitrooleoyl)-sn-3-glycerophosphatidyl penta-(O-methoxymethyl) inositol (β,γ-di-(9-nitrooleoyl)-L-α-phosphatidyl penta-O-methoxymethyl inositol 3e′

Reaction and scale as described for the synthesis of 3a using sn-3-glycerophosphatidyl penta-(O-methoxymethyl) inositol le (150 mg, 0.271 mmol) and 9-nitrooleic acid (266 mg, 0.812 mmol). Purification by column chromatography (silica gel, CH₂Cl₂/MeOH, gradient) or preparative HPLC (Phenomenex Gemini NX 5μ C18 110 Å, gradient MeOH/H₂O). Yield: 244.9 mg (0.209 mmol, 77%) 3e′ (purity control via HPLC, ¹H and ¹³C NMR spectroscopy).

### Synthesis of 1,2-di-(9-nitrooleoyl)-sn-3-glycerophosphatidylinositol (β,γ-di-(9-nitrooleoyl)-L-α-phosphatidylinositol) 3e

Reaction and scale as described for the synthesis of 3b using 1,2-di-(9-nitrooleoyl)-sn-3-glycerophosphatidyl penta-(O-methoxymethyl) inositol 3e′ (200 mg, 0.171 mmol). Purification by column chromatography (silica gel, CH₂Cl₂/MeOH, gradient) or preparative HPLC (Phenomenex Gemini NX 5μ C18 110 Å, gradient MeOH/H₂O). Yield: 160.8 mg (0.169 mmol, 99%) 3e (purity control via HPLC, ¹H and ¹³C NMR spectroscopy).

### Example G

### Synthesis of sn-3-glycero-di-tert.-butyl phosphate 1b in analogy to J. Brimacombe (J. Chem. Soc. Perkin Trans. I, 1995, 1673) and H. Brockerhoff, M. Yurkowski (Can. J. Biochem., 1965, 43, 1777)

The starting material di-(palmitoyl)-sn-3-glycero-di-tert.-butyl phosphate 9b is synthesized according P. Konradsson (J. Org. Chem. 2002, 67, 194). A solution of di-(palmitoyl)-sn-3-glycero-di-tert.-butyl phosphate 9b (600 mg, 0.871 mmol) in Et₂O/MeOH (50 mL, 1:1) was treated with a catalytic amount of sodium methoxide. After stirring for 1 h at 23° C. neutralization was performed with amberlit 1R120. The solvents were removed in vacuum and the residue was crystallized or purified via preparative column chromatography (silica gel, CHCl₃/MeOH gradient) or preparative

HPLC (Phenomenex Gemini NX 5μ C18 110 Å, gradient MeOH/H₂O). Yield: 204.5 mg (0.749 mmol, 86%) 1b (purity control via HPLC, ¹H and ¹³C NMR spectroscopy).

### Synthesis of 1,2-di-(9-nitrooleoyl)-sn-3-glycero-di-tert.-butyl phosphate (β,γ-di-(9-nitrooleoyl)-L-α-glycero-di-tert-butyl phosphate) 3b′ (according R. Salomon, Biorg. & Med. Chem. 2011, 19, 580)

Reaction and scale as described for the synthesis of 3a using sn-3-glycero-di-tert.-butyl phosphate 1b (150 mg, 0.549 mmol) and 9-nitrooleic acid (539 mg, 1.65 mmol). Purification by column chromatography (silica gel, CH₂Cl₂/MeOH, gradient) or preparative HPLC (Phenomenex Gemini NX 5μ C18 110 Å, gradient MeOH/H₂O). Yield: 413.4 mg (0.464 mmol, 58%) 3b′ (purity control via HPLC, ¹H and ¹³C NMR spectroscopy).

### Synthesis of 1,2-di-(9-nitrooleoyl)-sn-3-glycerophosphate (β,γ-di-(9-nitrooleoyl)-L-α-glycerophosphate) 3b (in analogy to P. Konradsson: J. Org. Chem. 2002, 67, 194 and B. Smith: J. Org: Chem. 2008, 73, 6058)

Removal of the protecting groups: 1,2-Di-(9-nitrooleoyl)-sn-3-glycero-di-tert.-butyl glycerophosphate (300 mg, 0.337 mmol) in CH₂Cl₂ (30 mL) was treated with trifluoroacetic acid (8 mL) with stirring at 23° C. After 30 min the reaction was stopped by adding MeOH (0.4 mL). The reaction mixture was diluted with toluene (80 mL) and the solvents were removed in vacuum. The residue was purified by column chromatography or preparative HPLC (Phenomenex Gemini NX 5μ C18 110 Å, gradient MeOH/H₂O). Yield: 264 mg (0.336 mmol, 99%) 3b (purity control via HPLC, ¹H and ¹³C NMR spectroscopy).

### Analytical Data of the Compound According to the Invention:

¹³C NMR data (in the case of ³¹P—¹³C couplings ²J, ³J the peak pattern is detailed as doublet “d”)

For ¹³C NMR spectra Bruker ARX400 and Bruker Avance II (AV) 400 spectrometers at 100.6 MHz were used, solvent: CDCl₃, 22° C., broadband proton decoupled)

### Standard Syntheses of Nitrocarboxylic Acids

The synthesis of nitrocarboxylic acid can be achieved using two different strategies. On one hand, a direct nitration of a commercially available carboxylic acid can be attempted. In this connection it is obvious, that mixtures of regioisomers are obtained in most cases. Therefore it is necessary to follow up with a careful separation of such isomers—except an esterification can be run with the mixture (M. D'Ischia, J. Org. Chem. 2000, 65, 4853). In particular, the use of multiple unsaturated starting materials requires carefully optimized preparation procedures. On the other hand, appropriate nitroalkanes and aldehydes can be coupled by means of a Henry-reaction, a subsequent condensation affords nitroalkenes. Now, the synthesis occurs more complicated and time consuming, but this strategy enables to avoid the formation of mixtures of regioisomers and al separations and purifications are easily achieved. Furthermore, the starting materials are commercially available and can be generated by simple transformation/degradation of appropriate fatty acids. All syntheses are adapted from literature procedures. All polyunsaturated fatty acids are sensitive in the presence of oxygen recommending the use of inert gas atmosphere. Nitroalkenes suffer from rapid additions of nucleophiles such as hydroxide, amines, etc.

### Example H1

### Radical Nitration of Linoleic Acid 1

Unsaturated and polyunsaturated carboxylic acids can be nitrated via radical reaction in analogy to Ishibashi (Org. Lett. 2010, 12, 124).

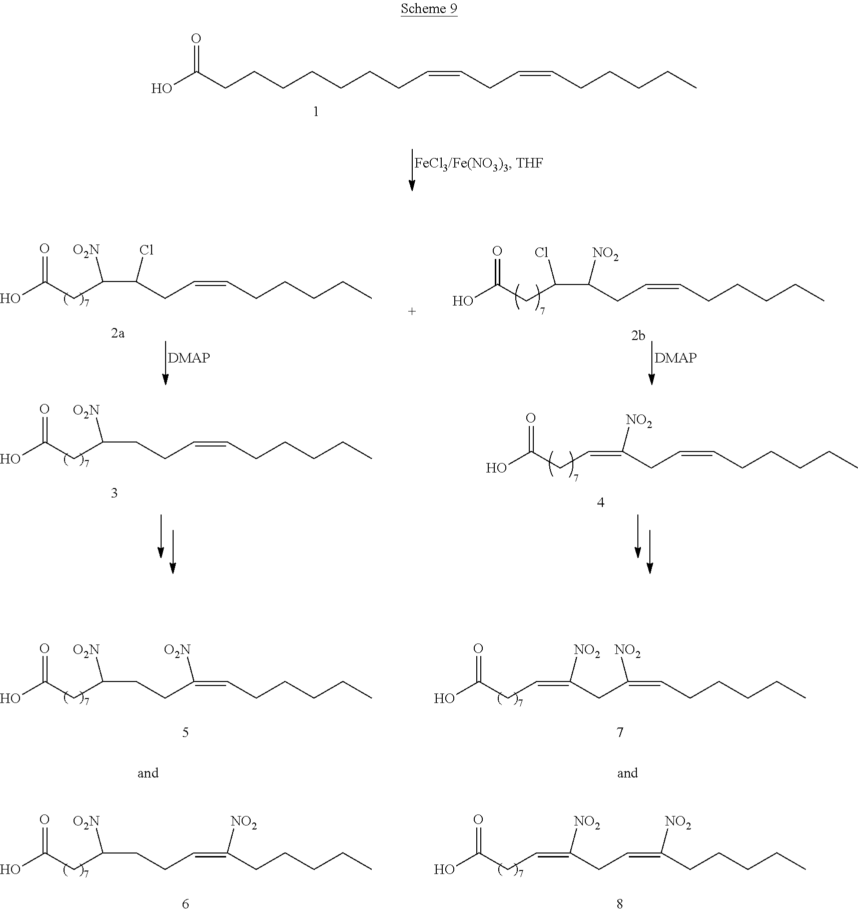

Here, the radical nitration of linoleic acid [(Z,Z)-9,12-octadecadienoic acid] is described affording a mixture of regioisomers. Generally, it is recommended to replace the toxic N₂O₄ (gas) by a reagent combination of FeCl₃/Fe(NO₃)₃.

Linoleic acid 1 (840 mg, 3 mmol) and FeCl₃ (730 mg, 4.5 mmol) were dissolved in THF (30 mL). Then, Fe(NO₃)₃×9 H₂O (1.46 mg, 3.6 mmol) was added and the mixture was heated to reflux for 2 h. After cooling to 23° C., a mixture of β-chloro-nitroalkanes 2 and nitroalkenes 3/4 was formed. For completion of HCl elimination, the reaction mixture was diluted with THF (20 mL) and N,N-dimethylaminopyridine (DMAP, 550 mg, 4.5 mmol) was added. After stirring at 23° C. overnight, dilution with ether caused the precipitation of the Fe and ammonium salts, which then were removed by filtration. The solvents were distilled off and the residue was pre-purified by preparative column chromatography (silica gel, hexanes/EtOAc gradient). Precision cleaning and separation was conducted with preparative HPLC (Phenomenex Gemini NX 5μ C18 110 Å, gradient MeOH/H₂O). Yield: 214.5 mg (0.66 mmol, 22%) 9-nitrolinoleic acid 3, 361 mg (1.11 mmol, 37%) 10-nitrolinoleic acid 4, 156 mg (0.48 mmol, 16%) 12-nitrolinoleic acid, 9-nitro-10,11-octadecadienic acid, 16 mg (0.05 mmol, 1.6%) and further minor isomers (purity control via HPLC, ¹H and ¹³C NMR spectroscopy).

Remark: The reaction of 9-nitrolinoleic acid 3 (50 mg, 0.154 mmol) afforded 9,12-dinitrolinoleic acid 5 (8 mg, 0.022 mmol, 13.9%), 9,13-dinitrolinoleic acid 6 (7 mg, 0.019 mmol, 12.1%) and additional products applying the conditions described above.

The reaction of 10-nitrolinoleic acid 4 (50 mg, 0.154 mmol) afforded 10,12-dinitrolinoleic acid 7 (8 mg, 0.022 mmol, 13.9%), 10,13-dinitrolinoleic acid 8 (9 mg, 0.024 mmol, 16%) and additional products applying the conditions described above.

### Example H2

### Radical Nitration of Arachidonic Acid 9

In analogy to M. d'Ischia (J. Org. Chem. 2000, 65, 4853), M. Balazy (Free Rad. Biol. & Med. 2008, 45, 269 and Free Rad. Biol. & Med. 2011, 50, 411) 5,8,11,14-eicosatetraenic acid (arachidonic acid) was nitrated under radicalic conditions.

Arachidonic acid 9 (100 mg, 0.33 mmol) was treated with a solution of NO₂ in hexane (0.7 mM, density 3.4 g/cm³) and the mixture was stirred at 23° C. for 15 min. Then, excess of NO₂ was removed by bubbling nitrogen through the solution and the residue was hydrolyzed with H₂O/EtOAc (1:1, 10 mL). The organic layer was repeatedly extracted with water, then, the solvents were distilled off. The residue was analyzed and separated by means of HPLC (Phenomenex Gemini NX 5μ C18 110 Å, Gradient MeOH/H₂O). Several mono nitrocarboxylic acids are found with low yields. Further minor compounds were not characterized.

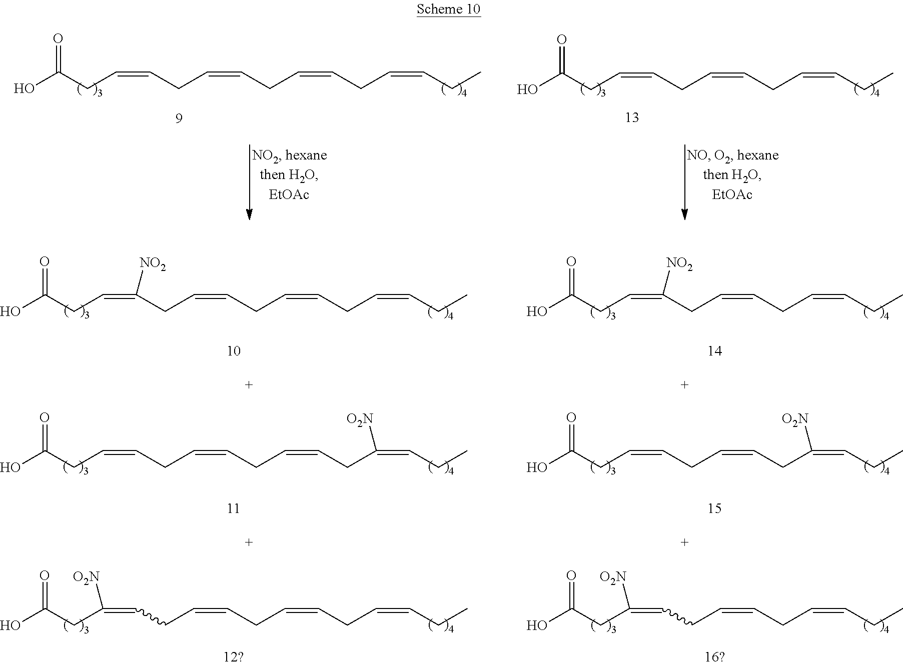

6-nitroarachidonic acid 10 (6 mg, 0.017 mmol, 5.2%), 14-nitroarachidonic acid 11 (8 mg, 0.023 mmol, 6.9%), 5-nitroarachidonic acid 12 (3 mg, 0.009 mmol, 2.6%) (purity control via HPLC, ¹H and ¹³C NMR spectroscopy).

### Example H3

### Radical Nitration of Arachidonic Acid 9

Reaction as described in Example H2 using NO (gas) and hexane saturated with oxygen. A mixture of 6-nitroarachidonic acid 14, 14-nitroarachidonic acid 15, 5-nitroarachidonic acid 16 was obtained, yield about 12%, ratio 4:5:3.

### Example H4

### Radical Nitration of γ-Linolenic Acid 13

Reaction as described in Example H2 using 6,9,12-octadecatrienic acid (□-linolenic acid) 13 (100 mg, 0.36 mmol). Analysis and separation via HPLC (Phenomenex Gemini NX 5μ C18 110 Å, gradient MeOH/H₂O). Several regioisomer mono nitroacids are found with low yields as well as further minor products (not fully characterized).

6-nitro-γ-linolenic acid 14 (8 mg, 0.025 mmol, 6.9%), 12-nitro-γ-linolenic acid 15 (8 mg, 0.025 mmol, 6.9%), 5-nitro-γ-linolenic acid 16 (2 mg, 0.006 mmol, 1.7%) structure not fully proved.

### Example H5

### Radical Nitration of DHA

Reaction as described in Example H2 using (Z,Z,Z,Z,Z,Z)-4,7,10,13,16,19-docosahexaenoic acid (DHA) (100 mg, 0.30 mmol). Analysis and separation via HPLC (Phenomenex Gemini NX 5μ C18 110 Å, gradient MeOH/H₂O). Several regioisomer mono nitroacids are found with low yields as well as further minor products (not fully characterized). A mixture of 4-nitro-DHA, 5-nitro-DHA, 19-nitro-DHA and 20-nitro-DHA was isolated with about 7.1% yield.

### Example H6

### Radical Nitration of Palmitoleic Acid

Reaction as described in Example H2 using cis-9-hexadecenoic acid (palmitoleic acid) (200 mg, 0.79 mmol). Analysis and separation via HPLC (Phenomenex Gemini NX 5μ C18 110 Å, gradient MeOH/H₂O). A mixture of 9-nitropalmitoleic acid (68 mg, 0.23 mmol, yield 29%) and 10-nitropalmitoleic acid (52 mg, 0.17 mmol, 22%). was obtained.

### Example I1

### Electrophilic Nitration of Linolic Acid 1

The electrophilic nitration of linolic acid 1 was carried out according M. d'Ischia (J. Org. Chem. 2008, 73, 7517).

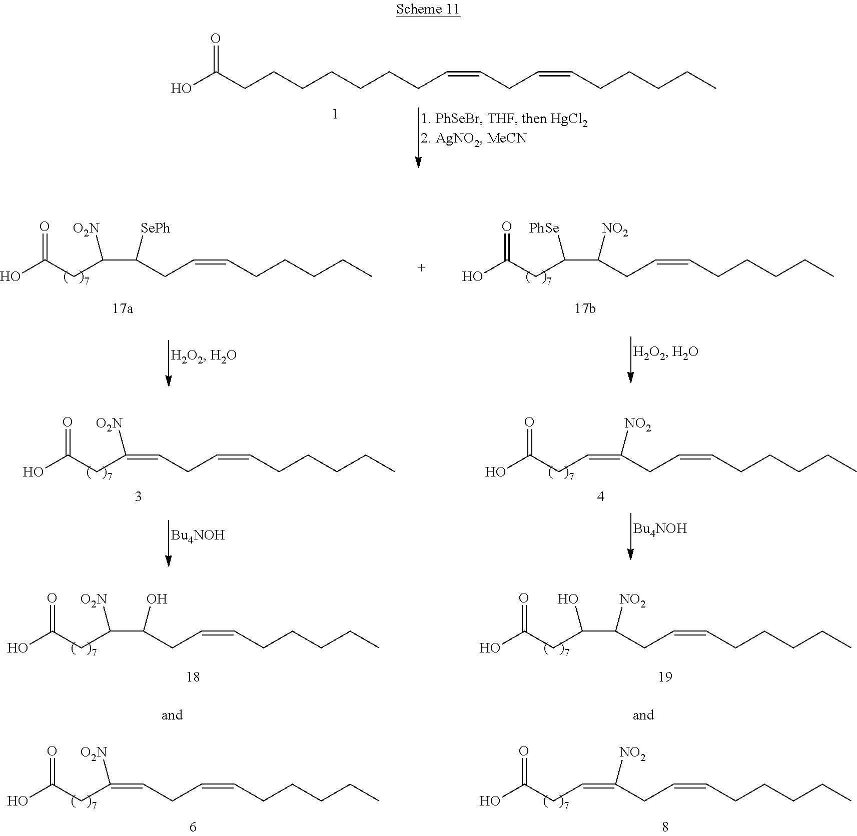

Under argon at −78° C., a solution of linoleic acid (1 g, 3.6 mmol) in dry THF (6 mL) was added dropwise to a solution of phenylselenyl bromide (843 mg, 3.6 mmol) in dry THF (10 mL) with stirring. After 20 min of reaction, HgCl₂ (1.26 g, 4.6 mmol) and a solution of AgNO₂ (550 mg, 3.6 mmol) in dry acetonitrile (15 mL via syringe pump within 1 h) were added subsequently. After 2 h of stirring at low temperatures and another 2 h at 23° C. the reaction was stopped. The solid (AgBr) was removed by filtration through a short celite column (careful elution with Et₂O). After removal of the solvents in vacuum the residue was dissolved in CH₂Cl₂, washed with brine (several times) and dried (Na₂SO₄). Again, the solvents were removed and the residue was purified using preparative HPLC (Phenomenex Gemini NX 5μ C18 110 Å, gradient MeCN/H₂O). The regioisomers 9/10-nitro-10/9-phenylselenyl fatty acid derivatives 17 were obtained as major fractions (mixtures of diastereomers). The proceeding elimination can be run using the mixture and the separated regioisomers, respectively. The fractions 17a and/or 17b were dissolved in CH₂Cl₂ (10 mL) and treated with an excess of aqueous H₂O₂ (about 8% in H₂O, at least 4 eq.) with vigorous stirring. After 1 h at 0° C. and 1 h at 23° C., the mixture was diluted with Et₂O and the layers were separated. The organic phase was intensely washed with brine and dried (Na₂SO₄). After removal of the solvent the residue was purified and separated via preparative HPLC (Phenomenex Gemini NX 5μ C18 110 Å, MeCN/H₂O). Yield: 115 mg (0.354 mmol, 10%) 10-nitrolinoleic acid 4 und 320 mg 9-nitrolinoleic acid 3 (0.98 mmol, 27%) (purity control via HPLC, ¹H and ¹³C NMR spectroscopy).

### Example I2

### Electrophilic Nitration of Gadoleic Acid

Reaction as described in Example I1 using cis-9-eicosenoic acid (gadoleic acid) (200 mg, 0.79 mmol). Analysis and separation via HPLC (Phenomenex Gemini NX 5μ C18 110 Å, gradient MeOH/H₂O). A mixture of 9-nitrogadoleic acid (42 mg, 0.12 mmol, yield 25%) and 10-nitrogadoleic acid (19 mg, 0.05 mmol, 11%). was obtained.

### Example I3

### Electrophilic Nitration of EPA

Reaction as described in Example I1 using (Z,Z,Z,Z,Z)-5,8,11,14,17-eicosapentaenoic acid (EPA) (100 mg, 0.33 mmol). Analysis and separation via HPLC (Phenomenex Gemini NX 5μ C18 110 Å, gradient MeOH/H₂O). A mixture of 5-nitro-EPA (17 mg, 0.05 mmol, yield 12%) and 6-nitro-EPA (7 mg, 0.02 mmol, 6%). was obtained.

### Example I4

### Electrophilic Nitration of α-Linolenic Acid

Reaction as described in Example I1 using (Z,Z,Z)-9,12,15-octadecatrienoic acid α-linolenic acid) (150 mg, 0.54 mmol). Analysis and separation via HPLC (Phenomenex Gemini NX 5μ C18 110 Å, gradient MeOH/H₂O). A mixture of 9-nitro-α-linolenic acid (37 mg, 0.11 mmol, yield 21%) and 10-nitro-α-linolenic acid (21 mg, 0.06 mmol, 12%). was obtained.

### Example J1

### Addition of Water to Nitrolinoleic Acid 1

The nitration was carried out as described for Example I1. In the presence of tetrabutylammonium hydroxide and CH₂Cl₂, H₂O is added to the nitroalkene moiety. 9-Hydroxy-10-nitro-12-octadecenoic acid 19 from 10-nitrolinoleic acid 4 and 10-hydroxy-9-nitro-12-octadecenoic acid 18 from 9-nitrolinoleic acid 3 were obtained, respectively. Work-up as described within Example I1. Separation of the diastereomers is possible but laborious. 9-Hydroxy-10-nitro-12-octadecenoic acid 19 was obtained with 10% yield and 10-hydroxy-9-nitro-12-octadecenoic acid 18 was obtained with 30% yield.

### Example J2

### Addition of Water to Nitrogadoleic Acid

Reaction as described in Example J1 using nitro-(E)-9-eicosenoic acid (nitrogadoleic acid, from Example I2) (150 mg, 0.48 mmol) and aqueous ammonium hydroxide in CH₂Cl₂. The addition of water delivered a mixture of 10-hydroxy-9-nitroeicosanoic acid and 9-hydroxy-10-nitroeicosanoic acid, ratio about 5:2, yield: 30% (54 mg, 0.86 mmol, mixture). Analysis and separation via HPLC (Phenomenex Gemini NX 5μ C18 110 Å, gradient MeOH/H₂O). The separation of the diastereomers was laborious and was omitted (not necessary in respect to the central aim of the present invention).

### Example J3

### Addition of Water to Nitro-EPA

Reaction as described in Example J1 using nitro-(Z,Z,Z,Z,Z)-5,8,11,14,17-eicosapentaenoic acid (nitro-EPA from Example I3) (100 mg, 0.33 mmol) and aqueous ammonium hydroxide in CH₂Cl₂. The addition of water delivered a mixture of 6-hydroxy-5-nitro-EPA (14 mg) and 5-hydroxy-6-nitro-EPA (5 mg), ratio about 5:2, yield: 16% (0.05 mmol, mixture). Analysis and separation via HPLC (Phenomenex Gemini NX 5μ C18 110 Å, gradient MeOH/H₂O). The separation of the diastereomers was laborious and omitted (not necessary in respect to the central aim of the present invention).

### Example J4

### Addition of Water to Nitro-α-Linolenic Acid

Reaction as described in Example J1 using nitro-(Z,Z,Z)-9,12,15-octadecatrienoic acid (α-linolenic acid) (150 mg, 0.54 mmol) and aqueous ammonium hydroxide in CH₂Cl₂. The addition of water delivered a mixture of 10-hydroxy-9-nitro-α-linolenic acid and 9-hydroxy-10-nitro-α-linolenic acid, ratio about 2:1, yield: 26% (48 mg, 0.14 mmol, mixture). Analysis and separation via HPLC (Phenomenex Gemini NX 5μ C18 110 Å, gradient MeOH/H₂O). The separation of the diastereomers was laborious and omitted (not necessary in respect to the central aim of the present invention).

### Example K1

### Electrophilic Double Nitration of Linoleic Acid 1

Upon running the nitration as described in Example I1a second time employing the nitration products generated in Example I1 the di-nitrolinoleic acids 5-8 are obtained as shown in Scheme 9. Within this second nitration step the more electron rich olefin reacted regioselectively. The nitration of 10-nitrolinoleic acid 4 and 9-nitrolinoleic acid 3 affords a mixture of products: 9,12-dinitrolinoleic acid, 9,13-dinitrolinoleic acid, 10,12-dinitrolinoleic acid and 10,13-dinitrolinoleic acid.

### Example K2

### Electrophilic Double Nitration of Gadoleic Acid

Upon running the nitration as described in Example I2 a second time employing the nitration products generated in Example I2 the introduction of a second nitro group failed. Here, the electron withdrawing effect of the first nitro group presumably suppressed the second electrophilic addition.

### Example K3

### Electrophilic Double Nitration of EPA

Upon running the nitration as described in Example I3a second time employing the nitration products generated in Example I3 the di-nitro-EPA products are obtained. A mixture of 5-nitro-EPA and 6-nitro-EPA (24 mg, 0.07 mmol) had been used. A mixture of regioisomers 5,17-dinitro-EPA, 5,18-dinitro-EPA, 6,17-dinitro-EPA and 6,18-dinitro-EPA had been obtained with an overall yield of 10% (3 mg, 0.007 mmol).

### Example K4

### Electrophilic Double Nitration of α-Linolenic Acid

Upon running the nitration as described in Example I4 a second time employing the nitration products generated in Example I4 the di-nitrolinoleic acids. The nitration of a mixture of 9-nitro-α-linolenic acid and 10-nitro-α-linolenic acid (50 mg, 0.15 mmol) affords a mixture of products: 9,15-dinitro-α-linolenic acid, 9,16-dinitro-α-linolenic acid, 10,15-dinitro-α-linolenic acid and 10,16-dinitro-α-linolenic acid are isolated with 9% yield (5 mg, 0.014 mmol) overall.

### Example L1

### Henry-Reaction of Nitroalkanes and Aldehydes

Within the present example the Henry-reaction of nitroalkanes and aldehydes according B. King (Org. Lett. 2006, 8, 2305) and B. Branchaud (Org. Lett. 2006, 8, 3931) is described.

### Addition:

A mixture of methyl 9-nitrononanoate 20 (4.2 g, 19.33 mmol) and nonanal 21a (2.75 g, 19.33 mmol) is treated with DBU (0.3 g, 1.93 mmol, 10 mol %) with stirring at 0° C. The mixture was stirred at 23° C. overnight. Then, 20 mL of Et₂O and 20 mL 0.1 N aqueous HCl were added. The aqueous layer was extracted with Et₂O and the combined organic phases were washed with brine and dried (Na₂SO₄). After removal of the solvents and purification of the residue via column chromatography (silica gel, EtOAc/hexanes 1:3) the β-hydroxynitroalkane 22a (6.56 g, 17.01 mmol, 88% yield) is obtained (purity control via ¹H and ¹³C NMR spectroscopy).

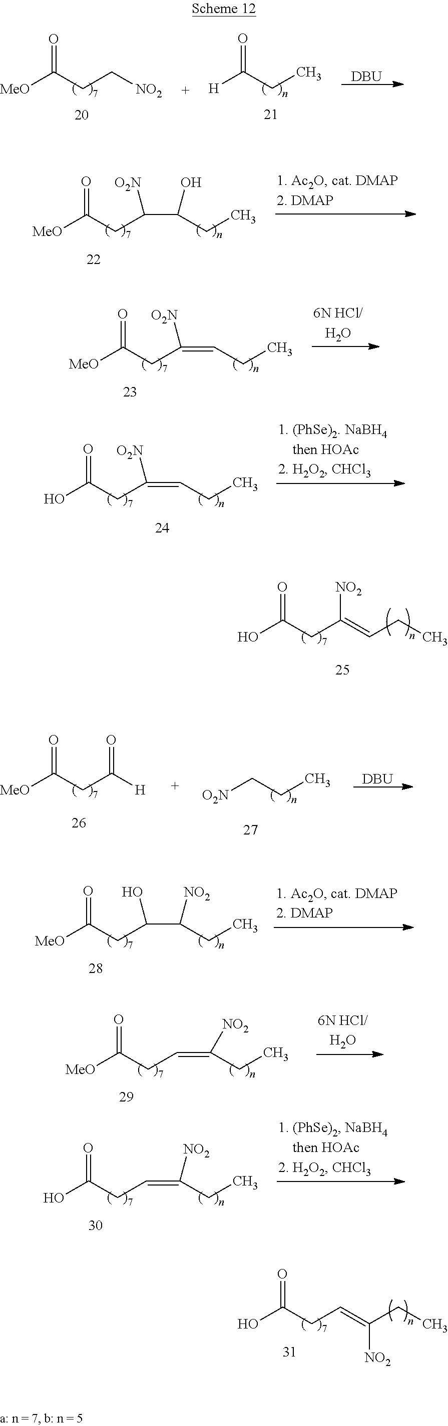

### Condensation:

A solution of β-hydroxynitroalkane 22a (6.56 g, 17.01 mmol), and DMAP (25.9 mg, 0.17 mmol) in Et₂O (40 ml) is treated with Ac₂O (1.91 g, 18.71 mmol). The mixture was stirred at 23° C. for 5 h. Then, Et₂O was evaporated under reduced pressure, the residue was dissolved in CH₂Cl₂ (40 mL) and DMAP (2.49 g, 20.4 mmol) was added. The mixture was stirred at 23° C. for 2 h. After dilution with CH₂Cl₂ (300 mL), the organic layer was washed with water, 0.1N aqueous HCl and brine. After drying (MgSO₄) the solvent was removed and the residue was purified by column chromatography (silica gel) and preparative HPLC (Polygosil 60-5, 1.3% EtOAc in hexane). Yield of methyl 9-nitrooleate 23a: 3.89 g (11.4 mmol, 67%) (purity control via ¹H and ¹³C NMR spectroscopy).

### Ester Hydrolysis:

methyl 9-nitrooleate 23a (3.89 g, 11.4 mmol) and 6 M aqueous HCl (100 ml) were heated to reflux for 18 h. After cooling down to 23° C. the mixture was thoroughly extracted with EtOAc. The organic layers were washed with brine and dried (MgSO₄). After removal of the solvent the residue was purified by column chromatography (silica gel) and preparative HPLC (Phenomenex Gemini NX 5μ C18 110 Å, MeCN/H₂O). Yield of E-9-nitrooleic acid 24a: 2.57 g (7.87 mmol, 69%) (purity control via ¹H and ¹³C NMR spectroscopy).

The stereoisomer Z-9-nitrooleic acid 24a can be obtained as the methyl ester Z-24a as a minor compound (389 mg, 10% yield) within the condensation step (purity control via ¹H and ¹³C NMR spectroscopy). An E/Z isomerization succeeds according a procedure published by Branchaud (1. PhSeSePh, NaBH₄, then HOAc, 2. H₂O₂) starting from acid 24a (207 mg, 1 mmol). Yield: 71% as an E/Z mixture, ratio 1:3. Separation of the nitro olefins via preparative HPLC as described for “ester hydrolysis” (purity control via ¹H and ¹³C NMR spectroscopy).

### Example L2

### Synthesis of E-10-nitrooleic acid 30a

The sequence according Example L1 starting from methyl 9-oxononanoate 26 (1.0 g, 5.38 mmol) and 1-nitrononane 27a (0.93 g, 5.38 mmol) afforded 10-nitrooleic acid 30a in 44.5% yield (784 mg, 2.39 mmol) over all steps. Purification via preparative HPLC (Phenomenex Gemini NX 5μ C18 110 Å, MeCN/H₂O) (purity control via ¹H and ¹³C NMR spectroscopy).

### Example L3

### Synthesis of E-9-nitropalmitoleic acid 24b

The sequence according Example L1 starting from methyl 9-nitrononanoate 20 (1.0 g, 4.6 mmol) and heptanal 21b (524.4 mg, 4.6 mmol) afforded E-9-nitropalmitoleic acid 24b in 41.6% yield (573 mg, 1.91 mmol) over all steps. Purification via preparative HPLC (Phenomenex Gemini NX 5μ C18 110 Å, MeCN/H₂O) (purity control via ¹H and ¹³C NMR spectroscopy).

### Example L4

### Synthesis of E-10-nitropalmitoleic acid 30b

The sequence according Example L1 starting from methyl 9-oxononanoate 26 (1.0 g, 5.38 mmol) and 1-nitroheptane 27b (780 mg, 5.38 mmol) afforded E-10-nitropalmitoleic acid 30b in 38.5% yield (620 mg, 2.07 mmol) over all steps. Purification via preparative HPLC (Phenomenex Gemini NX 5μ C18 110 Å, MeCN/H₂O) (purity control via ¹H and ¹³C NMR spectroscopy).

### Example M1

### Semi-Syntheses Starting from α-Linolenic Acid 32

Reactions starting from α-linolenic acid 32: α-Linolenic acid 32 (commercially available) is degraded via epoxide 33 according a procedure published by A. Makriyannis (J. Lab. Comp. Radiopharm. 2003, 46, 645) and W. Boland (Tetrahedron 2003, 59, 135) to give aldehyde 34. Adapting the sequence of B. Branchaud (Org. Lett. 2006, 8, 3931) the aldehyde 34 is converted into the nitroester 36. The Henry reaction was carried out as described for Example L1 using propanal (R═CH₃) to give nitroester 38. In contrast to the procedure described above, the ester hydrolysis was run according a procedure published by G. Zanoni and G. Vidari (J. Org. Chem. 2010, 75, 8311):

(Enzymatic ester cleavage): Methyl E-15-nitro-α-linolenate 38 (120 mg, 0.356 mmol, R=Me) was dissolved in tert-butylmethyl ether (35 mL) and water (0.178 ml, 0.178 mmol). Solid-supported Candida antarctica lipase B (CAL-B, 30 mg) was added and the mixture was stirred at 35° C. for 18 h. After filtering off the enzyme (filter carefully washing with MeCN/tert-butylmethyl ether) the solvents were removed under vacuum at temperatures below 15° C. The residue was purified via preparative HPLC (Phenomenex Gemini NX 5μ C18 110 Å, MeCN/H₂O). Yield: E-15-Nitro-α-linolenic acid 41: 106.9 mg (0.331 mmol, 93%, R=Me) (purity control via HPLC, ¹H and ¹³C NMR spectroscopy).

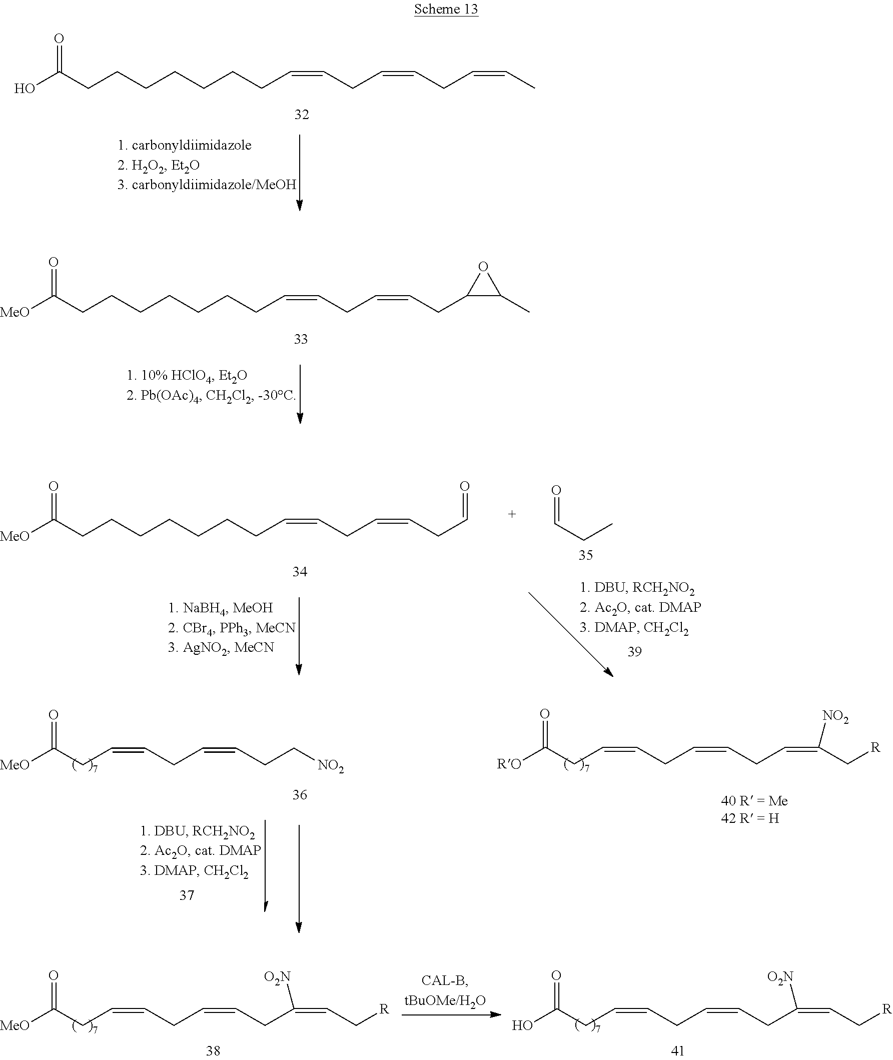

### Example M2

### Synthesis of E-15-nitro-α-linolenic acid 41 (R═CH₃)

The sequence according Example M1 starting from methyl 15-nitro-9,12-pentadecadienoate 36 (198 mg, 0.667 mmol) and propanal 37 (38.7 mg, 0.667 mmol, R═CH₃) afforded E-15-nitro-α-linolenic acid 41 in 49.6% yield (106.9 mg, 0.331 mmol, R═CH₃) over all steps. Purification via preparative HPLC (Phenomenex Gemini NX 5μ C18 110 Å, MeCN/H₂O) (purity control via ¹H and ¹³C NMR spectroscopy).

### Example M3

### Synthesis of E-16-nitro-α-linolenic acid 42 (R═CH₃)

The sequence according Example M1 starting from methyl 15-oxo-9,12-pentadecadienoate 34 (201 mg, 0156 mmol) and 1-nitropropane 39 (67.3 mg, 0.756 mmol, R═CH₃) and the enzymatic ester cleavage 40 in 42 afforded E-16-nitro-α-linolenic acid 42 in 54.1% yield (138.2 mg, 0.41 mmol, R═CH₃) over all steps. Purification via preparative HPLC (Phenomenex Gemini NX 5μ C18 110 Å, MeCN/H₂O) (purity control via ¹H and ¹³C NMR spectroscopy).

### Example M4

### Synthesis of E-15-nitro-9,12,15-eicosatrienic acid 41 (R═C₃H₇)

The sequence according Example M1 starting from methyl 15-nitro-9,12-pentadecadienoate 36 (201 mg, 0.756 mmol) and pentanal 37 (57.4 mg, 0.667 mmol, R═C₃H₇) and the enzymatic ester cleavage 38 in 41 afforded E-15-nitro-9,12,15-eicosatrienic acid 42 in 48.1% yield (112.6 mg, 0.321 mmol, R═O₃H₇) over all steps. Purification via preparative HPLC (Phenomenex Gemini NX 5μ C18 110 Å, MeCN/H₂O) (purity control via ¹H and ¹³C NMR spectroscopy).

### Example M5

### Synthesis of E-16-nitro-9,12,15-docosatetraenic acid 42 (R═C₅H₁₁)

The sequence according Example M1 starting from methyl 15-oxo-9,12-pentadecadienoate 34 (151 mg, 0.568 mmol) and 1-nitroheptane 39 (64.7 mg, 0.568 mmol, R═C₅H₁₁) and the enzymatic ester cleavage 40 in 42 afforded E-16-nitro-9,12,15-docosatetraenic acid 42 in 42.4% yield (91.3 mg, 0.241 mmol, R═C₅H₁₁) over all steps. Purification via preparative HPLC (Phenomenex Gemini NX 5μ C18 110 Å, MeCN/H₂O) (purity control via ¹H and ¹³C NMR spectroscopy).

### Example N1

### Synthesis of a mixture of 1,2-di-(9-nitrolinoleoyl)-sn-3-glycerophosphocholine, 1,2-di-(10-nitrolinoleoyl)-sn-3-glycerophosphocholine, 1-(9-nitrolinoleoyl)-2-(10-nitrolinoleoyl)-sn-3-glycerophosphocholine and 1-(10-nitrolinoleoyl)-2-(9-nitrolinoleoyl)-sn-3-glycerophosphocholine

Following the procedure for Example B sn-glycero-3-phosphatidylcholine 1a (103 mg, 0.4 mmol) was reacted with a mixture of 9-nitrolinoleic acid 3 and 10-nitrolinoleic acid 4 (mixture, ratio 1:1, 0.4 g, 1.2 mmol, obtained as described for Example H1). A mixture of 1,2-di-(9-nitrolinoleoyl)-sn-3-glycerophosphocholine, 1,2-di-(10-nitrolinoleoyl)-sn-3-glycerophosphocholine, 1-(9-nitrolinoleoyl)-2-(10-nitrolinoleoyl)-sn-3-glycerophosphocholine and 1-(10-nitrolinoleoyl)-2-(9-nitrolinoleoyl)-sn-3-glycerophosphocholine was obtained, yield 68% (237 mg, 0.27 mmol) (purity control via HPLC, ¹H and ¹³C NMR spectroscopy).

### Example N2

### Nitrolinoleic acid ester from sn-glycero-3-phosphatidylcholine 1a

Following the procedure for Example B sn-glycero-3-phosphatidylcholine 1a (0.13 g, 0.5 mmol) was reacted with a mixture of 9-nitrolinoleic acid 3,10-nitrolinoleic acid 4, 12-nitrolinoleic acid and 9-nitro-11,12 octadecadienic acid (mixture, ratio 7:12:5:2, 0.5 g, 1.5 mmol, obtained as described for Example H1). A mixture containing several acyl-derived 1,2-di-(nitrolinoleoyl)-sn-glycero-3-phosphatidylcholines could be generated with 61% yield (212 mg, 0.24 mmol), (purity control via HPLC, ¹H and ¹³C NMR spectroscopy).

### Example N3

### Nitroarachidonic acid ester from sn-glycero-3-phosphatidylcholine 1a

Following the procedure for Example B sn-glycero-3-phosphatidylcholine 1a (130 mg, 0.5 mmol) was reacted with a mixture of 6-nitroarachidonic acid 10, 14-nitroarachidonic acid 11 and 5-nitroarachidonic acid 12 (mixture, ratio 8:6:3, 0.45 g, 1.5 mmol, obtained as described for Example H2). A mixture containing several acyl-derived 1,2-di-(nitroarachidonoyl)-sn-glycero-3-phosphatidylcholines could be generated with 53% yield (244 mg, 0.27 mmol), (purity control via HPLC, ¹H and ¹³C NMR spectroscopy).

### Example N4

### Nitroarachidonic acid ester from sn-glycero-3-phosphatidylcholine 1a

Following the procedure for Example B sn-glycero-3-phosphatidylcholine 1a (130 mg, 0.5 mmol) was reacted with a mixture of 6-nitroarachidonic acid 14, 14-nitroarachidonic acid 15 and 5-nitroarachidonic acid 16 (mixture, ratio 4:5:3, 0.45 g, 1.5 mmol, obtained as described for Example H3). A mixture containing several acyl-derived 1,2-di-(nitroarachidonoyl)-sn-glycero-3-phosphatidylcholines could be generated with 55% yield (253 mg, 0.28 mmol), (purity control via HPLC, ¹H and ¹³C NMR spectroscopy).

### Example N5

### Nitro-γ-linolenic acid ester from sn-glycero-3-phosphatidylcholine 1a

Following the procedure for Example B sn-glycero-3-phosphatidylcholine 1a (130 mg, 0.5 mmol) was reacted with a mixture of 6-nitro-γ-linolenic acid 14, 12-nitro-γ-linolenic acid 15 and 5-nitro-γ-linolenic acid 16 (mixture, ratio 4:4:1, 0.485 g, 1.5 mmol, obtained as described for Example H4). A mixture containing several acyl-derived 1,2-di-(nitro-γ-linolenoyl)-sn-glycero-3-phosphatidylcholines could be generated with 59% yield (257 mg, 0.3 mmol), (purity control via HPLC, ¹H and ¹³C NMR spectroscopy).

### Example N6

### Nitro-DHA ester from sn-glycero-3-phosphatidylcholine 1a

Following the procedure for Example B sn-glycero-3-phosphatidylcholine 1a (130 mg, 0.5 mmol) was reacted with a mixture of 4-nitro-DHA, 5-nitro-DHA, 19-nitro-DHA and 20-nitro-DHA (0.56 g, 1.5 mmol, obtained as described for Example H5). A mixture containing several acyl-derived 1,2-di-(nitro-DHA)-sn-glycero-3-phosphatidylcholines could be generated with 31% yield (150 mg, 0.16 mmol), (purity control via HPLC, ¹H and ¹³C NMR spectroscopy).

### Example N7

### Nitropalmitoleic acid ester from sn-glycero-3-phosphatidylcholine 1a

Following the procedure for Example B sn-glycero-3-phosphatidylcholine 1a (130 mg, 0.5 mmol) was reacted with a mixture of 9-nitropalmitoleic acid and 10-nitropalmitoleic acid (mixture, ratio 4:3, 0.45 g, 1.5 mmol, obtained as described for Example H6). A mixture containing 1,2-di-(9-nitropalmitoleoyl)-sn-3-glycerophosphocholine, 1,2-di-(10-nitropalmitoleoyl)-sn-3-glycerophosphocholine, 1-(9-nitropalmitoleoyl)-2-(10-nitropalmitoleoyl)-sn-3-glycerophosphocholine and 1-(10-nitropalmitoleoyl)-2-(9-nitropalmitoleoyl)-sn-3-glycerophosphocholine could be generated with 77% yield (315 mg, 0.39 mmol), (purity control via HPLC, ¹H and ¹³C NMR spectroscopy).

### Example N8

### Nitrolinoleic acid ester from sn-glycero-3-phosphatidylcholine 1a

Following the procedure for Example B sn-glycero-3-phosphatidylcholine 1a (130 mg, 0.5 mmol) was reacted with a mixture of 10-nitrolinoleic acid 4 and 9-nitrolinoleic acid 3 (mixture, ratio 1:3, 0.49 g, 1.5 mmol, obtained as described for Example I1). A mixture containing 1,2-di-(9-nitrolinoleoyl)-sn-3-glycerophosphocholine, 1,2-di-(10-nitrolinoleoyl)-sn-3-glycerophosphocholine, 1-(9-nitrolinoleoyl)-2-(10-nitrolinoleoyl)-sn-3-glycerophosphocholine and 1-(10-nitrolinoleoyl)-2-(9-nitrolinoleoyl)-sn-3-glycerophosphocholine could be generated with 79% yield (323 mg, 0.4 mmol), (purity control via HPLC, ¹H and ¹³C NMR spectroscopy). Separation of the compounds to assemble spectroscopic data:

### 1,2-Di-(9-nitrolinoleoyl)-sn-3-glycerophosphatidylcholine

173.5, 173.0 (C═O), 150.0, 149.0 (2×C—NO₂), 133.5, 133.0 (2×HC═), 131.5, 131.0 (2×HC═), 125.0, 124.5 (2×HC═), 71.0 (d), 66.5 (d), 64.0 (d), 62.5, 59.5 (d), 54.5 (NMe₃), 35.0 (2x=CH₂═), 34.5-20.5 (22×CH₂), 14.0, 13.5 (2×CH₃).

### 1,2-Di-(10-nitrolinoleoyl)-sn-3-glycerophosphatidylcholine

173.5, 173.0 (C═O), 152.0, 151.5 (2×C—NO₂), 134.5, 134.0 (2×HC═), 132.5, 132.0 (2×HC═), 124.0, 123.5 (2×HC═), 71.0 (d), 66.5 (d), 64.5 (d), 63.0, 60.0 (d), 54.5 (NMe₃), 37.5, 37.0 (2×=CH₂═), 34.5-21.5 (22×CH₂), 14.0 (2×CH₃).

### Example N9

### Nitrogadoleic acid ester from sn-glycero-3-phosphatidylcholine 1a

Following the procedure for Example B sn-glycero-3-phosphatidylcholine 1a (130 mg, 0.5 mmol) was reacted with a mixture of 9-nitrolgadoleic acid and 10-nitrogadoleic acid (mixture, ratio 1:2, 0.53 g, 1.5 mmol, obtained as described for Example I2). A mixture containing 1,2-di-(9-nitrogadleoyl)-sn-3-glycerophosphocholine, 1,2-di-(10-nitrolinoleoyl)-sn-3-glycerophosphocholine, 1-(9-nitrolinoleoyl)-2-(10-nitrogadoleoyl)-sn-3-glycerophosphocholine and 1-(10-nitrogadoleoyl)-2-(9-nitrogadoleoyl)-sn-3-glycerophosphocholine could be generated with 67% yield (310 mg, 0.34 mmol), (purity control via HPLC, ¹1-1 and ¹³C NMR spectroscopy).

### Example N10

### Nitro-EPA ester from sn-glycero-3-phosphatidylcholine 1a

Following the procedure for Example B sn-glycero-3-phosphatidylcholine 1a (130 mg, 0.5 mmol) was reacted with a mixture of 5-nitro-EPA und 6-nitro-EPA (mixture, ratio 5:2, 0.46 g, 1.5 mmol, obtained as described for Example I3). A mixture containing 1,2-di-(5-nitroeicosapentaenoyl)-sn-3-glycerophosphocholine, 1,2-di-(6-nitroeicosapentaenoyl)-sn-3-glycerophosphocholine, 1-(5-nitroeicosapentaenoyl)-2-(6-nitroeicosapentaenoyl)-sn-3-glycerophosphocholine and 1-(6-nitroeicosapentaenoyl)-2-(5-nitroeicosapentaenoyl)-sn-3-glycerophosPhocholine could be generated with 49% yield (224 mg, 0.25 mmol), (purity control via HPLC, ¹H and ¹³C NMR spectroscopy).

### Example N11

### Nitro-α-linolenic acid ester from sn-glycero-3-phosphatidylcholine 1a

Following the procedure for Example B sn-glycero-3-phosphatidylcholine 1a (130 mg, 0.5 mmol) was reacted with a mixture of 9-nitro-α-linolenic acid and 10-nitro-α-linolenic acid (mixture, ratio 1:2, 0.485 g, 1.5 mmol, obtained as described for Example I4). A mixture containing 1,2-di-(9-nitro-α-linolenoyl)-sn-3-glycerophosphocholine, 1,2-di-(10-nitro-α-linolenoyl)-sn-3-glycerophosphocholine, 1-(9-nitro-α-linolenoyl)-2-(10-nitro-α-linolenoyl)-sn-3-glycerophosphocholine and 1-(10-nitro-α-linolenoyl)-2-(9-nitro-α-linolenoyl)-sn-3-glycerophosphocholine could be generated with 54% yield (235 mg, 0.27 mmol), (purity control via HPLC, ¹H and ¹³C NMR spectroscopy).

### Example N12

### Dinitrolinoleic acid ester from sn-glycero-3-phosphatidylcholine 1a

Following the procedure for Example B sn-glycero-3-phosphatidylcholine 1a (130 mg, 0.5 mmol) was reacted with a mixture of 9,12-dinitrolinoleic acid, 9,13-dinitrolinoleic acid, 10,12-dinitrolinoleic acid and 10,13-dinitrolinoleic acid (0.56 g, 1.5 mmol, obtained as described for Example K1). A mixture containing several 1,2-di-(dinitrolinoleoyl)-sn-glycero-3-phosphatidylcholines was generated with 64% yield (310 mg, 0.32 mmol), (purity control via HPLC, ¹H and ¹³C NMR spectroscopy).

### Example N13

### Dinitro-EPA ester from sn-glycero-3-phosphatidylcholine 1a

Following the procedure for Example B sn-glycero-3-phosphatidylcholine 1a (130 mg, 0.5 mmol) was reacted with a mixture of 5,17-dinitro-EPA, 5,18-dinitro-EPA, 6,17-dinitro-EPA and 6,18-dinitro-EPA (0.59 g, 1.5 mmol, obtained as described for Example K2). A mixture containing several 1,2-di-(dinitroeicosapentaenoyl)-sn-glycero-3-phosphatidylcholines was generated with 33% yield (170 mg, 0.18 mmol), (purity control via HPLC, ¹H and ¹³C NMR spectroscopy).

### Example N14

### Dinitro-α-linolenic acid ester from sn-glycero-3-phosphatidylcholine 1a

Following the procedure for Example B sn-glycero-3-phosphatidylcholine 1a (130 mg, 0.5 mmol) was reacted with a mixture of 9,15-dinitro-α-linolenic acid, 9,16-dinitro-α-linolenic acid, 10,15-dinitro-α-linolenic acid and 10,16-dinitro-α-linolenic acid (0.55 g, 1.5 mmol, obtained as described for Example K4). A mixture containing several 1,2-di-(dinitro-α-linolenoyl)-sn-glycero-3-phosphatidylcholines was generated with 35% yield (170 mg, 0.18 mmol), (purity control via HPLC, ¹H and ¹³C NMR spectroscopy).

### Example N15

### Synthesis of 1,2-di-(E-9-nitrooleoyl)-sn-3-glycerophosphocholine

Following the procedure for Example B sn-glycero-3-phosphatidylcholine 1a (130 mg, 0.5 mmol) was reacted with Z-9-nitrooleic acid 25a (0.49 g, 1.5 mmol, obtained as described for Example L1). 1,2-Di-(E-9-nitrooleoyl)-sn-glycero-3-phosphatidylcholine was isolated with 75% yield (328 mg, 0.38 mmol), (purity control via HPLC, ¹H and ¹³C NMR spectroscopy).

### Example N16

### Synthesis of 1,2-di-(E-9-nitrooleoyl)-sn-3-glycerophosphocholine

Following the procedure for Example B sn-glycero-3-phosphatidylcholine 1a (130 mg, 0.5 mmol) was reacted with E-9-nitrooleic acid 24a (0.49 g, 1.5 mmol, obtained as described for Example L1). 1,2-Di-(E-9-nitrooleoyl)-sn-glycero-3-phosphatidylcholine was isolated with 74% yield (323 mg, 0.37 mmol), (purity control via HPLC, ¹H and ¹³C NMR spectroscopy).

### Example N17

### Synthesis of 1,2-di-(E-10-nitrooleoyl)-sn-3-glycero-phosphocholine

Following the procedure for Example B sn-glycero-3-phosphatidylcholine 1a (130 mg, 0.5 mmol) was reacted with E-10-nitrooleic acid 30a (0.49 g, 1.5 mmol, obtained as described for Example L2). 1,2-Di-(E-10-nitrooleoyl)-sn-glycero-3-phosphatidylcholine was isolated with 70% yield (306 mg, 0.35 mmol), (purity control via HPLC, ¹H and ¹³C NMR spectroscopy).

¹³C data: 173.5, 173.0 (C═O), 150.0 (2×C—NO₂), 134.0, 133.5 (2×HC═), 71.0 (d), 66.5 (d), 64.0 (d), 63.0, 59.5 (d), 54.5 (NMe₃), 34.5-20.5 (28×CH₂), 14.5 (2×CH₃).

### Example N18

### Synthesis of 1,2-di-(E-9-nitropalmitoleoyl)-sn-3-glycerophosphocholine

Following the procedure for Example B sn-glycero-3-phosphatidylcholine 1a (130 mg, 0.5 mmol) was reacted with E-9-nitropalmitoleoic acid 24b (0.45 g, 1.5 mmol, obtained as described for Example L3). 1,2-Di-(E-9-nitropalmitoleoyl)-sn-glycero-3-phosphatidylcholine was isolated with 72% yield (295 mg, 0.36 mmol), (purity control via HPLC, ¹H and ¹³C NMR spectroscopy).

### Example N19

### Synthesis of 1,2-di-(E-10-nitropalmitoleoyl)-sn-3-glycerophosphocholine

Following the procedure for Example B sn-glycero-3-phosphatidylcholine 1a (130 mg, 0.5 mmol) was reacted with E-10-nitropalmitoleoic acid 30b (0.45 g, 1.5 mmol, obtained as described for Example L4). 1,2-Di-(E-10-nitropalmitoleoyl)-sn-glycero-3-phosphatidylcholine was isolated with 70% yield (287 mg, 0.35 mmol), (purity control via HPLC, ¹H and ¹³C NMR spectroscopy).

### Example N20

### Synthesis of 1,2-di-(E-15-nitro-α-linolenoyl)-sn-3-glycerophosphocholine

Following the procedure for Example B sn-glycero-3-phosphatidylcholine 1a (130 mg, 0.5 mmol) was reacted with E-15-nitro-α-linolenic acid 41 (0.45 g, 1.5 mmol, obtained as described for Example M1). 1,2-Di-(E-15-nitro-α-linolenoyl)-sn-glycero-3-phosphatidylcholine was isolated with 65% yield (282 mg, 0.33 mmol), (purity control via HPLC, ¹H and ¹³C NMR spectroscopy).

### Example O1

### Synthesis of 1,2-di-(9-nitro-10-hydroxy-stearoyl)-sn-3-glycerophosphocholine (β,γ-di-(9-nitro-10-hydroxystearoyl)-L-α-glycerophosphatidylcholine) 3a1

The ester formation to introduce additional nitro fatty acids can be directly adapted from the procedure published by R. Salomon (Biorg. & Med. Chem. 2011, 19, 580).

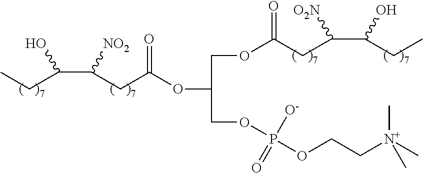

### 1,2-Di-(9-nitro-10-hydroxystearyl)-sn-3-glycero-phosphatidyl choline 3a1

A suspension of sn-Glycero-3-phosphocholin 1a (0.15 g, 0.585 mmol, 1 eq.) in dry CH₂Cl₂ (50 mL) was subsequently treated with 9-nitro-10-hydroxystearic acid (0.605 g, 1.755 mmol, 3 eq., mixture of diastereomers), 1-methyl imidazole (0.144 g, 0.141 mL, 1.755 mmol, 3 eq.) and 2,6-dichlorobenzoyl chloride (0.367 g, 1.755 mmol 3 eq.). The mixture was stirred at 23° C. for 3 d, within this period the suspension converted into a clear solution. The solvents were removed in vacuum and the residue was purified by column chromatography (silica gel) and preparative HPLC (Phenomenex Gemini NX 5μ C18 110 Å, 95% MeOH/H₂O). Yield: 250.5 mg (0.275 mmol, 47%) of 3a1 (purity control via HPLC, ¹H and ¹³C NMR spectroscopy). The product formed is a mixture of diasteromers (separation difficult) concerning the stereogenic centres within the fatty acid moiety and was used as is in further applications.

### Example O2

### Synthesis of 1-palmitoyl-2-(9-nitro-10-hydroxy-stearoyl)-sn-3-glycerophosphocholine (β-(9-nitro-10-hydroxystearoyl)-γ-palmitoyl-L-α-phosphatidylcholine) 6a1

The ester formation to introduce additional nitro fatty acids can be directly adapted from the procedure published by R. Salomon (Biorg. & Med. Chem. 2011, 19, 580).

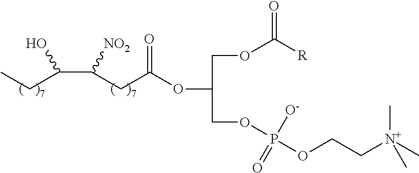

### 1-Palmitoyl-2-(9-nitro-10-hydroxystearyl)-sn-3-glycero-phosphatidyl choline 6a1

A solution of 9-nitro-10-hydroxystearic acid (0.142 g, 0.412 mmol, 2.04 eq., racemate, 3:1 mixture of diastereomers) and 1-palmitoyl-2-lyso-sn-3-glycerophosphocholine 5a (0.1 g, 0.202 mmol) in dry CH₂Cl₂ (10 mL) was treated with 1-methyl imidazole (0.05 g, 0.05 mL, 0.6 mmol, 2.97 eq.) and 2,6-dichlorobenzoyl chloride (0.14 g, 0.01 ml, 0.67 mmol 3.32 eq.). The mixture was stirred at 23° C. for 3 d. The solvents were removed in vacuum and the residue was purified by column chromatography (silica gel) and preparative HPLC (Phenomenex Gemini NX 5μ C18 110 Å, 95% MeOH/H₂O). Yield: 86.1 mg (0.105 mmol, 52%) of 6a1 as a white solid (purity control via HPLC, ¹H and ¹³C NMR spectroscopy). The product formed is a mixture of diastereomers (separation difficult) concerning the stereogenic centres within the fatty acid moiety, ratio about 3:3:1:1. It was used as is in further applications.

¹³C NMR data of 6a1: 173.5 (2×C═O), 91.0 (CH—NO₂), 72.5 (HC—OH), 71.0 (d), 66.0 (d), 63.0 (d), 62.5, 59.5 (d), 54.0 (NMe₃), 34.5-21.0 (28×CH₂), 14.0 (2×CH₃).

### Example O3

Reaction as described for Example O1 replacing racemic 9-nitro-10-hydroxystearic acid (one regioisomer) by a mixture of 9-nitro-10-hydroxystearic acid and 10-nitro-9-hydroxystearic acid, ratio 1:3, obtained from the synthesis as described for Example J1. Single regioisomers and mixtures of regioisomers are characterised by similar reactivity. 1,2-Di-(9-hydroxy-10-nitrostearoyl)-sn-3-glycerophosphocholine, 1-(9-hydroxy-10-nitrostearoyl)-2-(10-hydroxy-9-nitrostearoyl)-sn-3-glycerophosphocholine, 1-(10-hydroxy-9-nitrostearoyl)-2-(9-hydroxy-10-nitrostearoyl)-sn-3-glycerophosphocholine and 1,2-di-(10-hydroxy-9-nitrostearoyl)-sn-3-glycerophosphocholine are obtained with an overall yield of 45% (240 mg, 0.263 mmol). Again, mixtures of diasteromers are formed.

### Example O4

Reaction as described for Example O2 replacing racemic 9-nitro-10-hydroxystearic acid (one regioisomer) by a mixture of 9-nitro-10-hydroxystearic acid and 10-nitro-9-hydroxystearic acid, ratio 1:3, obtained from the synthesis as described for Example J1. Single regioisomers and mixtures of regioisomers are characterised by similar reactivity. 1-Palmitoyl-2-(9-hydroxy-10-nitrostearoyl)-sn-3-glycerophosphocholine and 1-palmitoyl-2-(10-hydroxy-9-nitrostearoyl)-sn-3-glycerophosphocholine are obtained with an overall yield of 48% (79.5 mg, 0.097 mmol). Again, mixtures of diastereomers are formed.

¹³C data of 6a2: 173.5, 173.0 (2×C═O), 92.0 (CH—NO₂), 73.0 (HC—OH), 70.5 (d), 66.5 (d), 63.5 (d), 63.0, 59.5 (d), 54.5 (NMe₃), 34.5-21.0 (28×CH₂), 14.0 (2×CH₃).

### Example O5

### Synthesis of 1,2-di-(hydroxyl, nitro-8,11,14,17-eicosatetraenoyl)-sn-3-glycerophosphocholine

According Example J3 a mixture of 6-hydroxy-5-nitro-8,11,14,17-eicosatetraenic acid and 5-hydroxy-6-nitro-8,11,14,17-eicosatetraenic acid, ratio about 5:2 was synthesized. According Example O3 the mixture was reacted with sn-glycero-3-phosphocholine 1a (0.15 g, 0.585 mmol, 1 eq.). 1,2-Di-(5-hydroxy-6-nitro-8,11,14,17-eicosatetraenoyl)-sn-3-glycerophosphocholine, 1-(5-hydroxy-6-nitro-8,11,14,17-eicosatetraenoyl)-2-(6-hydroxy-5-nitro-8,11,14,17-eicosatetraenoyl)-sn-3-glycerophosphocholine, 1-(6-hydroxy-5-nitro-8,11,14,17-eicosatetraenoyl)-2-(5-hydroxy-6-nitro-8,11,14,17-eicosatetraenoyl)-sn-3-glycerophosphocholine and 1,2-di-(6-hydroxy-5-nitro-8,11,14,17-eicosatetraenoyl)-sn-3-glycerophosphocholine were obtained with an overall yield of 35% (195 mg, 0.208 mmol). Again, mixtures of diasteromers are formed.

### Example O6

According Example J4 a mixture of 10-hydroxy-9-nitro-α-linolenic acid and 9-hydroxy-10-nitro-α-linolenic acid, ratio about 2:1 was synthesized. According Example O4 the mixture was reacted with 1-palmitoyl-2-lyso-sn-3-glycerophosphocholine 5a (0.1 g, 0.202 mmol). 1-Palmitoyl-2-(10-hydroxy-9-nitro-α-linolenoyl)-sn-3-glycerophosphocholine and 1-palmitoyl-2-(9-hydroxy-10-nitro-α-linolenoyl)-sn-3-glycerophosphocholine were obtained with an overall yield of 37% (61 mg, 0.075 mmol). Again, mixtures of diasteromers are formed.

### Example P1

### Synthesis of a mixture of 1-oleoyl-2-(9-nitropalmitoleoyl)-sn-3-glycerophosphocholine and 1-oleoyl-2-(10-nitropalmitoleoyl)-sn-3-glycerophosphocholine

Following the preparation procedure described in Example C, sn-glycero-3-phosphatidylcholin 1a (1.0 g, 3.9 mmol) and dibutyltin oxide (1.1 g, 4.3 mmol, 1.1 eq.) suspended in isopropanol (100 mL) were heated to reflux. Then, the first sn-1 esterification was run using Et₃N (0.25 mL, 7.8 mmol, 1.1 eq.) and oleic acid (2.35 g, 7.8 mmol, 2 eq.) to afford 1-oleoyl-2-lyso-sn-3-glycerophosphocholine in 44% yield (0.89 g, 1.72 mmol). According Example C the second acylation was carried out. I-oleoyl-2-lyso-sn-3-glycerophosphocholine and a mixture of 9-nitropalmitoleic acid and 10-nitropalmitoleic acid (ratio 4:3, 1.03 g, 3.44 mmol, obtained as described for Example H6) in dry CH₂Cl₂ were treated with 1-methyl imidazole (5.1 mmol, 3.0 eq.) and 2,6-dichlorobenzoyl chloride (5.7 mmol, 3.3 eq.). A mixture of 1-oleoyl-2-(9-nitropalmitoleoyl)-sn-3-glycerophosphocholine and 1-oleoyl-2-(10-nitropalmitoleoyl)-sn-3-glycerophosphocholine was obtained with 73% yield (1.01 g, 1.26 mmol) (purity control via HPLC, ¹H and ¹³C NMR spectroscopy).

### Example P2

### Synthesis of a mixture of 1-stearoyl-2-(10-nitrolinoleoyl)-sn-3-glycerophosphocholine and 1-stearoyl-2-(9-nitrolinoleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first sn-1 esterification was run using stearoyl chloride (2.36 g, 7.8 mmol, 2 eq.) to afford 1-stearoyl-2-lyso-sn-3-glycerophosphocholine in 40% yield (0.82 g, 1.56 mmol). According Example C the second acylation was carried out. 1-stearoyl-2-lyso-sn-3-glycerophosphocholine and a mixture of 10-nitrolinoleic acid 4 und 9-nitrolinoleic acid 3 (ratio 1:3, 1.13 g, 3.44 mmol, obtained as described for Example I1) were reacted to give a mixture of 1-stearoyl-2-(10-nitrolinoleoyl)-sn-3-glycerophosphocholine and 1-stearoyl-2-(9-nitrolinoleoyl)-sn-3-glycerophosphocholine with 64% yield (0.78 g, 1.0 mmol) (purity control via HPLC, ¹H and ¹³C NMR spectroscopy).

### Example P3

### Synthesis of a mixture of 1-erucinyl-2-(9-nitrogadoleoyl)-sn-3-glycerophosphocholine and 1-erucinyl-2-(10-nitrogadoleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using erucinoyl chloride (2.79 g, 7.8 mmol, 2 eq.) to afford 1-erucinoyl-2-lyso-sn-3-glycerophosphocholine in 36% yield (0.86 g, 1.48 mmol). According Example C the second acylation was carried out. 1-Erucinoyl-2-lyso-sn-3-glycerophosphocholine (0.86 g, 1.48 mmol) and a mixture of 9-nitrogadoleic acid und 10-nitrogadoleic acid (ratio 5:2, 1.58 g, 4.44 mmol, obtained as described for Example I2) were reacted to give a mixture of 1-erucinoyl-2-(9-nitrogadoleoyl)-sn-3-glycerophosphocholine and 1-erucinoyl-2-(10-nitrogadoleoyl)-sn-3-glycerophosphocholine with 60% yield (0.77 g, 0.89 mmol). (purity control via HPLC, ¹H and ¹³C NMR spectroscopy).

### Example P4

### Synthesis of a mixture of 1-eicosapentaenoyl-2-(5-nitro-EPA)-sn-3-glycerophosphocholine and 1-eicosapentaenoyl-2-(6-nitro-EPA)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using eicosapentaenoyl chloride (2.50 g, 7.8 mmol, 2 eq.) to afford 1-eicosapentaenoyl-2-lyso-sn-3-glycerophosphocholine in 33% yield (0.70 g, 1.29 mmol). According Example C the second acylation was carried out. 1-Eicosapentaenoyl-2-lyso-sn-3-glycerophosphocholine (0.70 g, 1.29 mmol) and a mixture of 5-nitro-EPA und 6-nitro-EPA (ratio 5:2, 1.34 g, 3.87 mmol, obtained as described for Example I3) were reacted to give a mixture of 1-eicosapentaenoyl-2-(9-nitroeicosapentaenoyl)-sn-3-glycerophosphocholine and 1-eicosapentaenoyl-2-(10-nitroeicosapentaenoyl)-sn-3-glycerophosphocholine with 52% yield (0.58 g, 0.67 mmol) (purity control via HPLC, ¹H and ¹³C NMR spectroscopy).

### Example P5

### Synthesis of a mixture of 1-(5,8,11-eicosatrienoyl)-2-(9-nitro-α-linolenoyl)-sn-3-glycerophosphocholine and 1-(5,8,11-eicosatrienoyl)-2-(10-nitro-α-linolenoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using 5,8,11-eicosatrienoyl chloride (2.54 g, 7.8 mmol, 2 eq.) to afford 1-(5,8,11-eicosatrienoyl)-2-lyso-sn-3-glycerophosphocholine in 50% yield (1.06 g, 1.95 mmol). According Example C the second acylation was carried out. 1-(5,8,11-eicosatrienoyl)-2-lyso-sn-3-glycerophosphocholine (1.06 g, 1.95 mmol) and a mixture of 9-nitro-α-linolenic acid and 10-nitro-α-linolenic acid (ratio 2:1, 1.89 g, 5.85 mmol, obtained as described for Example I4) were reacted to give a mixture of 1-(5,8,11-eicosatrienoyl)-2-(9-nitro-α-linolenoyl)-sn-3-glycerophosphocholine and 1-(5,8,11-eicosatrienoyl)-2-(10-nitro-α-linolenoyl)-sn-3-glycerophosphocholine with 59% yield (0.98 g, 1.15 mmol). (purity control via HPLC, ¹H and ¹³C NMR spectroscopy).

### Example P6

### Synthesis of a mixture of 1-linoleoyl-2-(dinitrolinoleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using linoleoyl chloride (2.33 g, 7.8 mmol, 2 eq.) to afford 1-linoleoyl-2-lyso-sn-3-glycerophosphocholine in 57% yield (1.15 g, 2.22 mmol). According Example C the second acylation was carried out. 1-linoleoyl-2-lyso-sn-3-glycerophosphocholine (1.15 g, 2.22 mmol) and a mixture of 9,12-dinitrolinoleic acid, 9,13-dinitrolinoleic acid, 10,12-dinitrolinoleic acid and 10,13-dinitrolinoleic acid (2.49 g, 6.66 mmol, obtained as described for Example K1) were reacted to give a mixture of 1-linoleoyl-2-(9,12-dinitrolinoleoyl)-sn-3-glycerophosphocholine, 1-linoleoyl-2-(9,13-dinitrolinoleoyl)-sn-3-glycerophosphocholine, 1-linoleoyl-2-(10,12-dinitrolinoleoyl)-sn-3-glycerophosphocholine and 1-linoleoyl-2-(10,13-dinitrolinoleoyl)-sn-3-glycerophosphocholine with 40% yield (0.77 g, 0.89 mmol). (purity control via HPLC, ¹H and ¹³C NMR spectroscopy).

### Example P7

### Synthesis of 1-palmitoyl-2-(9-nitrolinoleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using palmitoyl chloride (2.14 g, 7.8 mmol, 2 eq.) to afford 1-palmitoyl-2-lyso-sn-3-glycerophosphocholine in 40% yield (0.77 g, 1.56 mmol). According Example C the second acylation was carried out. 1-Palmitoyl-2-lyso-sn-3-glycerophosphocholine (0.77 g, 1.56 mmol) and E-9-nitrolinoleic acid (1.19 g, 3.12 mmol, obtained as described for Example I1) were reacted to give 1-palmitoyl-2-(9-nitrolinoleoyl)-sn-3-glycerophosphocholine with 66% yield (0.83 g, 1.03 mmol). (purity control via HPLC, ¹H and ¹³C NMR spectroscopy).

¹³C data of 1-palmitoyl-2-(9-nitrolinoleoyl)-3-glycerophosphatidyl choline: 173.5, 173.0 (0=0), 149.0 (C—NO₂), 133.0 (HC═), 131.5 (HC═), 125.0 (HC═), 70.5 (d), 65.0 (d), 63.5 (d), 63.0, 59.5 (d), 54.5 (NMe₃), 35.0 (═CH—CH₂—), 34.5-21.0 (25×CH₂), 14.5, 14.0 (2×CH₃).

### Example P8

### Synthesis of 1-palmitoyl-2-(E-9-nitrooleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using palmitoyl chloride (2.14 g, 7.8 mmol, 2 eq.) to afford 1-palmitoyl-2-lyso-sn-3-glycerophosphocholine in 40% yield (0.77 g, 1.56 mmol). According Example C the second acylation was carried out. 1-Palmitoyl-2-lyso-sn-3-glycerophosphocholine (0.77 g, 1.56 mmol) and E-9-nitrooleic acid 24a (1.02 g, 3.12 mmol, obtained as described for Example L1) were reacted to give 1-palmitoyl-2-(E-9-nitrooleoyl)-sn-3-glycerophosphocholine with 63% yield (0.79 g, 0.98 mmol) (purity control via HPLC, ¹H and ¹³C NMR spectroscopy).

### Example P9

### Synthesis of 1-liponoyl-2-(E-10-nitrooleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using liponoyl chloride (2.14 g, 7.8 mmol, 2 eq.) to afford 1-liponoyl-2-lyso-sn-3-glycerophosphocholine in 31% yield (0.54 g, 1.21 mmol). According Example C the second acylation was carried out. 1-liponoyl-2-lyso-sn-3-glycerophosphocholine (0.54 g, 1.21 mmol) and E-10-nitrooleic acid 30a (0.79 g, 2.24 mmol, obtained as described for Example L2) were reacted to give 1-liponoyl-2-(E-10-nitrooleoyl)-sn-3-glycerophosphocholine with 51% yield (0.44 g, 0.62 mmol). (purity control via HPLC, ¹H and ¹³C NMR spectroscopy).

### Example P10

### Synthesis of 1-behenoyl-2-(E-9-nitropalmitoleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using behenoyl chloride (2.8 g, 7.8 mmol, 2 eq.) to afford 1-behenoyl-2-lyso-sn-3-glycerophosphocholine in 30% yield (0.68 g, 1.17 mmol). According Example C the second acylation was carried out. 1-behenoyl-2-lyso-sn-3-glycerophosphocholine (0.68 g, 1.17 mmol) and E-9-nitropalmitoleic acid 24b (0.71 g, 2.34 mmol, obtained as described for Example L3) were reacted to give 1-behenoyl-2-(E-9-nitropalmitoleoyl)-sn-3-glycerophosphocholine with 42% yield (0.42 g, 0.49 mmol) (purity control via HPLC, ¹H and ¹³C NMR spectroscopy).

### Example P11

### Synthesis of 1-DHA-2-(E-10-nitropalmitoleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using docosahexaenoyl chloride (2.71 g, 7.8 mmol, 2 eq.) to afford 1-docosahexaenoyl-2-lyso-sn-3-glycerophosphocholine in 25% yield (0.55 g, 0.98 mmol). According Example C the second acylation was carried out. 1-docosahexaenoyl-2-lyso-sn-3-glycerophosphocholine (0.55 g, 0.98 mmol) and E-10-nitropalmitoleic acid 30b (0.67 g, 2.2 mmol, obtained as described for Example L4) were reacted to give 1-docosahexaenoyl-2-(E-10-nitropalmitoleoyl)-sn-3-glycerophosphocholine with 31% yield (0.26 g, 0.3 mmol) (purity control via HPLC, ¹H and ¹³C NMR spectroscopy).

### Example P12

### Synthesis of 1-linoleoyl-2-(E-15-nitro-α-linolenoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using linoleoyl chloride (2.33 g, 7.8 mmol, 2 eq.) to afford 1-linoleoyl-2-lyso-sn-3-glycerophosphocholine in 60% yield (1.21 g, 2.34 mmol). According Example C the second acylation was carried out. 1-linoleoyl-2-lyso-sn-3-glycerophosphocholine (1.21 g, 2.34 mmol) and E-15-nitro-α-linolenic acid 41 (0.77 g, 2.4 mmol, obtained as described for Example M1) were reacted to give 1-linoleoyl-2-(E-15-nitro-α-linolenoyl)-sn-3-glycerophosphocholine with 55% yield (0.55 g, 0.67 mmol) (purity control via HPLC, ¹H and ¹³C NMR spectroscopy).

### Example P13

### Synthesis of 1-stearoyl-2-(E-9-nitrolinoleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using stearoyl chloride (2.36 g, 7.8 mmol, 2 eq.) to afford 1-stearoyl-2-lyso-sn-3-glycerophosphocholine in 40% yield (0.82 g, 1.56 mmol). According Example C the second acylation was carried out. 1-stearoyl-2-lyso-sn-3-glycerophosphocholine (0.82 g, 1.56 mmol) and E-9-nitrolinoleic acid (0.89 g, 2.34 mmol, obtained as described for Example I1) were reacted to give 1-stearoyl-2-(E-9-nitrolinoleoyl)-sn-3-glycerophosphocholine with 61% yield (0.79 g, 0.95 mmol) (purity control via HPLC, ¹H and ¹³C NMR spectroscopy).

### Example P14

### Synthesis of 1-stearoyl-2-(E-9-nitrooleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using stearoyl chloride (2.36 g, 7.8 mmol, 2 eq.) to afford 1-stearoyl-2-lyso-sn-3-glycerophosphocholine in 40% yield (0.82 g, 1.56 mmol). According Example C the second acylation was carried out. 1-stearoyl-2-lyso-sn-3-glycerophosphocholine (0.82 g, 1.56 mmol) and E-9-nitrooleic acid (0.77 g, 2.34 mmol, obtained as described for Example L1) were reacted to give 1-stearoyl-2-(E-9-nitrooleoyl)-sn-3-glycerophosphocholine with 64% yield (0.83 g, 1.0 mmol) (purity control via HPLC, ¹H and ¹³C NMR spectroscopy).

### Example P15

### Synthesis of 1-oleoyl-2-(E-9-nitrooleoyl)-sn-3-glycerophosphocholine

Following the preparation procedure described in Example P1, sn-glycero-3-phosphatidylcholine 1a (1.0 g, 3.9 mmol) was activated with dibutyltin oxide. Then, the first esterification was run using oleoyl chloride (2.35 g, 7.8 mmol, 2 eq.) to afford 1-oleoyl-2-lyso-sn-3-glycerophosphocholine in 44% yield (0.89 g, 1.72 mmol). According Example C the second acylation was carried out. 1-oleoyl-2-lyso-sn-3-glycerophosphocholine (0.89 g, 1.72 mmol) and E-9-nitrooleic acid (1.13 g, 3.44 mmol, obtained as described for Example L1) were reacted to give 1-oleoyl-2-(E-9-nitrooleoyl)-sn-3-glycerophosphocholine with 69% yield (0.99 g, 1.19 mmol) (purity control via HPLC, ¹H and ¹³C NMR spectroscopy).

¹³C data of 1-oleoyl-2-(E-9-nitrooleoyl)-sn-3-glycerophosphocholine: 173.5, 173.0 (C═O), 150.0 (C—NO₂), 134.0 (HC═), 130.0 (HC═), 129.5 (HC═), 71.0 (d), 66.0 (d), 64.0 (d), 63.0, 60.0 (d), 54.5 (NMe₃), 34.5-22.0 (28×CH₂), 14.5, 14.0 (2×CH₃).

### Example Q

### 1-(9-Nitrooleoyl)-2-(palmitoyl)-sn-3-glycero-phosphocholine

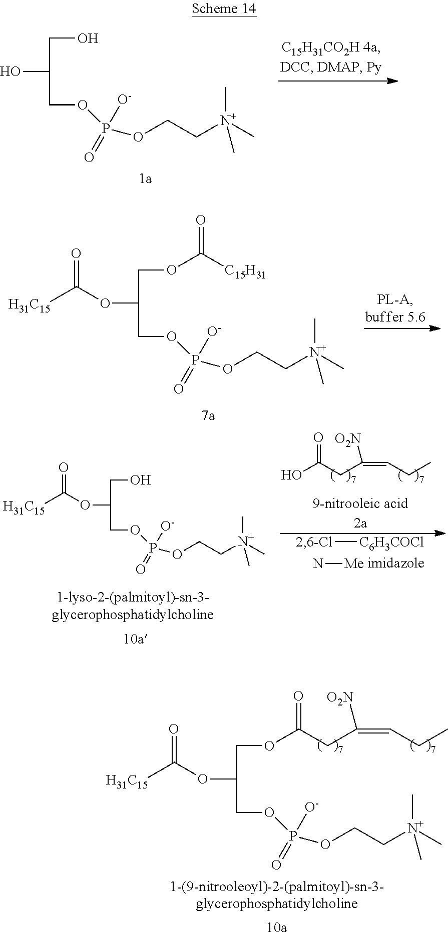

### Synthesis of 1-lyso-2-palmitoyl-sn-3-glycerophosphatidylcholine 10a′ (in analogy to J. Sakakibara, Tetrahedron Lett. 1993, 34, 2487)

A solution of 1,2-dipalmitoyl-sn-3-glycerophosphatidylcholine 7a (500 mg, 0.571 mmol), Mucor javanicus lipase (300 mg) and Triton X-100 (500 mg) in a boric acid/borax buffer (0.05 M, 90 ml) were stirred at 37° C. for 2 h. The reaction was stopped by adding 5% acetic acid and ethanol. After removal of the solvents in vacuum the residue was purified by column chromatography (silica gel, CH₂Cl₂/MeOH, gradient) or preparative HPLC (Phenomenex Gemini NX 5μ C18 110 Å, gradient MeOH/H₂O). Caution: intramolecular transesterification has to be excluded! Yield: 307.8 mg (0.543 mmol, 95%) of 10a′ (purity control via HPLC, ¹H and ¹³C NMR spectroscopy).

### Synthesis of 1-(9-nitrooleoyl)-2-(palmitoyl)-sn-3-glycerophosphatidylcholine (β-(palmitoyl)-γ-(9-nitrooleoyl)-L-α-glycerophosphocholine) 10a (in analogy to R. Salomon (Biorg. & Med. Chem. 2011, 19, 580):

Reaction and scale as described for the synthesis of 6a using 1-lyso-2-palmitoyl-sn-3-glycero-phosphatidylcholine 10a′ (300 mg, 0.645 mmol) and E-9-nitrooleic acid (211 mg, 0.645 mmol). Purification via column chromatography (silica gel, CH₂Cl₂/MeOH, gradient) or preparative HPLC (Phenomenex Gemini NX 5μ C18 110 Å, gradient MeOH/H₂O). Yield: 394.4 mg (0.51 mmol, 79%) of 10a (purity control via HPLC, ¹H and ¹³C NMR spectroscopy).

### Example R

### 1-Stearoyl-2-(E-9-nitrolinoleoyl)-sn-3-glycerophosphatidylethanolamine

sn-3-Glycerophosphatidyl-N-(boc)-ethanolamine 1c has been synthesized as described in Example D. Following the preparation procedure described in Example P1, sn-glycero-3-phosphatidyl-N-(boc)-ethanolamine 1c (1.04 g, 3.3 mmol) was activated with dibutyltin oxide. Then, the first esterification was run using stearoyl chloride (2.0 g, 6.6 mmol, 2 eq.) to afford 1-stearoyl-2-lyso-sn-3-glycerophosphatidyl-N-(boc)-ethanolamine in 48% yield (0.92 g, 1.58 mmol). According Example C the second acylation was carried out. 1-stearoyl-2-lyso-sn-3-glycerophosphatidyl-N-(boc)-ethanolamine (0.92 g, 1.58 mmol) and E-9-nitrolinoleic acid (0.89 g, 2.34 mmol, obtained as described for Example I1) were reacted to give 1-stearoyl-2-(E-9-nitrolinoleoyl)-sn-3-glycerophosphatidyl-N-(boc)-ethanolamine with 55% yield (0.78 g, 0.87 mmol). Finally, protecting group removal succeeded applying the procedure as described for Example D. 1-stearoyl-2-(E-9-nitrolinoleoyl)-sn-3-glycerophosphatidylethanolamine was isolated with 93% yield (0.64 g, 0.81 mmol) (purity control via HPLC, ¹H and ¹³C NMR spectroscopy).

### Example S

### 1-Palmitoyl-2-(E-9-nitrooleoyl)-sn-3-glycero-phosphatidylethanolamine

sn-3-Glycerophosphatidyl-N-(boc)-ethanolamine 1c has been synthesized as described in Example D. Following the preparation procedure described in Example P1, sn-glycero-3-phosphatidyl-N-(boc)-ethanolamine 1c (1.04 g, 3.3 mmol) was activated with dibutyltin oxide. Then, the first esterification was run using palmitoyl chloride (1.81 g, 6.6 mmol, 2 eq.) to afford 1-palmitoyl-2-lyso-sn-3-glycerophosphatidyl-N-(boc)-ethanolamine in 50% yield (0.91 g, 1.65 mmol). According Example C the second acylation was carried out. 1-Palmitoyl-2-lyso-sn-3-glycerophosphatidyl-N-(boc)-ethanolamine (0.91 g, 1.65 mmol) and E-9-nitrooleic acid (0.98 g, 3.0 mmol, obtained as described for Example L1) were reacted to give 1-palmitoyl-2-(E-9-nitrooleoyl)-sn-3-glycerophosphatidyl-N-(boc)-ethanolamine with 54% yield (0.77 g, 0.89 mmol). Finally, protecting group removal succeeded applying the procedure as described for Example D. 1-palmitoyl-2-(E-9-nitrooleoyl)-sn-3-glycerophosphatidylethanolamine was isolated with 90% yield (0.62 g, 0.80 mmol) (purity control via HPLC, ¹H and ¹³C NMR spectroscopy).

### Experiment Examples

### Example 1

### Investigation of Feasibility of Dip Coating of Nitrated Phospholipids to Improve Surface Properties

A commercially available stent made of medical stainless steel 316 LVM was degreased (15 min) in an ultrasonic bath with acetone and ethanol and dried at 100° C. in a compartment dryer. Then, the stent was gently dipped in a 1% solution of phosphatidylcholine esterified with 9-nitro-cis-oleic acid (50%) and oleic acid (50%) in a mixture of ethanol/diethyl ether (50/50 (v/v)) for 7 minutes and then dried for 10 min at 100° C. The diving operation and the subsequent drying were repeated two more times. Finally, the stent was washed in ethanol (70%) over night and dried for 15 min at 100° C.

An amorphous uniform coating of the whole surface of the stent was achieved.

Conclusion: Nitro-carboxylic acid (s)-containing phospholipids allow fast and complete coverage of surfaces. The mixture of nitrated and native phospholipids most notably improves the coating quality of coverage by yielding a higher completeness and a reduction of multilayer formation as well as an improved adhesion of the coating.

### Example 2

### Investigation of the Feasibility of Physiosorption of Nitrated Phospholipids on a Surface that has been Made Hydrophobic in Order to Improve Resulting Surface Properties

(1) A stent was washed and degreased using a solution of anhydrous methanol, then of methanol/chloroform (1:1, vol:vol) and then was dipped in a Teflon beaker with anhydrous chloroform for 5 minutes in an ultrasonic bath. The stent was then kept in dry chloroform.

(2) A mixture of decalin/carbon tetrachloride/and chloroform (7:2:1, vol.: vol.: vol.) was prepared in a Teflon beaker and the stent was removed from the anhydrous chloroform and immersed in this mixture. Then, 1% (vol.) OTS (trichloroctadecylsilane) was mixed herein and the stent was placed in this mixture for 12 h.

It is also possible to use one of the following silanes instead of trichloroctadecylsilane such as n-octyltriethoxysilane, n-butyltrimethoxysilane, n-decyltriethoxy silane, hexadecyltrimethoxysilane, isooctyltrimethoxysilane, 13-(trichlorosilylmethyl)-heptacosane, n-phenylaminomethyltrimethoxysilane, n-cyclohexylaminomethyltri-ethoxysilane, isooctyltriethoxysi lane, hexadecyltrimethoxysilane, phenyltriethoxysilane, or dicyclopentyldimethoxysilane.

Then the stent was removed, immersed in a Teflon beaker containing chloroform, then in methanol/chloroform (1:1, vol.: vol.) and dipped finally in methanol and treated with ultrasound for 5 minutes.

The stent is coated with a silane is highly water repellent. The suchlike coated stent was then dipped in a solution of phosphatidylcholine (0.007 mmol per ml), wherein the two fatty acid residues consisted of nitro oleic acid, solved in 5 ml of chloroform, for 15 minutes. It was then removed and dried in a stream of nitrogen under rotation of the stent. The coating process was repeated another 2 times. The coating result is studied by confocal laser microscopy using fluoresceinisothiocyanate as a fluorescent for the amine group of the choline residue.

Result: A complete and uniform coating without visible gaps was obtained.

### Example 3

### Study of Radiolabelled Nitro-Phosphatidylcholine to Evaluate their Partition from SAM Coatings and Release of Nitrogen Monoxide

In order to determine the amount of phospholipids that diffuse from the coating into a surrounding aqueous medium (bovine serum), as well as to determine the amount of phospholipids that are absorbed by the cells from the aqueous medium or by direct contact with the surface, slides that have been coated with phospholipids as described in example 1, however containing a radioactively labelled fatty acid, were prepared. For this purpose, phosphatidylcholines were synthesized, the H³-labeled palmitic acid at the SN-1 position and nitrated or native oleic acid at the SN-2 position. 10% of the radiolabel phospholipids were added to otherwise identical synthetic phospholipids. From this mixture, slides were coated. The slides were repeatedly washed with alcoholic solutions and finally transferred in a bath of a 0.9% NaCl solution for one hour. Thereafter the slides were placed in dishes with 20% FCS, in which they were kept for 1, 3 and 7 days, respectively, during a continuous slight movement of the dishes.

In another set of experiments the slides were placed in Petri dishes and 2500×10⁵/ml fibroblasts were suspended in the culture dish, where they were allowed to grow for 3 or 7 days. After completion of the cell culture studies, the cells were carefully washed twice and displaced by trypsin. Then the cells were homogenized and prepared for scintillation measurement. Measurements of cell lysates and representative serum samples were performed with liquid scintillation counter (LSC-5000, Aloka, Japan) after adding the scintillation fluid (ACSII, Amersham, UK) to the scintillation vessels.

Results: Radioactively marked phospholipids were detected in serum samples of nitrated and non-nitrated phospholipid coatings. However, the content of radiolabelled molecules tended to be lower in samples form coatings with nitrated phospholipids compared to samples of phospholipid coatings with native fatty acids. The amount of nitrated phospholipids, which were released from the coatings after 24 hours were determined to be less than 0.5%, which rose to 0.7% on day 3 and 0.8% on day 7, respectively. In phospholipid coatings containing native fatty acids the amount of released phospholipids was 0.8%, 1.0% and 1.2%, respectively. Measurements of cell lysates showed that the content of nitrated phospholipids that had been taken up by the cells amounted to 0.1% of the calculated total content of phospholipids that was used for the coating. On day 3, the content determined was 0.2% and on day 7 of 0.26%. In lysates of cells that were grown on non-nitrated phospholipids the content of the phospholipids that had been taken up was 0.4%, 0.6% and 0.7%, respectively.

Conclusion: Physiosorbed phospholipids are released to a small part in a serum-containing environment with decaying release kinetics. The release of the nitrated phospholipid-form coatings thereof tends to be less than that in non-nitrated phospholipid coatings, probably due to the higher intermolecular adherence. Cells growing on phospholipid coatings take up released phospholipids; however, the amount of phospholipids that were taken up was negligible.

### Example 4

### Quantification of Nitric Oxide of the Release and its Possible Influence on the Bio-Passivation Effects

The concentration of nitric oxide in the culture medium and in adherent cells was measured to detect whether nitric oxide derived from nitrated phospholipids is released. 1,2-diaminoanthraquinone (Invitrogen) was used for the determination of accumulated nitric oxide in the culture medium and DAF-FM (Invitrogen) was used as nitric oxide indicator for the amount of produced nitric oxide within cells. Fibroblasts were transferred to a 1% DMSO solution to achieve a cell density of 2500×10⁵/ml. Cells were incubated for 30 min with DAF-FM, which was added to reach a concentration of 5 μmol. Then the cells were washed and transported in a culture dish, which was previously coated as in example 2, with native or nitrated phosphatidylcholine, a Petri dishes without a coating served as control. Cell cultures were allowed to grow for one or 3 days in 5% FCS under standard conditions. Then the cells were displaced by trypsin and suspended in a 2% DMSO solution to measure the accumulated amount of nitrogen monoxide and cumulative NO production, respectively. The content on intracellular nitric oxide, as well as that in the culture medium was determined by means of a confocal laser-scanning microscope (FluoView 300, Olympus Europe) and a photomultiplier-based micro fluorimetry (Seefelder Messtechnik, Germany), respectively.

Results: The measured nitrogen oxide accumulation and production, respectively, were significantly higher in both intracellular and in the culture medium in cell cultures grown on an uncoated media, than in cultures grown on coated glass slides. Compared to cultures grown on nitro-carboxylic acid-containing phospholipids nitrogen oxide accumulation and production tended to be higher in cultures grown on phospholipid coatings with native fatty acids.

Interpretation: The higher content of nitric oxide in cultures grown on uncoated synthetic surfaces as compared to cultures grown on biocompatible surfaces can be explained by the response to the contact to the foreign surface and proliferation induction of fibroblasts which results in endogenous nitric oxide production. Since NO accumulation in cultures that were grown on nitro-carboxylic acid containing phospholipids was comparable with those that were grown on phospholipids without nitration, the release of a clinically relevant amount of NO from the nitro-carboxylic acid-containing phospholipids can be excluded.

### Example 5

### Study on Adhesion of Proteins and Organic Molecules on Surfaces with Coatings of Nitrated and Native Phospholipids

To determine the adsorption of organic bio-molecules on surfaces coated with SAM from native and nitro-carboxylic acid-containing phospholipids, substrates were prepared as described in example 1. For this purpose templates were coated with a nitro-carboxylic acid (s)-containing phospholipids according to example N1 which are mixtures of 1,2-di-(9-nitrolinoleoyl)-sn-3-glycero-phosphocholine, 1,2-di-(10-nitrolinoleoyl)-sn-3-glycero-phosphocholine, 1-(9-nitrolinoleoyl)-2-(10-nitrolinoleoyl)-sn-3-glycero-phosphocholine and according to example D, namely 1-(10-nitrolinoleoyl)-2-(9-nitrolinoleoyl)-sn-3-glycero-phosphocholine, and example E, namely (1,2-di-(9-nitrooleoyl)-sn-3-glycero-phosphatidylserin), and example F, namely (1,2-di-(9-nitro-oleoyl)-sn-3-glycero-phosphatidylinositol), and example G, namely (1,2-di-(9-nitrooleoyl)-sn-3-glycero-phosphate), and example 01, namely (1,2-di-(9-nitro-10-hydroxy-stearoyl)-sn-3-glycero-phosphocholine), respectively. Coated and uncoated substrates were placed in Petri dishes. Solutions of 2% bovine albumin or bovine serum with or without the addition of fibronectin and laminin, and a 0.9% saline which served as control, were given in the Petri dishes. These were gently shaken over a period of 24 or 72 hours. At the end of the exposure time substrates were carefully washed twice with 0.9% NaCl solution. The surfaces were investigated with an antibody staining to demonstrate protein absorption.

Results: The surfaces of the native substrates exhibited a homogeneous layer of albumin with the exception of substrates, which were incubated in NaCl solution only. Addition of fibronectin and laminin resulted in denser layers of protein. Complement factors were present on the surface of control substrates, as demonstrated by selective staining. Substrates that were coated with native phosphatidylcholine showed negligible amounts of albumin, laminin, fibronectin or complement. Substrates, which were coated with a combination of 80% native phosphatidylcholine and 20% phosphatidylserine, showed adhesion of albumin that was comparable with that found in uncoated substrates and compared to those adsorption of fibronectin and laminin was increased. The content of complement, which adhered to those substrates, was higher than in native substrates. All substrates that were coated with. nitro-carboxylic acid-containing phosphohatidylcholins showed a significantly lower adsorption of albumin as found on native substrates. However, the levels of albumin, fibronectin, and laminin were slightly higher than on substrates with native phosphatidylcholines, while the complement content was the same. Coatings with a combination of nitro-carboxylic acid-containing phosphatidylcholine (80%) and phosphatidylserine (20%) showed a significantly lower absorption of albumin, laminin, fibronectin, and complement than for coatings with a comparable combination of native phospholipids. The content was also lower than that found on uncoated substrates. The results were stable for both observation periods.

Conclusion: Coating of artificial surfaces with native phosphatidylcholine almost completely inhibits the absorption of proteins and biomolecules. Nitration of a fatty acid residue of phosphatidylcholine slightly reduced this adherence effect for albumin, fibronectin and laminin as compared to an uncoated substrate; however the anti-adhesive effect for complement remains. For combinations of phospholipids which enhance adsorption of serum proteins, addition of nitrated phospholipids results in a significant anti-adhesive effect.

### Example 6

### Studies on the Effect of Nitro-Carboxylic Acid-Containing Phospholipids on Adhesion, Propagation, and Growth of Endothelial Cells

To investigate the cell homing of endothelial cells onto phospholipid-coated artificial surfaces, metal grids with a cobalt-chromium alloy such as has been described in example 2 were coated. Coatings were performed with nitro-carboxylic acid-containing phosphatidylcholine according to the examples N2-N6 and O6. These were special product mixtures of different esterified 1,2-di(nitro linoleoyl)-sn-glycero-3-phosphatidylcholines (N2), from different esterified 1,2-di(nitro arachidonoyl)-sn-glycero-3-phosphatidylcholines (N3), from different esterified 1,2-di(nitroarachidonoyl)-sn-glycero-3-phosphatidylcholines (N4), different esterified 1,2-di(nitro-γ-linolenoyl)-sn-glycero-3-phosphatidylcholines (N5) or different esterified 1,2-di(nitro-DHA)-sn-glycero-3-phosphatidylcholines (N6) and mixture of 1-palmitoyl-2-(10-hydroxy-9-nitro-α-linolenoyl)-sn-3-glycero-phosphocholine and 1-palmitoyl-2-(9-hydroxy-10-nitro-α-linolenoyl)-sn-3-glycero-phosphocholine (06). Uncoated metal grids served as controls. The grids were placed in a culture dish containing a gel matrix on which human umbilical venous endothelial cells (HUVEC) were grown to confluence. The culture media consisted of 5% FCS, and was changed every second day. Cultivation was performed according to standard procedures. The culture dishes were carefully washed superficially several times after 3, 7, and 14 days with saline. Thereafter the surface was stained with methylene blue. Using an incident light microscope, the samples were examined immediately by evaluating the following parameters: propagation of cells from the edge of the grid to the grid centre, cell density, multi-layer formation, and cell shape.

Results: Propagation of cells was the fastest on uncoated grids, which resulted in a complete coverage at day 3. Almost no cell attachment was observed on phosphatidylcholine-coated grids, whereas the spaces between the stent struts were fully covered by cells after 3 days. Islands of adherent cells were formed on the surfaces covered with nitro-carboxylic acid-containing phosphatidylcholine. This finding was consistent in all coatings of nitro-carboxylic acid (s)-containing phospholipids with a choline head group. Multilayer formation between the stent struts in cultures with non-coated metal grids was observed at day 7 which had progressed further at day 14, and then a multilayer formation was also observed on the struts. However, the cell coverage on phosphatidylcholine-coated struts remained incomplete up to day 14 with a few areas of multi-layer formation located between the struts. In contrast, nitro-carboxylic acid-containing phosphatidylcholine-covered struts were partial and finally completely covered in endothelial cells at day 7 and day 14, respectively. Mixtures according to the examples of N2 and N6 showed a slight tendency for a stronger growth of cells here; however this was not statistically significant. Furthermore, no multi-layer formation was observed on the struts, or in the interspaces.

Conclusion: An uncoated cobalt-chromium alloy of metal grids allows a fast cell homing of endothelial cells. However, it comes to a gradual proliferation of these cells on and inbetween the stent struts. Phosphatidylcholine coatings delay cell homing, but the coating seems to have no effect on multi-layer formation by endothelial cells between the struts. The culture results with metal struts, which were coated with nitro-carboxylic acid-containing phosphatidylcholines, documented a faster homing of endothelial cells on the suchlike-coated surfaces when compared to a coating with native phosphatidylcholine, while a multi layer formation by endothelial cells is essentially absent.

### Example 7

### Investigation on the Effect of Nitro-Carboxylic Acid Containing Phospholipids on Immunological Cell Effects

The survival rate and cytokine production of adhering macrophages were examined to evaluate the bio-passivating properties of various nitro-carboxylic acid containing phospholipids.

Templates made of glass were coated with nitro-carboxylic acid-containing and native phosphatidylcholines and with an admixture of 50% phosphatidylcholine and phosphatidyletholamine, as described in example 2. Further coatings were done with mixtures of native phosphatidylcholine- and nitro-carboxylic acid (s)-containing phospholipids according to examples N12-N14, E, Q, O₂, 06 and P3-P7. The glass slides were placed in Teflon bowl, uncoated slides served as control. Murine macrophages (RAW 264.7) were cultured to a cell density of 5×10⁵. Cell suspensions were added to the culture dishes, so that macrophages could attach on the slides under standard culture conditions for 24 to 48 hours. Samples of cell culture supernatants were taken at the beginning and at the end of the experiments and analyzed with assays for IL6, IL8 and macrophage chemo-attractant protein 1 (MCP-1). The cell survival rate was investigated using an MTT assay.

Results: A significant increase of cytokines was observed in uncoated glass slides during the observation period. In contrast, almost no change was found in cultures with slides that were coated with native phosphatidylcholine, after 24 hours; however, after 48 hours a moderate increase was observed. Cultures grown on slides that were coated with a mixture of natural phospholipids, exhibited a progressive increase in cytokine concentrations; the concentrations of which were approximately the same as in experiments with native glass surfaces. In supernatants from cultures of slides that were coated with nitro-carboxylic acid-containing phosphatidylcholines, IL-8 was minimally increased after 24 h, while the other cytokines stayed at a low level. After 48 h, a small increase was observed for all cytokines. However, the concentrations of which were significantly lower than in experiments with native phosphatidycholine coatings. In experiments where the glass substrates were coated with a mixture of nitro-carboxylic acid-containing phospholipids, there was an insignificant increase of all cytokines, which however was less than the increase found for coatings with an analogue mixture of native phospholipids. The used product mixtures according to the examples N12-N14, E, O2, O6, Q and P3-P7 exhibited only minimal differences.

Cells that adhered on uncoated slides remained vital to a high percentage (95% after 24 h and 90% after 48 h, respectively). On slides coated with native phosphatidylcholines, viability of cells was about the same as on uncoated glass slides after 24 h, but significantly lower (75%) after 48 h. In experiments with a mixed phospholipid coating a rapid loss of viability (50% after 24 h and 70% after 48 h) was observed. In experiments, with slides coated with nitrated phospholipids, the viability was significantly higher than for coatings with native phospholipids, namely 95% for nitro-carboxylic acids-containing phosphatidylcholine after 48 h, and 90% for nitro-carboxylic acids-containing phospholipid mixtures after 48 h. Viability values for the nitro-carboxylic acid-containing phosphatidylcholine mixtures prepared according to the examples N12-N14 and E ranged between 85% and 95% after 48 h, while there was no other trend was seen for an individual product mixtures. PL coating with a choline head group according to examples O2 and P4-P7 yield viability levels ranging between 90% and 95% after 48 h and, and for those according to examples Q, 06 and P3 the values ranged between 95% and 98% after 48 h, respectively.

Conclusion: Macrophages getting in contact with uncoated glass surfaces become activated. This activation is reduced to a minimum by a surface coating with phospholipids. However, the reduced adhesion of macrophages conditioned increased apoptosis, which causes cytokine production in the further course. Addition of phosphatidyletholamine to a PL coating results in a differentiated cytokine release, probably due to a chance in the surface charge. This effect is reduced by nitro-carboxylic acid-containing phospholipids. The viability of macrophages that are cultured on nitro-carboxylic acid-containing phospholipid coatings is higher than that of macrophages that are cultivated on similar coatings without phospholipids-containing nitro-carboxylic acids.

### Example 8

### Studies on Effects of Nitro Carboxylic Acid-Containing Phospholipids on Cell Physiology

Physiologically occurring phospholipids can be taken up by virtually all cell lines in large quantities. It is known that phospholipids that contain non-physiologically occurring fatty acid residues can cause cell lyses or death. Three cell lines (HeLa, HUVEC and L929 fibroblast) were cultured in a suitable culture medium of 5% FCS at 37° C. and 5% CO₂ concentrations to a subconfluent concentration of 1.5×10⁵ cells.

SOPC, DOPC, POPC, ONOPC, PNLPC, and the free fatty acids of OA, LA, NOOA, and NOLA were dissolved in 0.5% DMSO. Experiments were performed also with nitro-carboxylic acid-containing phospholipids according to examples N15-N20, G, O3, O5, P2, P5, P8, P9, P11 and Q. PL prepared as aqueous suspensions were added the culture flasks in order to achieve PL concentrations in the culture medium from 10 μmol to 1 mmol. Cell cultures were incubated for 24 h and 48 h. Thereafter, the culture medium was removed and the cells washed twice. Following this, cells suspensions were given in four vials to perform the following analysis:

1 Lipid staining using Nile red staining;
2. Cell viability with the MTT test;

### 3. Volumetry;

4. Stability test of the cells.

Nile red staining was performed by adding a Nile red solution (1 μmol in PBS) to the cell suspension. After 15 minutes the solution was decanted and the cell suspensions were washed twice with PBS. For quantification of fat accumulation, fluorescence microscopy was performed after 1 h, 12 h and 24 h.

For the determination of viability, phenol red was added to the suspended cells. After 4 h, the medium was renewed and 10 μl of the MTT solution was added. The cells were cultured for 4 hours, and then a 10% SDS solution was pipetted. After 24 h, absorption of formazan crystals was measured at 500 nm using a power wave X (bio-tek instruments, Inc., USA). For the quantitative comparison of toxicity, the EC50 was determined.

Volumetric measurement of cells was done in a Histopenz (Sigma) solution with an effective NaCl concentration of 0.9% (approximately 290 mOsm) heated to 37° C. in which the cells were suspended; thereafter the solution was sonified for 2 minutes. Measurements were done using a Coulter counter Z2.

Studies of cell viability were carried out with the live/dead assay (Molecular Probes). For this purpose, the cells were washed twice with PBS solution and then seeded into a culture medium. Incubation with the staining solution (in 0.1% DMSO) was carried out over 30 minutes in the dark. The viability analyses were performed using fluorescence microscopy.

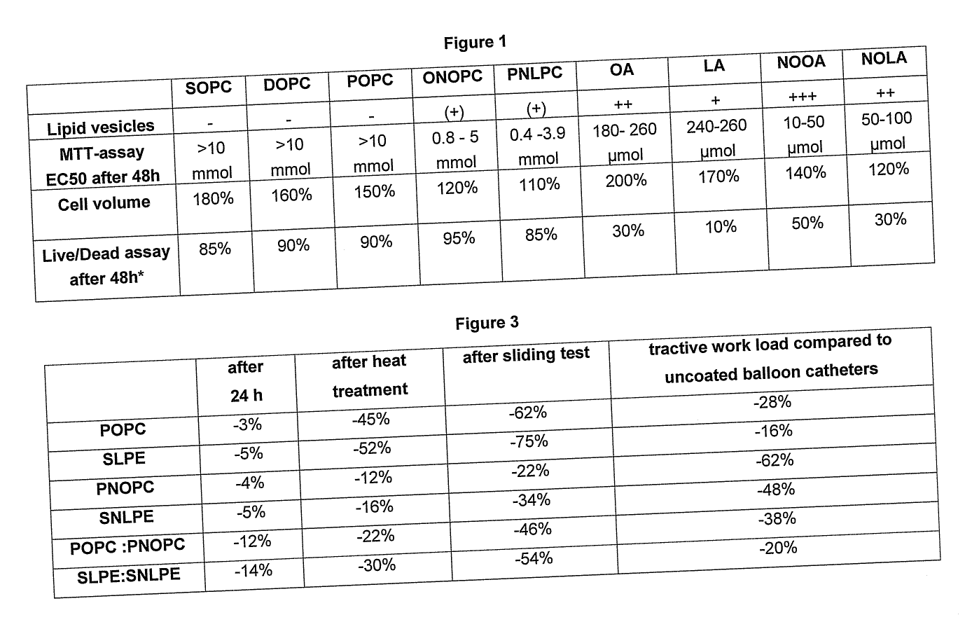

Results (these are Grouped Together in the Table of FIG. 1):

Incubation with free fatty acids led to a time-dependent, cellular up-take of fatty acids, which could be detected by the occurrence of fatty vesicles within the cytoplasm. The volumes of the fatty vesicles correlated with the concentration of the fatty acid of in the incubation solutions. The up-take was faster and higher for the two nitro fatty acids than that of the native fatty acids. After incubation with phospholipids fatty vesicles were visible in the cytoplasm only after incubation with nitrated phospholipids after 24 hours, the volumes of which correlated with the concentration of the nitrated PL.

Viability as quantified by the MTT assay was only slightly decreased after incubation with SOPC DOPC, POPC at the tested concentrations, so that the EC50 could not be calculated from the concentrations used. After incubation with ONOPC and PNLPC, a moderate cytotoxicity was evident after 24 hours at the highest concentrations (total viability 83% and 75%, respectively). After 48 hours, an EC50 could be determined that was for ONOPC at a concentration between 0.8-5 mmol and for PNLPC between 0.4-3.9 mmol. The free fatty acids showed a significantly greater effect on the viability according to the MTT assay, for the different cell lines the EC50 concentration were between 10-50 μmol for nitro oleic acid, between 50-100 μmol for nitro-linoleic acid, between 180-260 μmol for oleic acid and between 240-260 μmol for linoleic acid after 48 hours, respectively.

Determination of the cell volumes revealed that cells which had been incubated with the natural phospholipids DOPC, POCP and SOPC showed a time-dependent increase in size to 180%, 160% and 150%, respectively, which was also the case after incubation with the oleic acid (200%), linoleic acid (170%), nitro-oleic acid, (140%) and nitro-linoleic acid (120%). Cells that have been incubated with the nitrated phospholipids showed a non-significant increase of their cell volumes up to 110-120%.

The results of the live/dead staining only partially agreed with the results of the MTT viability test. In accordance with the MTT assay, cells that were incubated with the natural phospholipids SOPC DOPC, POPC showed a high rate of viability. At a concentration of native or nitrated fatty acids, which resulted in a 50% loss of viability in the MTT assay, viability of cells which have been incubated with native fatty acids was significantly lower, and in cells incubated with nitrated fatty acids moderately lower according to the live/dead assay. A complete mismatch in assessing the viability of cells was found in cells that have been incubated with nitro-fatty acid-containing phospholipids; here the cells exhibited almost the same viability as after incubation with native phospholipids.

The experiments with nitro-carboxylic acid (s)-containing phospholipids according to examples N15-N20, G, O3, O5, P2, P5, P8, P9, P11 and Q showed no significant differences in the comparison to the incubation with ONOPC and PNLPC. Uniformly, a small amount of lipid vesicles within the cytoplasm was observed, as well as a low cytotoxicity according to the MTT assay (at concentrations between 0.6 and 4.5 mmol) and viability according to the live/dead assay was virtually unchanged (85-95%). Cell volume was also almost unchanged and showed a slight tendency to enlarge, as it was already noted after incubation with ONOPC and PNLPC. Thus, all phospholipid-containing nitro-carboxylic acid residues showed similar effects in the assays used with no significant differences between the various nitro-carboxylic acid (s)-containing phospholipids tested.

Interpretation: Phospholipids containing a nitrated fatty acid were taken up by cells to a lower extent than naturally occurring phospholipids as well as native or nitrated free fatty acids. Cellular uptake of nitro-fatty acid-containing phospholipids causes a reduction in the activity of cellular metabolism. Nitrated free fatty acids also lead to a reduction of metabolic cell activity at concentrations which intersects with their toxic effects. Although both the nitrated free fatty acid- and the nitro-carboxylic acid (s)-containing phospholipids reduce metabolic activity of cells after they have been taken up, cells incubated with nitrated phospholipids still remained vital.

Results indicate that nitro-carboxylic acid (s)-containing phospholipids exhibit a significantly wider concentration range in which they are non-toxic, unlike the case with free fatty acids irrespective of whether they are native or nitrated. Despite only a seemingly small amount of nitrated phospholipids having been taken up, a considerable reduction of cell metabolism was reached (as opposed to native phospholipids), suggesting anti-proliferative effects.

### Example 9

### Studies on Effects of Nitro-Carboxylic Acid-Containing Phospholipids on the Adhesion, Migration, and Proliferation of Cells

Phospholipids can be readily absorbed by cells in their outer membrane leaflet which changes properties of the cell membrane. Therefore, it should be investigated whether phospholipids that contain at least a nitro-carboxylic acid, lead to biological effects on cells, which absorb them.

POPC, DOPC, POPE, ONOPC, PNLPC, PNOPE (1-palmitoyl-2-(E-9-nitrooleoyl)-sn-3-glycero-phosphatidylethanolamine), as well as the free fatty acids oleic acid, E-9 nitro oleic acid and E-9 nitro linoleic acid were dissolved in 0.5% DMSO.

Furthermore, nitro-carboxylic acid (s)-containing phospholipids according to examples N7-N9, F, O2-O4, P1, P2, P5, P6, P8-P10, P15 were prepared and investigated.

The investigations were carried out with human umbilical venous endothelial cells (HUVEC) as well as human smooth vascular muscle cells (SMC) and fibroblasts from mice. The cells were incubated with various concentrations of the aforementioned PLs and fatty acids over 2 hours before staring the particular test.

For the studies on cell proliferation the two cell lines were cultured in 5% FCS at 37° C. and 5% CO₂. The cell suspensions were divided and filled into the incubation vessels with a cell density of 1.5×10⁵ in a 4-fold approach and POPC, DOPC, POPE, ONOPC, PNLPC, PNOPE or one of the nitro-carboxylic acid (s)-containing phospholipids or phospholipid according to the examples N7-N9, F, O2-O4, P1, P2, P5, P6, P8-P10, P15 and Q were added, so that the final concentrations were 10 μmol and 100 μmol. In one approach, only a 0.5% DMSO solution was added, this sample served as the control. Cells were washed twice with PBS and cultured under the above mentioned standard conditions for 24 h, 48 h and 96 h. Cells were displaced with a trypsin-ethylenediaminetetraacetate solution. The detached cells were isolated and the activity was stopped by adding of trypsin to the culture medium. Aliquots were taken for determining the cell number using a CASY 1 cell counter and analyzer system, model TTC (Scharfe System, Reutlingen, Germany), where in addition to the cell count, cell diameter and volume were also determined.

For determination of cell adhesion 6-well plates which have been fully coated with collagen XXII were used. Cell suspensions, which have been treated with 10 μmol and 100 μmol of above listed phospholipids and native fatty acids, having a cell count of 2.5-3.5×10⁵ were cultured in the 6-well plates under standard conditions for 24 h and 72 h. Thereafter; the culture medium was replaced by a 0.05% trypsin-EDTA solution. Continuing previous cultivation conditions the wells were gently shaken by a shaking plate and supernatants were collected from 2 wells after 10, 30 and 60 minutes and replenished with PBS. Finally a 2% solution of trypsin was is added and incubated for another hour and then the suspension was emptied. Cell suspensions drawn were analyzed with regard to the cell count and cell volume using an automated cell counter (see above).

Cell migration was investigated using an established wound closure test set-up. All three cell lines as described previously were incubated with the above stated phospholipids and fatty acids. After twice washing with PBS, 2−3×10⁵ cells were placed in Ibidi culture dishes, which were on agar plates; cells were cultured under standard conditions for 24 h. After this, the punch which had a width of 500 μm was removed. This was followed by a further cultivation for three days. A photo documentation of the culture area was performed every eight hours. The images were evaluated using software for automatic detection of cell surfaces which allowed calculation of the surface areas that were covered or left uncovered by cells.

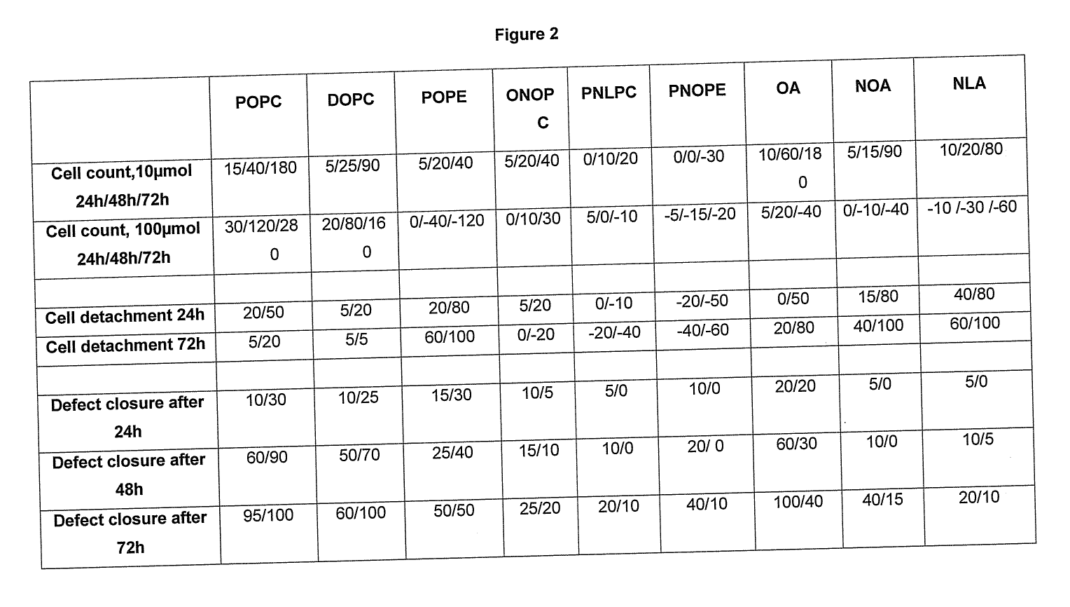

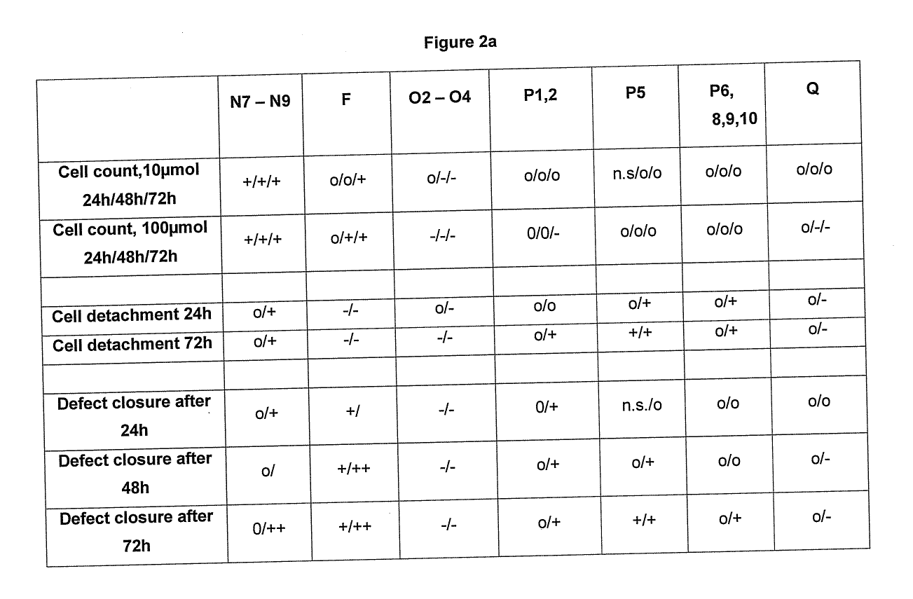

Results (these are Grouped Together in a Table Depicted in FIGS. 2 and 2a):

In comparison with the control groups, natural phospholipids as well as the native oleic acid cause cell proliferation. When cells were incubated with phospholipids having a choline head group, cell proliferation is increased at high concentrations while the proliferation decreases when phospholipids with an etholamine head group were used. Nitrated free fatty acids had no significant effect on cell proliferation compared to the control group when used at low concentrations, but a clear anti-proliferative effect at high concentrations. Nitro fatty acids at all concentrations exhibited a significant anti-adhesive effect, which is stronger than that of oleic acid. The decrease of proliferation is accompanied by increasing cell detachment, as well as by a reduced defect closure. The increase in proliferation due to phospholipids with a choline head group is accompanied by reduced cell detachment and more rapid and complete closure of the defect.

In contrast, cells that were incubated with nitrated phospholipids showed a slight reduction of cell proliferation at lower concentrations and a significant reduction at high concentration. The effect remained stable throughout the duration of the investigation. When compared to the calculated detachment rate of cells derived from experiments from the control group or the groups that were incubated with phosphocholine phospholipids, there was no significant difference in the rate of cell detachment. The wound closure was most reduced at all times as compared to the other groups, when cells were incubated with high concentrations of nitrated phospholipids.

The nitro-carboxylic acid (s)-containing phospholipids and phospholipid mixtures according to the examples N7-N9, F, O2-O4, P1, P2, P5, P6, P8-P10, P15 and Q showed this very homogeneous and consistent results overall, which did not significantly differ from those obtained with ONOPC, PNLPC or PNOPE. Thus can be concluded that even the mixtures nitro-carboxylic acid (s)-containing phospholipids show uniform effects, which do not differ from those individual a pure nitro-carboxylic acid (s)-containing phospholipids.

### Interpretation:

Incubation of cells with nitro-carboxylic acid (s)-containing phospholipids results in a significant reduction of cell proliferation over the investigated concentration range, the extent of which was reached only by incubation with a high concentration of nitrated fatty acids.

At the same time a considerably stronger adherence of cells to surfaces was present when incubated with the inventive nitrated phospholipids; this effect was also stronger that this was the case in cells incubated with nitrated fatty acids. Thus, it can be concluded that cell growth is reduced by nitro-carboxylic acid (s)-containing phospholipids but at the same time cell adhesion is promoted. Overall, these characteristics favor a physiological defect closure and cell homing, respectively. It is expected that these properties have a beneficial impact on the healing of a similarly coated implants.

### Example 10

### Studies on Effects of Fatty Acids and Phospholipids that Contain Native or Nitrated Carboxylic Acids on Phospholipid Model Membranes and Membrane Proteins

Incorporation of phospholipids into cell membranes can lead to a change of their physico-chemical properties. Therefore, effects of nitrated alkyl chains in phospholipids on membrane properties should be investigated. The lateral membrane pressure, the degree of anisotropy and the phase transition temperature can be estimated in model membranes of unilaminar vesicles by the use of the reporter molecule Laurdan.

Many membrane proteins achieve their functionality only after arranging engagement of their subunits. The folding process of some membrane proteins is mediated through the lateral interaction of neighboring trans-membrane helices. The change of the Frster resonance energy transfer (FRET) of fluorescence-labelled peptides with α helical trans-membrane structure in a double lipid layer can be used for determination of the concentration of monomeric and dimeric trans-membrane helices and for quantitative determination of helix-helix interactions. Variation of the lipid composition of a lipid layer thereby changes their physical properties which has an impact on the formation of dimers.

FRET measurements were carried out with fluorescence-marked Glycophorin A (GpA) peptides. The transmembrane GpA forms dimers, whereby the energy transfer can be measured. 5-carboxyfluorescein and tetramethyl-6-carboxyrhodamine were used as chromophores.

For the determination of membrane anisotropy, the native phospholipids SOPC SLPC and the analogue nitrated phospholipids SNOPC and SNPLC each alone, and mixed with the phospholipid DSPC 1:1 (w/w) were investigated. Mono-laminar vesicles from DSPC were examined as a reference. Additionally, phospholipids according to the examples of N1-N5, N15-N20, O1, O2, P1-P4, D, F, Q, R and S were examined in further examinations.

Degree of dimerization of the model protein was measured in a direct comparison between native phospholipids and the corresponding phospholipids with a nitrated alkyl chain for the given concentrations of phospholipids (10-50%, mol/mol) admixed to the DSPC vesicles.

Phospholipids were dissolved in a CHCl₃/MeOH mixture (1:1), the end concentration of the phospholipids was always 1 mM. Laurdan (in EtOH) was added to the lipid mixtures in the ratio of 1:500 (2 μM), this mixture was homogenized. The solvent was evacuated from the samples using vacuum evaporation. Then samples were hydrated with 250 μl HEPES buffer (150 mM NaCl, 10 mM HEPES, pH 7.4) and incubated at 65° C. while mixing the samples at 1400 rpm for at least 30 minutes, allowing formation of multi-lamellar vesicles. To form mono-lamellar vesicles, the samples were initially frozen in liquid nitrogen, then thawed at 65° C. in a water bath and homogenized at 1400 rpm for 1 min; this procedure was performed for five cycles. From the samples 200 ml were taken and measured using a fluorescence spectrometer type Horiba scientific FluoroMax-4, equipped with digital temperature control (Horiba scientific F-3004).

For determination of the dimerization, the peptides of FL GpAwt and TAMRA GpAwt were weighed and dissolved in TFE. The measurements were carried out on a fluorescence spectrometer of type Aminco Bowman series 2 (Thermo Spectronic).

Results: (The results are summarized in FIGS. 10a and 10b).

The native phospholipids showed a uniform behavior in the gel phase, the phase transition temperature was between 52° and 54° C. With higher temperatures the degree of anisotropy increased. In contrast, the degree of anisotropy was lower in the gel phase by adding nitrated phospholipids and the phase transition temperature was significantly moved to the left (40-42° C.). Furthermore, the degree of anisotropy at higher temperatures was lower as compared to native PL.

For substances from examples D and F, there was a stronger effect on the membrane melting point (10-20% reduction) and on the degree of anisotropy (increase), for substances from examples N-N5, N15-N20, O1, O2, P1-P4, Q the found effects were comparable with the results of SNOPC and SNLPC. For substances from examples F, Q, R and S the effects described above were 15% to 30% less.

The addition of phospholipids with one unsaturated fatty acid reduced the degree of dimerization of the model membrane protein. Analogue phospholipids with a nitro residue on the unsaturated fatty acid resulted in a significantly greater reduction of dimerization (P13, SNLPC; P14, SNOPC). (The value of the DSPC FRET measurement without the addition of other phospholipids was normalized as 100%). For the nitrated PLs according to the examples of N1-N5, N15-N20, O1, O2, P1-P4, D, F, Q, R and S degree of dimerization was reduced, comparable to the values found for substance from examples P13 and P14.

Interpretation: Phospholipids containing nitrated fatty acids have a much stronger effect on the fluidity and thus the membrane melting point than the corresponding native phospholipids. Furthermore, a reduction of the degree of order was found within the range of the membrane temperatures of the liquid crystalline phase, while in the temperature range of the gel phase and especially at higher temperatures the degree of order is significantly greater than in membranes with admixed native phospholipids. The increase in the degree of order is most likely the cause for the found reduction of dimerization of model membrane proteins. This effect is significantly stronger in phospholipids with nitrated fatty acids than in those of native phospholipids. Thus, the inclusion of nitro-carboxylic acid (s)-containing phospholipids in a model cell membrane leads to an increase of their stability at physiological temperatures, and a decrease of membrane fluidity, respectively. Because noziception of cells depends on the fluidity of the cell membrane to a large extent, a reduction of perceptions against mechanical, chemical and osmotic alterations due to incorporation of nitrated phospholipids can be assumed.

### Example 11

### Studies on Effects of Fatty Acids and Phospholipids that Contain Native or Nitrated Fatty Acids on Induction of Fibrosis Due to Fibroblast Activation

Scar formation as well as fibrosis are a consequence of nonphysiological production of collagen derived from activated fibroblasts. The effects of incubating fibroblasts with native PL and nitrated PL (SNOPC, PNOPC, PNLPC) as well as that of the native fatty acids oleic acid and linoleic acid and the nitrated fatty acids nitro oleic acid and nitro linoleic acid, respectively, were investigated by the use of an in-vitro cell model.

In further experiments the nitro-carboxylic acid (s)-containing phospholipids and PL mixtures according to the examples N4N8, N10, N11, D, B, O2, O4, O6, P8P12, P15 and Q were also tested.

Fibroblasts from mice were used that have been sequenced 5 times. Those cells were incubated with the native and nitrated PL as well as the native and nitrated fatty acids as stated above with a final concentration in the cell suspensions of 10, 100 and 200 μmol for the PL, and 10 and 100 μmol for the fatty acids, for 24 h. Aliquots containing approximately 1.5×10⁴ fibroblasts were given in a 8 chamber glass slide (Lab-Tek II, Nunc) and cultured in this manner for 3, 5 and 7 days after adding 2% FCS solution. In further experiments performed in the same fashion, TGF-β was applied to the wells. Collagen synthesis was determined semi-quantitatively by means of immune-histo staining. Immuno-histochemical marking (DAKO, LSAB2 system, USA) was performed after cultures were washed with PBS and fixed in ethanol/acetone (99:1 v/v) for 10 minutes. Then the wells were washed with 0.05 M TRIS/HCl buffer (Merck; pH 7.2-7.6) and incubated with 3% H₂O₂ solution. After final washing, monoclonal anti-collagen I antibody (MAB3391, Chemicon) was added for 10 minutes. Thereafter the samples were washed and the secondary biotinylated link antibody (anti-mouse- and anti-rabbit immunoglobulins, DAKO) was incubated for 20 minutes, which was followed by a washing step. The Streptavidin was incubated with peroxidase (DAKO) for 10 minutes. After further washing, substrate chromogen AEC (3-amino-9-ethyl, DAKO) was added for 10 minutes. This was followed by counterstaining with haematoxylin for 5 minutes. The preparations were evaluated by light microscope.

Results: Fibroblasts of the control group showed a linear increase of the amount of collagen matrix throughout the duration of the investigation; collagen production was disproportionately increased during stimulation with TGF-β. Native fatty acids had no significant effect on collagen synthesis at the low concentration; at high concentrations, the collagen synthesis was reduced compared with the control at day 3 and increased as compared to control at day 7. Nitro fatty acid at the low concentration decreased collagen synthesis up to day 5. At the high concentration, the collagen synthesis was significantly reduced as compared to the control throughout the investigation. Stimulation with TGF-β, resulted in exaggerated collagen synthesis at all times periods when low concentrations of native fatty acids were added. High concentration of native fatty acids in the presence of TGF-β resulted in a reduction of the collagen content on day 3, which then increased significantly. The nitro fatty acids showed similar effects on the synthesis of collagen, but the reduction of collagen synthesis was stronger (n.s.) on day 3; on day 7, there was no difference in the amount of collagen found in native fatty acids.

Native phospholipids had no effect on collagen synthesis at the concentrations of 10 μmol and 100 μmol. At the highest concentration, a reduction in collagen synthesis was observed on day 3 and an increase on day 7, as compared to the control. Stimulation with TGF-β resulted in a significant increase of collagen synthesis as compared to controls in all native phospholipids, with the exception of the groups with the highest concentration at day 3. The incubation with the nitro-carboxylic acid (s)-containing phospholipids at a concentration of 10 μmol resulted in a reduction of collagen synthesis, which was significant at day 3 and trended to be lower as in controls at day 5 and 7. With the use of high concentrations of the nitrated PL there was an almost complete elimination of the collagen synthesis at all time points. After stimulation with TGF-β, in samples with a low concentration of the nitro-carboxylic acid (s)-containing phospholipids, there was no difference in the results without TGF-β-stimulation, but a significant decrease as compared to the control arm at all time points. Incubation with a concentration of 100 μmol while stimulating with TGF-β, however, resulted in an almost complete inhibition of collagen synthesis. These results were obtained in all tested nitro-carboxylic acid (s)-containing phospholipids according to the examples of N4-N8, N10, N11, D, B, O2, O4, O6, P8-P12, P15 and Q regardless of whether mixtures or pure substances. Thus, it seems crucial that a nitrated alkyl residue exists, independent of the type of phospholipid used.

Interpretation: Native and nitrated fatty acids can reduce collagen synthesis; however, they can not suppress collagen synthesis that is induced by cytokine stimulation. Incubation with native phospholipids has no relevant influence on collagen synthesis. In contrast, nitro-carboxylic acid (s)-containing phospholipids cause a strong inhibition of collagen synthesis. Unlike the nitrated fatty acids this effect is maintained during stimulation with cytokines. Thus, the documented effects are suitable to prevent excessive, cytokine-mediated fibrosis. This can lead, for example, to a marked reduction in fibrosis that is induced by an implant.

### Example 12

### Studies on the Stability of Nitro-Carboxylic Acid-Containing Phospholipids and their Effects on Phospholipid Mixtures

Coatings of medical devices should not undergo alteration of their chemical structure or their physico-chemical properties during sterilization procedures, and also exhibit long-term stability. The stability of a phospholipid layers in air is limited, as known in the art. Stability is influenced by intermolecular bonding forces, as well as the water content of the polar head groups. In addition, it is known that oxidation of unsaturated fatty acids in phospholipids can occur.

The natural phospholipids POPC and SLPE, as well as the analogous nitrated phospholipids as shown in example C(PNOPC) and R(SNLPE), as well as the phospholipids according to examples B, D, E, F, G, N1, O1, P2, P5, Q, and S as a mono substance and as a combination of the native phospholipids and the corresponding nitro-carboxylic acid (s)-containing phospholipids mixed in a ratio of 1:1 were used for this investigation.

Balloon catheters were coated by means of the Langmuir-Schaefer procedure where the balloon segment was aligned longitudinally and coaxial to the solution surface, dipping into the liquid for approx. 1 mm over the entire length. Then the catheter was slowly rotated around its axis 5 times. Phospholipids were dissolved at a concentration of 3 mmol in an ion-free aqueous solution at 50° C. The solution was cooled and given to the coating solution then. In case of incomplete dissolution of the phospholipids or if they separated when cooled, DMSO was added at a concentration of up to 20 vol.-%, preferably up to 10 vol.-%. After coating, the catheters were vacuum dried for 8 hours in order to remove residual solvent.

The coating stability was tested with regard to its abrasion stability by inserting the balloon catheters, which had an outer diameter of 0.85 mm, in a PTFE tubing that had an internal diameter of 1.0 mm and was embedded in a silicon model that also fixed the silicon tubing employing multiple consecutive angulations thereof of up to 60° in four directions consecutively, and then the catheters were pulled by means of an automated cable traction device at a constant speed of 3 cm/s through this tubing. The tubing was filled with a 10% human albumin solution at a temperature of 35° C. The cable system, which had previously been brought through the tubing was connected to the catheter tip and tracked outside the tubing by reversing pulleys, which allowed exactly vertically traction of the cable that was connected to a motorized winch. The winch was mounted on a digital precision balance. The catheters mounted in this manner were pulled through the tubing system and the weight changes measured by the balance, which represented the traction work load achieved for the passage of the catheter, was recorded continuously. These readings were integrated over time. The resultant values allow estimation of the total shear force that occurred during the passage of the catheter through the tubing system.

The abrasion or loss of coating layers was determined by weighing the catheter before and after coating as well as after the mechanical stability test in the tubing system as described above using a high precision balance. In order to examine the long-term stability of the coating, catheters coated with phospholipids were sealed with polyethylene glycol 1000 (Roth, Germany). For that purpose PEG 1000 was melted and mixed with a 10% ethanol solution at 50° C. A portion of the phospholipid-coated balloon catheter was coated by means of dip-coating in this PEG solution at 50° C., and dried thereafter for 8 hours.

Stability of cis-conformity of unsaturated fatty acids of the phospholipids investigated was determined after heat treatment at 60° C. for three hours in a heating cabinet. After 24 hours and after 2 months the phospholipids coated on the balloon surface were detached and analyzed. Detachment was carried out in 50 ml of a chloroform:methanol mixture (3:1, v: v). A 10 μl aliquot was used for a FTIR spectroscopy. For this purpose the samples were dropped on a ZnSe ATR crystal and then the solvent was evaporated. Degree of transisomerization was determined using the integral of intensity of proton resonance in comparison to that measured from a cis configuration of the reference substances. Measurements were performed 24 hours after coating, heat treatment and after 2 months of storage at 25° C. under sterile room air conditions.

Results (these are Grouped Together in the Table of FIG. 3):

The proportion of trans-fatty acids was small (<5%) in all of the synthesised phospholipids. There was a trend to a higher proportion of trans-fatty acids in natural phospholipids as compared to the nitrated phospholipids in measurements 24 hours after applying the phospholipids on a catheter. After heat treatment, the proportion of trans-fatty acids rose to 80% (POPC) or 86% (SLPE), respectively, in the group of natural phospholipids. In contrast, the proportion of trans-fatty acids of nitrated phospholipids was 25% and 28% (PNOPL, SNLPE); the difference was statistically significant. Degree of transisomerization in measurements from coatings after 2 months revealed a proportion of 95% and 98% in the natural PLs and of 30% and 32% in the nitrated phospholipids. Figures derived from coatings with mixtures of natural and nitrated PL documented that the degree of transisomerization found in mixtures of natural and nitrated PLs was significantly lower, as calculated from the previous investigations using singe substance classes.

Top coating with PEG had only a slight effect on the degree of transisomerization which was found to be only slightly reduced in natural phospholipids to 86% and 92% in POPL and SLPE coatings after 2 month, and remained virtually unchanged in the nitrated phospholipids (32% PNOPL, 33% SNLPE). However, when coatings with mixtures form natural and nitrated PLs were top coated, the measured degree of transisomerization was significantly lower than for their combination without an additional coating.

After drying of the coating substance loss was minimal. Heat treatment resulted in a significant loss of coating substance with the use of natural phospholipids. In contrast, the substance loss of nitrated phospholipids was low. With the combination of PLs with natural and nitrated fatty acids, a stronger substance loss as for the measurements at the same time with the pure substance classes was observed in measurements after 24 hours. After heat treatment, the substance loss was not significantly lower than the calculated means of the loss of the two pure substance classes.

The amount of mechanical removal of coating substance was significantly greater in coatings with natural phospholipids than in those using nitrated phospholipids according to substances of examples C(PNOPC) and R(SNLPE). The loss of coating material was slightly greater in mixtures of natural and nitrated phospholipids, than was calculated from the measurements with the pure substances. The workload to overcome friction energy while pulling the coated catheters through the PTFE tubing was proportional to the respective loss of coating material. Very similar results were also obtained for coatings with phospholipids according to examples B, D, E, F, G, N1, O1, P2, P5, Q and S.

Interpretation: Improvement of lubricity of an object/implant can contribute to a reduction of tissue trauma while transferring it in/into a body. In addition, the coating material should have sufficient resistance against premature abrasion during introduction in an organism. In addition, it should exhibit chemical and thermal stability. These requirements were found to be significantly better provided by nitro-carboxylic acid (s)-containing phospholipids than by native phospholipids.

### Example 13

### Investigation of Effects on Cell Membranes by Nitro-Carboxylic Acid (s)-Containing Phospholipids

The physico-chemical properties of cell membranes determine their strength of resistance to physical, chemical and immunological alterations. Human red blood cells and mast cells from mice dissolved in solutions of physiological NaCl containing the natural phospholipids SOPC and PLPC or the analogue nitrated phospholipids (1-stearoyl-2-(9-nitrooleoyl)-sn-PC (example P14) and 1-palmitoyl-2-(9-nitrolinoleoyl)-sn-PC) (example P7) at a concentration of 30 mmol/l were incubated for an hour in order to assess the effects of an up-take of phospholipids into the cell membranes with respect to resistance to external alterations. Similarly, cells incubated with mixtures of nitro-carboxylic acid (s)-containing phospholipids according to examples N2-N14, F, Q, S, O3-O6, P1-P3 and P6 were investigated.

For assessment of osmotic stability, cells were separated from serum by centrifugation first and then cleaned three times with physiological NaCl solution. The erythrocytes were resuspended in physiological saline solution and then suspended phospholipids were added. This stock suspension was moved on a vibrating plate with slow rotational speed at 30° C. for 1 hour. Aliquots of 3 ml were filled in glass tubes and centrifuged. After removal of the supernatant, distilled water or NaCl solutions with increasing concentrations from 0.1 to 1.0 g/dl were added. After incubation for 30 minutes under the above described conditions, the tubes were centrifuged and an aliquot was taken for photometric measurement of hemoglobin by absorbance at a wavelength of 546 nm at a temperature of 30° C.

For the examinations of mechanical stability, erythrocytes were prepared according to the procedure above. Curvets containing cell suspended saline solution were placed in an ultrasonic bath (BANDELIN DT 31 H, Berlin, Germany) and sonified with 10 Watts at a temperature of 30° and 50° C. for two to five minutes. Then, the samples were centrifuged and supernatants were analyzed as stated above.

In order to test storage stability of erythrocytes, samples, which were prepared as described above, were stored at 4° C. for 2 days. Then, they were rewarmed to 30° C., in each experiment one sample was not preincubated with PLs which served as blank. The rewarmed samples were moved on a shaking plate (BANDELIN, Sonoshake, Berlin, Germany) with a low rotation rate at 30° C. for 24 to 48 hours. This was followed by the preparation and analysis of samples as described above.

Cell membrane-stabilizing properties of phospholipids were tested using an in-vitro model of dog mast cells (C2 cells). The cells were cultured in 5% FCS media under standard conditions. The cells were washed several times in a calcium- and magnesium-free buffer solution and finally concentrated. The cells were distributed on 96-well plates and incubated with a NaCl solution containing the above listed phospholipids for one hour at 37° C. Then, Mastoparan (Sigma, Germany) was added in the concentrations of 5 μmol and 25 μmol. The Ca²⁺ influx was determined using a calcium ionophore (A23187, Sigma Germany). The calcium influx was normalized to measurements derived from the respective base measurements and expressed as a percentage change. The release of histamine from the C2 cells was determined using a histamine-ELISA (ILB, Germany).

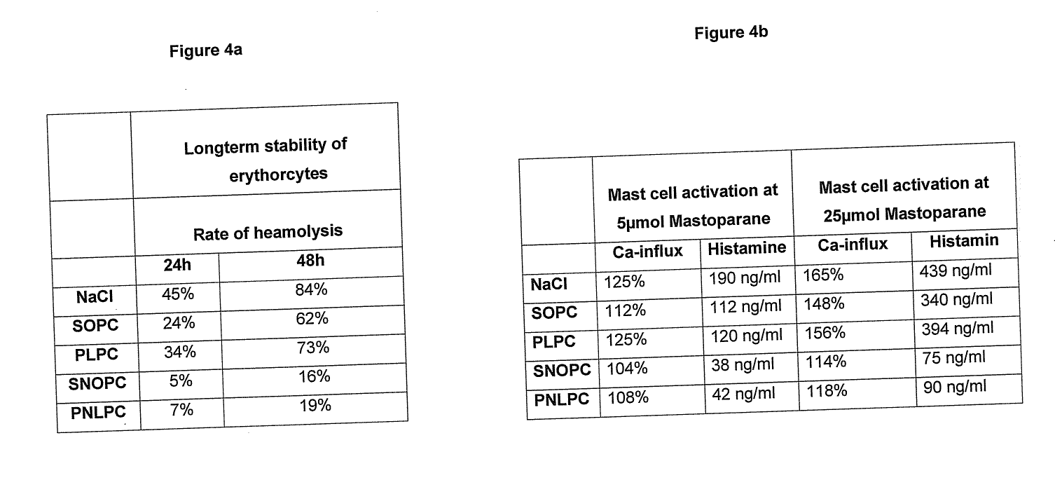

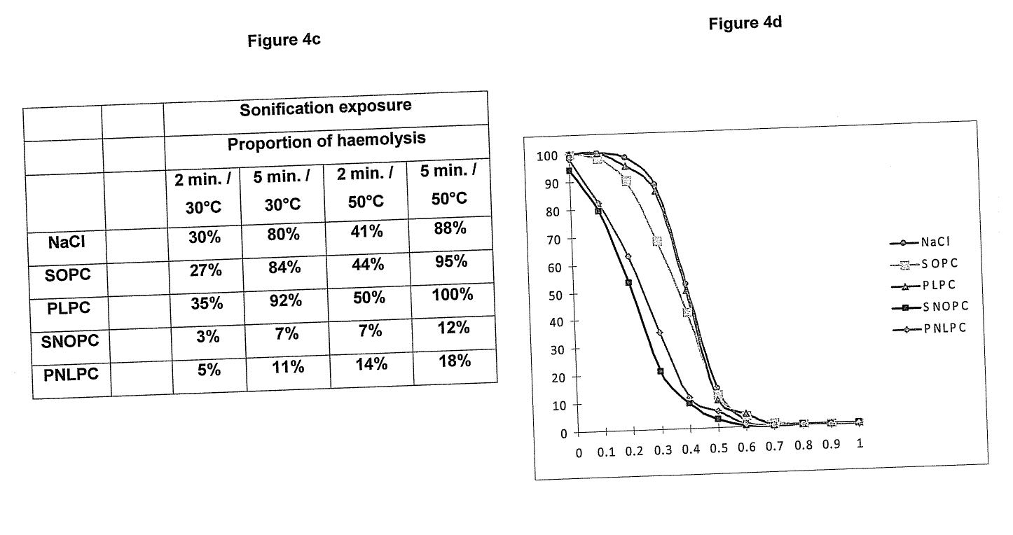

Results (these are Summarized in FIGS. 4a to 4d as a Table):

Incubation with natural phospholipids has little effect on membrane resistances toward an osmotic stress. Mechanical stability of membranes was reduced by natural PL having unsaturated fatty acid residues. Natural phospholipids have only a limited impact on the long-term stability of the erythrocyte membrane. In contrast, incubation of erythrocytes with nitrated phospholipids leads to a significant increase in cell stability towards osmotic and mechanical stresses; also the long-term stability is greatly increased. Finally, it has been shown that nitrated phospholipids stabilize the mast cell membrane and degranulation is largely prevented.

As already demonstrated in the other experiments, mixtures of different nitro-carboxylic acid (s)-containing phospholipids had in principle the same effect as the pure nitro-carboxylic acid (s)-containing phospholipids containing stereo isomers but no regioisomers. No significant differences to the effects of 1-stearoyl-2-(9-nitrooleoyl)-sn-PC and 1-palmitoyl-2-(9-nitrolinoleoyl)-sn-PC could be observed. It is also to mention that nitrated phosphatidylinositol (F) and the phosphatidylethanolamine (S) provided very similar values as the nitrated phosphocholines.

Interpretation: The improvement of cell membrane stability that can be achieved by nitro-carboxylic acid (s)-containing phospholipids, but not by natural phospholipids, thereby achieving an improved ex-vivo storability of blood which can be useful, e.g., when used for blood storage, or in-vivo to stabilize cells, e.g., when used during extracorporeal circulation. The effects can also be used to reduce cytokine-mediated changes of cell wall permeability. This can provide, e.g., anti-allergic effects.

### Example 14

### Investigation of Cell Protective Properties of Phospholipid-Containing Nitro-Carboxylic Acids

Toxicity of substances on cells can be mediated via various mechanisms of action: (1) damage of surface structures of the cell membrane with translation of the alteration by membrane proteins to the cell interior, (2) direct damage of the cell membrane, or (3) trans-membranous up-take of the substance into the cell. The degree of damage, however, largely depends on physico-chemical properties of the cell membrane and not on the basic principle of these types of damage. Therefore, it should be investigated whether cytotoxicity of well-known cytotoxic substances that are mediated through one or more of these mechanisms are attenuated by the membrane-stabilizing effects of nitrated phospholipids.

As tubular and vascular endothelial cells react especially sensitive towards cytotoxic substances, they were used under in-vitro culture conditions to study cytoticic effects. A LLC-PKI cell line suspended in a medium (D-MEM medium) with 10% fetal calf serum (FCS) and sodium bicarbonate (26 mmol/l) in 5% CO₂ atmosphere was cultured.

Cell suspensions with a cell count of 1.5×10⁵ were either incubated with saline or the natural phospholipids SOPC and PLPC, or the analogue phospholipids with nitrated unsaturated fatty acids (1-stearoyl-2-(9-nitrooleoyl)-sn-PC and 1-palmitoyl-2-(9-nitrolinoleoyl)-sn-PC) at concentrations of 10 to 50 μmol/l or with the nitro fatty acids nitro oleate (NOA) and nitro linolate (NLA) at concentrations of 10 or 30 μmol. The cultures were moved at a slow speed by a shaking plate for 2 hours. Then cisplatin (25 and 50 μmol/l) or cyclosporine (50 and 100 μmol/l) was added to the cell suspensions which were cultured for another 24 hours while slowly agitating the samples.

Furthermore, murine endothelial cells were cultured in standard medium containing 10% FCS. Incubation with the aforementioned substances was performed as described previously. Lipopolysaccharide from Escherichia coli (4 and 8 μg/ml; Simga) was added to the cell suspensions which were further processed as previously described.

The cell suspensions were marked with two fluorescent dyes (LIVE/DEAD®, Molecular probes). This was followed by a flow cytometry (FACSCalibur, Becton & Dickinson). The percentage of necrotic cells was calculated from the ratio of red fluorescent cells and the total number of identified cells.

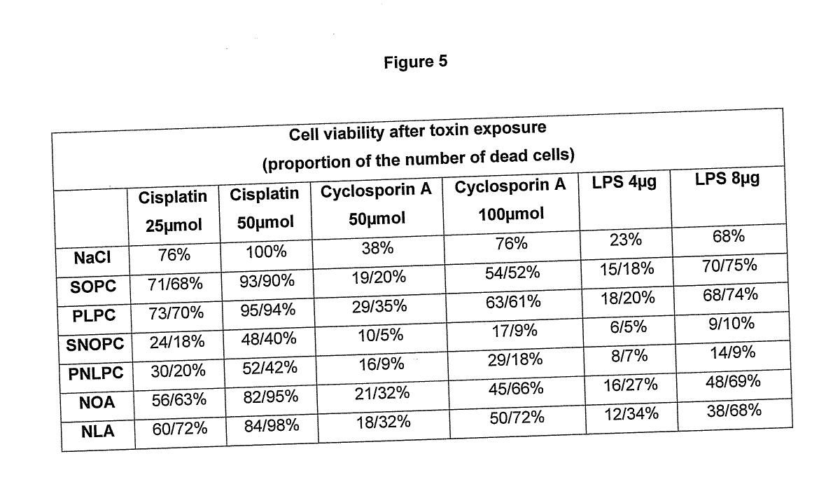

Results (these are Grouped Together in the Table of FIG. 5):

Incubation of endothelial and tubule cells with natural phospholipids has a minimal effect on the cytotoxicity of substances which had different pathomechanisms. Incubation with nitrated fatty acids at a low concentration exhibited a tendency to reduce cytotoxic effects. However, a significant reduction in the toxicity for all investigated toxins was accomplished by pretreatment with nitrated phospholipids. Thus, it could be shown that a pretreatment with nitro-carboxylic acid (s)-containing phospholipids increases resistance against cell toxins. The inventive phospholipids can thus provide beneficial effects in exogenous and endogenous intoxications, which could be, e.g., the case if there is an entry or production of toxins during wound healing, but effects can also be beneficial in patients who suffer from systemic poisoning.

### Example 15

### Investigation of Cell Protective Properties of Nitro-Carboxylic Acid (s)-Containing Phospholipids in Barotrauma

Freshly extracted iliac artery segments from pigs were dissected in order to uncover adventitial layer and were cultured then in PBS containing 1% FCS for three days. Segments 5 mm in length were separated atraumatically and placed in culture vessels that contained solutions of natural phospholipids POPC and SLPC, as well as the analogue phospholipids with nitrated unsaturated fatty acids (1-palmitoyl-2-(9-nitrooleoyl)-sn-PC and 1-stearoyl-2-(9-nitrolinoleoyl)-sn-PC) each at a concentration of 50 mmol, as well as the nitrated free fatty acids nitro oleic acid (NOA) and nitro linoleic acid (NLA) at a concentration of 30 μmol, for another two days. Also the inventive phospholipids according to the examples N1-N5, N11, N15-N17, N20, O2-O5, P4-P6, E, G and Q were tested in the same manner. After changing of culture medium, the vessels were brought into a pressure chamber and were exposed to air pressure at 15 bar for one hour. Then the pressure was reduced quickly within about 5 seconds. The culture flasks were slowly moved on a vibrating plate under standard culture conditions for 7 days. The culture medium was changed after the 3rd day and an aliquot thereof was analyzed further for determination of microparticles. The vascular segments were finally fixed and embedded. A TUNEL staining (in situ cell death detection kit, AP, Boehringer, Germany) was performed 48 hours after air drying of the specimens. The analysis was carried out using a light microscope; without differentiation between apoptosis and necrosis, the number of dead cells was set in relation to the total cell count within a given field of view.

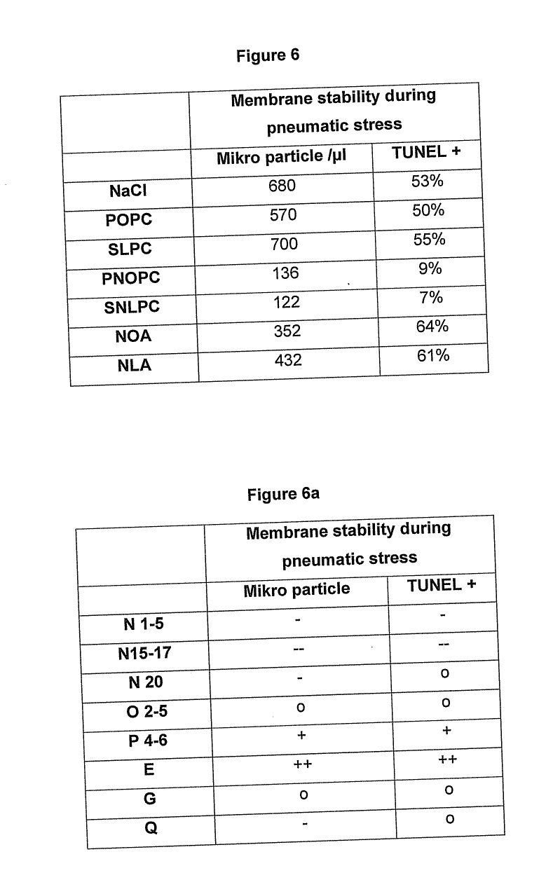

Annexin V-allophycocyanin (3 μl) (APC) (BD Pharmingen) and then 100 μl of Annexin-binding buffer solution (BD Pharmingen; 1:10 vol/vol in distilled water) were given to aliquots of 50 μl of the culture medium. The number of microparticles was then measured using flow cytometry (FACSCanto, Becton & Dickinson). The detection window was set to 0.3-1.0 μl.
Results (these are Grouped Together in the Table of FIGS. 6 and 6a):

Barotrauma of vessel segments caused extended apoptosis (necrosis). While preincubation with natural phospholipids did not have an influence, a slight increase of apoptotic cells was observed after pre-treatment with nitrated fatty acids. Pre-incubation with nitrated phospholipids, however, led to a significant decrease in the apoptosis (necrosis) rate. These results were in accordance with the results found for the count of microparticles, where fewer microparticles were found after pretreatment with nitrated phospholipids, and an equal amount as compared to the control group was found after pretreatment with natural phospholipids. Pre-incubation with nitrated fatty acids resulted in a considerable reduction of microparticle formation also; however, this was less than after incubation with nitro-carboxylic-acid (s)-containing phospholipids. These results could also be reproduced with the nitro-carboxylic acid (s)-containing phospholipids according to examples N1-N5, N11, N15-N17, N20, O2-O5, P4-P6, E, G, and Q. Here no significant differences arose as compared to 1-palmitoyl-2-(9-nitrooleoyl)-sn-PC and 1-stearoyl-2-(9-nitrolinoleoyl)-sn-PC. Thus, it appears that the effect is independent of the used phospholipid, i.e., independent of the head group of the phospholipid and regardless of whether the inventive nitro-carboxylic acid (s)-containing phospholipids are used as mixtures. It seems to be that the decisive factor is the existence of at least a nitrated carboxylic acid or nitrated fatty acid in the phospholipid.

Interpretation: Barotrauma occurs in particular during angioplasty, for example a balloon catheter is advanced to the narrowed segment in the blood vessel, where it is inflated using considerable pressure (up to 20 bar), thereby squeezing the vessel wall. The resulting damage to the vessel wall causes tissue responses, which contribute to a re-narrowing of the vessel with clinical appearance of a restenosis. The nitro-carboxylic acid (s)-containing phospholipids can counteract this effect and are therefore suitable for all indications where cells/tissues are exposed to a pneumatic/compressing stress.

### Example 16

### Investigation of Cell Protective Effects of Nitro-Carboxylic Acid (s)-Containing Phospholipids in Hypoxia and in Re-Perfusion Damage

Both apoptosis and necrosis as a result of a severe cell ischemia is closely associated with changes in cell membrane properties. To determine whether the membrane-stabilizing properties of nitrated phospholipids can lead to attenuation of effects due to an ischemic event or the re-perfusion damage, cortical neurons and cardiac myocytes were investigated. These neurons were prepared from young mice, according to a published procedure (Goldberg M P, Choi D W. Combined oxygen and glucose deprivation in cortical cell culture: calcium dependent and calcium-independent mechanisms of neuronal injury. J Neurosci 1993; 13:3510-3524).

Freshly prepared cortex tissue was separated with Papain and tissue suspensions were cultured in culture vessels (Prim ARA; BD Biosciences, USA) with Neurobasal A/B27 medium (Invitrogen) for 10-12 days. Cell suspensions were divided, and replenished with suspensions of the natural phospholipids SOPC and PLPC, as well as the analogue phospholipids with nitration of unsaturated fatty acids (1-stearoyl-2-(9-nitrooleoyl)-sn-PC and 1-palmitoyl-2-(9-nitrolinoleoyl)-sn-PC) at concentrations of 10 to 50 μmol/l in NaCl (0.9%) were added. In other sets of experiments, the inventive phospholipids according to examples C, D, F, N3-N7, N13-N19, O1, O2, O5, O6, P3, P8 and Q were tested in the same manner. After 4 hours, the cells were washed three times with buffered saline and buffered NaCl solution with the addition of MgCl₂ and CaCl₂ in an anaerobic atmosphere (85% N₂, 5% O₂, 10% CO₂; at 35° C.) for 10 and 30 minutes. Then, the cells were washed once and grown in culture medium under aerobic conditions for 24 hours. Then the cells were separated, according to a published technology (Meller R, Skradski S L, Simon R P, Henshall D C. Expression, proteolysis and activation of caspases 6 and 7 during rat C6 glioma cell apoptosis, Neurosci Lett 2002; 324:33-36). Cells were stained with annexin V-FITC and propidium iodide (Molecular probes, USA). The vital and necrotic cells were measured by flow cytometry (FACSCalibur, Becton & Dickinson). The volume of culture medium was determined and an aliquot was taken for determination of the LDH (lactate dehydrogenase) concentration.

Cardiac muscle cells derived from neonatal rat heart (Nitobe J, et al, Reactive oxygene species regulate FLICE inhibitory protein and susceptibility to FAS-mediated apoptosis in cardiac myocytes. Cardiovasc RES 2003, 119-28). The heart muscle cells were cultured in Dulbecco/Eagle medium (DMEM) with 10% FCS added under standard conditions for two days. Incubation with the natural and nitrated phospholipids, as well as the hypoxia experiments were carried out as described above. Incubated cells were grown in culture medium under standard conditions for 24 hours. This is followed by a vitality staining as described above. Immediately after the end of hypoxia and 10 and 30 minutes thereafter a portion of the cell suspensions was frozen in liquid nitrogen. The frozen cells were thawed up to −4° C. and homogenized for subsequent NAD⁺ analyses. By means of differential centrifuge, pellets containing mitochondria were separated (Di Lisa, et al, 1993; Am. J. Physiol. 264, H2188-H2197). The NAD⁺ content was determined by fluoroscopy, and normalized to the protein content (Veloso, D., and Veech, R. L., 1974;) Anal. Biochem., 449-450).

Results: Cortical cells exhibit a viability of 35% and 0% after a 10- and 30-minutes ischemia measured after 24 hours in the control group. This was accompanied by an increase in LDH that was 350 times and 800 times higher than the values of a culture done in parallel under normoxemia. Viability of cells incubated with the natural phospholipids was not significantly different from that of the control group (SOPC: 33% and 0%; PLPC: 30% or 0%). The same was the case for LDH release (SOPC: 280 or 640 fold; PLPC: 380 and 630 fold). Cells incubated with the nitrated phospholipids, however, had significantly higher viability (SNOPC: 68% and 54%; PNLPC: 70% and 52%, respectively) and led to a lower increase of LDH (SNOPC: 120 or 230 fold; PNLPC: ×130 and 290 fold).

Heart muscle cells that were grown under aerobic conditions showed only a minimal loss of viability (<3%) and served as the control group. Hypoxia resulted in a decrease in the viability to 16% and 43%, respectively. Heart muscle cells that were treated with the natural phospholipids SOPC and PLPC had a viability that was comparable to that of the control group (SOPC: 14% and 40%; PNPC: 16% and 42%, respectively). Heart muscle cells that were incubated with the nitrated phospholipids showed a significantly lower loss of viability (SNOPC: 6% and 16%; PNLPC: 5% and 18%, respectively). The mitochondrial NAD⁺ content of heart muscle cells decreased rapidly and progressively (2.8 and 0.9 nmol/mg protein) in the hypoxia control group as compared to the baseline value (5 nmol/mg protein). The same hold true for pre-incubation with SOPC and PLPC (3.0 and 1.1; 2.4 and 0.7 nmol/mg protein, respectively). After pre-incubation with SNOPC or PNLPC, significantly higher values were found for NAD⁺ (3.8 and 3.3; 3.6 and 3.1 nmol/mg protein, respectively).

The experiments performed with 1-palmitoyl-2-(E-9-nitrooleoyl)-sn-3-glycero-phosphocholine (C) and 1-palmitoyl-2-(Z-9-nitrooleoyl)-sn-3-glycero-phosphocholine (P8) did not show relevant differences compared with the experiments done with 1-stearoyl-2-(9-nitrooleoyl)-sn-PC(P14) and 1-palmitoyl-2-(9-nitrolinoleoyl)-sn-PC(P7). Experiment performed with phospholipids according to examples D, F N3-N7, N13-N19, O2 and Q resulted even in slightly better values than found for 1-palmitoyl-2-(E-9-nitrooleoyl)-sn-3-glycero-phosphocholine, whereas phospholipids according to examples, O1, O5 and O6 exhibited slightly worse results compared to 1-palmitoyl-2-(E-9-nitrooleoyl)-sn-3-glycero-phosphocholine.

Interpretation: Incubation of cells with nitro-carboxylic acid (s)-containing phospholipids makes them less susceptible to the negative effects of hypoxia and reperfusion. These characteristics are suitable to protect tissues and organs from the effects of a lack of blood and oxygen supply and make them suitable to reduce tissue/organ infarction and destruction.

### Example 17

### Investigation on Properties of Nitro-Carboxylic Acid (s)-Containing Phospholipids on Intra Cellular Calcium Homeostasis

Separation as well as supply of calcium is an important prerequisite for the proper functioning of most cells. This applies particularly to heart muscle cells. As a consequence of membrane leakage of the sarcoplasmatic reticulum, leading to an increased cytosolic calcium concentration, the force of contraction will be reduced, as well as disturbances in membrane repolarization can occur, which can lead to arrhythmias. It is also known that a β-adrenergic stimulation may increase the outward current leakage of calcium that is induced by a membrane defect, a finding that explains the pro-arrhythmogenic effect of β-receptor stimulation during hypoxia. Hypoxia-induced acidosis can be a cause for such a disturbance of calcium homeostasis.

Heart muscle cells were taken from rabbit hearts according to a method described elsewhere (Shannon T R, Ginsburg K S, Bers D M. Quantitative assessment of the SRCa²⁺ leak-load relationship. Circ Res. 2002; 91:594-600). The prepared cells were cultured for 72 hours. The natural phospholipids SOPC and PLPC, as well as the analogue phospholipids with nitration of unsaturated fatty acids (1-stearoyl-2-(9-nitrooleoyl)-sn-PC(P14) and 1-palmitoyl-2-(9-nitrolinoleoyl)-sn-PC) (P7), as well as the nitro-carboxylic acid (s)-containing phospholipids according to examples B, C, D, F, N2, N4-N7, N10-N16, O1-O3, P8-P12 were added to the culture medium at a concentration of 80 mmol/l and incubated for 2 hours.

The cytosolic Ca²⁺ concentrations were determined with fluo-4 fluorescence. Therefore the cells were incubated with 10 μmol fluo-4 AM (1%) for 30 minutes and washed thereafter three times with PBS. The cells were stimulated in a Tryode solution on a platinum electrode with a frequency of 0.5 Hz over 20 cycles. Then the ambient medium was changed and an electrolyte-free colloidal solution was added. Determination of the Ca concentration was performed after each series of stimulation over 20 cycles. Calcium content was estimated by the use of confocal laser scanning microscopy (Olympus). At least 10 cells per experiment were evaluated in a post-systolic interval of 1000 ms. The measurements were repeated at least three times. β-adrenergic stimulation was performed with 250 nmol/L isoproterenol, which was given to the medium. The hypoxia tests were performed in an anaerobic atmosphere (85% N₂, 5% O₂, 10% CO₂; 35° C.) for 30 minutes.

Results: In the control group the post systolic (PS) cytosolic calcium values increased from 20 nmol to 40 nmol at the end of diastole (ED). β-Adrenergic stimulation with isoprenalin resulted in a small reduction of PS and a small increase of ED calcium levels as compared to the control group. After hypoxia, the calcium level was significantly higher (PS 80 nmol, ED 360 nmol). Due to stimulation the ED calcium level increased to 450 nmol. Pre-incubation with natural phospholipids had no effect on the level of calcium under normoxic conditions. After hypoxia, ED calcium levels tended to be lower than in the control group (SOPC 300 nmol; PLPC 320 nmol), while there was no relevant difference after additional stimulation with isoproterenol as compared to the control group (SOPC: ED 440 nmol; PLPC: ED 430 nmol). After pre-incubation with the nitrated phospholipids, calcium values were slightly lower under normoxic conditions (SNOPC as well as PNLPC: PS 10 nmol, ED 30 nmol). However, cytosolic calcium levels after hypoxia were significantly lower after pre-incubation with the nitrated phospholipids as compared to the control (SNOPC: PS 40 nmol, ED 80 nmol; PNLPC: PS 30 nmol, ED 70 nmol). In addition, there was only minimal rise of calcium levels after additional stimulation with isoprenalin (SNOPC: ED 90 nmol; PNLPC ED 100 nmol). Incubation with the nitro-carboxylic acid (s)-containing phospholipids according to examples B, C, D, F, N2, N4-N7, N10-N16, O1-O3 and P8-P12 showed no significant differences of these effects as compared to incubation with 1-stearoyl-2-(9-nitrooleoyl)-sn-PC and 1-palmitoyl-2-(9-nitrolinoleoyl)-sn-PC.

Interpretation: Ca²⁺ flux into cells or cell compartments is necessary for a wide variety of physiological processes. In this way, cells respond to external stimuli, in particular to physical alterations, with result in an increased Ca²⁺ influx. This can entail initiation of an apoptosis or necrosis, due to an intracellular Ca²⁺ overload or malfunctions of the intracellular Ca²⁺ compartmentalization. Incubation with nitro-carboxylic acid-containing phospholipids reduces Ca²⁺ accumulation and therefore is useful in clinical situations where the calcium level should be controlled. These effects can, for example, be beneficial to reduce of cell damage caused by reperfusion after a tissue or organ ischemia. Thus, is expected that the thereby extent of an organ infarction is reduced. Furthermore, reduction of reperfusion-induced heart rhythm disturbances, arising in the context of myocardial ischemia, can also be expected. The effects found on calcium homeostasis can also be used to stabilize consequences of exaggerated cell stimulation which can be beneficial, e.g, to prevent mast cell degranulation; thus, there is a potency for a hypo-allergenic effect.

### Example 18

### Studies on the Membrane-Stabilizing Effects of Nitro Carboxylic Acid-Containing Phospholipids in the Cryopreservation of Tissues

Many cells can be frozen under certain conditions and regain their function after rewarming. The ambient medium plays a crucial role in maintaining viability and functionality of cryopreserved cells. For this reason, cryopreservation of a tissue block or whole organs is still problematic. Therefore it should be examined whether pretreatment with nitrated phospholipids leads to a reduction of tissue damage after a cryopreservation procedure.

The femoral artery and great saphenous vein of rabbits (New Zealand White rabbits, 2.0-3.0 kg) were skeletonized, rinsed with PBS and bathed in DMEM. The arterial and venous segments were cut atraumatically to exactly 5 mm long segments. This was followed by a culturing of those segments in Dulbecco/Eagle medium (DMEM) with 1% FCS for two days under standard conditions.

In each series of investigations two segments deriving from the identical vessels were compared, whereby two segments were not frozen which served as controls. The vessel segments were placed in a saline solution with the dissolved natural phospholipids SOPC and PLPC, as well as the analogue phospholipids with nitration of unsaturated fatty acids (1-stearoyl-2-(9-nitrooleoyl)-sn-PC and 1-palmitoyl-2-(9-nitrolinoleoyl)-sn-PC) at a concentration of 200 mmol/l in which they were incubated for one hour. Afterwards, the medium was replaced (DMEM with 2.5% chondroitin sulfate and 10% FCS) fully covering the vessel segments. The samples were rapidly cooled (A 30° C./min) to −70° C. After 12 hours, the samples were rewarmed in a water bath until a tissue temperature of 37° C. was reached, which was achieved within 5 minutes. These samples were cultured for two days. The experiments were repeated with the substances according to examples B, F, N10-N15 and G.

Function of the vascular segments was examined by isometric force development during stimulation with noradrenaline (arteries) and histamine (veins) and the vascular dilatation capacity was investigated by application of acetylcholine. The segments placed within an organ bath were fixed on two oppositely located support brackets which were connected to force transducers, allowing changes in length to also be measured (HSE F30, B40, bridge Coupler, Sachs electronics, Germany). Vessel segments were fully covered with oxygenated Krebs-Henseleit solution at 37° C. Contraction of vein segments was induced by adding histamine (3×10⁻⁵ mol, Sigma, Germany) to the medium. Thereafter the organ bath was flushed twice with final addition of potassium (50 mmol). Then acetylcholine (250 mmol, Sigma, Germany) was added. The hereby achieved maximum increase in diameter within 5 to 10 minutes was registered.

Contracture of the artery segments was induced with norepinephrine (5×10⁻⁵ mol). Vasodilatation was performed analogous to the above described procedure. After completion of the functional studies the vascular segments were fixed, embedded, cut and stained (H & E) and examined by light microscopy with regard to the integrity of the vessel wall layers.

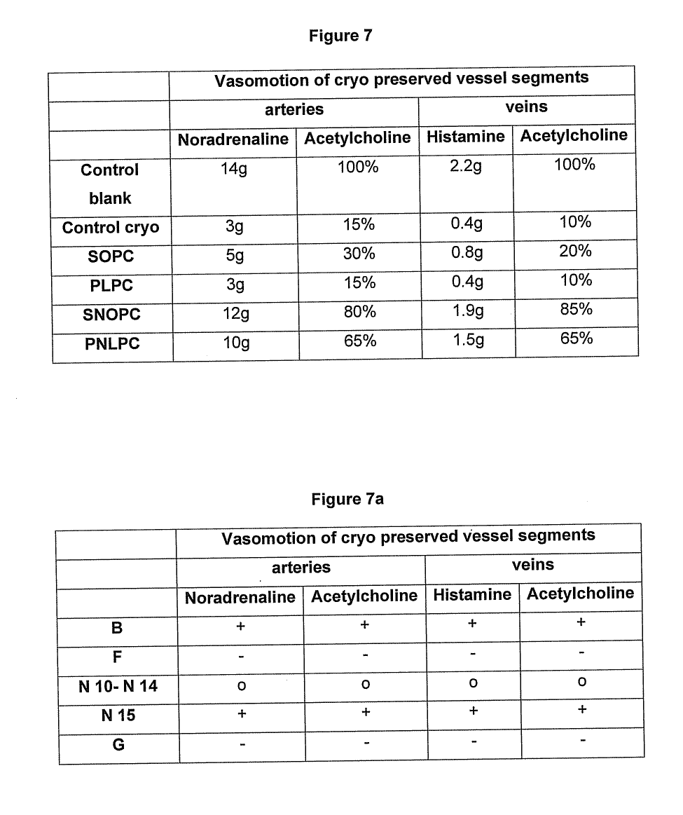

Results (these are Grouped Together as Table in FIGS. 7 and 7a):

Untreated artery and venous segments showed only a minimal response to vasoconstrictive and vasodilating stimulation after cryoconservation. Pretreatment with native phospholipids accomplished only a trend to a better preserved responsiveness of the vascular segments. In contrast, vasomotion of both the venous and the arterial segments was only slightly reduced after pretreatment with the nitro-carboxylic acid (s)-containing phospholipids. A partial detachment of the intima was observed in the untreated thawed arteries by light microscopy. Intimal detachment tended to be lower in vessels pretreated with SOPC. After pretreatment with the nitro-carboxylic acid of containing phospholipids, separation of the intima and the lamina elastica interna could be observed only occasionally, complete delamination was not found.

Interpretation: Cooling of intact cells below the freezing point leads to considerable mechanical stress in the cell membrane, the same applies to the rewarming phase, causing a significant loss of functionality, but also of viability of cells or tissues. Using pretreatment with nitrated phospholipids, a significant reduction of tissue damage was detected after cryopreservation. The responsiveness of treated vessel segments was preserved as comparable to that of unfrozen vessel segments. Thus, pretreatment of tissues with nitro-carboxylic acid (s)-containing phospholipids can be used to preserve structural integrity and functionality of cells and tissues during cryopreservation.

### Example 19

### Investigation on Effects of Phospholipids Containing Nitro-Carboxylic Acid on Membrane Receptors of the TRP Protein Family

Physico-chemical properties of the cell membrane affect the geometry of membrane proteins, as well as the location of their subunits; therefore changes in the membrane fluidity can affect the function of membrane proteins. It should be investigated whether phospholipids containing nitro-carboxylic acid affect the functionally of receptors of the TRP receptor family.

An established in-vitro model of Xenopus oocytes (Dascal N, 1987 The use of Xenopus oocytes. CRC critical reviews in biochemistry 22, 317-387) was used to study the influence of incubation with PL on ion channels regulated by membrane receptors. Defolliculized oocytes were transfected with cRNA (rat) of TRPV1, 2 and 4, as well as of TRPA1, stored in tissue culture plates and incubated in culture medium (ND 96) at 15° C. under constant motion in the incubator for 7 to 10 days.

The oocytes were exposed to the natural phospholipids SOPC and PLPC, as well as the analogous nitrated phospholipids SNOPC (example P14) and PNLPC (example P7) and the phospholipids according to examples B, D, E, F, P8 and S at a concentration of 50 mmol/l, as well as to the natural fatty acids OA and LA, and to the nitrated fatty acids NOA and NLA, each at a concentration of 30 μmol, immediately before the measurements for 10 and 60 minutes duration.

Induced leakage currents through activation of the TRP receptors was determined by means of double electrode voltage-clamp technique (Ahern G P, Brooks I M, Miyares R L &Wang X B, 2005, Extracellular cations sensitize and gate capsaicin receptor TRPV1 modulating pain signalling. J Neurosci 25, 5109-5116). Ooocytes were probed with borosilicate glass capillaries (Clark) and connected to a feedback amplifier (Gene clamp Amplifier, Axon instruments). The membrane potential of oocytes was clamped to −60 or −90 mV. The presence of a directive outward current was compared to non-transfected controls. The investigations were performed in a miniature organ bath in calcium-free buffered (Hepes, pH 7.3) NaCl solution. The TRPV1 receptor excitation was initiated by rapidly changing of the bath solution against a capsaicin solution (10 μmol). Results of non-pretreated oocytes were used for normative comparison of subsequent measurements of incubatesd cells. Investigations with incubated cells were carried out in the same way for oocytes transfected with the TRPV2, and TRPV4 and TRPA1 receptors, wherein the receptor agonists cannabidiol (10 μmol) and 4α-PDD (4α-phorbol 12,13-didecanoate, 50 μmol) and innamaldehyde (50 μmol) were used instead of capsaicin.

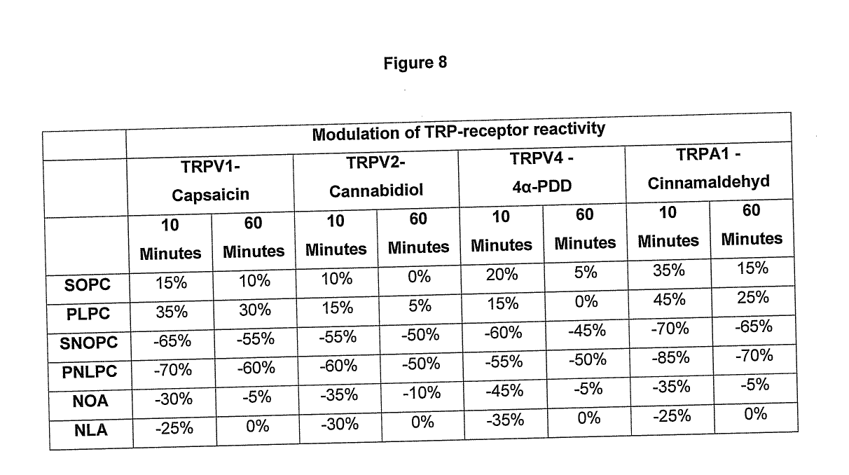

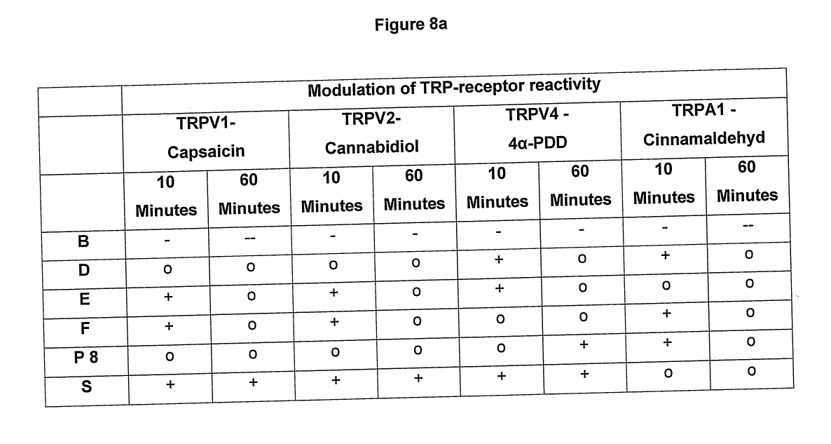

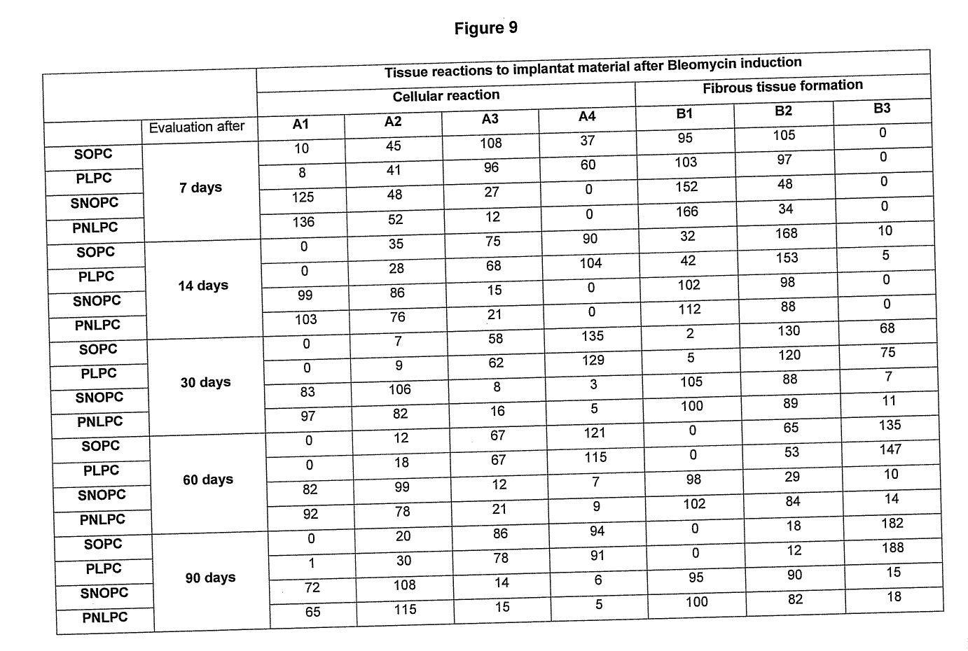

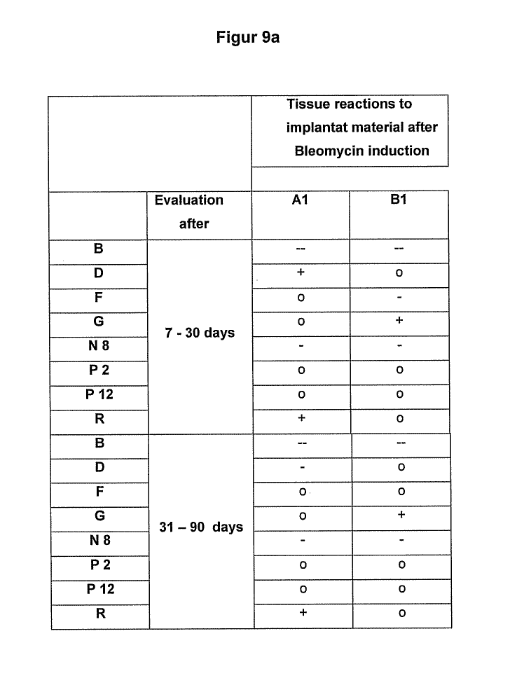

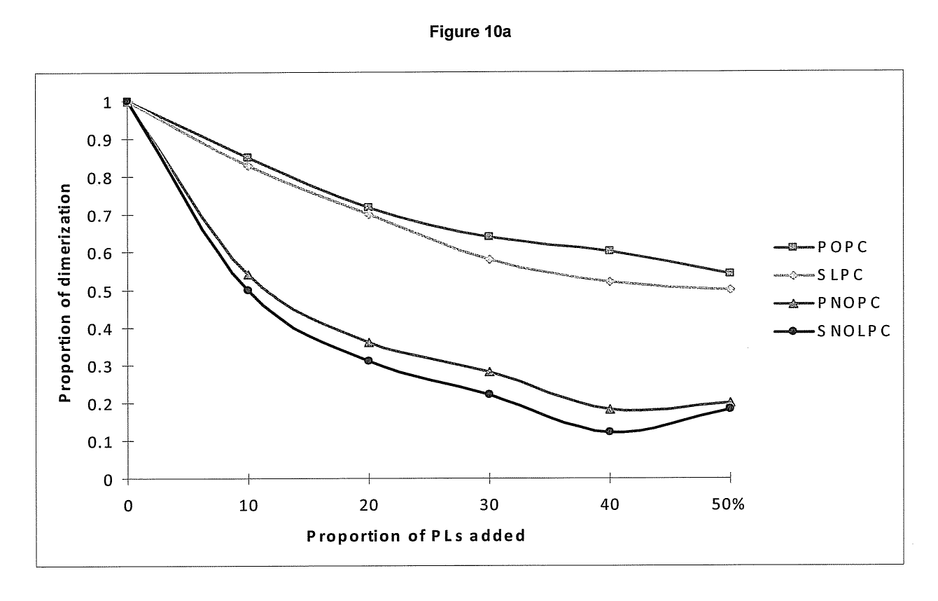

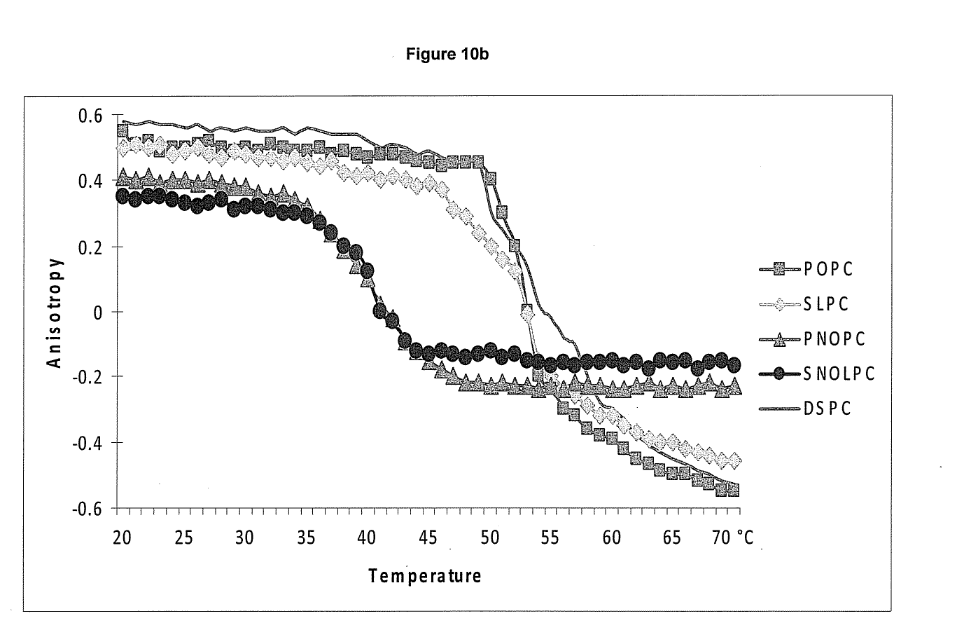

Results (these are Grouped Together as the Table in FIGS. 8 and 8a):

Incubation with natural phospholipids led to an increase of stimulation-induced membrane channel activity. In contrast, nitrated fatty acids as well as the nitro-carboxylic acid-containing phospholipids led to a significant decrease of the inducible membrane channel activity. This effect was significantly more pronounced and longer lasting after incubation with the nitro-carboxylic acid-containing phospholipids than after incubation with the nitro fatty acids.

Pretreatment with the nitro-carboxylic acid (s)-containing phospholipids according to examples B, D, E, F, P7, P8, P14 and S resulted in a significant decrease of inducible membrane channel activity of TRP channels.

### Interpretation:

The investigated membrane protein family senses and transmits different cell stressors (temperature, pH, osmolarity) raising different cell responses. Of paramount importance is the induction of pain by a stimulus via this receptor family, therefore an analgetic effect can be ascribed to an incubation of cells with nitro-carboxylic acid (s)-containing phospholipids.

### Example 20

### Investigations on Effects of Coatings of Soft Tissue of Implant Material with Nitro Carboxylic Acid-Containing Phospholipids and on the In-Vivo Tissue Response

Induction of fibrotic tissue production by connective tissue activation as a consequence of implantation of foreign material was examined in an in-vivo animal model. As implant materials, sterile silicone cushions having a diameter of 1 cm were used. They were prepared using spray coating of two layers with the natural phospholipids SOPC and PLPC, as well as with the nitrated phospholipids SNOPC (example P14) and PNLPC (example P7) and with phospholipids according to examples B, D, F, G, N8, P2, P12, and R or were left untreated.

In male Wistar rats (190-240 g) that were fed and housed under identical conditions for one week, general anaesthesia was performed by intramuscular injection of 0.008 mL/100 g ketamine and 0.004 mL/100 g xylocaln chloride 2%. Then the back of the animals was shaved, sterilized and infiltrated with xylocalne chloride. A paravertebral incision was made on both sides with a length of 1.5 cm followed by preparation of the subcutaneous tissue, so that on both sides approx. 2×2 cm cavities were preformed. Hemostasis was achieved by cauterization, if necessary. Then balls of cotton saturated with Bleomycin (cell-pharm GmbH, Hannover, Germany: 1% in 0.9% NaCl solution) were inserted into the cavities for 3 minutes and then removed. Without rinsing the cavities, the implants coated with a nitrated phospholipid were inserted into the cavity on the left side and the implants coated with natural phospholipid into the cavity on the right side. Thereafter, a layer by layer wound closure was performed and a sterile adhesive bandage was applied.

The animals were kept according to standard conditions for 7, 14, 30, 60 and 90 days. At any study time endpoint, two animals were euthanized. After death the well visible and palpable implants were removed using en-block resection technique. The resected specimens were fixed and embedded in paraffin. Then they were cut in half and the liquid silicone was removed. The specimens were embedded again and cut into thin slices which were stained with H & E and Sirius red, and evaluated by light microscopy. Evaluation was done according to the following criteria:

### A) Cellular Reaction:

A1: none; A2: occasional monocytic cells or lymphocytes; A3: moderate numbers or groups of monocytic cells or lymphocytes; A4: dense infiltration of monocytes, eosinophils, or giant cells.

### B) Fibrous Tissue Formation:

B1: none; B2: collagen-rich layer around the implant; B3: thick (>1 mm) and dense collagen-rich tissue formation around the implant.

In every 10th serial section 10 fields of view uniformly distributed over the circumference of the implant were evaluated and the herein predominant expression was assigned to an event table. In total, 200 fields of view were analyzed per implant

Results (these are Grouped Together as Table in FIGS. 9 and 9a):

After induction of fibrosis with bleomycin, in soft tissue implants coated with natural phospholipids rapid formation of an inflammatory cell infiltrate was observed. This has been accompanied by a significant fibrosis of the tissue. Tissue reactions were significantly less when implants coated with nitrated phospholipids were implanted.

At the same time the degree of fibrous tissue formation was reduced. Coatings with the nitro-carboxylic acid (s)-containing phospholipids according to examples B, D, F, G, N8, P2, P7, P12, P14, and R improved biocompatibility of implants several-fold as compared to natural PLs as assed in-vivo.

Interpretation: Adverse reactions of the body to implants, such as for example hyperproliferation, fibrosis, hence rejection or pathological overgrowth, are some of the greatest problems after insertion of foreign material in the body. This generally also applies to materials that are not brought within a body, but come in close contact with cells and tissues, e.g. in wounds. Thus, phospholipids containing nitro-carboxylic acid coatings showed excellent properties here, which could not be attained by phospholipids which have no nitrated fatty acids.

### Example 21

### Investigation of Cryopreservating Effects of Nitro Carbon Acid-Containing Phospholipids on Viability and Maintenance of In-Vivo Tissue Functions

Cryopreservation is a common technique for the long term preservation of autologous tissues and is especially used for storage of reproductive tissues. For this purpose, tissue textures but also their metabolic functionality and the ability for cell division must be preserved.

Ovar ectomy was performed in female sheep; resected tissues were cut into 60 ovarian tissue strips. The ovarian strips were either cultured directly (n=4) or prepared with a usual solution for vitrification (VS group; Vitrification Kit, Sage, USA) or incubated with nitrated PLs (NPL group). For this purpose, the tissue strips were bathed in an equibrilation solution (ART-8025-A) which consisted of MOPS-buffered amino acids and gentamicin sulfate (10 mg/l), and 7.5% (v/v) of each DMSO and ethylene glycol and 12 mg/ml human serum albumin for 30 minutes. Thereafter the strips of the VS group were placed in the vitrification solution (ART-8025-B), which is a MOPS-buffered solution containing amino acids and gentamicin sulfate, that contain 15% (v/v) each DMSO and ethylenglycol, 12 mg/ml human albumin and 0.6 M sucrose. The strips of the NPL group were placed in a solution containing 25% (v/v) nitrated PL SNOPC or PNLPC in a PBS-buffered 1% DMSO solution containing 5% human albumin for 60 minutes. The treated tissue strips were then immersed into liquid nitrogen and kept here for 14 days. Then removal and warming of the tissue pieces was performed in AIM-V medium (Gibco, Invitrogen) in a sterile culture dish, first up to 5° C. within 20 minutes, then heated further in a water bath up to 37° C., within the following 60 minutes. The culture medium was replaced and specimens cultured for 14 days. After this, the tissue pieces were fixed, embedded in paraffin and cut, followed by histological examination. The quantification of primordial, primary and secondary follicles was performed according to a method described elsewhere (Paynter S J, Cooper A, Fuller B J, Shaw R W, 1999; Cryopreservation of bovine ovarian tissue: structural normality of follicles after thawing and culture in vitro. Cryobiology 38 301-309). The total number of intact follicles in the control group was taken as a reference to the number of follicles in the cryopreserved groups.

The culture medium has been exchanged by every 2nd day; samples for hormone analysis were taken and frozen at that time. Determination of 17 β-estradiol and progesterone were performed with a radio-immuno assay (diagnostic systems laboratories, USA). These investigations were also performed with the phospholipids according to examples N1-N6, N9-N13, O2-O4, P2-P5, P8-P12 and Q.

Results: Compared to the control group, a significantly lower number of intact follicles were found in the VL Group (56%). On the contrary, the rate of intact follicles in the NPL group was comparable to that of the control group (SNPOC: 96%; PNLPC: 98%).

Very similar results were found for phospholipids according to examples N1-N6, N9-N13, O2-O4, P2-P5, P8-P12 and Q, which are summarized in the following table:

| | | | | | | | |
|---|---|---|---|---|---|---|---|
| N1 | 93% | N2 | 94% | N3 | 94% | N4 | 92% |
| N5 | 89% | N6 | 92% | N9 | 90% | N10 | 98% |
| N11 | 95% | N12 | 88% | N13 | 91% | O2 | 87% |
| O3 | 91% | O4 | 93% | P2 | 85% | P3 | 97% |
| P4 | 93% | P5 | 90% | P8 | 96% | P9 | 90% |
| P11 | 97% | P12 | 91% | Q | 96% | | |

Compared to the control group, there was a significantly lower increase in estradiol (8.2 vs. 26.5 ng/ml) and a significant increase of progesterone (6.2 vs. 0.8 ng/ml) in the VL group. No relevant differences in the values of the control group were found in the NPL group (estradiol: SNOPC 8.6 ng/ml, PNLPC 8.3 ng/ml; progesterone:
SNOPC 0.6 ng/ml, PNLPC 0.4 ng/ml). Very similar results were also obtained for phospholipids according to examples N1-N6, N9-N13, O2-O4, P2-P5, P8-P12 and Q, where hormone levels were in the range of 0.2 ng/ml and 0.9 ng/ml (progesterone), and 7.8 ng/ml and 8.9 ng/ml (estradiol), respectively.

Interpretation: Functional integrity of ovarian tissues depends essentially on an adequate production of hormones. It could be demonstrated that not only the production but also the ratio of steroids themselves is not altered when ovarian tissue was incubated with nitrated PL before a cryopreservation procedure.

### Example 22

### Investigation on the Viability of Cryopreserved Nerve Grafts after Pretreatment with Nitro Carboxylic Acid-Containing Phospholipids

Functioning of peripheral nerve grafts depends to a large extent from the vitality of the Schwann cells. Cryopreservation destroys a large part of those epineurium cells. Therefore, it should be investigated whether incubation with PL or nitrated PL is able to improve survival of this cell population after cryopreservation.

The sciatic nerve was prepared and sectured in anesthetized rats. The nerve pieces were freed from connective tissue and divided into 3 parts. Specimens were placed in culture DMEM medium containing low a concentration of glucose (1 g/L) with L-glutamine (PAA, Pasching Austria) for 24 hours. Three nerve segments of different animals were incubated with each natural PL SOPC, POPI and SLPE as well as with the nitrated PL SNOPC, PNOPI or SNLPE as well as with the PLs according to examples B, D, F, Q, R, S O6 P1-P4, N2, N6, N9, N10, N14, N19 and N20, by placing them in a 100 mmolar solution thereof for 1 hour. Nerve specimens from the same animals that received no pretreatment served as controls. After incubation, the specimens were cultured for 24 hours. In a further control group, specimens were cryopreserved without any pretreatment. The specimens were covered with a sterile gauze pad moistened with culture medium. Thereafter the temperature was gradually cooled to −30° C. After 72 hours, the nerve pieces were gradually rewarmed to 37° C. After this, renewed culturing was done for 48 hours. The nerve pieces were dissected enzymatically then (collagenase A and D, Roche, Mannheim, Germany) followed by a mechanical separation through multiple uptake and release in a glass pipette. The isolated cells were washed and marked with a living/death staining (Annexin-V-Fluos staining Kit, Roche Diagnostics, Mannheim, Germany) and quantified using FACS analysis. The number of living cells identified was set in relation to the total cells count. The ratio found in non-frozen cells was used as the reference value and result of the other investigation were set in relation hereto.

Results: Compared to the native control only a few cells of the frozen nerve pieces in the control group (5%) were vital. The proportion of nerve cells that were alive after incubation with natural PL was 20% in POPI, 15% in SLPE and was 26% in SOPC. When nerve specimens were incubated with the nitrated PLs a significantly higher viability was found which accounted for 82% in PNOPI, 80% in SNLPE and 86% on SNOPC. Similar values were also obtained for the other PLs according to examples B, D, F, Q, R, S, O6, P1-P4, N2, N6, N9, N10, N14, N19 and N20, shown in the following table:

| | | | | | | | |
|---|---|---|---|---|---|---|---|
| B | 83% | D | 89% | F | 88% | O6 | 78% |
| N2 | 79% | N6 | 72% | N9 | 79% | N10 | 82% |
| N14 | 81% | N19 | 84% | N20 | 82% | P1 | 90% |
| P2 | 82% | P3 | 90% | P4 | 74% | | |
| Q | 80% | R | 75% | S | 85% | | |

### Example 24

### Preparation of Nitro-Carboxylic Acid (s)-Containing Phospholipids Impregnated Wound Pads

A commercially available Tabotamp® sponge was submerged for 6 minutes in the impregnation solution manufactured according to example 21. After drying the immersion operation was repeated another two times. Alternatively, the impregnation solution can be applied with a syringe. This process can be repeated several times until the desired loading of the sponge is reached.

### Example 25

### Medical Cellulose-Based Materials Coated with Nitro-Carboxylic Acid (s)-Containing Phospholipids

A 3 cm wide and 6 cm-long piece of a wound dressing such as, for example, SeaSorb soft made by the company Coloplast consisting of calciumalginate and sodium carboxymethyl cellulose, was sprayed with approx. 1 ml of impregnating solution as shown in example 21, 5 times and after each spraying operation dried in the air for about 20 minutes. Alternatively, the impregnation solution can be applied with a syringe. This process can be repeated several times until the desired loading of the pulp is reached.

### Example 26

### Preparation of Suture Materials Impregnated with Nitro-Carboxylic Acid (s)-Containing Phospholipids

At room temperature, 300 mg dequalinium chloride (Solmag) and 300 mg of ONOPC were dissolved in 28.800 g methanol (Fluka). A clear solution is obtained. A 50 cm-long piece of a woven polyglycolide thread (USP 2.0) is dipped in this solution. Then the methanol was allowed to evaporate at room temperature. The amount of coating deposited on the thread is measured gravimetrically. The weight of the coating was determined to be 0.5 mg.

### Example 27

### Preparation of a Wound Rinsing Solution with Nitro-Carboxylic Acid (s)-Containing Phospholipids

To a Ringer solution (electrolyte composition sodium chloride: 8.6 g, potassium chloride: 0.3 g and calcium chloride: 0.33 g per 1 L solution; pH 7.0), 0.5 g polyhexanide, 0.3 g PEG, and 0.6 g 1-stearoyl-2-(9-nitrooleoyl)-sn-PC (or in a different approach, 0.7 g 1-palmitoyl-2-(9-nitrolinoleoyl)-sn-PC) were added. The solution was stirred and then sterilized.

1. Use of nitro-carboxylic acid (s)-containing phospholipids of the general structure (I) 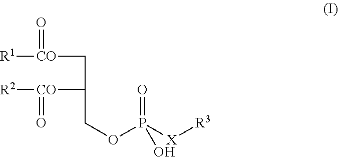
wherein
X is O or S;
R¹ and R² are selected independently of each other from the group comprising: linear nitroalky residues with 5-30 carbon atoms, branched nitroalkyl residues with 5-30 carbon atoms, linear nitroalkenyl residues with 5-30 carbon atoms, branched nitroalkenyl residues with 5-30 carbon atoms, linear nitroalkynyl residues with 5-30 carbon atoms, branched nitroalkynyl residues with 5-30 carbon atoms, nitroalkyl residues with 5-30 carbon atoms, wherein the nitroalkyl residue contains a cycloalkyl residue or a heterocycloalkyl residue or a carbonyl group,
linear alkyl residues with 5-30 carbon atoms, branched alkyl residues with 5-30 carbon atoms, linear alkenyl residues with 5-30 carbon atoms, branched alkenyl residues with 5-30 carbon atoms, linear alkynyl residues with 5-30 carbon atoms, branched alkynyl residues with 5-30 carbon atoms, alkyl residues with 5-30 carbon atoms, wherein the alkyl residue contains a cycloalkyl residue or a heterocycloalkyl residue or a carbonyl group,
wherein the alkyl residue, alkenyl residue and alkynyl residue can be substituted with one, two or three hydroxy groups, thiol groups, halogen residues, carboxylate groups, C₁-C₅-alkoxycarbonyl groups, C₁-C₅-alkylcarbonyloxy groups, C₁-C₅-alkoxy groups, C₁-C₅-alkylamino groups, C₁-C₅-dialkylamino groups and/or amino groups and wherein the nitroalkyl residue, nitroalkenyl residue and nitroalkynyl residue can be substituted with one, two or three hydroxy groups, thiol groups, halogen residues, carboxylate groups, C₁-C₅-alkoxycarbonyl groups, C₁-C₅-alkylcarbonyloxy groups, C₁-C₅-alkoxy groups, C₁-C₅-alkylamino groups, C₁-C₅-dialkylamino groups and/or amino groups,
wherein at least one of the residues R¹ and R² must contain at least one nitro group,
R³ represents one of the following residues: —H, —CH₂—CH(COO⁻)—NH₃⁺, —CH₂—CH₂—NH₃⁺, —CH₂—CH₂—N(CH₃)₃⁺, 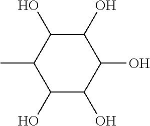
—CR⁴R⁵R⁶, —CR⁴R⁵—CR⁶R⁷R⁸, —CR⁴R⁵—CR⁶R⁷—CR⁸R⁹R¹⁰, —CR⁴R⁵—CR⁶R⁷—CR⁸R⁹—CR¹⁰R¹¹R¹², —CR⁴R⁵—CR⁶R⁷—CR⁸R⁹—CR¹⁰R¹¹—CR¹²R¹³R¹⁴;
R⁴-R¹⁴ represent independently of each other
—H, —OH, —OP(O)(OH)₂, —P(O)(OH)₂, —P(O)(OCH₃)₂, —P(O)(OC₂H₅)₂, —OCH₃, —OC₂H₅, —OC₃H₇, —O-cyclo-C₃H₅, —OCH(CH₃)₂, —OC(CH₃)₃, —OC₄H₉, —OC₄H₉, —OC₅H₁₁, —OCH₂CH(CH₃)₂, —OCH(CH₃)C₂H₅, —OC₆H₁₃, —O-cyclo-C₄H₇, —O-cyclo-C₅H₉, —OPh, —OCH₂-Ph, —OCPh₃, —SH, —SCH₃, —SC₂H₅, —F, —Cl, —Br, —I, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CON(CH₃)₂, —CON(C₂H₅)₂, —CON(C₃H₇)₂, —NH₂, —NO₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —NH-cyclo-C₄H₇, —NH-cyclo-C₅H₁₁, —NH-cyclo-C₆H₁₃, —N(cyclo-C₄H₇)₂, —N(cyclo-C₅H₁₁)₂, —N(cyclo-C₆H₁₃)₂, —NH(Ph), —NPh₂, —SOCH₃, —SOC₂H₅, —SOC₃H₇, —SO₂CH₃, —SO₂C₂H₅, —SO₂C₃H₇, —SO₃H, —SO₃CH₃, —SO₃C₂H₅, —SO₃C₃H₇, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—N(CH₃)₂, —NH—CO—N(C₂H₅)₂, —O—CO—NH₂, —O—CO—NHCH₃, —O—CO—NHC₂H₅, —O—CO—NHC₃H₇, —O—CO—N(CH₃)₂, —O—CO—N(C₂H₅)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₃, —C₂H₅, —C₃H₇, -cyclo-C₃H₅, —CH(CH₃)₂, —C(CH₃)₃, —C₄H₉, -cyclo-C₄H₇, -cyclo-C₅H₉, -cyclo-C₆H₁₁, —CH₂—CH(CH₃)₂, —CH(CH₃)—C₂H₅, —C₅H₁₁, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH; 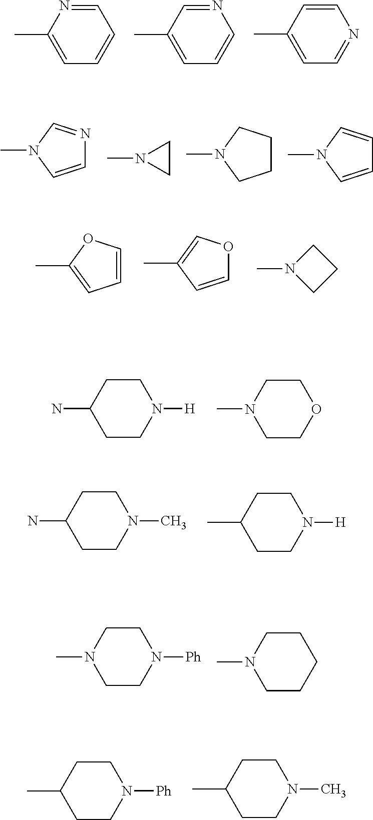
as well as salts, solvates, hydrates, enantiomeres, diastereomeres, racemates, mixtures of enantiomeres, mixtures of diastereomeres ofsthe aforementioned compounds for the manufacture of medical compositions and for the coating of medical devices.

2. Use according to claim 1, wherein the medical compositions are bio-passivating compositions, rinsing solutions for medical apparatuses, rinsing solutions for wounds, impregnation solutions for dressing, wound and suture materials, coating solutions for medical devices, cryoprotection solutions, cryopreservation media, lyoprotection solutions, contrast agent solutions, preservation and perfusion solutions for cells, tissues and organs.

3. Use according to claim 1, wherein at least one of the residues R¹COO— and R²COO—, represented as a free acid group R¹COOH and R²COOH, is a nitrated carboxylic acid selected from the following group: Hexanoic acid, Octanoic acid, decanoic acid, dodecanoic acid, tetradecanoic acid, hexadecanoic acid, heptadecanoic acid, Octadecanoic acid, Eicosanoic acid, docosanoic acid, tetracosanoic acid, cis-9-tetradecenoic acid, cis-9-hexadecenoic acid, cis-6-Octadecenoic acid, cis-9-Octadecenoic acid, cis-11-Octadecenoic acid, cis-9-Eicosenoic acid, cis-11-Eicosenoic acid, cis-13-docosenoic acid, cis-15-tetracosenoic acid, t9-Octadecenoic acid, t11-Octadecenoic acid, t3-hexadecenoic acid, 9,12-Octadecadienoic acid, 6,9,12-Octadecatrienoic acid, 8,11,14-Eicosatrienoic acid, 5,8,11,14-Eicosatetraenoic acid, 7,10,13,16-Docosatetraenoic acid, 4,7,10,13,16-Docosapentaenoic acid, 9,12,15-Octadecatrienoic acid, 6,9,12,15-Octadecatetraenoic acid, 8,11,14,17-Eicosatetraenoic acid, 5,8,11,14,17-Eicosapentaenoic acid, 7,10,13,16,19-Docosapentaenoic acid, 4,7,10,13,16,19-Docosahexaenoic acid, 5,8,11-Eicosatrienoic acid, 9c11t13t-Octadecatrienoic acid, 8t10t12c-Octadecatrienoic acid, 9c11t13c-Catalpinic acid, 4,7,9,11,13,16,19-Docosaheptaenoic acid, Taxoleic acid, Pinolenic acid, Sciadonic acid, 6-Octadecynoic acid, t11-Octadecen-9-ynoic acid, 9-Octadecynoic acid, 6-Octadecen-9-ynoic acid, t10-Heptadecen-8-ynoic acid, 9-Octadecen-12-ynoic acid, t7,t11-Octadecadien-9-ynoic acid, t8,t10-Octadecadien-12-ynoic acid, 5,8,11,14-Eicosatetraynoic acid, Retinoic acid, Isopalmitic acid, Pristanic acid, 3,7,11,15-Tetramethylhexadecanoic acid, 11,12-Methyleneoctadecanoic acid, 9,10-Methylene-hexadecanoic acid, Coronaric acid, (R,S)-Liponic acid, (S)-Liponic acid, (R)-Liponic acid, 6,8-(methylsulfanyl)-octanoic acid, 4,6-Bis(methylsulfanyl)-hexanoic acid, 2,4-bis(methylsulfanyl)-butanoic acid, 1,2-Dithiolan-carboxylic acid, (R,S)-6,8-dithiane octanoic acid, (S)-6,8-dithiane octanoic acid, 6,9-Octadecenynoic acid, t8,t10-Octadecadien-12-ynoic acid, Hydroxytetracosanoic acid, 2-Hydroxy-15-tetracosenoic acid, 12-Hydroxy-9-octadecenoic acid, 14-Hydroxy-11-eicosenoic acid, Pimelic acid, Suberic acid, Azelaic acid, Sebacic acid, Brassylic acid and Thapsic acid.

4. Nitro-carboxylic acids-containing phospholipids of the general structure (I) 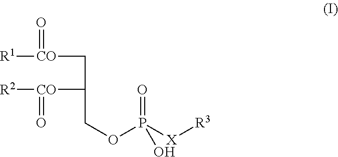
wherein
X is O or S;
R¹ and R² are selected independently of each other from the group, comprising: linear nitroalkyl residues with 5-30 carbon atoms, branched nitroalkyl residues with 5-30 carbon atoms, linear nitroalkenyl residues with 5-30 carbon atoms, branched nitroalkenyl residues with 5-30 carbon atoms, linear nitroalkynyl residues with 5-30 carbon atoms, branched nitroalkynyl residues with 5-30 carbon atoms, nitroalkyl residues with 5-30 carbon atoms, wherein the nitroalkyl residue contains a cycloalkyl residue or a heterocycloalkyl residue or a carbonyl group,
linear alkyl residues with 5-30 carbon atoms, branched alkyl residues with 5-30 carbon atoms, linear alkenyl residues with 5-30 carbon atoms, branched alkenyl residues with 5-30 carbon atoms, linear alkynyl residues with 5-30 carbon atoms, branched alkynyl residues with 5-30 carbon atoms, alkyl residues with 5-30 carbon atoms, wherein the alkyl residue contains a cycloalkyl residue or a heterocycloalkyl residue or a carbonyl group,
wherein the alkyl residue, alkenyl residue and alkynyl residue can be substituted with one, two or three hydroxy groups, thiol groups, halogen residues, carboxylate groups, C₁-C₅-alkoxycarbonyl groups, C₁-C₅-alkylcarbonyloxy groups, C₁-C₅-alkoxy groups, C₁-C₅-alkylamino groups, C₁-C₅-dialkylamino groups and/or amino groups and wherein the nitroalkyl residue, nitroalkenyl residue and nitroalkynyl residue can be substituted with one, two or three hydroxy groups, thiol groups, halogen residues, carboxylate groups, C₁-C₅-alkoxycarbonyl groups, C₁-C₅-alkylcarbonyloxy groups, C₁-C₅-alkoxy groups, C₁-C₅-alkylamino groups, C₁-C₅-dialkylamino groups and/or amino groups,
wherein at least one of the residues R¹ and R² must contain at least one nitro group,
R³ represents one of the following residues: —H, —CH₂—CH(COO⁻)—NH₃⁺, —CH₂—CH₂—NH₃⁺, —CH₂—CH₂—N(CH₃)₃⁺, 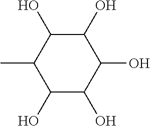
—CR⁴R⁵R⁶, —CR⁴R⁵—CR⁶R⁷R⁸, —CR⁴R⁵—CR⁶R⁷—CR⁸R⁹R¹⁰, —CR⁴R⁵—CR⁶R⁷—CR⁸R⁹—CR¹⁰R¹¹R¹², —CR⁴R⁵—CR⁶R⁷—CR⁸R⁹—CR¹⁰R¹¹—CR¹²R¹³R¹⁴;
R⁴-R¹⁴ represent independently of each other
—OH, —OP(O)(OH)₂, —P(O)(OH)₂, —P(O)(OCH₃)₂, —P(O)(OC₂H₅)₂, —OCH₃, —OC₂H₅, —OC₃H₇, —O-cyclo-C₃H₅, —OCH(CH₃)₂, —OC(CH₃)₃, —OC₄H₉, —OC₄H₉, —OC₅H₁₁, —OCH₂CH(CH₃)₂, —OCH(CH₃)C₂H₅, —OC₆H₁₃, —O-cyclo-C₄H₇, —O-cyclo-C₅H₉, —OPh, —OCH₂-Ph, —OCPh₃, —SH, —SCH₃, —SC₂H₅, —F, —Cl, —Br, —I, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CON(CH₃)₂, —CON(C₂H₅)₂, —CON(C₃H₇)₂, —NH₂, —NO₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(CH₃)₃⁺, —N(C₂H₅)₃⁺, —N(C₃H₇)₃⁺, —N(cyclo-C₃H₅)₃⁺, —N[CH(CH₃)₂]₃⁺, —N(cyclo-C₃H₅)₂, —N[CH(CH₃)₂]₂, —N[C(CH₃)₃]₂, —NH-cyclo-C₄H₇, —NH-cyclo-C₅H₁₁, —NH-cyclo-C₆H₁₃, —N(cyclo-C₄H₇)₂, —N(cyclo-C₅H₁₁)₂, —N(cyclo-C₆H₁₃)₂, —NH(Ph), —NPh₂, —SOCH₃, —SOC₂H₅, —SOC₃H₇, —SO₂CH₃, —SO₂C₂H₅, —SO₂C₃H₇, —SO₃H, —SO₃CH₃, —SO₃C₂H₅, —SO₃C₃H₇, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—N(CH₃)₂, —NH—CO—N(C₂H₅)₂, —O—CO—NH₂, —O—CO—NHCH₃, —O—CO—NHC₂H₅, —O—CO—NHC₃H₇, —O—CO—N(CH₃)₂, —O—CO—N(C₂H₅)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₃, —C₂H₅, —C₃H₇, -cyclo-C₃H₅, —CH(CH₃)₂, —C(CH₃)₃, —C₄H₉, -cyclo-C₄H₇, -cyclo-C₅H₉, -cyclo-C₆H₁₁, —CH₂—CH(CH₃)₂, —CH(CH₃)—C₂H₅, —C₅H₁₁, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH; 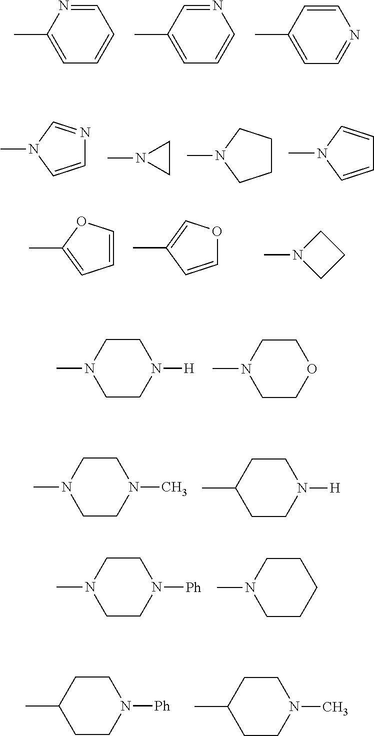
as well as salts, solvates, hydrates, enantiomeres, diastereomeres, racemates, mixtures of enantiomeres, mixtures of diastereomeres of the aforementioned compounds, with the proviso that excluded mixtures of nitrocarboxylic acid containing phospholipids of the general formula (I) with
R¹COO—=9-hydroxy-10-nitrooctadecanoyl, R²COO—=hexadecanoyl und R³=—CH₂CH(COO⁻)—NH₃⁺;
R¹COO—=10-hydroxy-9-nitrooctadecanoyl, R²COO—=hexadecanoyl und R³=—CH₂—CH(COO⁻)—NH₃⁺;
R¹COO—=trans-9-nitro-9-octadecenoyl, R²COO—=hexadecanoyl und R³=—CH₂—CH(COO⁻)—NH₃⁺;
R¹COO—=trans-10-nitro-9-octadecenoyl, R²COO—=hexadecanoyl und R³=—CH₂—CH(COO⁻)—NH₃⁺;
R¹COO—=cis-9-nitro-9-octadecenoyl, R²COO—=hexadecanoyl und R³=—CH₂—CH(COO⁻)—NH₃⁺;
R¹COO—=cis-10-nitro-9-octadecenoyl, R²COO—=hexadecanoyl und R³=—CH₂—CH(COO⁻)—NH₃⁺;
R¹COO—=trans-10-nitro-8-octadecenoyl, R²COO—=hexadecanoyl und R³=—CH₂—CH(COO⁻)—NH₃⁺;
R¹COO—=trans-9-nitro-10-octadecenoyl, R²COO—=hexadecanoyl und R³=—CH₂—CH(COO⁻)—NH₃⁺;
R¹COO—=cis-10-nitro-8-octadecenoyl, R²COO—=hexadecanoyl und R³=—CH₂—CH(COO⁻)—NH₃⁺; and
R¹COO—=cis-9-nitro-10-octadecenoyl, R²COO—=hexadecanoyl und R³=—CH₂—CH(COO⁻)—NH₃^{±}.

5. Nitro-carboxylic acids-containing phospholipids according to claim 4, wherein at least one of the residues R¹COO— and R²COO— represented as a free acid group R¹COOH and R²COOH, is a nitrated carboxylic acid selected from the following group:
Hexanoic acid, Octanoic acid, decanoic acid, dodecanoic acid, tetradecanoic acid, hexadecanoic acid, heptadecanoic acid, Octadecanoic acid, Eicosanoic acid, docosanoic acid, tetracosanoic acid, cis-9-tetradecenoic acid, cis-9-hexadecenoic acid, cis-6-Octadecenoic acid, cis-9-Octadecenoic acid, cis-11-Octadecenoic acid, cis-9-Eicosenoic acid, cis-11-Eicosenoic acid, cis-13-docosenoic acid, cis-15-tetracosenoic acid, t9-Octadecenoic acid, t11-Octadecenoic acid, t3-hexadecenoic acid, 9,12-Octadecadienoic acid, 6,9,12-Octadecatrienoic acid, 8,11,14-Eicosatrienoic acid, 5,8,11,14-Eicosatetraenoic acid, 7,10,13,16-Docosatetraenoic acid, 4,7,10,13,16-Docosapentaenoic acid, 9,12,15-Octadecatrienoic acid, 6,9,12,15-Octadecatetraenoic acid, 8,11,14,17-Eicosatetraenoic acid, 5,8,11,14,17-Eicosapentaenoic acid, 7,10,13,16,19-Docosapentaenoic acid, 4,7,10,13,16,19-Docosahexaenoic acid, 5,8,11-Eicosatrienoic acid, 9c11t13t-Octadecatrienoic acid, 8t10t12c-Octadecatrienoic acid, 9c11t13c-Catalpinic acid, 4,7,9,11,13,16,19-Docosaheptaenoic acid, Taxoleic acid, Pinolenic acid, Sciadonic acid, 6-Octadecynoic acid, t11-Octadecen-9-ynoic acid, 9-Octadecynoic acid, 6-Octadecen-9-ynoic acid, t10-Heptadecen-8-ynoic acid, 9-Octadecen-12-ynoic acid, t7,t11-Octadecadien-9-ynoic acid, t8,t10-Octadecadien-12-ynoic acid, 5,8,11,14-Eicosatetraynoic acid, Retinoic acid, Isopalmitic acid, Pristanic acid, 3,7,11,15-Tetramethylhexadecanoic acid, 11,12-Methyleneoctadecanoic acid, 9,10-Methylene-hexadecanoic acid, Coronaric acid, (R,S)-Liponic acid, (S)-Liponic acid, (R)-Liponic acid, 6,8-(methylsulfanyl)-octanoic acid, 4,6-Bis(methylsulfanyl)-hexanoic acid, 2,4-bis(methylsulfanyl)-butanoic acid, 1,2-Dithiolan-carboxylic acid, (R,S)-6,8-dithiane octanoic acid, (S)-6,8-dithiane octanoic acid, 6,9-Octadecenynoic acid, t8,t10-Octadecadien-12-ynoic acid, Hydroxytetracosanoic acid, 2-Hydroxy-15-tetracosenoic acid, 12-Hydroxy-9-octadecenoic acid, 14-Hydroxy-11-eicosenoic acid, Pimelic acid, Suberic acid, Azelaic acid, Sebacic acid,
Brassylic acid and Thapsic acid.

6. Nitro-carboxylic acids-containing phospholipids according to claim 4 for the use in the treatment of cuts, dissections, resections, wound closures, tissue sutures, cauterization, drainages, graft inserts, implant inserts, osteosyntheses, hyperthermia, irradiation, light/laser radiation, cryo-ablation and tissue welding.

7. Nitro-carboxylic acids-containing phospholipids according to claim 4 for the use for bio-passivation of damaged cells, tissues and organs, wherein the damage originated from a physical, chemical, osmotic, or electrical trauma.

8. Nitro-carboxylic acids-containing phospholipids according to claim 4 for the use for cryopreservation of cells and tissues and for the protection of cells, tissues and organs from damage caused by toxins, pathogenic bio-molecules, allergens, hypoxia, cryo-thermia, hyperthermia, barotrauma, irradiation, light/laser radiation, and reperfusion.

9. Medical device coated with at least one nitro-carboxylic acids-containing phospholipid according to claim 4.

10. Medical device according to claim 9, wherein the coating is a monolayer, bilayer or a multilayer.

11. Medical device according to claim 9, wherein the coating has bio-passivating, bio-compatibility increasing and/or proliferation reducing properties.

12. Medical device according to claim 9, wherein the coating contains additionally excipients and/or pharmacologically active substances.

13. Medical device according to claim 9, wherein the coating has an improved stability against detachment and/or enables an improved adhesion of cells.

14. Medical device according to claim 9, wherein the medical device represents medical apparatuses, implants, medical objects temporally insertable into the body, wound materials and suture materials.

15. Medical device according to claim 14, wherein the medical device represents a biological or artificial transplant or implant, artificial or natural blood vessels, blood conduits, blood pumps, dialysers, dialysing machines, heart valves, soft tissue implants, breast implants, facial implants, stents, catheter ballons or, catheter balloons with a crimped stent.

16. Medical device according to claim 9, wherein the coating of the at least one nitro-carboxylic acids-containing phospholipid additionally contains at least one anti-restenotic active substance.

17. Bio-passivating compositions, rinsing solutions for medical apparatuses, rinsing solutions for wounds, impregnation solutions for dressing, wound and suture materials, coating solutions for medical devices, cryoprotection solutions, cryopreservation media, lyoprotection solutions, contrast agent solutions, preservation and perfusion solutions for cells, tissues and organs containing at least one nitro-carboxylic acid-containing phospholipid as defined in claim 4.
